(19) United States
(12) Patent Application Publication
(10) Pub. No.: US 2025/0388919 A1
(43) Pub. Date: Dec. 25, 2025

(54) METHODS AND COMPOSITIONS FOR MODIFYING PEANUT SEED OIL

(71) Applicant: INGATEYGEN LLC, Durham, NC (US)

(72) Inventors: Hortense W. DODO, Durham, NC (US); John SALMERON, Hillsborough, NC (US); Koffi N. KONAN, Durham, NC (US); Rajesh PERIANAYAGAM, Carmel, IN (US)

(73) Assignee: INGATEYGEN LLC, Durham, NC (US)

(21) Appl. No.: 19/129,285

(22) PCT Filed: Nov. 13, 2023
(86) PCT No.: PCT/US2023/079499

§ 371 Date: May 12, 2025

Related U.S. Application Data
(60) Provisional application No. 63/383,359, filed on Nov. 11, 2022.

(51) Int. Cl. C12N 15/82; C12N 9/10

(52) U.S. Cl. CPC C12N 15/8247; C12N 9/1029

(57) ABSTRACT

This invention is directed to peanut plants or parts thereof comprising at least one mutation in one or more β-ketoa-cyl-ACP synthetase II (KASII) genes encoding a KasII polypeptide and methods and compositions for making and using the same.

### CROSS REFERENCE TO RELATED APPLICATIONS

This application claims priority from U.S. Provisional Application No. 63/383,359 filed on Nov. 11, 2022, hereby incorporated by reference in its entirety.

### SEQUENCE LISTING

The instant application contains a Sequence Listing which has been submitted electronically in XML format and is hereby incorporated by reference in its entirety. Said XML copy, created on Nov. 10, 2023, is named, 96351-397226PCT_SeqListing.xml and is 399 kilo bytes in size.

### FIELD OF THE INVENTION

This invention relates to compositions and methods for altering peanut seed oil through modification of the KASII gene in the peanut plant, optionally wherein the peanut seed oil is modified to increase the levels of palmitic acid. The invention further relates to peanut plants produced using the methods and compositions of the invention.

### BACKGROUND OF THE INVENTION

The peanut plant (Arachis hypogaea), a crop grown worldwide, is an annual plant belonging to the family Leguminosae, originally native to South America. Peanuts are commercially grown in the Southeastern regions of the United States, specifically in Alabama, Florida, Georgia, North Carolina, and Virginia, and in many other countries of the world. In the United States, four types are grown, including Virginia, Spanish, Valencia and Runner varieties. Runner peanuts make up more than 80% of the peanuts grown in the U.S and they are most frequently used for oil production and peanut butter. The kernels of runner peanuts are uniform in size, thus results in even roasting and a consistent taste in every jar. Runner peanuts are grown mostly in Georgia. Virginia peanuts are considered the “gourmet” peanut variety and are used primarily for whole kernel and inshell peanuts and confections. They have large kernels and are grown mostly in Virginia and the Carolinas. Spanish peanuts are typically used in candy, confections, and peanut butter, and most of the organic peanuts produced in the U.S. are of the Spanish variety. A smaller sized kernel, they are known for their red skins and nutty flavor profile. They have a slightly higher oil content, which adds to their flavor when roasted. Spanish peanuts are almost exclusively grown in Texas, Oklahoma and New Mexico. Valencia peanuts are commonly used for all-natural peanut butter and boiled peanuts. They typically have three or more kernels per shell. They have a sweet flavor and are grown mainly in Texas and New Mexico. Valencia peanuts account for less than one percent of U.S. production. The most widely cultivated commercial peanut cultivar in the USA in 2020 is Georgia-06G.

The most common use of peanuts in the United States is as peanut butter with 60% of the U.S. peanut crop, equating to approximately 1,500,000,000 pounds, being processed into a peanut butter product. The peanut butter dollar sales in the U.S. amounted to approximately 2.3 billion U.S. dollars in 2019.

Peanut butter can be labelled “natural” or “regular”. The main difference resides in the type and amount of added ingredients. Natural peanut butter contains roasted peanuts and salt, while regular peanut butter contains additional ingredients, including sugar, corn syrup, stabilizers, and hydrogenated vegetable oil to ensure that its consistency is not affected by time or temperature, but at the same time increasing the calories and sugar and saturated fat content.

Natural peanut butter is favored by the health-conscious consumer because of its many health benefits such as having fewer additives and no added hydrogenated oils. However, it is susceptible to oil separation which leads to lipid peroxidation and the development of off-flavor and rancidity. Oil separation may also affect textural quality of peanut butter in terms of spreadability. It is for these reasons that stabilizers are used in commercial production of peanut butter to improve its texture and mouthfeel and to create a shelf-stable, homogeneous product. Common stabilizers are hydrogenated cottonseed, canola, and palm oils; addition of these stabilizers together with the mixing process adds cost to the manufacture of peanut butter, with an estimate of this cost to be upwards of $80,000,000 annually. Additionally, “natural” peanut butter carries a layer of oil separated at the top which is not appealing to the eyes and must be mixed in with each use. This aspect of natural peanut butter presents a problem to the producers.

The present invention overcomes the shortcomings in the art by providing improved methods and compositions for modifying the seed oil content of peanuts.

### SUMMARY OF THE INVENTION

One aspect of the invention provides a peanut plant or part thereof (e.g., seed) comprising at least one (one or more than one) mutation in one or more β-ketoacyl-ACP synthetase II (KAS II) (also known as 3-oxoacyl-acyl carrier protein synthase II) genes.

Another aspect of the invention provides a nucleic acid encoding a mutated KAS II gene from peanut, optionally wherein the mutation is a base substitution, a base deletion, and/or a base insertion in a peanut KAS II gene that is endogenous to the peanut plant or part thereof.

An additional aspect of the invention provides peanut seed oil produced from the seed of the peanut plant of the invention, the peanut seed oil comprising at least about 13% to about 16.5% total palmitic acid and/or a ratio of palmitic acid to stearic acid of about 4:1 to about 6:1.

A further aspect of the invention provides a guide nucleic acid that binds within a target site in a KAS II gene, the target site comprising a nucleotide sequence having at least 80% sequence identity to any one SEQ ID NOs:5-31, 32-51, 52-69, 70-83 and/or 275-298, and/or SEQ ID NOs:122-148, 149-168, 169-188, 189-208, 209-212, and/or 299-326, or a portion of consecutive nucleotides thereof.

Also provided is an expression cassette comprising (a) a polynucleotide encoding CRISPR-Cas effector protein comprising a cleavage domain and (b) a guide nucleic acid that binds to a target site in an KAS II gene, wherein the guide nucleic acid comprises a spacer sequence that is complementary to and binds to: (i) a portion of consecutive nucleotides from a nucleic acid having at least 80% sequence identity to any one of SEQ ID NOs:1, 2 and/or 116-119; (ii) a portion of consecutive nucleotides from a nucleic acid sequence having at least 80% sequence identity to any one of SEQ ID NOs:5-83 and/or 275-298 or SEQ ID NOs:122-212 and/or 299-326; and/or (iii) a portion of a consecutive nucleotides from a nucleic acid encoding an amino acid sequence having at least 80% sequence identity to any one of SEQ ID NO:4 or SEQ ID NO:121.

The invention further provides a method of producing a peanut plant having seed oil with increased levels of palmitic acid, the method comprising: (a) contacting a peanut plant cell comprising an KAS II gene with a nuclease targeting the KAS II gene, wherein the nuclease is linked to a nucleic acid binding domain (e.g., editing system) that binds to a target site in the KAS II gene, wherein the KAS II gene: (i) comprises a nucleotide sequence having at least 80% sequence identity to any one of SEQ ID NO:1, SEQ ID NO:2, SEQ ID NO:116, SEQ ID NO:117, SEQ ID NO:118, and/or SEQ ID NO:119, (ii) comprises a region of consecutive nucleotides having at least 80% sequence identity to any one of the nucleotide sequences of SEQ ID NOs:5-31, 32-51, 52-69, 70-83 and/or 275-298, and/or SEQ ID NOs:122-148, 149-168, 169-188, 189-208, 209-212, and/or 299-326, and/or (iii) encodes an amino acid sequence having at least 80% sequence identity to SEQ ID NO:4 and/or SEQ ID NO:121; and (b) growing the peanut plant cell into a peanut plant, wherein the peanut plant comprises a mutation in the KAS II gene, thereby producing a peanut plant having seed oil with an increased level of palmitic acid.

Another aspect of the invention provides a method for increasing the level of palmitic acid in the seed oil of a peanut plant, the method comprising: (a) contacting a peanut plant cell comprising an KAS II gene with a nuclease targeting the KAS II gene, wherein the nuclease is linked to a nucleic acid binding domain (e.g., editing system) that binds to a target site in the KAS II gene, wherein the KAS II gene: (i) comprises a nucleotide sequence having at least 80% sequence identity to SEQ ID NO:1, SEQ ID NO:2, SEQ ID NO:116, SEQ ID NO:117, SEQ ID NO:118, and/or SEQ ID NO:119, (ii) comprises a region of consecutive nucleotides having at least 80% sequence identity to any one of the nucleotide sequences of SEQ ID NOs:5-31, 32-51, 52-69, 70-83 and/or 275-298, and/or SEQ ID NOs:122-148, 149-168, 169-188, 189-208, 209-212, and/or 299-326, and/or (iii) encodes an amino acid sequence having at least 80% sequence identity to SEQ ID NO:4 and/or SEQ ID NO:121; and (b) growing the peanut plant cell into a peanut plant comprising the mutation in the KAS II gene, thereby producing a peanut plant have a mutated KAS II gene and comprising seed oil with an increased level of palmitic acid.

An additional aspect of the invention provides a method of reducing oil separation in peanut butter, the method comprising preparing peanut butter from the seed of the plant of the invention.

In a further aspect, a method for reducing oil separation in peanut butter is provided, the method comprising (a) contacting a peanut plant comprising an KAS II gene with a nuclease targeting the KAS II gene, wherein the nuclease is linked to a nucleic acid binding domain (e.g., editing system) that binds to a target site in the KAS II gene, wherein the KAS II gene: (i) comprises a nucleotide sequence having at least 80% sequence identity to SEQ ID NO:1, SEQ ID NO:2, SEQ ID NO:116, SEQ ID NO:117, SEQ ID NO:118, and/or SEQ ID NO:119, (ii) comprises a region of consecutive nucleotides having at least 80% sequence identity to any one of the nucleotide sequences of SEQ ID NOs:5-31, 32-51, 52-69, 70-83 and/or 275-298, and/or SEQ ID NOs:122-148, 149-168, 169-188, 189-208, 209-212, and/or 299-326, and/or (iii) encodes an amino acid sequence having at least 80% sequence identity to SEQ ID NO:4 and/or SEQ ID NO:121; and (b) growing the peanut plant cell into a peanut plant comprising the mutation in the KAS II gene, thereby producing a plant have a mutated KAS II gene and exhibiting an increase level of palmitic acid in the seed oil of the peanut plant; and preparing peanut butter from the seed, wherein the peanut butter has reduced oil or no oil separation.

In another aspect, a method of providing peanut oil with increased levels of palmitic acid is provided, the method comprising preparing peanut oil from the seed of the peanut plant of the invention.

In an additional aspect, a method of providing peanut oil with increased levels of palmitic acid is provided, the method comprising (a) contacting a peanut plant comprising an KAS II gene with a nuclease targeting the KAS II gene, wherein the nuclease is linked to a nucleic acid binding domain (e.g., editing system) that binds to a target site in the KAS II gene, wherein the KAS II gene: (i) comprises a nucleotide sequence having at least 80% sequence identity to SEQ ID NO:1, SEQ ID NO:2, SEQ ID NO:116, SEQ ID NO:117, SEQ ID NO:118, and/or SEQ ID NO:119, (ii) comprises a region of consecutive nucleotides having at least 80% sequence identity to any one of the nucleotide sequences of SEQ ID NOs:5-31, 32-51, 52-69, 70-83 and/or 275-298, and/or SEQ ID NOs:122-148, 149-168, 169-188, 189-208, 209-212, and/or 299-326, and/or (iii) encodes an amino acid sequence having at least 80% sequence identity to SEQ ID NO:4 and/or SEQ ID NO:121, and (b) growing the peanut plant cell into a peanut plant comprising the mutation in the KAS II gene, thereby producing a plant have a mutated KAS II gene and exhibiting an increase level of palmitic acid in the seed oil of the peanut plant.

Further provided are peanut plants comprising in their genome one or more β-ketoacyl-ACP synthetase II (KAS II) genes having a mutation produced by the methods of the invention as well as polypeptides, polynucleotides, nucleic acid constructs, expression cassettes and vectors for making a plant of this invention.

These and other aspects of the invention are set forth in more detail in the description of the invention below.

### BRIEF DESCRIPTION OF THE SEQUENCES

SEQ ID NO:1 is the KASII genomic sequence (A genome) of peanut varieties TifRunner (NCBI Gene ID No. LOC112696350), GA06G, GA09B, 10X29-6-1-3-1-RunnerNormal, and AC13321-RunnerHO.

SEQ ID NO:2 is a KASII genomic sequence (A genome) of the peanut variety Walton-CA-HO.

SEQ ID NO:3 is the coding sequence for SEQ ID NO:1 and SEQ ID NO:2.

SEQ ID NO:4 is the KasII polypeptide sequence encoded by SEQ ID NOs:1-3.

SEQ ID NOs:5-83 and 275-298 are example target regions in the KASII genomic sequences of SEQ ID NOs:1-2 (SEQ ID NOs:5-31 in the upstream region (regulator region, 5′ of the start codon ATG)) of the KASII gene; SEQ ID NOs:32-51 in Exon 1 of the KASII gene; SEQ ID NOs:52-69 in Exon 2 of the KASII gene; SEQ ID NOs:70-83 in Exon 7 of the KASII gene; and SEQ ID NOs:275-298 in the 3′ end of the KASII gene).

SEQ ID NOs:84-95 and 257-262 are example spacers useful with guide nucleic acids for targeting KASII genomic sequences (SEQ ID NOs:84-86 (Exon 1); SEQ ID NOs:87-89 (Exon 2); SEQ ID NOs:90-91 (Exon 7), SEQ ID NOs:92-95 (upstream region; region 5′ of the start codon, ATG) and SEQ ID NOs:257-262 (3′ region)).

SEQ ID NOs:96-115 are example primer sequences useful with this invention (SEQ ID NOs:96-100 (Exon 1); SEQ ID NOs:101-104 (Exon 2); SEQ ID NOs:105-107 (Exon 7); and SEQ ID NOs:108-115 (upstream (promoter/regulator) region, region that is 5′ of the start codon)).

SEQ ID NO:116 is the KASII genomic sequence (B genome) of peanut varieties TifRunner (NCBI Gene ID No. LOC112740759) and Walton.

SEQ ID NO:117 is the KASII genomic sequence (B genome) of peanut varieties GA06G and GA09.

SEQ ID NO:118 is the KASII genomic sequence (B genome) of peanut variety 10X29-6-1-3-1-RunnerNormal.

SEQ ID NO:119 is the KASII genomic sequence (B genome) of peanut variety

AC13321-RunnerHO.

SEQ ID NO:120 is the coding sequence for SEQ ID NOs:116-119.

SEQ ID NO:121 is the KasII polypeptide sequence encoded by SEQ ID NOs:116-120.

SEQ ID NOs:122-212, 292-294 and 299-326 are example target regions in the KASII genomic sequences of SEQ ID NOs:116-119 (SEQ ID NOs:122-148 in the upstream region (regulator region, region 5′ of the start codon, ATG) of the KASII gene; SEQ ID NOs:149-168 in Exon 1 of the KASII gene; SEQ ID NOs:169-188 in Exon 2 of the KASII gene; SEQ ID NOs:189-208 in Exon 3 of the KASII gene; SEQ ID NOs:209-212 in Exon 7 of the KASII gene; SEQ ID NOs:292-294 and 299-326 in the 3′ end of the KASII gene).

SEQ ID NOs:85, 88, 91, 213-224 and 257-262 are example spacers useful with guide nucleic acids for targeting KASII genomic sequences (SEQ ID NOs:213, 85, 214 (Exon 1); SEQ ID NOs:215, 216, 88 (Exon 2); SEQ ID NOs:217-219 (Exon 3); SEQ ID NOs:91, 224 (Exon 7) and SEQ ID NOs:220-223 (upstream region, region 5′ of the start codon, ATG)).

SEQ ID NOs:96, 97, 99, 107, 222-241 and 257-246 are example primer sequences useful with this invention (SEQ ID NOs:96, 97, 222, 99, 223, 99 (Exon 1); SEQ ID NOs:224, 96, 225, 226, 227, 96 (Exon 2); SEQ ID NOs:228-231 (Exon 3); SEQ ID NOs:232-238 (Exon 7); SEQ ID NOs:239, 240, 107, 241 (upstream (promoter/regulator) region)); and SEQ ID NOs:257-262 (3′ region)).

### BRIEF DESCRIPTION OF THE DRAWINGS

FIG. 1 provides an example vector for editing in peanut. LB: T-DNA left border; Pnos-hpt-tnos: Positive selection cassette for the selection of transformed plants using the antibiotic hygromycin; FCY-UPP: a negative selection marker fluorocytosine deaminase (FCY) linked to the bacterial uracyl phosphoribosyl transferase (converts 5-flurocytosine (5-FC) into 5-fluororouracyl, a toxic compound); P2x35S-zCas9i: Expression cassette for the intronized zCas9 nuclease; ccdB cassette: replaced by the sgRNA TUs in cloning reactions; RB: T-DNA right border.

FIG. 2 provides deep sequencing data showing Cas9 mediated edits in KASII gene (Genome A) of peanut cultivar Walton. WT: reference sequence. Numbers 159, 284 etc.: represent PCR amplicons. Bold: spacer sequence of guide RNA (gRNA). Bold underline: PAM sequence.

FIG. 3 provides deep sequencing data showing Cas9 mediated edits in KASII gene (Genome B) of peanut cultivar Walton. WT: reference sequence. Numbers 824, 966 etc.: represent PCR amplicons. Bold: spacer sequence of guide RNA (gRNA). Bold underline: PAM sequence.

FIG. 4 provides PCR amplicons of the KAS II gene prepared for Next Generation Sequencing (NGS) after bombardment of peanut callus with a vector construct containing guide RNAs 1 and 5.

FIG. 5 provides deletions obtained in the peanut KAS II gene generated by Cas9 nuclease+KAS II guide RNA (guides 1, 5). The spacer sequence is in bold and the PAM underlined.

FIG. 6 provides Leg 14 RNP-mediated deletions in KASII gene.

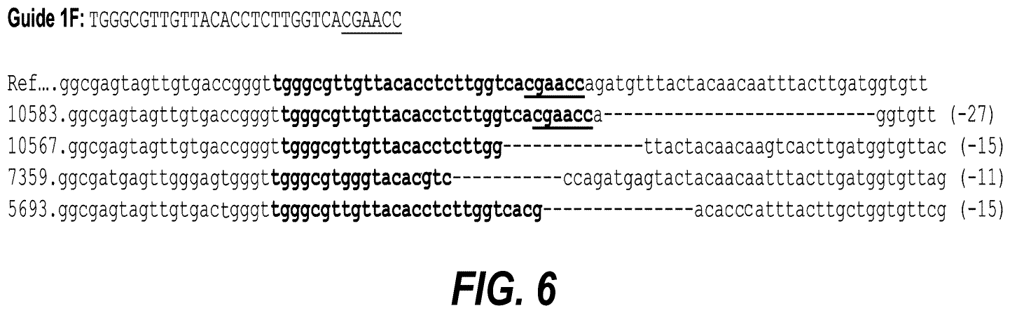

### DETAILED DESCRIPTION

The present invention now will be described hereinafter with reference to the accompanying drawings and examples, in which embodiments of the invention are shown.

This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measurable value may include any other range and/or individual value therein.

Unless otherwise defined, the term “at least one” has the same meaning as “one or more” (e.g., 1, 2, 3, 4, 5 and the like).

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

Recitation of ranges of values herein are merely intended to serve as a shorthand method of referring individually to each separate value falling within the range, unless otherwise indicated herein, and each separate value is incorporated into the specification as if it were individually recited herein. For example, if the range 10 to 15 is disclosed, then 11, 12, 13, and 14 are also disclosed.

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

As used herein, the terms “increase,” “increasing,” “increased,” “enhance,” “enhanced,” “enhancing,” and “enhancement” (and grammatical variations thereof) describe an elevation of at least about 15%, 20%, 25%, 50%, 75%, 100%, 150%, 200%, 300%, 400%, 500% or more as compared to a control. As an example, the seed oil of a peanut plant of this invention may have an increased level or amount of palmitic acid, optionally an increased amount of palmitic acid from about 30% to about 65% (e.g., about 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, or 65%; e.g., from a total amount of palmitic acid of about 10% to an amount of about 13% to 16.5%, e.g., about 13, 13.1, 13.2, 13.3, 13.4, 13.5, 13.6, 13.7, 13.8, 13.9, 14, 14.1, 14.2, 14.3, 14.4, 14.5, 14.6, 14.7, 14.8, 14.9, 15, 15.1, 15.2, 15.3, 15.4, 15.5, 15.6, 15.7, 15.8, 15.9, 16, 16.1, 16.2, 16.3, 16.4, or 16.5% as compared to a peanut plant or part thereof devoid of the at least one mutation.

As used herein, the terms “reduce,” “reduced,” “reducing,” “reduction,” “diminish,” and “decrease” (and grammatical variations thereof), describe, for example, a decrease of at least about 5%, 10%, 15%, 20%, 25%, 35%, 50%, 75%, 80%, 85%, 90%, 95%, 96%, 97%, 98%, 99%, 99.5%, 99.6%, 99.7%, 99.8%, 99.9%, or 100% as compared to a control. In some embodiments, the reduction can result in no or essentially no (i.e., an insignificant amount, e.g., less than about 10% or even 5%) detectable activity or amount. As an example, peanut plant or part thereof the invention comprising at least one mutation in a KASII gene as described herein, may have a modified KAS II polypeptide that exhibits reduced activity or is devoid of activity, optionally wherein a reduction in activity may be about 5% to about 95% (e.g., a reduction in activity of about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95%, optionally a reduction of about 96, 97, 98, 99%, optionally no activity, e.g., a 100% reduction in activity.

As used herein, the terms “express,” “expresses,” “expressed” or “expression,” and the like, with respect to a nucleic acid molecule and/or a nucleotide sequence (e.g., RNA or DNA) indicates that the nucleic acid molecule and/or a nucleotide sequence is transcribed and, optionally, translated. Thus, a nucleic acid molecule and/or a nucleotide sequence may express a polypeptide of interest or, for example, a functional untranslated RNA.

A “heterologous” or a “recombinant” nucleotide sequence is a nucleotide sequence not naturally associated with a host cell into which it is introduced, including non-naturally occurring multiple copies of a naturally occurring nucleotide sequence.

A “native” or “wild type” nucleic acid, nucleotide sequence, polypeptide or amino acid sequence refers to a naturally occurring or endogenous nucleic acid, nucleotide sequence, polypeptide or amino acid sequence. Thus, for example, a “wild type mRNA” is an mRNA that is naturally occurring in or endogenous to the reference organism.

As used herein, the term “heterozygous” refers to a genetic status wherein different alleles reside at corresponding loci on homologous chromosomes.

As used herein, the term “homozygous” refers to a genetic status wherein identical alleles reside at corresponding loci on homologous chromosomes.

As used herein, the term “allele” refers to one of two or more different nucleotides or nucleotide sequences that occur at a specific locus.

A “null allele” is a nonfunctional allele caused by a genetic mutation that results in a complete lack of production of the corresponding protein or produces a protein that is non-functional.

A “locus” is a position on a chromosome where a gene or marker or allele is located. In some embodiments, a locus may encompass one or more nucleotides.

As used herein, the terms “target allele” and/or “allele of interest” are used interchangeably to refer to an allele associated with a desired trait. In some embodiments, a target allele may be associated with either an increase or a decrease (relative to a control) of or in a given trait, depending on the nature of the desired phenotype.

A marker is “associated with” a trait when said trait is linked to it and when the presence of the marker is an indicator of whether and/or to what extent the desired trait or trait form will occur in a plant/germplasm comprising the marker. Similarly, a marker is “associated with” an allele or chromosome interval when it is linked to it and when the presence of the marker is an indicator of whether the allele or chromosome interval is present in a plant/germplasm comprising the marker.

As used herein, the terms “backcross” and “backcrossing” refer to the process whereby a progeny plant is crossed back to one of its parents one or more times (e.g., 1, 2, 3, 4, 5, 6, 7, 8, etc.). In a backcrossing scheme, the “donor” parent refers to the parental plant with the desired gene or locus to be introgressed. The “recipient” parent (used one or more times) or “recurrent” parent (used two or more times) refers to the parental plant into which the gene or locus is being introgressed. For example, see Ragot, M. et al. Marker-assisted Backcrossing: A Practical Example, in TECHNIQUES ET UTILISATIONS DES MARQUEURS MOLECULAIRES LES COLLOQUES, Vol. 72, pp. 45-56 (1995); and Openshaw et al., Marker-assisted Selection in Backcross Breeding, in PROCEEDINGS OF THE SYMPOSIUM “ANALYSIS OF MOLECULAR MARKER DATA,” pp. 41-43 (1994). The initial cross gives rise to the F1 generation. The term “BC1” refers to the second use of the recurrent parent, “BC2” refers to the third use of the recurrent parent, and so on.

As used herein, the terms “cross” or “crossed” refer to the fusion of gametes via pollination to produce progeny (e.g., cells, seeds or plants). The term encompasses both sexual crosses (the pollination of one plant by another) and selfing (self-pollination, e.g., when the pollen and ovule are from the same plant). The term “crossing” refers to the act of fusing gametes via pollination to produce progeny.

As used herein, the terms “introgression,” “introgressing” and “introgressed” refer to both the natural and artificial transmission of a desired allele or combination of desired alleles of a genetic locus or genetic loci from one genetic background to another. For example, a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele may be a selected allele of a marker, a QTL, a transgene, or the like. Offspring comprising the desired allele can be backcrossed one or more times (e.g., 1, 2, 3, 4, or more times) to a line having a desired genetic background, selecting for the desired allele, with the result being that the desired allele becomes fixed in the desired genetic background. For example, a marker associated with increased yield under non-water stress conditions may be introgressed from a donor into a recurrent parent that does not comprise the marker and does not exhibit increased yield under non-water stress conditions. The resulting offspring could then be backcrossed one or more times and selected until the progeny possess the genetic marker(s) associated with increased yield under non-water stress conditions in the recurrent parent background.

A “genetic map” is a description of genetic linkage relationships among loci on one or more chromosomes within a given species, generally depicted in a diagrammatic or tabular form. For each genetic map, distances between loci are measured by the recombination frequencies between them. Recombination between loci can be detected using a variety of markers. A genetic map is a product of the mapping population, types of markers used, and the polymorphic potential of each marker between different populations. The order and genetic distances between loci can differ from one genetic map to another.

As used herein, the term “genotype” refers to the genetic constitution of an individual (or group of individuals) at one or more genetic loci, as contrasted with the observable and/or detectable and/or manifested trait (the phenotype). Genotype is defined by the allele(s) of one or more known loci that the individual has inherited from its parents. The term genotype can be used to refer to an individual's genetic constitution at a single locus, at multiple loci, or more generally, the term genotype can be used to refer to an individual's genetic make-up for all the genes in its genome. Genotypes can be indirectly characterized, e.g., using markers and/or directly characterized by nucleic acid sequencing.

As used herein, the term “germplasm” refers to genetic material of or from an individual (e.g., a plant), a group of individuals (e.g., a plant line, variety, or family), or a clone derived from a line, variety, species, or culture. The germplasm can be part of an organism or cell or can be separate from the organism or cell. In general, germplasm provides genetic material with a specific genetic makeup that provides a foundation for some or all of the hereditary qualities of an organism or cell culture. As used herein, germplasm includes cells, seed or tissues from which new plants may be grown, as well as plant parts that can be cultured into a whole plant (e.g., leaves, stems, buds, roots, pollen, cells, etc.). As used herein, the terms “cultivar” and “variety” refer to a group of similar plants that by structural or genetic features and/or performance can be distinguished from other varieties within the same species.

As used herein, the term “hybrid” in the context of plant breeding refers to a plant that is the offspring of genetically dissimilar parents produced by crossing plants of different lines or breeds or species, including but not limited to the cross between two inbred lines.

As used herein, the term “inbred” refers to a substantially homozygous plant or variety. The term may refer to a plant or plant variety that is substantially homozygous throughout the entire genome or that is substantially homozygous with respect to a portion of the genome that is of particular interest.

A “haplotype” is the genotype of an individual at a plurality of genetic loci, i.e., a combination of alleles. Typically, the genetic loci that define a haplotype are physically and genetically linked, i.e., on the same chromosome segment. The term “haplotype” can refer to polymorphisms at a particular locus, such as a single marker locus, or polymorphisms at multiple loci along a chromosomal segment.

As used herein, the term “heterologous” refers to a nucleotide/polypeptide that originates from a foreign species, or, if from the same species, is substantially modified from its native form in composition and/or genomic locus by deliberate human intervention.

As used herein, the terms “nucleic acid,” “nucleic acid molecule,” “nucleotide sequence” and “polynucleotide” refer to RNA or DNA that is linear or branched, single or double stranded, or a hybrid thereof. The term also encompasses RNA/DNA hybrids. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made.

As used herein, the term “nucleotide sequence” refers to a heteropolymer of nucleotides or the sequence of these nucleotides from the 5′ to 3′ end of a nucleic acid molecule and includes DNA or RNA molecules, including cDNA, a DNA fragment or portion, genomic DNA, synthetic (e.g., chemically synthesized) DNA, plasmid DNA, mRNA, and anti-sense RNA, any of which can be single stranded or double stranded. The terms “nucleotide sequence” “nucleic acid,” “nucleic acid molecule,” “nucleic acid construct,” “oligonucleotide” and “polynucleotide” are also used interchangeably herein to refer to a heteropolymer of nucleotides. Nucleic acid molecules and/or nucleotide sequences provided herein are presented herein in the 5′ to 3′ direction, from left to right and are represented using the standard code for representing the nucleotide characters as set forth in the U.S. sequence rules, 37 CFR §§ 1.821-1.825 and the World Intellectual Property Organization (WIPO) Standard ST.25. A “5′ region” as used herein can mean the region of a polynucleotide that is nearest the 5′ end of the polynucleotide. Thus, for example, an element in the 5′ region of a polynucleotide can be located anywhere from the first nucleotide located at the 5′ end of the polynucleotide to the nucleotide located halfway through the polynucleotide. A “3′ region” as used herein can mean the region of a polynucleotide that is nearest the 3′ end of the polynucleotide. Thus, for example, an element in the 3′ region of a polynucleotide can be located anywhere from the first nucleotide located at the 3′ end of the polynucleotide to the nucleotide located halfway through the polynucleotide.

A peanut plant in which at least one (e.g., one or more) KAS11 gene is modified as described herein (e.g., comprises a modification as described herein) may have seed oil with increased palmitic acid as compared to a peanut plant that does not comprise the modification in the at least one KASII gene.

As used herein a “control plant” means a peanut plant that does not contain an edited KASII gene or genes (edited as described herein) that imparts an altered phenotype such as increased levels of palmitic acid in the seed of the peanut plant comprising the edited KASII gene. A control peanut plant is used to identify and select a plant edited as described herein and that has an altered phenotype. A suitable control plant can be a peanut plant of the parental line used to generate a peanut plant comprising a mutated KASII gene(s), for example, a wild type peanut plant devoid of an edit in a KASII gene as described herein. A suitable control plant can in some cases be a progeny of a heterozygous or hemizygous transgenic plant line that is devoid of the mutated KASII gene.

As used herein with respect to nucleic acids, the term “fragment” or “portion” refers to a nucleic acid that is reduced in length relative (e.g., reduced by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 400, 450, 500, 550, 600, 650, 700, 750, 800, 850, 900, 950 or 1000 or more nucleotides or any range or value therein) to a reference nucleic acid and that comprises, consists essentially of and/or consists of a nucleotide sequence of contiguous nucleotides identical or almost identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical) to a corresponding portion of the reference nucleic acid. As an example, a repeat sequence of guide nucleic acid of this invention may comprise a portion of a wild type CRISPR-Cas repeat sequence (e.g., a wild Type CRISR-Cas repeat; e.g., a repeat from the CRISPR Cas system of, for example, a Cas9, Cas12a (Cpf1), Cas12b, Cas12c (C2c3), Cas12d (CasY), Cas12e (CasX), Cas12g, Cas12h, Cas12i, C2c4, C2c5, C2c8, C2c9, C2c10, Cas14a, Cas14b, and/or a Cas14c, and the like). As a further example, a “fragment” or “portion” (or region) of a nucleic acid encoding a KasII polypeptide may be about 10, 15, 20, 25 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 310, 320, 330, 340, 350, 360, 370, 380, 390, 400, 420, 440, 460, 480, 500, 520, 540, 560, 580, 600, 650, 700, 750, 800, 850, 900, 950, 1000, 1100, 1200, 1300, 1400, 1500, 1600, 1700, 1800, 1900, 2000, 2100, 2200, 2300, 2400, 2500, 3000, 3500, 4000, 4500, 5000, 5500, 6000, 6500, 7000 or more consecutive nucleotides of a KASII nucleic acid, or any range or value therein, optionally wherein the fragment, portion or region may be targeted for editing to provide a peanut plant having increased palmitic acid in the seed oil of the peanut plant. Such a nucleic acid fragment may be, where appropriate, included in a larger polynucleotide of which it is a constituent.

A “region” of a polynucleotide or a polypeptide refers to a portion of consecutive nucleotides or consecutive amino acid residues of that polynucleotide or a polypeptide, respectively. In some embodiments, a nucleic acid fragment or portion (or region) may comprise, consist essentially of or consist of about 10, 15, 20, 25, 30, 35, 40, 45, 50, 60, 70, 80, 90, 100, 150, 200, 250, 300, 350, 400, 450, 500, 550, 600, 660, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2300, 2400, 2500, 3000, 3500, 4000, 4500, 5000, 5500, 6000, 6500, 7000 or more consecutive nucleotides of a KASII nucleic acid, which fragment or portion may comprise a target for editing of the KASII gene as described herein in order to provide a peanut plant having increased levels of palmitic acid in its seed oil. In some embodiments, a portion or region of a KASII gene that may be targeted for editing may be from about nucleotide 1, 10, 20, 30, 40, 50, 100, 150, 200, or 250 to about nucleotide 300, 350, 400, 450, 500, 1000, 1500, 2000, 2500, 3000, 3500, 4000, 4500, 5000, 5100, or 5150 with reference to the nucleotide numbering of SEQ ID NO:1, SEQ ID NO:2, and/or SEQ ID NOs:116-119, from about nucleotide 250 to about nucleotide 950, from about nucleotide 1720 to about nucleotide 2190, from about nucleotide 3565 to about nucleotide 3925, or from about nucleotide 4800 to about nucleotide 5150 with reference to the nucleotide numbering of SEQ ID NO:1 or SEQ ID NO:2, or from about nucleotide 330, 340, 350, 360, 370, or 380 to about nucleotide 650, 660, 670, 680, 690, 700, 710 or 715, from about nucleotide 1060, 1080, 1100 or 1110 to about nucleotide 1425, 1450, or 1475, from about nucleotide 1760, 1780, 1800, or 1810 to about nucleotide 2165, 2180, 2200, or 2215, from about nucleotide 3580, 3600 or 3630 to about nucleotide 3870, 3880, 3900 or 3910, from about nucleotide 3905, 3920, 3940, or 3960 to about nucleotide 4170, 4190, 4200, or 4220, from about nucleotide 4860 to about nucleotide 5110 with reference to the nucleotide numbering of SEQ ID NOs:116-119. In some embodiments, a portion or region of a KASII gene that may be targeted for editing may be the upstream or regulatory region (region 5′ of the start codon, ATG), the first exon, the second exon, the third exon or the seventh exon, optionally wherein the upstream or regulatory region is located from about nucleotide 1 to about 1500, the first exon is located from about nucleotide 1790 to 2130, the second exon is located from about nucleotide 3646 to 3851, and the seventh exon is located from about nucleotide 4930 to 5010 with reference to the nucleotide numbering of SEQ ID NO:1 or SEQ ID NO:2 and/or the upstream or regulatory region is located from about nucleotide 1 to about 1500, the first exon is located from about nucleotide 1815 to 2151, the second exon is located from about nucleotide 3657 to 3862, the third exon is located from about nucleotide 3938 to 4141 and the seventh exon is located from about nucleotide 4939 to 5018 with reference to the nucleotide numbering of SEQ ID NOs:116-119.

In some embodiments, a nucleic acid fragment or portion (or region) may be edited as described herein, wherein the edit results in a deletion. In some embodiments, the edit may be in a KASII nucleic acid in which 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 36, 37, 38, 39, 40 or 45 to about 50, 55, 60, 65, 70, 75, 80, 85, 90, 95 or 100 or more consecutive nucleotides may be deleted from the KASII nucleic acid, optionally about 500 to 6000 consecutive nucleotides from the 3′ end of the gene. In some embodiments, a deletion of nucleotides from a KASII gene as described herein may result in truncated KasII polypeptide or no KasII polypeptide, which when comprised in a peanut plant can result in increased palmitic acid in the seed oil of the peanut plant as compared to a peanut plant not comprising the deletion.

As used herein with respect to polypeptides, the term “fragment” or “portion” may refer to a polypeptide that is reduced in length relative to a reference polypeptide and that comprises, consists essentially of and/or consists of an amino acid sequence of contiguous amino acids identical or almost identical (e.g., 90%, 91%, 92%, 93%, 94%, 95%, 96%, 97%, 98%, 99% identical) to a corresponding portion of the reference polypeptide. Such a polypeptide fragment may be, where appropriate, included in a larger polypeptide of which it is a constituent. In some embodiments, the polypeptide fragment comprises, consists essentially of or consists of at least about 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 260, 270, 280, 290, 300, 350, 400 or more consecutive amino acids of a reference polypeptide.

In some embodiments, a “sequence-specific nucleic acid binding domain” (e.g., a sequence-specific DNA binding domain; e.g., a sequence-specific DNA binding polypeptide/protein) may bind to a KASII gene (e.g., SEQ ID NOs:1, 2, 116, 117, 118, or 119) and/or to one or more fragments, portions, or regions of a KASII nucleic acid (e.g., portions or regions of the regulatory (upstream region), the first, second, third or seventh exons and/or in the 3′ region of the KASII gene as described herein)).

As used herein with respect to nucleic acids, the term “functional fragment” refers to nucleic acid that encodes a functional fragment of a polypeptide. A “functional fragment” with respect to a polypeptide is a fragment of a polypeptide that retains one or more of the activities of the native reference polypeptide.

The term “gene,” as used herein, refers to a nucleic acid molecule capable of being used to produce mRNA, antisense RNA, miRNA, anti-microRNA antisense oligodeoxyribonucleotide (AMO) and the like. Genes may or may not be capable of being used to produce a functional protein or gene product. Genes can include both coding and non-coding regions (e.g., introns, regulatory elements, promoters, enhancers, termination sequences and/or 5′ and 3′ untranslated regions). A gene may be “isolated” by which is meant a nucleic acid that is substantially or essentially free from components normally found in association with the nucleic acid in its natural state. Such components include other cellular material, culture medium from recombinant production, and/or various chemicals used in chemically synthesizing the nucleic acid.

The term “mutation” refers to point mutations (e.g., missense, or nonsense, or insertions or deletions of single base pairs that result in in-frame shifts), insertions, deletions, and/or truncations. When the mutation is a substitution of a residue within an amino acid sequence with another residue, or a deletion or insertion of one or more residues within a sequence, the mutations are typically described by identifying the original residue followed by the position of the residue within the sequence and by the identity of the newly substituted residue. In some embodiments, a deletion or an insertion is an in-frame or out-of-frame deletion or an in-frame or out-of-frame insertion, e.g., an in-frame or out-of-frame deletion or an in-frame or out-of-frame insertion in an endogenous KASII nucleic acid. In some embodiments, a deletion or an insertion may be an in-frame or out-of-frame deletion or an in-frame or out-of-frame insertion, e.g., an in-frame or out-of-frame deletion or an in-frame or out-of-frame insertion in an upstream/regulatory region (region 5′ of the start codon, ATG), a first, second or third exon of an endogenous KASII gene, and/or in a 3′ region of the KASII gene. In some embodiments, a mutation in a KASII gene of a peanut plant may result in increased amounts of palmitic acid in the seed oil of the peanut plant comprising the mutation, optionally an increased ratio of palmitic acid to stearic acid.

The terms “complementary” or “complementarity,” as used herein, refer to the natural binding of polynucleotides under permissive salt and temperature conditions by base-pairing. For example, the sequence “A-G-T” (5′ to 3′) binds to the complementary sequence “T-C-A” (3′ to 5′). Complementarity between two single-stranded molecules may be “partial,” in which only some of the nucleotides bind, or it may be complete when total complementarity exists between the single stranded molecules. The degree of complementarity between nucleic acid strands has significant effects on the efficiency and strength of hybridization between nucleic acid strands.

“Complement,” as used herein, can mean 100% complementarity with the comparator nucleotide sequence or it can mean less than 100% complementarity (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and the like, complementarity, e.g., substantial complementarity) to the comparator nucleotide sequence.

Different nucleic acids or proteins having homology are referred to herein as “homologues.” The term homologue includes homologous sequences from the same and from other species and orthologous sequences from the same and other species. “Homology” refers to the level of similarity between two or more nucleic acid and/or amino acid sequences in terms of percent of positional identity (i.e., sequence similarity or identity). Homology also refers to the concept of similar functional properties among different nucleic acids or proteins. Thus, the compositions and methods of the invention further comprise homologues to the nucleotide sequences and polypeptide sequences of this invention. “Orthologous,” as used herein, refers to homologous nucleotide sequences and/or amino acid sequences in different species that arose from a common ancestral gene during speciation. A homologue of a nucleotide sequence of this invention has a substantial sequence identity (e.g., at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100%) to said nucleotide sequence of the invention.

As used herein “sequence identity” refers to the extent to which two optimally aligned polynucleotide or polypeptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned. In some embodiments, “percent sequence identity” can refer to the percentage of identical amino acids in an amino acid sequence as compared to a reference polypeptide.

As used herein, the phrase “substantially identical,” or “substantial identity” in the context of two nucleic acid molecules, nucleotide sequences or polypeptide sequences, refers to two or more sequences or subsequences that have at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100% nucleotide or amino acid residue identity, when compared and aligned for maximum correspondence, as measured using one of the following sequence comparison algorithms or by visual inspection. In some embodiments of the invention, the substantial identity exists over a region of consecutive nucleotides of a nucleotide sequence of the invention that is about 10 nucleotides to about 20 nucleotides, about 10 nucleotides to about 25 nucleotides, about 10 nucleotides to about 30 nucleotides, about 15 nucleotides to about 25 nucleotides, about 30 nucleotides to about 40 nucleotides, about 50 nucleotides to about 60 nucleotides, about 70 nucleotides to about 80 nucleotides, about 90 nucleotides to about 100 nucleotides, about 100 nucleotides to about 200 nucleotides, about 100 nucleotides to about 300 nucleotides, about 100 nucleotides to about 400 nucleotides, about 100 nucleotides to about 500 nucleotides, about 100 nucleotides to about 600 nucleotides, about 100 nucleotides to about 800 nucleotides, about 100 nucleotides to about 900 nucleotides, or more nucleotides in length, and any range therein, up to the full length of the sequence. In some embodiments, nucleotide sequences can be substantially identical over at least about 20 consecutive nucleotides (e.g., about 20, 21, 22, 23, 24, 25, 26, 27, 28, 29, 30, 31, 32, 33, 34, 35, 36, 37, 38, 39, 40, 50, 60, 70, 80, 90, 100, 200, 300, 400, 500, 600, 700, 800, 900, 1000, 1100, 1200, 1300, 1400, 1500, 1600, 1700, 1800, 1900, 2000, 2100, 2200, 2300, 2500, 3000, 3500, 4000 or more nucleotides). In some embodiments, two or more KASI genes may be substantially identical to one another over at least about 10, 20, 30, 40, 50, 100, 200, 300, 400, 500, 600, 700, 800, 900, 1000, 1100, 1200, 1300, 1400, or 1500 to about 2000, 2050, 2100, 2150, 2200, 2250, 2300, 2350, 2400, 2450, 2500, 2510, 2520, 2530, 2540, 2550, 2600, 2650, 2700, 2750, 2800, 2850, 2900, 2950, 3000, 3050, 3100, 3150, 3200, 3250, 3300, 3350, 3400, 3450, 3490, 3500, 3600, 3700, 3800, 3900, 4000, 4100, 4200, 4300, 4400, 4500, 4600, 4700, 4800, 4900, 5000, 5100, 5200, 5300, 5500, 6000, 6500 or more consecutive nucleotides of a KAISII gene, e.g., SEQ ID NOs:1, 2, 116, 117, 118, or 119, optionally over about 10, 20, 30, 40, 50, 60, 70, 80, 90, 100, 110, 120, 130, 140, 150, 200, 210, 220, 230, 240, 250, 260, 270, 280, 290, 300, 310, 320, 330, 340, 350, 360, 370, 380, 390, 400, 420, 440, 460, or 480 consecutive nucleotides to about 500, 520, 540, 560, 580, 600, 650, 700, 750, 800, 850, 900, 950, 1000, 1100, 1200, 1300, 1400, 1500, 1600, 1700, 1800, 1900, 2000, 2100, 2200, 2300, 2400, or 2500 or more consecutive nucleotides of a KASII gene, e.g., SEQ ID NOs:1, 2, 116, 117, 118, or 119.

In some embodiments of the invention, the substantial identity exists over a region of consecutive amino acid residues of a polypeptide of the invention that is about 3 amino acid residues to about 10, 11, 12, 13, 14, 15, 16, 17, 18, 19, or 20 amino acid residues, about 5 amino acid residues to about 25, 30, 35, 40, 45, 50 or 60 amino acid residues, about 15 amino acid residues to about 30 amino acid residues, about 20 amino acid residues to about 40 amino acid residues, about 25 amino acid residues to about 40 amino acid residues, about 25 amino acid residues to about 50 amino acid residues, about 30 amino acid residues to about 50 amino acid residues, about 40 amino acid residues to about 50 amino acid residues, about 40 amino acid residues to about 70 amino acid residues, about 50 amino acid residues to about 70 amino acid residues, about 60 amino acid residues to about 80 amino acid residues, about 70 amino acid residues to about 80 amino acid residues, about 90 amino acid residues to about 100 amino acid residues, or more amino acid residues in length, and any range therein, up to the full length of the sequence. In some embodiments, polypeptide sequences can be substantially identical to one another over at least about 8, 9, 10, 11, 12, 13, 14, or more consecutive amino acid residues (e.g., about 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 130, 140, 150, 175, 200, 225, 250, 275, 300, 325, 350, 400, 450, 500, or more amino acids in length or more consecutive amino acid residues). In some embodiments, two or more KasII polypeptides may be substantially identical to one another over at least about 10 to about 500 consecutive amino acid residues of the amino acid sequence of, for example, SEQ ID NO:4 or SEQ ID NO:121. In some embodiments, a substantially identical nucleotide or protein sequence may perform substantially the same function as the nucleotide (or encoded protein sequence) to which it is substantially identical.

For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., San Diego, CA). An “identity fraction” for aligned segments of a test sequence and a reference sequence is the number of identical components which are shared by the two aligned sequences divided by the total number of components in the reference sequence segment, e.g., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

Two nucleotide sequences may also be considered substantially complementary when the two sequences hybridize to each other under stringent conditions. In some embodiments, two nucleotide sequences considered to be substantially complementary hybridize to each other under highly stringent conditions.

“Stringent hybridization conditions” and “stringent hybridization wash conditions” in the context of nucleic acid hybridization experiments such as Southern and Northern hybridizations are sequence dependent and are different under different environmental parameters. An extensive guide to the hybridization of nucleic acids is found in Tijssen Laboratory Techniques in Biochemistry and Molecular Biology—Hybridization with Nucleic Acid Probes part I chapter 2 “Overview of principles of hybridization and the strategy of nucleic acid probe assays” Elsevier, New York (1993). Generally, highly stringent hybridization and wash conditions are selected to be about 5° C. lower than the thermal melting point (Tₘ) for the specific sequence at a defined ionic strength and pH.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleotide sequences which have more than 100 complementary residues on a filter in a Southern or northern blot is 50% formamide with 1 mg of heparin at 42° C., with the hybridization being carried out overnight. An example of highly stringent wash conditions is 0.1 5M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example of a medium stringency wash for a duplex of, e.g., more than 100 nucleotides, is 1×SSC at 45° C. for 15 minutes. An example of a low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleotide sequences that do not hybridize to each other under stringent conditions are still substantially identical if the proteins that they encode are substantially identical. This can occur, for example, when a copy of a nucleotide sequence is created using the maximum codon degeneracy permitted by the genetic code.

A polynucleotide and/or recombinant nucleic acid construct of this invention (e.g., expression cassettes and/or vectors) may be codon optimized for expression. In some embodiments, the polynucleotides, nucleic acid constructs, expression cassettes, and/or vectors of the editing systems of the invention (e.g., comprising/encoding a sequence-specific DNA binding domain/protein (e.g., a sequence-specific DNA binding domain/protein from a polynucleotide-guided endonuclease, a zinc finger nuclease, a transcription activator-like effector nuclease (TALEN), an Argonaute protein, and/or a CRISPR-Cas endonuclease (e.g., CRISPR-Cas effector protein) (e.g., a Type I CRISPR-Cas effector protein, a Type II CRISPR-Cas effector protein, a Type III CRISPR-Cas effector protein, a Type IV CRISPR-Cas effector protein, a Type V CRISPR-Cas effector protein or a Type VI CRISPR-Cas effector protein)), a nuclease (e.g., an endonuclease (e.g., Fok1), a polynucleotide-guided endonuclease, a CRISPR-Cas endonuclease (e.g., CRISPR-Cas effector protein), a zinc finger nuclease, and/or a transcription activator-like effector nuclease (TALEN)), may be codon optimized for expression in a plant. In some embodiments, the codon optimized nucleic acids, polynucleotides, expression cassettes, and/or vectors of the invention have about 70% to about 99.9% (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%. 99.9% or 100%) identity or more to the reference nucleic acids, polynucleotides, expression cassettes, and/or vectors that have not been codon optimized.

A polynucleotide or nucleic acid construct of the invention may be operatively associated with a variety of promoters and/or other regulatory elements for expression in a plant and/or a cell of a plant. Thus, in some embodiments, a polynucleotide or nucleic acid construct of this invention may further comprise one or more promoters, introns, enhancers, and/or terminators operably linked to one or more nucleotide sequences. In some embodiments, a promoter may be operably associated with an intron (e.g., Ubi1 promoter and intron). In some embodiments, a promoter associated with an intron maybe referred to as a “promoter region”.

By “operably linked” or “operably associated” as used herein in reference to polynucleotides, it is meant that the indicated elements are functionally related to each other and are also generally physically related. Thus, the term “operably linked” or “operably associated” as used herein, refers to nucleotide sequences on a single nucleic acid molecule that are functionally associated. Thus, a first nucleotide sequence that is operably linked to a second nucleotide sequence means a situation when the first nucleotide sequence is placed in a functional relationship with the second nucleotide sequence. For instance, a promoter is operably associated with a nucleotide sequence if the promoter effects the transcription or expression of said nucleotide sequence. Those skilled in the art will appreciate that the control sequences (e.g., promoter) need not be contiguous with the nucleotide sequence to which it is operably associated, as long as the control sequences function to direct the expression thereof. Thus, for example, intervening untranslated, yet transcribed, nucleic acid sequences can be present between a promoter and the nucleotide sequence, and the promoter can still be considered “operably linked” to the nucleotide sequence.

As used herein, the term “linked,” in reference to polypeptides, refers to the attachment of one polypeptide to another. A polypeptide may be linked to another polypeptide (at the N-terminus or the C-terminus) directly (e.g., via a peptide bond) or through a linker. The term “linker” is art-recognized and refers to a chemical group, or a molecule linking two molecules or moieties, e.g., two domains of a fusion protein. A linker may be comprised of a single linking molecule or may comprise more than one linking molecule. In some embodiments, the linker can be an organic molecule, group, polymer, or chemical moiety such as a bivalent organic moiety. In some embodiments, the linker may be an amino acid, or it may be a peptide. In some embodiments, the linker is a peptide.

As used herein, the term “linked,” or “fused” in reference to polynucleotides, refers to the attachment of one polynucleotide to another. In some embodiments, two or more polynucleotide molecules may be linked by a linker that can be an organic molecule, group, polymer, or chemical moiety such as a bivalent organic moiety. A polynucleotide may be linked or fused to another polynucleotide (at the 5′ end or the 3′ end) via a covalent or non-covenant linkage or binding, including e.g., Watson-Crick base-pairing, or through one or more linking nucleotides. In some embodiments, a polynucleotide motif of a certain structure may be inserted within another polynucleotide sequence (e.g., extension of the hairpin structure in the guide RNA). In some embodiments, the linking nucleotides may be naturally occurring nucleotides. In some embodiments, the linking nucleotides may be non-naturally occurring nucleotides.

A “promoter” is a nucleotide sequence that controls or regulates the transcription of a nucleotide sequence (e.g., a coding sequence) that is operably associated with the promoter. The coding sequence controlled or regulated by a promoter may encode a polypeptide and/or a functional RNA. Typically, a “promoter” refers to a nucleotide sequence that contains a binding site for RNA polymerase II and directs the initiation of transcription. In general, promoters are found 5′, or upstream, relative to the start of the coding region of the corresponding coding sequence. A promoter may comprise other elements that act as regulators of gene expression; e.g., a promoter region. These include a TATA box consensus sequence, and often a CAAT box consensus sequence (Breathnach and Chambon, (1981) Annu. Rev. Biochem. 50:349). In plants, the CAAT box may be substituted by the AGGA box (Messing et al., (1983) in Genetic Engineering of Plants, T. Kosuge, C. Meredith and A. Hollaender (eds.), Plenum Press, pp. 211-227).

Promoters useful with this invention can include, for example, constitutive, inducible, temporally regulated, developmentally regulated, chemically regulated, tissue-preferred and/or tissue-specific promoters for use in the preparation of recombinant nucleic acid molecules, e.g., “synthetic nucleic acid constructs” or “protein-RNA complex.” These various types of promoters are known in the art.

The choice of promoter may vary depending on the temporal and spatial requirements for expression, and also may vary based on the host cell to be transformed. Promoters for many different organisms are well known in the art. Based on the extensive knowledge present in the art, the appropriate promoter can be selected for the particular host organism of interest. Thus, for example, much is known about promoters upstream of highly constitutively expressed genes in model organisms and such knowledge can be readily accessed and implemented in other systems as appropriate.

In some embodiments, a promoter functional in a plant may be used with the constructs of this invention. Non-limiting examples of a promoter useful for driving expression in a plant include the promoter of the RubisCo small subunit gene 1 (PrbcS1), the promoter of the actin gene (Pactin), the promoter of the nitrate reductase gene (Pnr) and the promoter of duplicated carbonic anhydrase gene 1 (Pdca1) (See, Walker et al. Plant Cell Rep. 23:727-735 (2005); Li et al. Gene 403:132-142 (2007); Li et al. Mol Biol. Rep. 37:1143-1154 (2010)). PrbcS1 and Pactin are constitutive promoters and Pnr and Pdca1 are inducible promoters. Pnr is induced by nitrate and repressed by ammonium (Li et al. Gene 403:132-142 (2007)) and Pdca1 is induced by salt (Li et al. Mol Biol. Rep. 37:1143-1154 (2010)). In some embodiments, a promoter useful with this invention is RNA polymerase II (Pol II) promoter. In some embodiments, a U6 promoter or a 7SL promoter from Zea mays may be useful with constructs of this invention. In some embodiments, the U6c promoter and/or 7SL promoter from Zea mays may be useful for driving expression of a guide nucleic acid. In some embodiments, a U6c promoter, U6i promoter and/or 7SL promoter from Glycine max may be useful with constructs of this invention. In some embodiments, the U6c promoter, Ubi promoter and/or 7SL promoter from Glycine max may be useful for driving expression of a guide nucleic acid.

Examples of constitutive promoters useful for plants include, but are not limited to, cestrum virus promoter (cmp) (U.S. Pat. No. 7,166,770), the rice actin 1 promoter (Wang et al. (1992) Mol. Cell. Biol. 12:3399-3406; as well as U.S. Pat. No. 5,641,876), CaMV 35S promoter (Odell et al. (1985) Nature 313:810-812), CaMV 19S promoter (Lawton et al. (1987) Plant Mol. Biol. 9:315-324), nos promoter (Ebert et al. (1987) Proc. Natl. Acad. Sci USA 84:5745-5749), Adh promoter (Walker et al. (1987) Proc. Natl. Acad. Sci. USA 84:6624-6629), sucrose synthase promoter (Yang & Russell (1990) Proc. Natl. Acad. Sci. USA 87:4144-4148), and the ubiquitin promoter. The constitutive promoter derived from ubiquitin accumulates in many cell types. Ubiquitin promoters have been cloned from several plant species for use in transgenic plants, for example, sunflower (Binet et al., 1991. Plant Science 79: 87-94), maize (Christensen et al., 1989. Plant Molec. Biol. 12: 619-632), and arabidopsis (Norris et al. 1993. Plant Molec. Biol. 21:895-906). The maize ubiquitin promoter (UbiP) has been developed in transgenic monocot systems and its sequence and vectors constructed for monocot transformation are disclosed in the patent publication EP 0 342 926. The ubiquitin promoter is suitable for the expression of the nucleotide sequences of the invention in transgenic plants, especially monocotyledons. Further, the promoter expression cassettes described by McElroy et al. (Mol. Gen. Genet. 231: 150-160 (1991)) can be easily modified for the expression of the nucleotide sequences of the invention and are particularly suitable for use in monocotyledonous hosts.

In some embodiments, tissue specific/tissue preferred promoters can be used for expression of a heterologous polynucleotide in a plant cell. Tissue specific or preferred expression patterns include, but are not limited to, green tissue specific or preferred, root specific or preferred, stem specific or preferred, flower specific or preferred or pollen specific or preferred. Promoters suitable for expression in green tissue include many that regulate genes involved in photosynthesis and many of these have been cloned from both monocotyledons and dicotyledons. In one embodiment, a promoter useful with the invention is the maize PEPC promoter from the phosphoenol carboxylase gene (Hudspeth & Grula, Plant Molec. Biol. 12:579-589 (1989)). Non-limiting examples of tissue-specific promoters include those associated with genes encoding the seed storage proteins (such as β-conglycinin, cruciferin, napin and phaseolin), zein or oil body proteins (such as oleosin), or proteins involved in fatty acid biosynthesis (including acyl carrier protein, stearoyl-ACP desaturase and fatty acid desaturases (fad 2-1)), and other nucleic acids expressed during embryo development (such as Bce4, see, e.g., Kridl et al. (1991) Seed Sci. Res. 1:209-219; as well as EP Patent No. 255378). Tissue-specific or tissue-preferential promoters useful for the expression of the nucleotide sequences of the invention in plants, particularly maize, include but are not limited to those that direct expression in root, pith, leaf or pollen. Such promoters are disclosed, for example, in WO 93/07278, herein incorporated by reference in its entirety. Other non-limiting examples of tissue specific or tissue preferred promoters useful with the invention the cotton rubisco promoter disclosed in U.S. Pat. No. 6,040,504; the rice sucrose synthase promoter disclosed in U.S. Pat. No. 5,604,121; the root specific promoter described by de Framond (FEBS 290:103-106 (1991); EP 0 452 269 to Ciba-Geigy); the stem specific promoter described in U.S. Pat. No. 5,625,136 (to Ciba-Geigy) and which drives expression of the maize trpA gene; the cestrum yellow leaf curling virus promoter disclosed in WO 01/73087; and pollen specific or preferred promoters including, but not limited to, ProOsLPS10 and ProOsLPS11 from rice (Nguyen et al. Plant Biotechnol. Reports 9(5):297-306 (2015)), ZmSTK2_USP from maize (Wang et al. Genome 60(6):485-495 (2017)), LAT52 and LAT59 from tomato (Twell et al. Development 109(3):705-713 (1990)), Zm13 (U.S. Pat. No. 10,421,972), PLA₂-δ promoter from arabidopsis (U.S. Pat. No. 7,141,424), and/or the ZmC5 promoter from maize (International PCT Publication No. WO1999/042587.

Additional examples of plant tissue-specific/tissue preferred promoters include, but are not limited to, the root hair-specific cis-elements (RHEs) (Kim et al. The Plant Cell 18:2958-2970 (2006)), the root-specific promoters RCc3 (Jeong et al. Plant Physiol. 153:185-197 (2010)) and RB7 (U.S. Pat. No. 5,459,252), the lectin promoter (Lindstrom et al. (1990) Der. Genet. 11:160-167; and Vodkin (1983) Prog. Clin. Biol. Res. 138:87-98), corn alcohol dehydrogenase 1 promoter (Dennis et al. (1984) Nucleic Acids Res. 12:3983-4000), S-adenosyl-L-methionine synthetase (SAMS) (Vander Mijnsbrugge et al. (1996) Plant and Cell Physiology, 37(8):1108-1115), corn light harvesting complex promoter (Bansal et al. (1992) Proc. Natl. Acad. Sci. USA 89:3654-3658), corn heat shock protein promoter (O'Dell et al. (1985) EMBO J. 5:451-458; and Rochester et al. (1986) EMBO J. 5:451-458), pea small subunit RuBP carboxylase promoter (Cashmore, “Nuclear genes encoding the small subunit of ribulose-1,5-bisphosphate carboxylase” pp. 29-39 In: Genetic Engineering of Plants (Hollaender ed., Plenum Press 1983; and Poulsen et al. (1986) Mol. Gen. Genet. 205:193-200), Ti plasmid mannopine synthase promoter (Langridge et al. (1989) Proc. Natl. Acad. Sci. USA 86:3219-3223), Ti plasmid nopaline synthase promoter (Langridge et al. (1989), supra), petunia chalcone isomerase promoter (van Tunen et al. (1988) EMBO J. 7:1257-1263), bean glycine rich protein 1 promoter (Keller et al. (1989) Genes Dev. 3:1639-1646), truncated CaMV 35S promoter (O'Dell et al. (1985) Nature 313:810-812), potato patatin promoter (Wenzler et al. (1989) Plant Mol. Biol. 13:347-354), root cell promoter (Yamamoto et al. (1990) Nucleic Acids Res. 18:7449), maize zein promoter (Kriz et al. (1987) Mol. Gen. Genet. 207:90-98; Langridge et al. (1983) Cell 34:1015-1022; Reina et al. (1990) Nucleic Acids Res. 18:6425; Reina et al. (1990) Nucleic Acids Res. 18:7449; and Wandelt et al. (1989) Nucleic Acids Res. 17:2354), globulin-1 promoter (Belanger et al. (1991) Genetics 129:863-872), α-tubulin cab promoter (Sullivan et al. (1989) Mol. Gen. Genet. 215:431-440), PEPCase promoter (Hudspeth & Grula (1989) Plant Mol. Biol. 12:579-589), R gene complex-associated promoters (Chandler et al. (1989) Plant Cell 1:1175-1183), and chalcone synthase promoters (Franken et al. (1991) EMBO J. 10:2605-2612).

Useful for seed-specific expression is the pea vicilin promoter (Czako et al. (1992) Mol. Gen. Genet. 235:33-40; as well as the seed-specific promoters disclosed in U.S. Pat. No. 5,625,136. Useful promoters for expression in mature leaves are those that are switched at the onset of senescence, such as the SAG promoter from Arabidopsis (Gan et al. (1995) Science 270:1986-1988).

In addition, promoters functional in chloroplasts can be used. Non-limiting examples of such promoters include the bacteriophage T3 gene 9 5′ UTR and other promoters disclosed in U.S. Pat. No. 7,579,516. Other promoters useful with the invention include but are not limited to the S-E9 small subunit RuBP carboxylase promoter and the Kunitz trypsin inhibitor gene promoter (Kti3).

Additional regulatory elements useful with this invention include, but are not limited to, introns, enhancers, termination sequences and/or 5′ and 3′ untranslated regions.

An intron useful with this invention can be an intron identified in and isolated from a plant and then inserted into an expression cassette to be used in transformation of a plant. As would be understood by those of skill in the art, introns can comprise the sequences required for self-excision and are incorporated into nucleic acid constructs/expression cassettes in frame. An intron can be used either as a spacer to separate multiple protein-coding sequences in one nucleic acid construct, or an intron can be used inside one protein-coding sequence to, for example, stabilize the mRNA. If they are used within a protein-coding sequence, they are inserted “in-frame” with the excision sites included. Introns may also be associated with promoters to improve or modify expression.

Non-limiting examples of introns useful with the present invention include introns from the ADHI gene (e.g., Adh1-S introns 1, 2 and 6), the ubiquitin gene (Ubi1), the RuBisCO small subunit (rbcS) gene, the RuBisCO large subunit (rbcL) gene, the actin gene (e.g., actin-1 intron), the pyruvate dehydrogenase kinase gene (pdk), the nitrate reductase gene (nr), the duplicated carbonic anhydrase gene 1 (Tdca1), the psbA gene, the atpA gene, or any combination thereof.

In some embodiments, a polynucleotide and/or a nucleic acid construct of the invention can be an “expression cassette” or can be comprised within an expression cassette. As used herein, “expression cassette” means a recombinant nucleic acid molecule comprising, for example, a one or more polynucleotides of the invention (e.g., a polynucleotide encoding a sequence-specific nucleic acid binding domain (e.g., sequence-specific DNA binding domain), wherein polynucleotide(s) is/are operably associated with one or more control sequences (e.g., a promoter, terminator and the like). Thus, in some embodiments, one or more expression cassettes may be provided, which are designed to express, for example, a nucleic acid construct of the invention (e.g., a polynucleotide encoding a sequence-specific nucleic acid binding domain, a polynucleotide encoding a nuclease polypeptide/domain, and the like, or comprising a guide nucleic acid, and the like). When an expression cassette of the present invention comprises more than one polynucleotide, the polynucleotides may be operably linked to a single promoter that drives expression of all of the polynucleotides or the polynucleotides may be operably linked to one or more separate promoters (e.g., three polynucleotides may be driven by one, two or three promoters in any combination). When two or more separate promoters are used, the promoters may be the same promoter or they may be different promoters. Thus, a polynucleotide encoding a sequence specific nucleic acid binding domain, a polynucleotide encoding a nuclease protein/domain, a polynucleotide encoding a CRISPR-Cas effector protein/domain, and/or a guide nucleic acid, when comprised in a single expression cassette may each be operably linked to a single promoter, or separate promoters in any combination.

An expression cassette comprising a nucleic acid construct of the invention may be chimeric, meaning that at least one of its components is heterologous with respect to at least one of its other components (e.g., a promoter from the host organism operably linked to a polynucleotide of interest to be expressed in the host organism, wherein the polynucleotide of interest is from a different organism than the host or is not normally found in association with that promoter). An expression cassette may also be one that is naturally occurring but has been obtained in a recombinant form useful for heterologous expression.

An expression cassette can optionally include a transcriptional and/or translational termination region (i.e., termination region) and/or an enhancer region that is functional in the selected host cell. A variety of transcriptional terminators and enhancers are known in the art and are available for use in expression cassettes. Transcriptional terminators are responsible for the termination of transcription and correct mRNA polyadenylation. A termination region and/or the enhancer region may be native to the transcriptional initiation region, may be native to, for example, a gene encoding a sequence-specific nucleic acid binding protein, a gene encoding a nuclease, a gene encoding a reverse transcriptase, a gene encoding a deaminase, and the like, or may be native to a host cell, or may be native to another source (e.g., foreign or heterologous to, for example, to a promoter, to a gene encoding a sequence-specific nucleic acid binding protein, a gene encoding a nuclease, a gene encoding a reverse transcriptase, a gene encoding a deaminase, and the like, or to the host cell, or any combination thereof).

An expression cassette of the invention also can include a polynucleotide encoding a selectable marker, which can be used to select a transformed host cell. As used herein, “selectable marker” means a polynucleotide sequence that when expressed imparts a distinct phenotype to the host cell expressing the marker and thus allows such transformed cells to be distinguished from those that do not have the marker. Such a polynucleotide sequence may encode either a selectable or screenable marker, depending on whether the marker confers a trait that can be selected for by chemical means, such as by using a selective agent (e.g., an antibiotic and the like), or on whether the marker is simply a trait that one can identify through observation or testing, such as by screening (e.g., fluorescence). Many examples of suitable selectable markers are known in the art and can be used in the expression cassettes described herein.

In addition to expression cassettes, the nucleic acid molecules/constructs and polynucleotide sequences described herein can be used in connection with vectors. The term “vector” refers to a composition for transferring, delivering or introducing a nucleic acid (or nucleic acids) into a cell. A vector comprises a nucleic acid construct (e.g., expression cassette(s)) comprising the nucleotide sequence(s) to be transferred, delivered or introduced. Vectors for use in transformation of host organisms are well known in the art. Non-limiting examples of general classes of vectors include viral vectors, plasmid vectors, phage vectors, phagemid vectors, cosmid vectors, fosmid vectors, bacteriophages, artificial chromosomes, minicircles, or Agrobacterium binary vectors in double or single stranded linear or circular form which may or may not be self-transmissible or mobilizable. In some embodiments, a viral vector can include, but is not limited, to a retroviral, lentiviral, adenoviral, adeno-associated, or herpes simplex viral vector. A vector as defined herein can transform a prokaryotic or eukaryotic host either by integration into the cellular genome or exist extrachromosomally (e.g., autonomous replicating plasmid with an origin of replication). Additionally included are shuttle vectors by which is meant a DNA vehicle capable, naturally or by design, of replication in two different host organisms, which may be selected from actinomycetes and related species, bacteria and eukaryotic (e.g., higher plant, mammalian, yeast or fungal cells). In some embodiments, the nucleic acid in the vector is under the control of, and operably linked to, an appropriate promoter or other regulatory elements for transcription in a host cell. The vector may be a bi-functional expression vector which functions in multiple hosts. In the case of genomic DNA, this may contain its own promoter and/or other regulatory elements and in the case of cDNA this may be under the control of an appropriate promoter and/or other regulatory elements for expression in the host cell. Accordingly, a nucleic acid or polynucleotide of this invention and/or expression cassettes comprising the same may be comprised in vectors as described herein and as known in the art.

As used herein, “contact,” contacting,” “contacted,” and grammatical variations thereof, refers to placing the components of a desired reaction together under conditions suitable for carrying out the desired reaction (e.g., integration, transformation, genome editing, nicking, cleavage, and the like). The methods and conditions for carrying out such reactions are well known in the art (See, e.g., Gasiunas et al. (2012) Proc. Natl. Acad. Sci. 109: E2579-E2586; M. R. Green and J. Sambrook (2012) Molecular Cloning: A Laboratory Manual. 4th Ed., Cold Spring Harbor Laboratory Press, Cold Spring Harbor, NY). As an example, a target nucleic acid may be contacted with a sequence-specific nucleic acid binding protein (e.g., polynucleotide-guided endonuclease, a CRISPR-Cas endonuclease (e.g., CRISPR-Cas effector protein), a zinc finger nuclease, a transcription activator-like effector nuclease (TALEN) and/or an Argonaute protein) and a CRISPR guide nucleic acid) and/or a nucleic acid construct encoding the same, under conditions whereby the sequence-specific nucleic acid protein is expressed and the sequence-specific nucleic acid binding protein binds to the target nucleic acid, thereby modifying the target nucleic acid.

As used herein, “modifying” or “modification” in reference to a target nucleic acid includes editing (e.g., mutating), covalent modification, exchanging/substituting nucleic acids/nucleotide bases, deleting, cleaving, nicking, and/or altering transcriptional control of a target nucleic acid. In some embodiments, a modification may include one or more single base changes (SNPs) of any type.

“Introducing,” “introduce,” “introduced” (and grammatical variations thereof) in the context of a polynucleotide of interest means presenting a nucleotide sequence of interest (e.g., polynucleotide, a nucleic acid construct, and/or a guide nucleic acid) to a plant, plant part thereof, or cell thereof, in such a manner that the nucleotide sequence gains access to the interior of a cell.

The terms “transformation” or transfection” may be used interchangeably and as used herein refer to the introduction of a heterologous nucleic acid into a cell. Transformation of a cell may be stable or transient. Thus, in some embodiments, a host cell or host organism (e.g., a plant, a peanut plant) may be stably transformed with a polynucleotide/nucleic acid molecule of the invention. In some embodiments, a host cell or host organism may be transiently transformed with a polynucleotide/nucleic acid molecule of the invention.

“Transient transformation” in the context of a polynucleotide means that a polynucleotide is introduced into the cell and does not integrate into the genome of the cell.

By “stably introducing” or “stably introduced” in the context of a polynucleotide introduced into a cell is intended that the introduced polynucleotide is stably incorporated into the genome of the cell, and thus the cell is stably transformed with the polynucleotide.

“Stable transformation” or “stably transformed” as used herein means that a nucleic acid molecule is introduced into a cell and integrates into the genome of the cell. As such, the integrated nucleic acid molecule is capable of being inherited by the progeny thereof, more particularly, by the progeny of multiple successive generations. “Genome” as used herein includes the nuclear and the plastid genome, and therefore includes integration of the nucleic acid into, for example, the chloroplast or mitochondrial genome. Stable transformation as used herein can also refer to a transgene that is maintained extrachromasomally, for example, as a minichromosome or a plasmid.

Transient transformation may be detected by, for example, an enzyme-linked immunosorbent assay (ELISA) or Western blot, which can detect the presence of a peptide or polypeptide encoded by one or more transgene introduced into an organism. Stable transformation of a cell can be detected by, for example, a Southern blot hybridization assay of genomic DNA of the cell with nucleic acid sequences which specifically hybridize with a nucleotide sequence of a transgene introduced into an organism (e.g., a plant, a peanut plant). Stable transformation of a cell can be detected by, for example, a Northern blot hybridization assay of RNA of the cell with nucleic acid sequences which specifically hybridize with a nucleotide sequence of a transgene introduced into a host organism. Stable transformation of a cell can also be detected by, e.g., a polymerase chain reaction (PCR) or other amplification reactions as are well known in the art, employing specific primer sequences that hybridize with target sequence(s) of a transgene, resulting in amplification of the transgene sequence, which can be detected according to standard methods. Transformation can also be detected by direct sequencing and/or hybridization protocols well known in the art.

Accordingly, in some embodiments, nucleotide sequences, polynucleotides, nucleic acid constructs, and/or expression cassettes of the invention may be expressed transiently and/or they can be stably incorporated into the genome of the host organism. Thus, in some embodiments, a nucleic acid construct of the invention (e.g., one or more expression cassettes comprising polynucleotides for editing as described herein) may be transiently introduced into a cell with a guide nucleic acid and as such, no DNA is maintained in the cell.

A nucleic acid construct of the invention may be introduced into a plant cell by any method known to those of skill in the art. Non-limiting examples of transformation methods include transformation via bacterial-mediated nucleic acid delivery (e.g., via Agrobacteria), viral-mediated nucleic acid delivery, silicon carbide or nucleic acid whisker-mediated nucleic acid delivery, liposome mediated nucleic acid delivery, microinjection, microparticle bombardment, calcium-phosphate-mediated transformation, cyclodextrin-mediated transformation, electroporation, nanoparticle-mediated transformation, sonication, infiltration, PEG-mediated nucleic acid uptake, as well as any other electrical, chemical, physical (mechanical) and/or biological mechanism that results in the introduction of nucleic acid into the plant cell, including any combination thereof. Procedures for transforming both eukaryotic and prokaryotic organisms are well known and routine in the art and are described throughout the literature (See, for example, Jiang et al. 2013. Nat. Biotechnol. 31:233-239; Ran et al. Nature Protocols 8:2281-2308 (2013)). General guides to various plant transformation methods known in the art include Miki et al. (“Procedures for Introducing Foreign DNA into Plants” in Methods in Plant Molecular Biology and Biotechnology, Glick, B. R. and Thompson, J. E., Eds. (CRC Press, Inc., Boca Raton, 1993), pages 67-88) and Rakowoczy-Trojanowska (Cell. Mol. Biol. Lett. 7:849-858 (2002)).

In some embodiments of the invention, transformation of a cell may comprise nuclear transformation. In other embodiments, transformation of a cell may comprise plastid transformation (e.g., chloroplast transformation). In still further embodiments, nucleic acids of the invention may be introduced into a cell via conventional breeding techniques. In some embodiments, one or more of the polynucleotides, expression cassettes and/or vectors may be introduced into a plant cell via Agrobacterium transformation.

A polynucleotide therefore can be introduced into a plant, plant part, plant cell in any number of ways that are well known in the art. The methods of the invention do not depend on a particular method for introducing one or more nucleotide sequences into a plant, only that they gain access to the interior the cell. Where more than polynucleotide is to be introduced, they can be assembled as part of a single nucleic acid construct, or as separate nucleic acid constructs, and can be located on the same or different nucleic acid constructs. Accordingly, the polynucleotide can be introduced into the cell of interest in a single transformation event, or in separate transformation events, or, alternatively, a polynucleotide can be incorporated into a plant as part of a breeding protocol.

β-ketoacyl-ACP synthetase II (Kas II) (also known as 3-oxoacyl-acyl carrier protein synthase II) is an enzyme in the fatty acid biosynthesis pathway, which catalyzes the elongation of C16:0 to C18:0 fatty acids in the plastids of the plant cell and controls the ratio of palmitic acid (16:0) to stearic acid (18:0) in oil seeds (Shanlin et al. 4th International Conference on Bioinformatics and Biomedical Engineering, 2010, pp. 1-5). A mutation in the KAS II gene in soybean was shown to result in elevated amount of palmitic acid levels (Aghoram, et, al., Crop Science 46:2453-2459 (2006)). In general, a higher level of palmitic acid can provide increased oxidative stability, thus can reduce rancidity upon storage of a seed. Higher palmitic acid levels can also increase the melting point of seed oil, which is favored in the making of margarine and confectionery products, and as such provides a good alternative to hydrogenated vegetable oils (Richie, H. and Roser, M. Palm Oil. Our World in Data. [Online] 2020. (ourworldindata.org/palm-oil)). Without wishing to be limited to any particular theory, the present inventors believe that increasing the palmitic acid level in peanuts maybe advantageous because peanut butter made from such peanuts may not need to be stabilized using the typical addition of trans fatty acids, which have been correlated to negative health impacts.

The most common use of peanuts in the United States is as peanut butter with about 60% of the U.S. peanut crop being processed into a peanut butter product (American Peanut Council, peanutsusa.com/about-apc/the-peanut-industry). “Natural” peanut butter is susceptible to oil separation, which leads to lipid peroxidation and the development of off-flavor and rancidity. Such oil separation may affect textural quality of peanut butter in terms of spreadability (Gills, L. A. and Resurreccion, A. V. A. J. Food Engineering, 65:173-180 (2000)). Addressing the stabilization of peanut butter as a food product is important for food quality, manufacturing cost, and health reasons. Therefore, in the commercial production of peanut butter, stabilizers are used to improve its texture and mouthfeel and to create a shelf-stable, homogeneous product. Common stabilizers such as palm oil, cottonseed and canola oils are expensive and because they are hydrogenated vegetable oil, they are known to be factors associated with heart disease. Addressing the stabilization of peanut butter as a food product is important for food quality, manufacturing cost, and health reasons.

Peanut oil is typically composed of unsaturated 18:1 oleic acid (45-53%), 18:2 linoleic acid (27-32%), and saturated 16:0 palmitic acid (10-14%) (Kamdar et al., Genetic Resources in Crop Evolution, 68:529-549 (2021)). Palmitic acid (16:0; C₁₆H₃₂O₂) is solid at room temperature due to its saturated nature. Therefore, increasing palmitic acid levels in peanuts may be helpful in reducing or preventing oil separation in peanut butter prepared from the peanuts.

Since β-ketoacyl-ACP synthetase II (KasII) controls the ratio of palmitic acid (16:0) to stearic acid (18:0) in oil seeds (Shanlin et al. 4th International Conference on Bioinformatics and Biomedical Engineering, 2010, pp. 1-5), the present inventors have targeted the KAS II gene for deletion or down regulation, thereby reducing or eliminating KasII enzyme activity with the expectation of increasing palmitic acid levels in peanut seed oil from about 30% to about 65%. A KASII gene may be modified by any means known in the art including, but not limited to, traditional breeding, molecular breeding, TILLING mutagenesis, RNAi, and CRISPR. Thus, for example, CRISPR-based technologies may be used to develop novel peanut varieties in which the seeds may be used to produce a peanut butter without oil separation and with other benefits. Reduction of enzyme activity is accomplished by editing the KAS II gene, either by deleting part of the coding sequence, modifying the promoter, or mutating the coding sequence of KAS II to change the position of certain amino acids. By reducing or eliminating the enzyme activity of KasII enzymes in the peanut plant, the total amount of palmitic acid in a seed oil from a peanut plant having a mutated KASII gene may be increased to about 13% to 16.5% as compared to a total amount of palmitic acid of about 10% palmitic acid that is observed in wild type peanut seed oil, i.e., the seed oil of a peanut plant or part thereof devoid of the at least one mutation.

Accordingly, in some embodiments, the present invention is directed to generating mutations in β-ketoacyl-ACP synthetase II (KAS II) genes, optionally wherein the mutation is in an upstream/regulatory region, Exon 1, Exon 2, Exon 3 or Exon 7 and/or in the 3′ region of the KASII gene. In some embodiments, when the mutation is in the upstream region of the KASII gene, the mutation may be in a promoter or other regulatory element, which mutation may disrupt the expression of the KASII gene and therefore disrupt the production of the KasII protein.

In some embodiments, the present invention provides a peanut plant or part thereof comprising at least one (e.g., one or more; e.g., 1, 2, 3, 4, 5, 6, 7, 8, 9, 10 or more) mutation in one or more β-ketoacyl-ACP synthetase II (KASII) (also known as 3-oxoacyl-acyl carrier protein synthase II) genes encoding a KasII polypeptide. In some embodiments, the KAS II gene encoding the KasII polypeptide (a) comprises a nucleotide sequence having at least 80% sequence identity to SEQ ID NO:1, SEQ ID NO:2, SEQ ID NO:116, SEQ ID NO:117, SEQ ID NO:118, and/or SEQ ID NO:119, (b) comprises a region of consecutive nucleotides having at least 80% sequence identity to any one of the nucleotide sequences of SEQ ID NOs:5-31, 32-51, 52-69, 70-83 and/or 275-298, and/or SEQ ID NOs:122-148, 149-168, 169-188, 189-208, 209-212, and/or 299-326, and/or (c) encodes an amino acid sequence having at least 80% sequence identity to SEQ ID NO:4 and/or SEQ ID NO:121. In some embodiments, the one or more KAS II genes may be in the A genome of the peanut plant or part thereof (GenBank Accession No. LOC112696350 (SEQ ID NO:1 or SEQ ID NO:2) and/or in the B genome of the peanut plant or part thereof (GenBank Accession No. LOC112740759 (SEQ ID NOs:116-119). In some embodiments, a mutation in a KASII gene in a peanut plant or part thereof results in reduced or no expression by the KASII gene, and/or reduced or no production of the encoded KasII polypeptide. In some embodiments, a mutation in a KASII gene in a peanut plant or part thereof results in a KasII polypeptide having reduced or no enzyme activity (e.g., reduced or no ability to convert palmitic acid to stearic acid). In some embodiments, a mutation in a KASII gene in a peanut plant or part thereof results in increased palmitic acid in the seed oil of the peanut plant as compared to the seed oil of a peanut plant or plant part (e.g., an isogenic peanut plant) not comprising the same mutation.

In some embodiments, a peanut plant comprising at least one mutation in at least one KASII gene encoding a KasII protein exhibits a phenotype of increased palmitic acid in its seed oil (peanut oil) compared to an isogenic peanut plant (e.g., wild type unedited plant or a null segregant) that does not comprise the mutation. In some embodiments, a peanut plant comprising at least one mutation as described herein produces a mutated KASII gene having at least 90% identity to any one of the mutated KASII nucleotide sequences described herein.

In some embodiments, a KASII gene (a) comprises a nucleotide sequence having at least 80% sequence identity to the nucleotide sequence of any one of SEQ ID NO:1, SEQ ID NO:2, SEQ ID NO:116, SEQ ID NO:117, SEQ ID NO:118, or SEQ ID NO:119; (b) comprises a region of consecutive nucleotides having at least 80% sequence identity to any one of the nucleotide sequences of SEQ ID NOs:5-31, 32-51, 52-69, 70-83 and/or 275-298, and/or SEQ ID NOs:122-148, 149-168, 169-188, 189-208, 209-212, and/or 299-326; and/or (c) encodes a polypeptide sequence having at least 80% identity to the amino acid sequence of any one of SEQ ID NO:4 or SEQ ID NO:121.

A mutation in a β-ketoacyl-ACP synthetase II (KASII) gene in a peanut plant may be introduced by any method, including traditional breeding, molecular breeding, TILLING mutagenesis, RNAi, and gene editing (e.g., CRISPR, TALENS, meganuclease, ZFN and the like), optionally wherein the mutation is introduced using gene editing. The mutation that is introduced into a KASII gene may be any type of mutation including, but not limited to, a point mutation, a base substitution, a base deletion and/or a base insertion, optionally wherein the at least one mutation results in a frame shift mutation (in-frame or out-of-frame). A mutation useful with this invention can include, but is not limited to, a substitution, a deletion and/or an insertion of one or more bases in a regulatory region (upstream region) or in the coding region, optionally located in Exon 1, Exon 2, Exon 3, and/or Exon 7, and/or in the 3′ region of the KAS II gene. In some embodiments, at least one mutation may comprise a base substitution to an A, a T, a G, or a C, which optionally, results in frameshift mutation in the gene and/or reduces the activity of the KasII polypeptide. In some embodiments, a peanut plant comprising a KASII gene that has at least one mutation in a KASII gene as described herein exhibits an increase in palmitic acid in the seed oil of the peanut plant, as compared to a peanut plant that does not comprise the at least one mutation in a KASII gene. In some embodiments, a peanut plant comprising a mutation in a KASII gene may be heterozygous for the mutation. In some embodiments, a peanut plant comprising a mutation in a KASII gene may be homozygous for the mutation.

In some embodiments, the at least one mutation in a KASII gene may be a deletion or insertion (e.g., a deletion of one or more consecutive base pairs, e.g., at least one or at least two or more (e.g., about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more) (e.g., 110, 120, 130, 140, 150, and the like) consecutive base pairs of SEQ ID NO:1, SEQ ID NO:2, SEQ ID NO:116, SEQ ID NO:117, SEQ ID NO:118, and/or SEQ ID NO:119). In some embodiments, such a deletion or insertion may be in the region of a KASII gene that is 5′ of the first exon (upstream/regulatory region) or in the coding sequence of the KASII gene (e.g., in one or more exons; e.g., Exon 1, Exon 2, Exon 3, and/or Exon 7) and/or in the 3′ region of the KASII gene.

In some embodiments, a deletion or insertion may be in a regulatory (upstream) region of the KASII gene, wherein the deletion may be located from nucleotide 1 to about nucleotide 1500 with reference to nucleotide numbering of any one of SEQ ID NO:1, SEQ ID NO:2, SEQ ID NO:116, SEQ ID NO:117, SEQ ID NO:118, and/or SEQ ID NO:119. In some embodiments, the mutation may be located in a portion of the upstream region of a KASII gene having at least 80% sequence identity to SEQ ID NOs:5-31 or 122-148. In some embodiments the at least one mutation in the upstream region of the KASII gene results in the KAS II gene having reduced expression, thereby reducing production of the encoded Kas II polypeptide. Without wishing to be bound by any particular theory, the mutation in the upstream region is believed to modify regulatory regions, e.g., promoter regions, thereby reducing the expression of the gene and production of the encoded polypeptide, which in turn results in a reduction of conversion of palmitic acid to stearic acid and thus an increase in palmitic acid in the seed oil.

In some embodiments, a deletion or insertion may be in the coding region of the KASII gene, wherein the deletion may be located in one or more exons of the KASII gene, optionally in Exon 1, Exon 2, Exon 3, and/or Exon 7 of the KASII gene having at least 80% sequence identity to any one of the nucleotide sequences of SEQ ID NO:1, SEQ ID NO:2, SEQ ID NO:116, SEQ ID NO:117, SEQ ID NO:118, and/or SEQ ID NO:119. In some embodiments, the mutation may be located in Exon 1, Exon 2 and/or Exon 7, or region thereof, of a KASII gene having at least 80% sequence identity to any one of the nucleotide sequences of SEQ ID NO:1 or SEQ ID NO:2, optionally in a region having at least 80% sequence identity to any one of the nucleotide sequences of SEQ ID NOs:32-51, 52-69, or 70-83) or Exon 1, Exon 2, and/or Exon 3, or region thereof, of any one of the nucleotide sequences of SEQ ID NOs:116-119, optionally in a region having at least 80% sequence identity to any one of the nucleotide sequences of SEQ ID NOs:149-168, 169-188, 189-208.

In some embodiments, a deletion or insertion may be in the 3′ region of the KASII gene, optionally wherein the deletion is adjacent to or within a coding region located in the 3′ region of the KASII gene, the KASII gene having at least 80% sequence identity to any one of the nucleotide sequences of SEQ ID NO:1, SEQ ID NO:2, SEQ ID NO:116, SEQ ID NO:117, SEQ ID NO:118, and/or SEQ ID NO:119. In some embodiments, the mutation may be located in region of a KASII gene having at least 80% sequence identity to any one of the nucleotide sequences of SEQ ID NOs:275-298 and/or 299-326.

In some embodiments the at least one mutation in the coding region and/or in the 3′ region of a KASII gene results in a modified KasII polypeptide, a reduced amount of KasII polypeptide and/or no KasII polypeptide, optionally wherein a reduction in activity may be a reduction of about 5% to about 95% (e.g., a reduction in activity of about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95%, optionally a reduction of about 96, 97, 98, 99%, optionally no activity, e.g., a 100% reduction in activity). In some embodiments, the activity that is reduced or devoid is conversion of palmitic acid to stearic acid. In some embodiments, a modified KasII polypeptide comprises a truncated KasII polypeptide, optionally wherein the truncated KasII polypeptide exhibits reduced or no activity or the truncation of the KasII polypeptide results in undetectable levels of the KasII polypeptide. In some embodiments, the activity that is reduced or absent is conversion of palmitic acid to stearic acid.

In some embodiments, the at least one mutation may result in an amino acid substitution, optionally wherein the at least one mutation that results in an amino acid substitution is located in Exon 7 of the KAS II gene, optionally the mutation that results in an amino acid substitution may be located in a region of a KASII gene having at least 80% sequence identity to anyone of the nucleotide sequences of SEQ ID NOs:70-83 or 209-212. In some embodiments, the mutation results in Kas II polypeptide comprising an amino acid substitution at L344 with reference to amino acid position numbering of SEQ ID NO:4 and/or SEQ ID NO:121. In some embodiments, the amino acid substitution may be L344F (Leu>Phe at residue 344). In some embodiments, the amino acid substitution results in a modified or mutated KasII polypeptide that exhibits reduced activity or is devoid of activity, optionally wherein a reduction in activity may be a reduction of about 5% to about 95% (e.g., a reduction in activity of about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95%, optionally a reduction of about 96, 97, 98, 99%, optionally no activity, e.g., a 100% reduction in activity). In some embodiments, the activity that is reduced or absent is conversion of palmitic acid to stearic acid.

In some embodiments, a peanut plant or part thereof having a mutation (e.g., a deletion, insertion, substitution or combination thereof) in a KASII gene as described herein may be heterozygous for the mutation. In some embodiments, a peanut plant or part thereof having a mutation (e.g., a deletion, insertion, substitution or combination thereof) in a KASII gene as described herein may be homozygous for the mutation.

In some embodiments, a peanut plant part may be a cell, the cell comprising at least one (e.g., one or more; e.g., 1, 2, 3, 4, 5, 6, 7, 8, 9, 10 or more) mutation in one or more β-ketoacyl-ACP synthetase II (KASII) (also known as 3-oxoacyl-acyl carrier protein synthase II) genes encoding a KasII polypeptide, optionally wherein the KAS II gene encoding the KasII polypeptide (a) comprises a nucleotide sequence having at least 80% sequence identity to SEQ ID NO:1, SEQ ID NO:2, SEQ ID NO:116, SEQ ID NO:117, SEQ ID NO:118, and/or SEQ ID NO:119, (b) comprises a region of consecutive nucleotides having at least 80% sequence identity to any one of the nucleotide sequences of SEQ ID NOs:5-31, 32-51, 52-69, 70-83 and/or 275-298, and/or SEQ ID NOs:122-148, 149-168, 169-188, 189-208, 209-212, and/or 299-326; and/or (c) encodes a polypeptide sequence having at least 80% identity to the amino acid sequence of any one of SEQ ID NO:4 or SEQ ID NO:121. In some embodiments, the one or more KAS II genes may be in the A genome of the peanut plant cell thereof (GenBank Accession No. LOC112696350 (SEQ ID NO:1 or SEQ ID NO:2) and/or in the B genome of the peanut plant or part thereof (GenBank Accession No. LOC112740759 (SEQ ID NOs:116-119). In some embodiments, a peanut plant part (e.g., plant cell) having a mutation (e.g., a deletion, insertion, substitution or combination thereof) in a KASII gene as described herein may be heterozygous for the mutation. In some embodiments, a peanut plant part (e.g., plant cell) having a mutation (e.g., a deletion, insertion, substitution or combination thereof) in a KASII gene as described herein may be homozygous for the mutation.

In some embodiments, a peanut plant cell may be regenerated into a peanut plant, wherein the plant exhibits in reduced or no expression by the KASII gene, and/or reduced or no production of the encoded KasII polypeptide. In some embodiments, the regenerated peanut plant having at least one mutation in a KASII gene may have reduced or no KasII enzyme activity (e.g., reduced or no ability to convert palmitic acid to stearic acid). In some embodiments, a mutation in a KASII gene in the regenerated peanut plant results in increased palmitic acid in the seed oil of the regenerated peanut plant as compared to the seed oil of a peanut plant or plant part (e.g., an isogenic peanut plant) not comprising the same mutation. In some embodiments, the peanut cell may not be regenerated into a plant.

In some embodiments, a peanut plant or part thereof comprising a mutation as described herein can be a peanut plant or part thereof, wherein the peanut plant or part thereof comprises at least one mutation in a β-ketoacyl-ACP synthetase II (KAS II) gene having the GenBank Accession No. LOC112696350 (SEQ ID NO:1 or SEQ ID NO:2) and/or GenBank Accession No. LOC112740759 (SEQ ID NOs:116-119). In some embodiments, a peanut plant or part thereof comprising a mutation as described herein may comprise the least one mutation in a KASII gene that is in the A genome and/or the B genome of the peanut plant.

A peanut plant comprising a mutation in a KASII gene as described herein which results in no or reduced KasII polypeptide and/or a modified KasII polypeptide having reduced or no activity results in an increase in palmitic acid in the peanut seed oil. In some embodiments, the increase in palmitic acid in the seed oil may be from about 30% to about 65% (e.g., about 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, or 65%). Thus, the total amount of palmitic acid may be increased from about 10% (the amount present in the seed oil of a peanut plant devoid of the mutation) to an amount of about 13% to 16.5% (e.g., about 13, 13.1, 13.2, 13.3, 13.4, 13.5, 13.6, 13.7, 13.8, 13.9, 14, 14.1, 14.2, 14.3, 14.4, 14.5, 14.6, 14.7, 14.8, 14.9, 15, 15.1, 15.2, 15.3, 15.4, 15.5, 15.6, 15.7, 15.8, 15.9, 16, 16.1, 16.2, 16.3, 16.4, or 16.5%) as compared to a control peanut plant or part thereof devoid of the at least one mutation. In some embodiments, a peanut plant comprising the at least one mutation may produces seed (peanuts) (e.g., part thereof) having oil (seed oil) with an increased ratio of palmitic acid to stearic acid, optionally wherein the increase is a doubling of the ratio of palmitic to stearic acid as compared to seed from a peanut plant devoid of the at least one mutation, optionally wherein the ratio of palmitic acid to stearic acid is increased from a ratio of 4:1 to a ratio of 8:1 (e.g., a ratio of palmitic acid to stearic acid of about 4:1, 5:1, 6:1, 7:1 or 8:1).

In some embodiments, a peanut plant or part thereof edited as described herein comprises a mutated KASII gene having at least 90% sequence identity to any one of the mutated KAS genes described herein. In some embodiments, a part of a peanut plant is a peanut cell and/or a peanut seed. In some embodiments, a peanut cell comprising a mutated KASII gene as described herein may be regenerated into a peanut plant. In some embodiments a peanut seed comprising a mutated KASII gene as described herein may be germinated and grown into a peanut plant.

In some embodiments, a method of producing a peanut plant having seed oil with increased levels of palmitic acid is provided, the method comprising: (a) contacting a peanut plant cell comprising an KASII gene with a nuclease targeting the KASII gene, wherein the nuclease is linked to a nucleic acid binding domain (e.g., editing system) that binds to a target site in the KASII gene, wherein the KASII gene: (i) comprises a nucleotide sequence having at least 80% sequence identity to any one of SEQ ID NO:1, SEQ ID NO:2, SEQ ID NO:116, SEQ ID NO:117, SEQ ID NO:118, and/or SEQ ID NO:119, (ii) comprises a region of consecutive nucleotides having at least 80% sequence identity to any one of the nucleotide sequences of SEQ ID NOs:5-31, 32-51, 52-69, 70-83 and/or 275-298, and/or SEQ ID NOs:122-148, 149-168, 169-188, 189-208, 209-212, and/or 299-326, and/or (iii) encodes an amino acid sequence having at least 80% sequence identity to SEQ ID NO:4 and/or SEQ ID NO:121; and (b) growing the peanut plant cell into a peanut plant, wherein the peanut plant comprises a mutation in the KASII gene, thereby producing a peanut plant having seed oil with an increased level of palmitic acid.

In some embodiments, a method of increasing the level of palmitic acid in the seed oil of a peanut plant is provided, the method comprising: (a) contacting a peanut plant cell comprising an endogenous KASII gene with a nuclease targeting the KASII gene, wherein the nuclease is linked to a nucleic acid binding domain (e.g., editing system) that binds to a target site in the KASII gene, wherein the KASII gene: (i) comprises a nucleotide sequence having at least 80% sequence identity to SEQ ID NO:1, SEQ ID NO:2, SEQ ID NO:116, SEQ ID NO:117, SEQ ID NO:118, and/or SEQ ID NO:119, (ii) comprises a region of consecutive nucleotides having at least 80% sequence identity to any one of the nucleotide sequences of SEQ ID NOs:5-31, 32-51, 52-69, 70-83 and/or 275-298, and/or SEQ ID NOs:122-148, 149-168, 169-188, 189-208, 209-212, and/or 299-326, and/or (iii) encodes an amino acid sequence having at least 80% sequence identity to SEQ ID NO:4 and/or SEQ ID NO:121; and (b) growing the peanut plant cell into a peanut plant comprising the mutation in the KASII gene, thereby producing a peanut plant have a mutated KASII gene and comprising seed oil with an increased level of palmitic acid.

Further provided is a method of reducing oil separation in peanut butter, the method comprising preparing peanut butter from the seed of a plant of the present invention comprising at least one mutation in a KASII gene as described herein.

In some embodiments, the invention provides a method of reducing oil separation in peanut butter, the method comprising (a) contacting a peanut plant comprising an endogenous KASII gene with a nuclease targeting the KASII gene, wherein the nuclease is linked to a nucleic acid binding domain (e.g., editing system) that binds to a target site in the KASII gene, wherein the KASII gene: (i) comprises a nucleotide sequence having at least 80% sequence identity to SEQ ID NO:1, SEQ ID NO:2, SEQ ID NO:116, SEQ ID NO:117, SEQ ID NO:118, and/or SEQ ID NO:119, (ii) comprises a region of consecutive nucleotides having at least 80% sequence identity to any one of the nucleotide sequences of SEQ ID NOs:5-31, 32-51, 52-69, 70-83 and/or 275-298, and/or SEQ ID NOs:122-148, 149-168, 169-188, 189-208, 209-212, and/or 299-326, and/or (iii) encodes an amino acid sequence having at least 80% sequence identity to SEQ ID NO:4 and/or SEQ ID NO:121; and (b) growing the peanut plant cell into a peanut plant comprising the mutation in the KASII gene, thereby producing a peanut plant have a mutated KASII gene and exhibiting an increase level of palmitic acid in the seed oil of the peanut plant; and preparing peanut butter from the seed, wherein the peanut butter has reduced oil or no oil separation. In some embodiments, the separation of the oil in a peanut butter is reduced by about 50% to about 90% or more (e.g., 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90%, optionally a reduction in separation of about 91, 92, 93, 94, 9596, 97, 98, 99%, optionally no separation, e.g., 100% reduction in separation.

In some embodiments, a method of providing peanut oil with increased levels of palmitic acid is provided, the method comprising preparing peanut oil from the seed of a peanut plant of the invention comprising at least one mutation in a KASII gene as described herein.

Additionally provided is a method of providing peanut oil with increased levels of palmitic acid, the method comprising (a) contacting a peanut plant comprising an endogenous KASII gene with a nuclease targeting the KASII gene, wherein the nuclease is linked to a nucleic acid binding domain (e.g., editing system) that binds to a target site in the KASII gene, wherein the KASII gene: (i) comprises a nucleotide sequence having at least 80% sequence identity to SEQ ID NO:1, SEQ ID NO:2, SEQ ID NO:116, SEQ ID NO:117, SEQ ID NO:118, and/or SEQ ID NO:119, (ii) comprises a region of consecutive nucleotides having at least 80% sequence identity to any one of the nucleotide sequences of SEQ ID NOs:5-31, 32-51, 52-69, 70-83 and/or 275-298, and/or SEQ ID NOs:122-148, 149-168, 169-188, 189-208, 209-212, and/or 299-326, and/or (iii) encodes an amino acid sequence having at least 80% sequence identity to SEQ ID NO:4 and/or SEQ ID NO:121, and (b) growing the peanut plant cell into a peanut plant comprising the mutation in the KASII gene, thereby producing a peanut plant have a mutated KASII gene and exhibiting an increase level of palmitic acid in the seed oil of the peanut plant.

In some embodiments, the methods of the invention provide an increase in palmitic acid in the seed oil of a peanut plant from about 13% to about 16.5% (e.g., about 13, 13.1, 13.2, 13.3, 13.4, 13.5, 13.6, 13.7, 13.8, 13.9, 14, 14.1, 14.2, 14.3, 14.4, 14.5, 14.6, 14.7, 14.8, 14.9, 15, 15.1, 15.2, 15.3, 15.4, 15.5, 15.6, 15.7, 15.8, 15.9, 16, 16.1, 16.2, 16.3, 16.4, or 16.5%) as compared to a peanut plant devoid of the mutation. In some embodiments, the methods of the invention provide an increased ratio of palmitic acid to stearic acid in the seed oil, optionally wherein the increase is a doubling of the ratio of palmitic to stearic acid as compared to seed from a peanut plant devoid of the at least one mutation, optionally wherein the ratio of palmitic acid to stearic acid is increased from a ratio of 4:1 to a ratio of 8:1 (e.g., a ratio of palmitic acid to stearic acid of about 4:1, 5:1, 6:1, 7:1 or 8:1).

In some embodiments, the target site is in a region of the KASII gene located from about nucleotide 1 to about nucleotide 1500 with reference to nucleotide numbering of SEQ ID NO:1, SEQ ID NO:2, SEQ ID NO:116, SEQ ID NO:117, SEQ ID NO:118, and/or SEQ ID NO:119. In some embodiments, the target site is in a coding region of the KASII gene, optionally in Exon 1, Exon 2, Exon 3, and/or Exon 7 of a KASII gene. In some embodiments, the target site is in the 3′ region of a KASII gene, optionally within or adjacent to a coding region, optionally in or adjacent to Exon 10, Exon 11, Exon 12, and/or Exon 13. In some embodiments, the target site is in a region of consecutive nucleotides of the KASII gene having at least 80% sequence identity to any one of the nucleotide sequences of SEQ ID NOs:5-31, 32-51, 52-69, 70-83 and/or 275-298, and/or SEQ ID NOs:122-148, 149-168, 169-188, 189-208, 209-212, and/or 299-326.

In some embodiments, a nuclease contacting a peanut plant cell, a population of peanut plant cells and/or a target site cleaves an KASII gene, thereby introducing a mutation into the KASII gene, optionally wherein the mutation is introduced into a region of the KASII gene that is in a regulatory element of the KASII gene, in the coding region of the KASII gene, optionally in Exon 1, Exon 2, Exon 3 or Exon 7 of the KASII gene, optionally wherein the mutation is introduced into a region of consecutive nucleotides of a KASII gene having at least 80% sequence identity to any one of the nucleotide sequences of SEQ ID NOs:5-31, 32-51, 52-69, 70-83 and/or 275-298, and/or SEQ ID NOs:122-148, 149-168, 169-188, 189-208, 209-212, and/or 299-326. In some embodiments, the mutation may be a base substitution, a base insertion and/or a base deletion. In some embodiments, the mutation results in a mutated KASII gene having at least 90% identity to any one of one of the mutated KASII genes as described herein.

A nuclease useful with the invention may be any nuclease that can be utilized to edit/modify a target nucleic acid. Such nucleases include, but are not limited to, a zinc finger nuclease, transcription activator-like effector nucleases (TALEN), endonuclease (e.g., Fok1) and/or a CRISPR-Cas effector protein. Likewise, any nucleic acid binding domain useful with the nuclease of the invention may be any DNA binding domain that can be utilized to edit/modify a target nucleic acid. Such DNA binding domains include, but are not limited to, a zinc finger, transcription activator-like DNA binding domain (TAL), an argonaute and/or a CRISPR-Cas effector DNA binding domain.

In some embodiments, a nucleic acid encoding a mutated KASII gene from peanut is provided, optionally wherein the mutation is a base substitution, a base deletion, and/or a base insertion in a peanut KASII gene. In some embodiments, the peanut KASII gene: (a) comprises a nucleotide sequence having at least 80% sequence identity to SEQ ID NO:1, SEQ ID NO:2, SEQ ID NO:116, SEQ ID NO:117, SEQ ID NO:118, and/or SEQ ID NO:119, (b) comprises a region of consecutive nucleotides having at least 80% sequence identity to any one of the nucleotide sequences of SEQ ID NOs:5-31, 32-51, 52-69, 70-83 and/or 275-298, and/or SEQ ID NOs:122-148, 149-168, 169-188, 189-208, 209-212, and/or 299-326, and/or (c) encodes an amino acid sequence having at least 80% sequence identity to SEQ ID NO:4 and/or SEQ ID NO:121. In some embodiments, the nucleic acid encoding a mutated KASII gene encodes an amino acid sequence as modified herein. In some embodiments, a peanut plant or part thereof is provided that comprises the nucleic acid encoding the mutated KASII gene.

In some embodiments, the present invention provides a guide nucleic acid (e.g., gRNA, gDNA, crRNA, crDNA) that binds to a target site in a β-ketoacyl-ACP synthetase II (KASII) gene, the KASII gene: (a) comprising a nucleotide sequence having at least 80% sequence identity to SEQ ID NO:1, SEQ ID NO:2, SEQ ID NO:116, SEQ ID NO:117, SEQ ID NO:118, and/or SEQ ID NO:119, (b) comprising a region of consecutive nucleotides having at least 80% sequence identity to any one of the nucleotide sequences of SEQ ID NOs:5-31, 32-51, 52-69, 70-83 and/or 275-298, and/or SEQ ID NOs:122-148, 149-168, 169-188, 189-208, 209-212, and/or 299-326, and/or (c) encoding an amino acid sequence having at least 80% sequence identity to SEQ ID NO:4 and/or SEQ ID NO:121. In some embodiments, the guide nucleic acid binds within a target site in a KASII gene, the target site having at least 80% sequence identity to any one of SEQ ID NOs:5-31, 32-51, 52-69, 70-83 and/or 275-298, and/or SEQ ID NOs:122-148, 149-168, 169-188, 189-208, 209-212, and/or 299-326, or a portion of consecutive nucleotides thereof. In some embodiments, the guide nucleic acid comprises a spacer comprising the nucleotide sequence of SEQ ID NOs:84-95, 213-224, 241-246 and/or 257-262 (e.g., SEQ ID NOs:84-86, 87-89, 90-91, 92-95, 213, 214 and 85, 215, 216 and 88, 217-219, 220-223, 224 and 91, 241-246 and/or 257-262).

Example spacer sequences useful with a guide of this invention may have complementarity to a fragment or portion (or region) of a nucleotide sequence (a) having at least 80% sequence identity to the nucleotide sequence of any one of SEQ ID NO:1, SEQ ID NO:2, SEQ ID NO:116, SEQ ID NO:117, SEQ ID NO:118, and/or SEQ ID NO:119; (b) having at least 80% sequence identity to a region of consecutive nucleotides of any one of SEQ ID NOs:5-31, 32-51, 52-69, 70-83 and/or 275-298, and/or SEQ ID NOs:122-148, 149-168, 169-188, 189-208, 209-212, and/or 299-326; and/or (d) encoding a polypeptide sequence having at least 80% identity to the amino acid sequence of any one of SEQ ID NO:4 and/or SEQ ID NO:121.

A target nucleic acid may be any KASII gene in a peanut plant or part thereof, which may be modified as described herein, resulting in the peanut plant exhibiting an increased level of palmitic acid in its seed oil (e.g., in the oil of the peanuts produced by the edited peanut plant). In some embodiments, a target site in a target nucleic acid may comprise a sequence having at least 80% sequence identity to a region, portion or fragment of any one of SEQ ID NO:1, SEQ ID NO:2, SEQ ID NO:116, SEQ ID NO:117, SEQ ID NO:118, and/or SEQ ID NO:119, (e.g., SEQ ID NOs:5-31, 32-51, 52-69, 70-83 and/or 275-298, and/or SEQ ID NOs:122-148, 149-168, 169-188, 189-208, 209-212, and/or 299-326).

In some embodiments, a guide nucleic acid comprises a spacer having the nucleotide sequence of any one of SEQ ID NOs:84-95, 213-224, 241-246 and/or 257-262 (e.g., SEQ ID NOs:84-86, 87-89, 90-91, 92-95, 213, 214 and 85, 215, 216 and 88, 217-219, 220-223, 224 and 91, 241-246 and/or 257-262)). See, Table 1 and Table 2.

Also provided herein are expression cassettes comprising (a) polynucleotide encoding CRISPR-Cas effector protein comprising a cleavage domain and (b) a guide nucleic acid that binds to a target site in an KASII gene, wherein the guide nucleic acid comprises a spacer sequence that is complementary to and binds to: (i) a portion of consecutive nucleotides from a nucleic acid having at least 80% sequence identity to any one of SEQ ID NOs:1, 2, and/or 116-119; (ii) a portion of consecutive nucleotides from a nucleic acid sequence having at least 80% sequence identity to any one of SEQ ID NOs:5-83 (e.g., A genome), SEQ ID NOs:122-212 (e.g., B genome), SEQ ID NOs:275-298 (e.g., A genome) or SEQ ID NOs:299-326 (e.g., B genome); and/or (iii) a portion of a consecutive nucleotides from a nucleic acid encoding an amino acid sequence having at least 80% sequence identity to any one of SEQ ID NO:4 or SEQ ID NO:121. In some embodiments, the target site in a KASII gene is in a region of consecutive nucleotides of the KASII gene having at least about 80% sequence identity to any one of SEQ ID NOs:5-31, 32-51, 52-69, 70-83 and/or 275-298 (e.g., A genome), and/or SEQ ID NOs:122-148, 149-168, 169-188, 189-208, 209-212, and/or 299-326 (e.g., B genome).

A system for editing a KASII gene can be any site-specific (sequence-specific) genome editing system now known or later developed, which system can introduce mutations in target specific manner. For example, an editing system (e.g., site- or sequence-specific editing system) can include, but is not limited to, a CRISPR-Cas editing system, a meganuclease editing system, a zinc finger nuclease (ZFN) editing system, a transcription activator-like effector nuclease (TALEN) editing system, and/or a base editing system, each of which can comprise one or more polypeptides and/or one or more polynucleotides that when expressed as a system in a cell can modify (mutate) a target nucleic acid in a sequence specific manner. In some embodiments, an editing system (e.g., site- or sequence-specific editing system) can comprise one or more polynucleotides and/or one or more polypeptides, including but not limited to a nucleic acid binding domain (DNA binding domain), a nuclease, and/or other polypeptide, and/or a polynucleotide, and/or a guide nucleic acid (comprising a spacer having substantial complementarity or full complementarity to a target site).

In some embodiments, an editing system can comprise one or more sequence-specific nucleic acid binding domains (e.g., sequence-specific DNA binding domains) that can be from, for example, a polynucleotide-guided endonuclease, a CRISPR-Cas endonuclease (e.g., CRISPR-Cas effector protein), a zinc finger nuclease, a transcription activator-like effector nuclease (TALEN) and/or an Argonaute protein. In some embodiments, an editing system can comprise one or more cleavage domains (e.g., nucleases) including, but not limited to, an endonuclease (e.g., Fok1), a polynucleotide-guided endonuclease, a CRISPR-Cas endonuclease (e.g., CRISPR-Cas effector protein), a zinc finger nuclease, and/or a transcription activator-like effector nuclease (TALEN).

In some embodiments, a sequence-specific nucleic acid binding domain (DNA binding domains) of an editing system useful with this invention can be from, for example, a polynucleotide-guided endonuclease, a CRISPR-Cas endonuclease (e.g., CRISPR-Cas effector protein), a zinc finger nuclease, a transcription activator-like effector nuclease (TALEN) and/or an Argonaute protein.

In some embodiments, a sequence-specific nucleic acid binding domain/protein may be a CRISPR-Cas effector protein, optionally wherein the CRISPR-Cas effector protein may be from a Type I CRISPR-Cas system, a Type II CRISPR-Cas system, a Type III CRISPR-Cas system, a Type IV CRISPR-Cas system, Type V CRISPR-Cas system, or a Type VI CRISPR-Cas system. In some embodiments, a CRISPR-Cas effector protein of the invention may be from a Type II CRISPR-Cas system or a Type V CRISPR-Cas system. In some embodiments, a CRISPR-Cas effector protein may be Type II CRISPR-Cas effector protein, for example, a Cas9 effector protein. In some embodiments, a CRISPR-Cas effector protein may be Type V CRISPR-Cas effector protein, for example, a Cas12 effector protein.

As used herein, a “CRISPR-Cas effector protein” is a protein or polypeptide or domain thereof that cleaves or cuts a nucleic acid, binds a nucleic acid (e.g., a target nucleic acid and/or a guide nucleic acid), and/or that identifies, recognizes, or binds a guide nucleic acid as defined herein. In some embodiments, a CRISPR-Cas effector protein may be an enzyme (e.g., a nuclease, endonuclease, nickase, etc.) or portion thereof and/or may function as an enzyme. In some embodiments, a CRISPR-Cas effector protein refers to a CRISPR-Cas nuclease polypeptide or domain thereof that comprises nuclease activity or in which the nuclease activity has been reduced or eliminated, and/or comprises nickase activity or in which the nickase has been reduced or eliminated, and/or comprises single stranded DNA cleavage activity (ss DNAse activity) or in which the ss DNAse activity has been reduced or eliminated, and/or comprises self-processing RNAse activity or in which the self-processing RNAse activity has been reduced or eliminated. A CRISPR-Cas effector protein may bind to a target nucleic acid.

In some embodiments, a CRISPR-Cas effector protein may include, but is not limited to, a Cas9, C2c1, C2c3, Cas12a (also referred to as Cpf1), Cas12b, Cas12c, Cas12d, Cas12e, Cas13a, Cas13b, Cas13c, Cas13d, Cas1, Cas1B, Cas2, Cas3, Cas3′, Cas3″, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4 (dinG), and/or Csf5 nuclease, optionally wherein the CRISPR-Cas effector protein may be a Cas9, Cas12a (Cpf1), Cas12b, Cas12c (C2c3), Cas12d (CasY), Cas12e (CasX), Cas12g, Cas12h, Cas12i, C2c4, C2c5, C2c8, C2c9, C2c10, Cas14a, Cas14b, and/or Cas14c effector protein.

In some embodiments, a CRISPR-Cas effector protein useful with the invention may comprise a mutation in its nuclease active site (e.g., RuvC, HNH, e.g., RuvC site of a Cas12a nuclease domain; e.g., RuvC site and/or HNH site of a Cas9 nuclease domain). A CRISPR-Cas effector protein having a mutation in its nuclease active site, and therefore, no longer comprising nuclease activity, is commonly referred to as “dead,” e.g., dCas. In some embodiments, a CRISPR-Cas effector protein domain or polypeptide having a mutation in its nuclease active site may have impaired activity or reduced activity as compared to the same CRISPR-Cas effector protein without the mutation, e.g., a nickase, e.g., Cas9 nickase, Cas12a nickase.

A CRISPR-Cas effector protein may be from a Type I, a Type II, a Type III, a Type IV, or a Type V system. In some embodiments, a CRISPR-Cas effector protein may be Cas9 effector protein or CRISPR Cas9 effector domain useful with this invention may be any known or later identified Cas9 polypeptide or a Cas9 nuclease.

In some embodiments, the CRISPR-Cas effector protein may be a Cas9 polypeptide derived from Streptococcus pyogenes and recognizes the PAM sequence motif NGG, NAG, NGA (Mali et al, Science 2013; 339(6121): 823-826). In some embodiments, the CRISPR-Cas effector protein may be a Cas9 polypeptide derived from Streptococcus thermophiles and recognizes the PAM sequence motif NGGNG and/or NNAGAAW (W=A or T) (See, e.g., Horvath et al, Science, 2010; 327(5962): 167-170, and Deveau et al, J Bacteriol 2008; 190(4): 1390-1400). In some embodiments, the CRISPR-Cas effector protein may be a Cas9 polypeptide derived from Streptococcus mutans and recognizes the PAM sequence motif NGG and/or NAAR (R=A or G) (See, e.g., Deveau et al, J BACTERIOL 2008; 190(4): 1390-1400). In some embodiments, the CRISPR-Cas effector protein may be a Cas9 polypeptide derived from Streptococcus aureus and recognizes the PAM sequence motif NNGRR (R=A or G). In some embodiments, the CRISPR-Cas effector protein may be a Cas9 protein derived from S. aureus, which recognizes the PAM sequence motif N GRRT (R=A or G). In some embodiments, the CRISPR-Cas effector protein may be a Cas9 polypeptide derived from S. aureus, which recognizes the PAM sequence motif N GRRV (R=A or G). In some embodiments, the CRISPR-Cas effector protein may be a Cas9 polypeptide that is derived from Neisseria meningitidis and recognizes the PAM sequence motif N GATT or N GCTT (R=A or G, V=A, G or C) (See, e.g., Hou et ah, PNAS 2013, 1-6). In the aforementioned embodiments, N can be any nucleotide residue, e.g., any of A, G, Cor T. In some embodiments, the CRISPR-Cas effector protein may be a Cas13a protein derived from Leptotrichia shahii, which recognizes a protospacer flanking sequence (PFS) (or RNA PAM (rPAM)) sequence motif of a single 3′ A, U, or C, which may be located within the target nucleic acid.

In some embodiments, the CRISPR-Cas effector protein may be derived from a Type V CRISPR system such as a Cas12a polypeptide, which is a Type V Clustered Regularly Interspaced Short Palindromic Repeats (CRISPR)-Cas nuclease

The nucleic acid constructs of the invention comprising a CRISPR-Cas effector protein may be used in combination with a guide RNA (gRNA, CRISPR array, CRISPR RNA, crRNA), designed to function with the encoded CRISPR-Cas effector protein or domain, to modify a target nucleic acid. A guide nucleic acid useful with this invention comprises at least one spacer sequence and at least one repeat sequence. The guide nucleic acid is capable of forming a complex with the CRISPR-Cas nuclease domain encoded and expressed by a nucleic acid construct of the invention and the spacer sequence is capable of hybridizing to a target nucleic acid, thereby guiding the complex to the target nucleic acid, wherein the target nucleic acid may be modified (e.g., cleaved or edited).

A “guide nucleic acid,” “guide RNA,” “gRNA,” “CRISPR RNA/DNA” “crRNA” or “crDNA” as used herein means a nucleic acid that comprises at least one spacer sequence, which is complementary to (and hybridizes to) a target DNA (e.g., protospacer), and at least one repeat sequence (e.g., a repeat of a Type V Cas12a CRISPR-Cas system, or a fragment or portion thereof; a repeat of a Type II Cas9 CRISPR-Cas system, or fragment thereof; a repeat of a Type V C2c1 CRISPR Cas system, or a fragment thereof; a repeat of a CRISPR-Cas system of, for example, C2c3, Cas12a (also referred to as Cpf1), Cas12b, Cas12c, Cas12d, Cas12e, Cas13a, Cas13b, Cas13c, Cas13d, Cas1, Cas1B, Cas2, Cas3, Cas3′, Cas3″, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4 (dinG), and/or Csf5, or a fragment thereof), wherein the repeat sequence may be linked to the 5′ end and/or the 3′ end of the spacer sequence. The design of a gRNA of this invention may be based on a Type I, Type II, Type III, Type IV, Type V, or Type VI CRISPR-Cas system.

In some embodiments, a guide nucleic acid may comprise more than one repeat sequence-spacer sequence (e.g., 2, 3, 4, 5, 6, 7, 8, 9, 10, or more repeat-spacer sequences) (e.g., repeat-spacer-repeat, e.g., repeat-spacer-repeat-spacer-repeat-spacer-repeat-spacer-repeat-spacer, and the like). The guide nucleic acids of this invention are synthetic, human-made and not found in nature. A gRNA can be quite long and may be used as an aptamer (like in the MS2 recruitment strategy) or other RNA structures hanging off the spacer.

A “repeat sequence” as used herein, refers to, for example, any repeat sequence of a wild-type CRISPR Cas locus (e.g., a Cas9 locus, a Cas12a locus, a C2c1 locus, etc.) or a repeat sequence of a synthetic crRNA that is functional with the CRISPR-Cas effector protein encoded by the nucleic acid constructs of the invention. A repeat sequence useful with this invention can be any known or later identified repeat sequence of a CRISPR-Cas locus (e.g., Type I, Type II, Type III, Type IV, Type V or Type VI) or it can be a synthetic repeat designed to function in a Type I, II, III, IV, V or VI CRISPR-Cas system. A repeat sequence may comprise a hairpin structure and/or a stem loop structure. In some embodiments, a repeat sequence may form a pseudoknot-like structure at its 5′ end (i.e., “handle”). Thus, in some embodiments, a repeat sequence can be identical to or substantially identical to a repeat sequence from wild-type Type I CRISPR-Cas loci, Type II, CRISPR-Cas loci, Type III, CRISPR-Cas loci, Type IV CRISPR-Cas loci, Type V CRISPR-Cas loci and/or Type VI CRISPR-Cas loci. A repeat sequence from a wild-type CRISPR-Cas locus may be determined through established algorithms, such as using the CRISPRfinder offered through CRISPRdb (see, Grissa et al. Nucleic Acids Res. 35(Web Server issue):W52-7). In some embodiments, a repeat sequence or portion thereof is linked at its 3′ end to the 5′ end of a spacer sequence, thereby forming a repeat-spacer sequence (e.g., guide nucleic acid, guide RNA/DNA, crRNA, crDNA).

In some embodiments, a repeat sequence comprises, consists essentially of, or consists of at least 10 nucleotides depending on the particular repeat and whether the guide nucleic acid comprising the repeat is processed or unprocessed (e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 to 100 or more nucleotides, or any range or value therein). In some embodiments, a repeat sequence comprises, consists essentially of, or consists of about 10 to about 20, about 10 to about 30, about 10 to about 45, about 10 to about 50, about 15 to about 30, about 15 to about 40, about 15 to about 45, about 15 to about 50, about 20 to about 30, about 20 to about 40, about 20 to about 50, about 30 to about 40, about 40 to about 80, about 50 to about 100 or more nucleotides.

A repeat sequence linked to the 5′ end of a spacer sequence can comprise a portion of a repeat sequence (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more contiguous nucleotides of a wild type repeat sequence). In some embodiments, a portion of a repeat sequence linked to the 5′ end of a spacer sequence can be about five to about ten consecutive nucleotides in length (e.g., about 5, 6, 7, 8, 9, 10 nucleotides) and have at least 90% sequence identity (e.g., at least about 90%, 91%, 92%, 93%, 94%, 95%, 96%, 97%, 98%, 99%, or more) to the same region (e.g., 5′ end) of a wild type CRISPR Cas repeat nucleotide sequence. In some embodiments, a portion of a repeat sequence may comprise a pseudoknot-like structure at its 5′ end (e.g., “handle”).

A “spacer sequence” as used herein is a nucleotide sequence that is complementary to a target nucleic acid (e.g., target DNA) (e.g., protospacer) (e.g., a portion of consecutive nucleotides of a β-ketoacyl-ACP synthetase II (KASII) gene, wherein the KASII gene (a) comprises a nucleotide sequence having at least 80% sequence identity to the nucleotide sequence of any one of SEQ ID NO:1, SEQ ID NO:2, SEQ ID NO:116, SEQ ID NO:117, SEQ ID NO:118, and/or SEQ ID NO:119; (b) comprises a region of consecutive nucleotides having at least 80% sequence identity to any one of the nucleotide sequences of SEQ ID NOs:5-31, 32-51, 52-69, 70-83 and/or 275-298, and/or SEQ ID NOs:122-148, 149-168, 169-188, 189-208, 209-212, and/or 299-326, and/or (c) encodes an amino acid sequence having at least 80% sequence identity to SEQ ID NO:4 and/or SEQ ID NO:121). A spacer sequence can be fully complementary or substantially complementary (e.g., at least about 70% complementary (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to a target nucleic acid. In some embodiments, the spacer sequence can have one, two, three, four, or five mismatches as compared to the target nucleic acid, which mismatches can be contiguous or noncontiguous. In some embodiments, the spacer sequence can have 70% complementarity to a target nucleic acid. In other embodiments, the spacer nucleotide sequence can have 80% complementarity to a target nucleic acid. In still other embodiments, the spacer nucleotide sequence can have 85%, 90%, 95%, 96%, 97%, 98%, 99% or 99.5% complementarity, and the like, to the target nucleic acid (protospacer). In some embodiments, the spacer sequence is 100% complementary to the target nucleic acid. A spacer sequence may have a length from about 15 nucleotides to about 30 nucleotides (e.g., 15, 16, 17, 18, 19, 20, 21, 22, 23, 24, 25, 26, 27, 28, 29, or 30 nucleotides, or any range or value therein), optionally about 20 to about 25 nucleotides in length. Thus, in some embodiments, a spacer sequence may have complete complementarity or substantial complementarity over a region of a target nucleic acid (e.g., protospacer) that is at least about 15 nucleotides to about 30 nucleotides in length, optionally about 20 to about 25 nucleotides (e.g., 20, 21, 22, 23, 24, or 25) in length, or any range or value therein. In some embodiments, the spacer is about 21, 22, 23 or 24 nucleotides in length. In some embodiments, a spacer sequence useful with this invention may comprise the nucleotide sequence of any one of SEQ ID NOs:84-95 and/or 241-246 (A genome) and/or SEQ ID NOs:213-224 and/or 257-262 (B genome), or any combination thereof (see, e.g., Tables 1 and 2).

In some embodiments, the 5′ region of a spacer sequence of a guide nucleic acid may be identical to a target DNA, while the 3′ region of the spacer may be substantially complementary to the target DNA (such as for a Type V CRISPR-Cas system), or the 3′ region of a spacer sequence of a guide nucleic acid may be identical to a target DNA, while the 5′ region of the spacer may be substantially complementary to the target DNA (such as for a Type II CRISPR-Cas system), and therefore, the overall complementarity of the spacer sequence to the target DNA may be less than 100%. Thus, for example, in a guide for a Type V CRISPR-Cas system, the first 1, 2, 3, 4, 5, 6, 7, 8, 9, 10 nucleotides in the 5′ region (i.e., seed region) of, for example, a 20 nucleotide spacer sequence may be 100% complementary to the target DNA, while the remaining nucleotides in the 3′ region of the spacer sequence are substantially complementary (e.g., at least about 70% complementary) to the target DNA. In some embodiments, the first 1 to 8 nucleotides (e.g., the first 1, 2, 3, 4, 5, 6, 7, 8, nucleotides, and any range therein) of the 5′ end of the spacer sequence may be 100% complementary to the target DNA, while the remaining nucleotides in the 3′ region of the spacer sequence are substantially complementary (e.g., at least about 50% complementary (e.g., 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to the target DNA.

As a further example, in a guide for a Type II CRISPR-Cas system, the first 1, 2, 3, 4, 5, 6, 7, 8, 9, 10 nucleotides in the 3′ region (i.e., seed region) of, for example, a 20 nucleotide spacer sequence may be 100% complementary to the target DNA, while the remaining nucleotides in the 5′ region of the spacer sequence are substantially complementary (e.g., at least about 70% complementary) to the target DNA. In some embodiments, the first 1 to 10 nucleotides (e.g., the first 1, 2, 3, 4, 5, 6, 7, 8, 9, 10 nucleotides, and any range therein) of the 3′ end of the spacer sequence may be 100% complementary to the target DNA, while the remaining nucleotides in the 5′ region of the spacer sequence are substantially complementary (e.g., at least about 50% complementary (e.g., at least about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more or any range or value therein)) to the target DNA.

In some embodiments, a seed region of a spacer may be about 8 to about 10 nucleotides in length, about 5 to about 6 nucleotides in length, or about 6 nucleotides in length.

As used herein, a “target nucleic acid”, “target DNA,” “target nucleotide sequence,” “target region,” or a “target region in the genome” refers to a region of a plant's genome that is fully complementary (100% complementary) or substantially complementary (e.g., at least 70% complementary (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to a spacer sequence in a guide nucleic acid of this invention. A target region useful for a CRISPR-Cas system may be located immediately 3′ (e.g., Type V CRISPR-Cas system) or immediately 5′ (e.g., Type II CRISPR-Cas system) to a PAM sequence in the genome of the organism (e.g., a plant genome). A target region may be selected from any region of at least 15 consecutive nucleotides (e.g., 16, 17, 18, 19, 20, 21, 22, 23, 24, 25, 26, 27, 28, 29, 30 nucleotides, and the like) located immediately adjacent to a PAM sequence.

A “protospacer sequence” refers to the target double stranded DNA and specifically to the portion of the target DNA (e.g., or target region in the genome) that is fully or substantially complementary (and hybridizes) to the spacer sequence of the CRISPR repeat-spacer sequences (e.g., guide nucleic acids, CRISPR arrays, crRNAs).

In the case of Type V CRISPR-Cas (e.g., Cas12a) systems and Type II CRISPR-Cas (Cas9) systems, the protospacer sequence is flanked by (e.g., immediately adjacent to) a protospacer adjacent motif (PAM). For Type IV CRISPR-Cas systems, the PAM is located at the 5′ end on the non-target strand and at the 3′ end of the target strand.

In the case of Type II CRISPR-Cas (e.g., Cas9) systems, the PAM is located immediately 3′ of the target region. The PAM for Type I CRISPR-Cas systems is located 5′ of the target strand. There is no known PAM for Type III CRISPR-Cas systems. Makarova et al. describes the nomenclature for all the classes, types and subtypes of CRISPR systems (Nature Reviews Microbiology 13:722-736 (2015)). Guide structures and PAMs are described in by R. Barrangou (Genome Biol. 16:247 (2015)).

Canonical Cas12a PAMs are T rich. In some embodiments, a canonical Cas12a PAM sequence may be 5′-TTN, 5′-TTTN, or 5′-TTTV. In some embodiments, canonical Cas9 (e.g., S. pyogenes) PAMs may be 5′ NGG-3′. In some embodiments, non-canonical PAMs may be used but may be less efficient.

Additional PAM sequences may be determined by those skilled in the art through established experimental and computational approaches. Thus, for example, experimental approaches include targeting a sequence flanked by all possible nucleotide sequences and identifying sequence members that do not undergo targeting, such as through the transformation of target plasmid DNA (Esvelt et al. 2013. Nat. Methods 10:1116-1121; Jiang et al. 2013. Nat. Biotechnol. 31:233-239). In some aspects, a computational approach can include performing BLAST searches of natural spacers to identify the original target DNA sequences in bacteriophages or plasmids and aligning these sequences to determine conserved sequences adjacent to the target sequence (Briner and Barrangou. 2014. Appl. Environ. Microbiol. 80:994-1001; Mojica et al. 2009. Microbiology 155:733-740).

In some embodiments, the present invention provides expression cassettes and/or vectors comprising the nucleic acid constructs of the invention (e.g., one or more components of an editing system of the invention). In some embodiments, expression cassettes and/or vectors comprising the nucleic acid constructs of the invention and/or one or more guide nucleic acids may be provided.

In some embodiments, the nucleic acid constructs, expression cassettes or vectors of the invention may be optimized for expression in a plant, wherein when optimized may be about 70% to 100% identical (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100%) to the nucleic acid constructs, expression cassettes or vectors comprising the same polynucleotide(s) but which have not been codon optimized for expression in a plant.

Further provided herein are cells comprising one or more polynucleotides, guide nucleic acids, nucleic acid constructs, expression cassettes or vectors of the invention.

The nucleic acid constructs of the invention (e.g., a construct comprising a sequence specific nucleic acid binding domain, a CRISPR-Cas effector domain, and/or a guide nucleic acid, etc.) and expression cassettes/vectors comprising the same may be used for editing and modifying target nucleic acids and/or their expression.

A KASII (3-oxoacyl-[acyl-carrier-protein] synthase II, chloroplastic) target nucleic acid of any Arachis hypogaea (peanut) plant or plant part may be modified (e.g., mutated, e.g., base edited, cleaved, nicked, etc.) using the polypeptides, polynucleotides, ribonucleoproteins (RNPs), nucleic acid constructs, expression cassettes, and/or vectors of the invention. A peanut plant and/or plant part that may be modified as described herein may be any peanut variety or cultivar. Peanut varieties useful with this invention include, but are not limited to, TifRunner, GA06G, GA09B, 10X29-6-1-3-1-RunnerNormal, AC13321-RunnerHO and Walton-VA-HO.

In some embodiments, the KASII genomic sequence from the A genome of TifRunner (NCBI Gene ID No. LOC112696350), GA06G, GA09B, 10X29-6-1-3-1-RunnerNormal, and AC13321-RunnerHO comprises a nucleic acid sequence of SEQ ID NO:1 and the respective coding sequence comprises a nucleic acid sequence of SEQ ID NO:3. In some embodiments, the KASII genomic sequence from the A genome of Walton-VA-HO comprises a nucleic acid sequence of SEQ ID NO:2 and the respective coding sequence comprises a nucleic acid sequence of SEQ ID NO:3. The KASII polypeptide encoded by SEQ ID NOs:1 and 2 (and the coding sequence of SEQ ID NO:3) comprises the amino acid sequence of SEQ ID NO:4. In some embodiments, a region of a KASII genomic sequence from TifRunner, GA06G, GA09B, 10X29-6-1-3-1-RunnerNormal, AC13321-RunnerHO and Walton-VA-HO for targeting comprises any one or more of the nucleotide sequences of SEQ ID NOs:5-83 and/or SEQ ID NOs:275-298, optionally wherein a region may be in the upstream region (e.g., SEQ ID NOs:5-31), in Exon 1 (e.g., SEQ ID NOs:32-51), Exon 2 (e.g., SEQ ID NOs:52-69) or Exon 7 (e.g., SEQ ID NOs:70-83) of the KASII gene (SEQ ID NO:1 or SEQ ID NO:2). Example spacers and primers useful with these KASII genes are provided in Table 1.

TABLE. 1
Spacers and primers for KASII (LOC11269350) (A genome)

| | SPACERS | Left Primer | Right Primer |
|---|---|---|---|
| Coding | GAGTCCCGGCGCGGTCA | ATGATACATTCCGCTGC | GAAGGAGCTTATGAGTCC |
| Exon 1 | GCGTGG SEQ ID NO: 84 | TTCAT SEQ ID NO: 96 | CTGA SEQ ID NO: 97 |
| | GTAGTCATCGCAGGGCTC | ACCTCCCAATTGAACAT | TTTATTGAGACGGGTGTGT |
| | GAAGG SEQ ID NO: 85 | GAGAT SEQ ID NO: 98 | CTG SEQ ID NO: 99 |
| | AGATTGATTTCTTCGCTCT | ATGATACATTCCGCTGC | AGTGTTGCAGTAGTCATC |
| | ACGG SEQ ID NO: 86 | TTCAT SEQ ID NO: 96 | GCAG SEQ ID NO: 100 |
| Coding | AAAGGCGAGTAGTTGTGA | AACCTGCACATGAAGTC | CAGCACAATCAAATGTCTC |
| Exon 2 | CCGGG SEQ ID NO: 87 | ACAAC SEQ ID NO: 101 | GAT SEQ ID NO: 102 |
| | ACATCTGGTTCGTGACCA | CACATGAAGTCACAACA | CAGCACAATCAAATGTCTC |
| | AGAGG SEQ ID NO: 88 | ACGAA SEQ ID NO: 103 | GAT SEQ ID NO: 102 |
| | AGGTGTAACAACGCCCAA | AACCTGCACATGAAGTC | GATCTCACTAATGCCGCTA |
| | CCCGG SEQ ID NO: 89 | ACAAC SEQ ID NO: 101 | ACA SEQ ID NO: 104 |
| Coding | CATGCAGAGCCCTATCAC | ACGGTTATCGTGCCATA | TGAAAGCCTCAGCAATAC |
| Exon 7 | AGAGG SEQ ID NO: 90 | AATCT SEQ ID NO: 105 | AAAA SEQ ID NO: 106 |
| | AAGGGCGTGAAGCTTTG | ATTCTTTTTCACCTGCAT | TGAAAGCCTCAGCAATAC |
| | GTAGGG SEQ ID NO: 91 | ACGG SEQ ID NO: 107 | AAAA SEQ ID NO: 106 |
| Up-stream | ATACCAGTGTCTCCTTTT | GCCTTCCATTCTCTTAC | CAAAAGAAATTGAAATTAA |
| region | GCTGG SEQ ID NO: 92 | CAAAA SEQ ID NO: 108 | TCCGT SEQ ID NO: 109 |
| | TAGCCAGCAAAAGGAGAC | TTTTCTTCAAACAAATC | AAATAAAGACCACAAAAGA |
| | ACTGG SEQ ID NO: 93 | GGACC SEQ ID NO: 110 | AAAGGA SEQ ID NO: 111 |
| | CAACTACTAGTTTACATAT | TTTTCTTTTCCTTTTCTT | AATGGGTGACAAAATGAC |
| | ATGG SEQ ID NO: 94 | TTGTGG SEQ ID NO: 112 | TTGA SEQ ID NO: 113 |
| | AAGTATTAACAGCTCATC | TCAAGTCATTTTGTCAC | AAACAAAAGCTATTTTTAA |
| | AGTGG SEQ ID NO: 95 | CCATT SEQ ID NO: 114 | CCGTAA SEQ ID NO: 115 |
| 3′ | GAAACTGACCTTTAATAG | TCGCATTAAACATGAGC | CAGGAAATGCCCTCTTTAG |
| region | AAAGG SEQ ID NO: 242 | AATATC SEQ ID NO: 247 | TTTT SEQ ID NO: 252 |
| | CCCCTTTTCCCTAAATGG | GCTGTTAAGGAAAAACC | TGTACCCAAATAGTTTGTG |
| | TCTGG SEQ ID NO: 243 | AAAGA SEQ ID NO: 248 | CAAT SEQ ID NO: 253 |
| | GGGGATGATTTTAATCTC | AGAAAGGCGGCAAATA | TTTTTCGCTGCCTTGTTCT |
| | TCAGG SEQ ID NO: 244 | AATCTC SEQ ID NO: 249 | ATT SEQ ID NO: 254 |
| | ACCATTTTAGTCACAAAA | TTACCACTCCTTGATCT | AAGCTGTAGCAACAGTCC |
| | CGCGG SEQ ID NO: 245 | GGGTT SEQ ID NO: 250 | AGGT SEQ ID NO: 255 |
| | AACATGAATGCAAGTTCC | TCAATGCAAAGTATAAC | TTGTCTCGACAATATGGAC |
| | GCAGG SEQ ID NO: 246 | GCCAG SEQ ID NO: 251 | CTG SEQ ID NO: 256 |

In some embodiments, the KASII genomic sequence from the B genome of TifRunner (NCBI Gene ID No. LOC112740759) and Walton-VA-HO comprises a nucleic acid sequence of SEQ ID NO:116 and the respective coding sequence comprises a nucleic acid sequence of SEQ ID NO:120. In some embodiments, the KASII genomic sequence from the B genome of GA06G and GA09B comprises a nucleic acid sequence of SEQ ID NO:117 and the respective coding sequence comprises a nucleic acid sequence of SEQ ID NO:120. In some embodiments, the KASII genomic sequence from the B genome of 10X29-6-1-3-1-RunnerNormal comprises a nucleic acid sequence of SEQ ID NO:118 and the respective coding sequence comprises a nucleic acid sequence of SEQ ID NO:120. In some embodiments, the KASII genomic sequence from the B genome of AC13321-RunnerHO comprises a nucleic acid sequence of SEQ ID NO:119 and the respective coding sequence comprises a nucleic acid sequence of SEQ ID NO:120. The KASII polypeptide encoded by SEQ ID NOs:116-119 (and the coding sequence of SEQ ID NO:120) comprises the amino acid sequence of SEQ ID NO:121. In some embodiments, a region of a KASII genomic sequence (B genome) from TifRunner, GA06G, GA09B, 10X29-6-1-3-1-RunnerNormal, AC13321-RunnerHO and Walton-VA-HO for targeting comprises any one or more of the nucleotide sequences of SEQ ID NOs:122-212 and/or SEQ ID NOs:299-326, optionally wherein a region may be in the upstream region (e.g., SEQ ID NOs:122-148), in Exon 1 (e.g., SEQ ID NOs:149-168), Exon 2 (e.g., SEQ ID NOs:169-188), Exon 3 (e.g., SEQ ID NOs:189-208) or Exon 7 (e.g., SEQ ID NOs:209-212) of the KASII gene (SEQ ID NOs:116-119). Example spacers and primers useful with these KASII genes are provided in Table 2.

TABLE. 2
Spacers and primers for KASII (LOC112740759) (B genome)

| | Spacers | Left Primer | Right Primer |
|---|---|---|---|
| Coding | GGACTCCAAAGGCAGCG | ATGATACATTCCGCTGCT | GAAGGAGCTTATGAGT |
| Exon 1 | CTTCGG SEQ ID NO: 213 | TCAT SEQ ID NO: 96 | CCCTGA SEQ ID NO: 97 |
| | GTAGTCATCGCAGGGCT | CCTCCCAATTGAACATGA | TTTATTGAGACGGGTG |
| | CGAAGG SEQ ID NO: 85 | GATT SEQ ID NO: 222 | TGTCTG SEQ ID NO: 99 |
| | GCTGTACGGATCCAGCA | ATCCAAGAGGTCAAGGAG | TTTATTGAGACGGGTG |
| | TTCAGG SEQ ID NO: 214 | GAC SEQ ID NO: 223 | TGTCTG SEQ ID NO: 99 |
| Coding | GCGAGTAGTTGTGACTG | AAAACAATGGCTGTAGCT | CAGCACAATCAAATGT |
| Exon 2 | GGTTGG SEQ ID NO: 215 | GTGG SEQ ID NO: 224 | CTCGAT SEQ ID NO: 96 |
| | TACTACAACAATTTACTT | AAACAATGGCTGTAGCTG | CGTTGGATATTCAGCA |
| | GATGG SEQ ID NO: 216 | TGG SEQ ID NO: 225 | CAATCA SEQ ID NO: 226 |
| | ACATCTGGTTCGTGACC | CAATGGCTGTAGCTGTGG | CAGCACAATCAAATGT |
| | AAGAGG SEQ ID NO: 88 | AAC SEQ ID NO: 227 | CTCGAT SEQ ID NO: 96 |
| Coding | GGTAAAAAGGCCTTAGTT | AGGATTGCTGGTGAGATC | GCCAATCAAAACTCCA |
| Exon 3 | GATGG SEQ ID NO: 217 | AAGT SEQ ID NO: 228 | CATTTT SEQ ID NO: 229 |
| | ATGCTCTATTTGCTAACA | AGGATTGCTGGTGAGATC | GCCAATCAAAACTCCA |
| | GCCGG SEQ ID NO: 218 | AAGT SEQ ID NO: 228 | CATTTT SEQ ID NO: 229 |
| | AGTAATTCCACCATCAAC | ATCAAGTCGTTCTCCACT | AGCCAATCAAAACTCC |
| | TAAGG SEQ ID NO: 219 | GATG SEQ ID NO: 230 | ACATTT SEQ ID NO: 231 |
| Up-stream | GCTCATCAGTAGCTTGCT | CAAGTCATTTTGTCACCC | GCATTAAAACTCAATTT |
| region | GATGG SEQ ID NO: 220 | ATTC SEQ ID NO: 232 | GCTCA SEQ ID NO: 233 |
| | TAAAATACTTATGAGATG | CAATCAAGTCATTTTGTCA | GCATTAAAACTCAATTT |
| | CCCGG SEQ ID NO: 221 | CCC SEQ ID NO: 234 | GCTCA SEQ ID NO: 233 |
| | TTGCGAAAAGGATTGATC | TTTGACTAAAGAAAATCT | TCCACTCCACCTGAAC |
| | CAAGG SEQ ID NO: 222 | GAAACCA SEQ ID NO: 235 | CTTTAC SEQ ID NO: 236 |
| | AGGTAAAGGTTCAGGTG | AGTGCATTAAATTGCGAA | CATCGCAAAGTTGATA |
| | GAGTGG SEQ ID NO: 223 | AAGG SEQ ID NO: 237 | GTCGAG SEQ ID NO: 238 |
| Coding | CATGCAGAGCCCTATCG | TTTCACCTGCATACGGTT | GCAGAAAGAATGAGCA |
| Exon 7 | CAGAGG same as '350 | ATCA SEQ ID NO: 239 | GGATTT SEQ ID NO: 240 |
| | SEQ ID NO: 224 | | |
| | AAGGGCGTGAAGCTTTG | ATTCTTTTTCACCTGCATA | AGCAGAAAGAATGAGC |
| | GTAGGG SEQ ID NO: 91 | CGG SEQ ID NO: 107 | AGGATT SEQ ID NO: 241 |
| 3′ | TTAACCTAACCATCAGCA | AAGAAGAAATTCCAGTGC | ATGCATATCATGTGACA |
| region | AATGG SEQ ID NO: 257 | CAAA SEQ ID NO: 263 | GAGCC SEQ ID NO: 269 |
| | GACTCTTAAACAAATCCA | AATAGAACAAGGCAGCGA | GAAATGGTTGGAGCAG |
| | CTTGG SEQ ID NO: 258 | AAAA SEQ ID NO: 264 | AAACTC SEQ ID NO: 270 |
| | GTTAAGTTACTAAGGTAG | TCACATCAAAGTGGGTCA | GATGACTGTGGATGTT |
| | CCAGG SEQ ID NO: 259 | AGTC SEQ ID NO: 265 | CCTTGA SEQ ID NO: 271 |
| | GGTTGGAGCAGAAACTC | TCACATCAAAGTGGGTCA | GATGACTGTGGATGTT |
| | TTGAGG SEQ ID NO: 260 | AGTC SEQ ID NO: 266 | CCTTGA SEQ ID NO: 272 |
| | ATGACCTGCTCATTATTT | TGTTCAAGGAACATCCAC | CAGGATACTTTTGTCAC |
| | AGAGG SEQ ID NO: 261 | AGTC SEQ ID NO: 267 | ACGGA SEQ ID NO: 273 |
| | GGTTTGCTCTCTCTTCGT | GATGGAATGATGCAACAA | CCAATGGATCACTTAG |
| | ACAGG SEQ ID NO: 262 | GAAA SEQ ID NO: 268 | TTCGGT SEQ ID NO: 274 |

The term “plant part” (i.e., peanut plant part), as used herein, includes but is not limited to reproductive tissues (e.g., petals, sepals, stamens, pistils, receptacles, anthers, pollen, flowers, fruits, flower bud, ovules, seeds, and/or embryos); vegetative tissues (e.g., petioles, stems, roots, root hairs, root tips, pith, coleoptiles, stalks, shoots, branches, bark, apical meristem, axillary bud, cotyledon, hypocotyls, and leaves); vascular tissues (e.g., phloem and xylem); specialized cells such as epidermal cells, parenchyma cells, chollenchyma cells, schlerenchyma cells, stomates, guard cells, cuticle, mesophyll cells; callus tissue; and cuttings. In some embodiments, the plant part is a peanut seed. The term “plant part” also includes plant cells, including plant cells that are intact in plants and/or parts of plants, plant protoplasts, plant tissues, plant organs, plant cell tissue cultures, plant calli, plant clumps, and the like. As used herein, “shoot” refers to the above ground parts including the leaves and stems. As used herein, the term “tissue culture” encompasses cultures of tissue, cells, protoplasts and callus.

As used herein, “plant cell” refers to a structural and physiological unit of the plant, which typically comprise a cell wall but also includes protoplasts. A plant cell of the present invention can be in the form of an isolated single cell or can be a cultured cell or can be a part of a higher-organized unit such as, for example, a plant tissue (including callus) or a plant organ. A “protoplast” is an isolated plant cell without a cell wall or with only parts of the cell wall. Thus, in some embodiments of the invention, a cell edited as described herein may be a cell of any peanut plant or plant part including, but not limited to, a root cell, a leaf cell, a tissue culture cell, a seed cell, a flower cell, a fruit cell, a pollen cell, and the like. In some aspects of the invention, the peanut plant part can be a peanut plant germplasm. In some aspects, a peanut plant cell can be non-propagating plant cell that does not regenerate into a plant.

“Plant cell culture” means cultures of plant units such as, for example, protoplasts, cell culture cells, cells in plant tissues, pollen, pollen tubes, ovules, embryo sacs, zygotes and embryos at various stages of development.

As used herein, a “plant organ” is a distinct and visibly structured and differentiated part of a plant such as a root, stem, leaf, flower bud, or embryo.

“Plant tissue” as used herein means a group of plant cells organized into a structural and functional unit. Any tissue of a plant in planta or in culture is included. This term includes, but is not limited to, whole plants, plant organs, plant seeds, tissue culture and any groups of plant cells organized into structural and/or functional units. The use of this term in conjunction with, or in the absence of, any specific type of plant tissue as listed above or otherwise embraced by this definition is not intended to be exclusive of any other type of plant tissue.

In some embodiments of the invention, an edited tissue culture or edited plant cell culture is provided, wherein the edited tissue or cell culture comprises an edited KASII target nucleic acid molecule. In some embodiments, a peanut plant of the invention may comprise transgenes conferring traits of interest. In some embodiments, the transgenes may be eliminated from a peanut plant developed from transgenic tissue or cell by breeding of the transgenic plant with a non-transgenic plant and selecting among the progeny for the plants comprising the desired gene edit and not the transgenes used in producing the edit.

The invention will now be described with reference to the following examples. It should be appreciated that these examples are not intended to limit the scope of the claims to the invention but rather are intended to be exemplary of certain embodiments. Any variations in the exemplified methods that occur to the skilled artisan are intended to fall within the scope of the invention.

### EXAMPLES

### Example 1

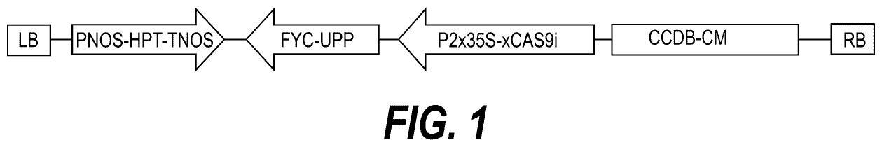

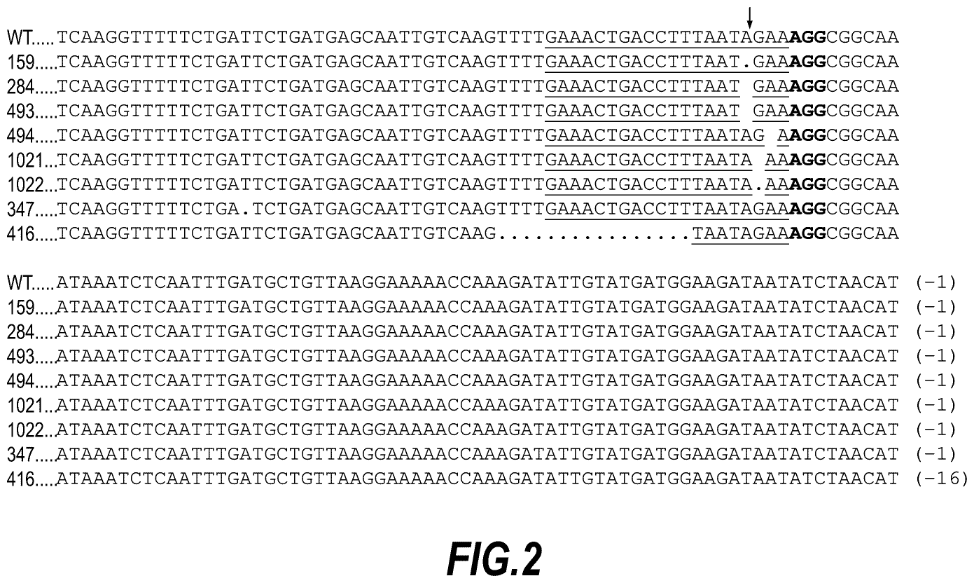

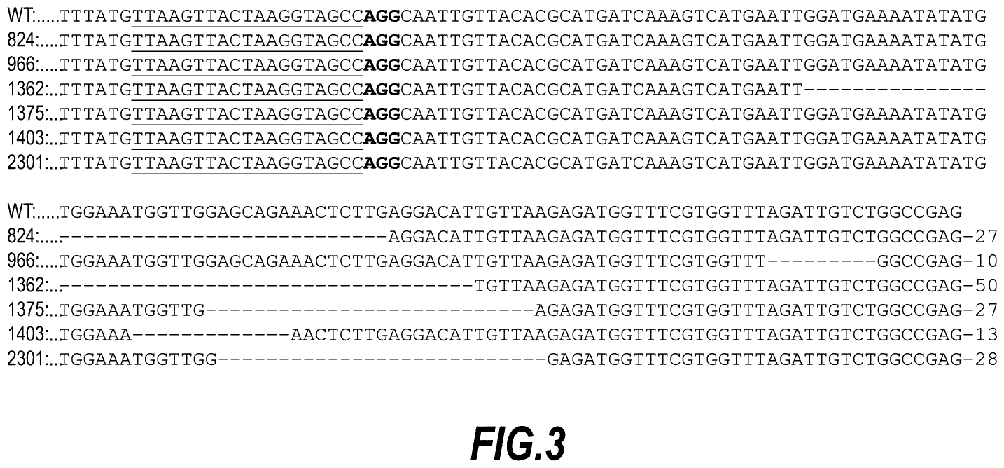

Selected peanut varieties are transformed with gene editing vectors, including TifRunner, GA06G, GA09B, 10X29-6-1-3-1-RunnerNormal, AC13321-RunnerHO and Walton. Embryogenic calluses are transformed using the vector described in FIG. 1 and subjected to hygromycin selection. TO plants are regenerated from hygromycin-resistant embryos. T1 seeds are harvested from TO plants and analyzed for edits in the KASII gene. T1 seeds with KASII gene edits are then planted to produce T1 plants. T2 seeds harvested from T1 plants are reconfirmed for KASII edits. They are planted to generate T2 plants which are subjected to transgene-free tests. Transgene-free KasII edited T2 plants are analyzed for homozygosity for the mutation in KASII. T3 seeds harvested from T2 plants are analyzed for reduction or absence of KASII enzyme activity and altered levels of palmitic acid in the seed oil. FIG. 2 and FIG. 3 show initial results from editing the peanut variety Walton as described herein.

SEQUENCES

KASII Genomic (Arachis hypogaea)
TTATAGAAGCTATAAATCAGAGGCACAGATTTGTTTTATTTTTTTCCAAACAATCACAAATCGAACG
GTTCAATCTGTGATTTCGAAAATAAAAAAATTTTAATGTTAAAATCGAACTGTCTGATTTTTATTAAA
AAATAAAAAAATAAAAAATATAAAACAGACCATGCGATTTGTAGTTTTTTTAAACAAATTGGACTCT
CTGATTTGTAGTTCATTTTTTCTTCAAACAAATCGGACCGTCCGATTTAAATAAAACACCATAAAAA
AAATACCTAATCTTCCAATAACAATATATTACACACATATTTAACCAATATAAAAAAAATTAGCCTTC
CATTCTCTTACCAAAAAAAAGTAATATTAATTTTCAATTTGCATCTCTGTTACATTATTTCAGGTCAC
GTAGCCAGCAAAAGGAGACACTGGTATGCACCAGCATTTTTCTTTTCCTTTTCTTTTGTGGTCTTT
ATTTTTTAGAATATATATGATAATATATACGGATTAATTTCAATTTCTTTTGTATCATATTCAACTACT
AGTTTACATATATGGGACTTTTTAATAATCATCAAGAGATAAAATTTAAATATTTTTATTTATCATAT
AATTAGAGATAAGATAAATTTTGATTCATGAATTTGATCTGATAATTAATATAAATTTTAAACATACT
TAATTTGATAATGAGTTTTATAACTATAATTCAATCAAGTCATTTTGTCACCCATTCTATTACTGTAT
TACATGGATACTTATGTGTGCAGATAACTATTAAGTATTAACAGCTCATCAGTGGCTTGCAGATAG
TAATGTCTCTCAACTTGTTTACATGACATTTAACTAAATGAGCATCTCATAAGTATTTTAAGCATTT
TATTAATTAGATTGATTATTAAAAAAATTAATTTGATTTACGGTTAAAAATAGCTTTTGTTTAAAATTT
AAAAGCCCAACTAATTATTAGATGAAAATTTATGAGCAAATTAGGTTTTAGTGTTGATTTGGATTAG
TTTTTTCGAGTGAAAAATATTTTTTTTTAAAAAAAAAATTAAAATATTTTTTATTCAATTCTAAATTTA
TTTAGATATATTTTTTAATTTATTAAAAAATTAAACAATTTACTTTTTTAAAAGTTAACAATATTTTAC
TTTTAAAAAAATAGAAACAAAAAACGTTTTTAATATTTAAATTTTTTAAAAATCTTATTTAAAAAAATA
AAAACTAAATATTTTTTTAAAAATCAATTTAAACTGATCTTTATCTCTTAATTATTAACTAATTTTGGT
ATTTACTTTCAAATTTTCGTAAAAAAAAGTCTTACTGAGAATAAAGTATTATTTTGACTAAAGAAAAT
CTGAAAGCAAGAATAAAATAAATGCTATGTCACGTTTTACACTAATGATGAAAAAAAATATACAATA
TAAGTAGAAAATATTGCATAAATAATGATGAATAGCGAAGAAAAAGAAGAGAGGTTAGTAGAAAAT
TAAATAAAATAAAATTAATAGGGAAAGCAAATGAAATGAGAAGCGAAGGAGAGAGTGAGAGTCAG
ATTGCAGAAAAAGCAGAGACTTTGAGACTCTGAGACTTCACAACGCCTCCCTTTTCCTTCTTCTCT
CAGATCGATTCTCTTTCTCACTCTCTCAACTCTCAACCTTCTTCAAAACCCCCACAACTCACTTTC
TTTTTCTTCTTCTTCTTCTTTGTATATACATACATACATACATACATGTACATGTATCTTCACTGAAA
CGTCCTCGATTCTCTTGCGAGCTTCTGCTTTAGACAACATGATACATTCCGCTGCTTCATCCATG
GCCTCACCCCTCTGCACGTGGCTGGTGGCAGCTTGCATGTCAGTCACGTGCCACGCTGACCGC
GCCGGGACTCCAAAGGCAGCGCTTCGCTCATCCAAGAGGTCAAGGAGGACGAAGGCGCTGCCA
CCAAACACCTCCCAATTGAACATGAGATTGATTTCTTCGCTCTACGGATCCAGCATTCAGGGACT
CATAAGCTCCTTCGAGCCCTGCGATGACTACTGCAACACTCACAATGCCTTCTCTTCTCTCTTTC
GATCCACAACTCCCAATCGCAGACACACCCGTCTCAATAAACTTGCTCACTCTGGTAAACTTTTTT
TTTTTTTTTAATTTTCCCCACTTAAATCACCTCAAGTTAAGATATTTCTGGTGCTTTTTGCAATTTTA
ATCTGTACCCCGTACTGGGTATTTCAATTTGATTCATTTATTTAATTTTCTTATATTAATATAATAAT
AATAATAGTTTTTTTATTATGGTTATGAAATCTGGGTTCTGATTTTCTGGTCTTCTGAAGGATGTGT
GTGTGTATGTGTGGCTTTTTTCCCTTTTTTTCTGAATTACTTAAAATGCACAAGTGTTAGCTTGGAT
CTTTGTCTTTTGGTTGGTCATCCTAATAAATGGGGTTGCTTGGTGGTTTAGTATGGAGGGTTGTTC
TACCTGCTCTGTTTAACTGTTTTGAGGGTACGGTTTATAATATTAGGAGTATGGGGGTAGTTGTTG
TTCTTTCTTTGCTGAGGTTGATGGTGCACCTGTGTCACTTAATTAGGACATGTTGCTACAACACTA
TGCCATTGCCCATGCTTTTTGCTTTCATTTCCCATTCCTACTTCATTTCCTTGTATTTATCGTACAC
TCAGGTATCTACATAAAGTTCCCATTAAATATTGGTTAATGTTCGCATCATCACTTTGAATTTCTTG
CAATCTCGTTGAGGTAGTTCAGCATATTCACAGAATCAACTCAAACAAAAATAAATTTCTTTTGACT
TTTATTTTTATTTTTTTTTTGCTTTTCTTCATGAAAATAATAATCACACTGAAGTGTTCTGAAGTTAA
CTACTATGTAAATATCGCCCCCACGTGGGATTACCTCTCTCTCCCTCTCTCTCTCGTTTTTTGTTT
CTCTATGTAAAATAATGGAGGTAAGTTATGCCTTTGTCATAAGCACACATAAGCAAGTCAGAGGC
TCCTTTTTTTTTTTTCCCTCAATTTTTTTTTTAAATTATCATGTAAAATAGATGATTCTGGCACAAAC
TATCCACAAGTTATTTTTGCTCCTAGTTCTTTAGTATGTTTTTAACACACTGGACAAGTCTACTACT
GCTGCATGCATGTCCCTGCCTGATGGGTTCTAGTATTCAGTGGTCTTGAGTGTTTTGATTTAAAG
CAAGGGATATAAGTTGGGTCTGATGAATTTTTCGAGGTTTCATAGCATTTTTGTTACCATAGTCTT
AGAATTACTACCTTGGATAAACCTAAGCATATTTTTGTTAATGGCTGGGGTTGCATTTCCTGCTCA
ACATTAATGTGCACCAAGGGTATGTTCTGTTAGGGGTTAAAAATGGGTTGGGAATTTACAATGGA
TGTGGCCCATGTGTAATTTCACTCATAGAACTTATTGTAAAAATTTCACCCCATTTCTACTTCCCAA
AAGAACATATCCTTATCATGATCATTGGAAACTACACATGCATCATGTTGAACTATAAGCTGTGGG
CCGCTCTTTCTTTGTATGCTTCTGATACGAATTTCTTGAATGATAGGCAGTTGCATGCAATTTTTAT
TATTGCTGACTTATTCCTCTGAATTGCATAACTATAAGGTAAAACAATGGCTGTAGCTGTGGAACC
TGCACATGAAGTCACAACAACGAAAAAACCTCCTACAAAACAAAGGCGAGTAGTTGTGACCGGG
TTGGGCGTTGTTACACCTCTTGGTCACGAACCAGATGTTTACTACAACAATTTACTTGATGGTGTT
AGCGGCATTAGTGAGATCGAGACATTTGATTGTGCTGAATATCCAACGGCAAGTTATATATAATT
GAAATTGTATTTTTTGTTGTCTGTTGTAGCTTTTGGTCTGAATATTCTATGTTTCCAGAGGATTGCT
GGTGAGATCAAGTCGTTCTCCACCGATGGCTGGGTTGCACCAAAACTTTCGAAGAGAATGGATA
AATTTATGCTCTATTTGCTAACAGCCGGTAAGAAGGCCTTAGTTGATGGTGGAATTACTGAAGAT
GTAATGGATGAGTTGAATAAGGAAAAATGTGGGGTTTTGATTGGCTCAGCAATGGGTGGCATGAA
GGTAACCTTGTCTTACTTTCATGTTGAGATACAAATCTTGTAAAACCAGCTGGTGTTGTGGACTGA
ATTTAATGCCACTGCAATCCAAATGGAGCAGAAGAGTGTGCTATATTTTCAACCTTGTGCCCTTGT
TTTATTGACTATGATTTTTGGTGCTTTTTCTATTGAGCAGGTTTTTAATGATGCCATTGAAGCTTTA
AGAGTATCATATAAGAAGATGAATCCTTTTTGTGTACCTTTTGCCACAACAAATATGGGATCTGCC
ATACTTGCGATGGATCTGGTCTGTTGTTGCATGGACACCATAAATTGGTGTTTTGTTTCGTACCAA
GCTTTTATTTTGGCTATGTTGTCTTCAGAGTCTAACAAACATTGTTATCTTTTATGGATTACAGGGA
TGGATGGGCCCTAATTATTCAATCTCTACAGCTTGTGCTACAAGTAACTTTTGTATATTGAATGCA
GCAAACCACATCATAAGAGGTGAAGCTGTAAGTATTCATGCTTTGATATTACTCTCATGATGGTGA
ATTTTTCGTATCCAATATTATACCTTCTATTTATTTATTTATTTAACTTTTTTTTCCTGAGACAGTTTG
GTGTAATGATATGGTCATTGCATCAAAATGTATTGATAAATTTAATATGAAAATATTCATGCAGGAT
GTGATGCTTTGTGGTGGCTCAGATGCTGCTATTATACCCATTGGTATGATCTCACCATTGATTCTT
TTTCACCTGCATACGGTTATCGTGCCATAAATCTAGCTGATATGAATCCACTAATCATTGGTACAG
GTTTGGGAGGTTTTGTGGCATGCAGAGCCCTATCACAGAGGAATACAGACCCTACCAAAGCTTC
ACGCCCTTGGGACATTGTAATACAACACCTGCTCATTCTTTCTGCTACTCACCATGTTTTGTATTG
CTGAGGCTTTCATCATTTTTTATTTTTGAAATAATACGACTATAATTATAATGAAATTTGATGACCAA
GTCTTGTATCCTTTCTTGTAATCTGAACAGCATAGTTTATTAAAAACTAACGTTACAATGGTCACAG
ATACAACTTTTCCTTTTGATCACAAAAATGCTGTCTTCTATGGATATGAGCTATGGCAAATTCAGTT
TTTCTAATTAGGCTGTTTATGATATTCCAGAACCGAGATGGATTTGTCATGGGGGAAGGAGCTGG
AGTTTTACTTTTGGAGGACTTGGAGCATGCAAAGGTACATGAAATCTGATCTAATAATTTTAGAAC
AGAACATCCCTTATGAAATGACAGTGCATCTTTTGATATTATTACTGCAGACTCGTGCATGTAATT
TATTTCGTTTAGTGCATCCTTCAGTTCCTTCAAACAGTATTAAAGGACTTCATACATCTGTTCTCTC
TTGAATTAGGATTGATTTTCAGGCTGCTGCTTGTCATAAAAATTCACCTGTTACTTCTCACATCAAA
TCTGCGCTTGACATTTGTCTTGCAACTGAATCAAATGCATCTCTTTTACATTGCAGAAAAGAGGTG
CAACCATATATGCTGAATTCCTTGGTGGAAGCTTCACCTGTGATGCATATCATGTGACAGAGCCA
CGTCCCGATGGTAAAATATCAAATCCTTTACTAGCAAAATTTAAAGCATTTATATGCATGCTATGAT
TACCATGCTTAAAATCCGAAGAGAACAAGGCAAAATAATGTGACAAGGGTATTCTTATATATTTAG
AATTATGTGTGCAATTTGCTGATGGTTAGGGTTAAGTATTTAGAAATATACTATATGTTGCATTTTT
CTTGTTTGGCACTGGAATTTCTTCTTCAGAGATCGTTATGGAATTTGAAACTGCATGTTGCGATCA
TGACTTGTCTCGACAATATGGACCTGGGATTTAATAATATGCATATATTAGTGGACCTAGTAATCC
TAGTCATAATTAGTATCTGCTTGTACTTTACCACATTTCCTTTATGATTCCATTCGGACTCTTCTTT
GAGTATTCTATGACTTGACAAGGAATGATTTGTATCTCCCTGCGGAACTTGCATTCATGTTTCTTT
ACTCCATCTTAAATAGGAGCTGGCGTTATACTTTGCATTGAAAAGGCATTAGCTCAGTCTGGAGT
ATCAAAAGAGGATGTGAACTACATAAATGCACATGCCACATCCACGCCTGCTGGTGATCTTAAGG
AGTTCCAGGCTCTGCTTCATTGTTTTGGTCAAAATCCTGAGGTACATCTATCTTTTTAAGTTACAC
CACTATTAACATTGTTATCTTGGTGATCAATTCTATGGCAAACATGATCTCTTGTTCTTCTGGAGTT
CATGTTGAGTAAAACACTGGTACTTCTGATATGGTCATCCATACTATTGTCTCTTGTTTTTTCAGCT
AAGAGTGAATTCTACAAAATCTATGATTGGTCATCTACTAGGAGCAGCTGGTGGCGTGGAAGCTG
TAGCAACAGTCCAGGTAAAACCCAATGGATCACTTAGTTCGGTACCCCGCGTTTTGTGACTAAAA
TGGTCCTGAGGTTTGCTCTCTCTTTATACAGGCAATTAGGACAGGGTGGGTGCATCCCAATATCA
ACCTGGAAAACCCAGATCAAGGAGTGGTAATGTTTCTTGTTGCATCATTCCATCCTTCATTTCAAA
ATTTTAATTTTAATCTTTTAATCTAACATGTCCAAATTGATGAGAAAAATTCAGGATGCCAAAGTGC
TTGTGGGATCAAAGAAAGAGAGACTAGATATCAAGGCTGCCCTATCCAATTCATTTGGTTTTGGG
GGCCACAATTCTTCAATCATATTTGCTCCGTATAAGTGAATGAATACATGTTTCAGAATAATACCTT
CCCTACCATTATACAGAAGGTATGTCTCTAAATATATCGTCCTAGTACGAGAAACATTTAATTTGG
ATGACTGTGGATGTTCCTTGAACATGTATTAAATTGTGTGTTGTTTTTATGTTAAGTTACTAAGGTA
GCCAGGCAATTGTTACACGCATGATCAAAGTCATGAATTGGATGAAAATATATGTGGAAATGGTT
GGAGCAGAAACTCTTGAGGACATTGTTAAGAGATGGTTTCGTGGTTTAGATTGTCTGGCCGAGAT
ACATGGTGGAGCCGGTTCTATCCACCGATGAAGAACATGAATGGCTGCTATTGCCTTTATAGAGT
AGGACTTGACCCACTTTGATGTGACTCTTAAACAAATCCACTTGGCAGTATATATAAGCTCTTCTG
TTTTATTTATTCCCTATGTACCCAAATAGTTTGTGCAATAACAAATTGCAATTTATTTTTTCGCTGC
CTTGTTCTATTGCCCTTAAACCAGACCATTTAGGGAAAAGGGGATGATTTTAATCTCTCAGGAAA
SEQ ID NO: 1
KASII genomic (Arachis hypogaea)
TTATAGAAGCTATAAATCAGAGGCACAGATTTGTTTTATTTTTTTCCAAACAATCACAAATCGAACG
GTTCAATCTGTGATTTCGAAAATAAAAAAATTTTAATGTTAAAATCGAACTGTCTGATTTTTATTAAA
AAATAAAAAAATAAAAAATATAAAACAGACCATGCGATTTGTAGTTTTTTTAAACAAATTGGACTCT
CTGATTTGTAGTTCATTTTTTCTTCAAACAAATCGGACCGTCCGATTTAAATAAAACACCATAAAAA
AAATACCTAATCTTCCAATAACAATATATTACACACATATTTAACCAATATAAAAAAAATTAGCCTTC
CATTCTCTTACCAAAAAAAAGTAATATTAATTTTCAATTTGCATCTCTGTTACATTATTTCAGGTCAC
GTAGCCAGCAAAAGGAGACACTGGTATGCACCAGCATTTTTCTTTTCCTTTTCTTTTGTGGTCTTT
ATTTTTTAGAATATATATGATAATATATACGGATTAATTTCAATTTCTTTTGTATCATATTCAACTACT
AGTTTACATATATGGGACTTTTTAATAATCATCAAGAGATAAAATTTAAATATTTTTATTTATCATAT
AATTAGAGATAAGATAAATTTTGATTCATGAATTTGATCTGATAATTAATATAAATTTTAAACATACT
TAATTTGATAATGAGTTTTATAACTATAATTCAATCAAGTCATTTTGTCACCCATTCTATTACTGTAT
TACATGGATACTTATGTGTGCAGATAACTATTAAGTATTAACAGCTCATCAGTGGCTTGCAGATAG
TAATGTCTCTCAACTTGTTTACATGACATTTAACTAAATGAGCATCTCATAAGTATTTTAAGCATTT
TATTAATTAGATTGATTATTAAAAAAATTAATTTGATTTACGGTTAAAAATAGCTTTTGTTTAAAATTT
AAAAGCCCAACTAATTATTAGATGAAAATTTATGAGCAAATTAGGTTTTAGTGTTGATTTGGATTAG
TTTTTTCGAGTGAAAAATATTTTTTTTTAAAAAAAAAATTAAAATATTTTTTATTCAATTCTAAATTTA
TTTAGATATATTTTTTAATTTATTAAAAAATTAAACAATTTACTTTTTTAAAAGTTAACAATATTTTAC
TTTTAAAAAAATAGAAACAAAAAACGTTTTTAATATTTAAATTTTTTAAAAATCTTATTTAAAAAAATA
AAAACTAAATATTTTTTTAAAAATCAATTTAAACTGATCTTTATCTCTTAATTATTAACTAATTTTGGT
ATTTACTTTCAAATTTTCGTAAAAAAAAGTCTTACTGAGAATAAAGTATTATTTTGACTAAAGAAAAT
CTGAAAGCAAGAATAAAATAAATGCTATGTCACGTTTTACACTAATGATGAAAAAAAATATACAATA
TAAGTAGAAAATATTGCATAAATAATGATGAATAGCGAAGAAAAAGAAGAGAGGTTAGTAGAAAAT
TAAATAAAATAAAATTAATAGGGAAAGCAAATGAAATGAGAAGCGAAGGAGAGAGTGAGAGTCAG
ATTGCAGAAAAAGCAGAGACTTTGAGACTCTGAGACTTCACAACGCCTCCCTTTTCCTTCTTCTCT
CAGATCGATTCTCTTTCTCACTCTCTCAACTCTCAACCTTCTTCAAAACCCCCACAACTCACTTTC
TTTTTCTTCTTCTTCTTCTTTGTATATACATACATACATACATACATGTACATGTATCTTCACTGAAA
CGTCCTCGATTCTCTTGCGAGCTTCTGCTTTAGACAACATGATACATTCCGCTGCTTCATCCATG
GCCTCACCCCTCTGCACGTGGCTGGTGGCAGCTTGCATGTCAGTCACGTGCCACGCTGACCGC
GCCGGGACTCCAAAGGCAGCGCTTCGCTCATCCAAGAGGTCAAGGAGGACGAAGGCGCTGCCA
CCAAACACCTCCCAATTGAACATGAGATTGATTTCTTCGCTCTACGGATCCAGCATTCAGGGACT
CATAAGCTCCTTCGAGCCCTGCGATGACTACTGCAACACTCACAATGCCTTCTCTTCTCTCTTTC
GATCCACAACTCCCAATCGCAGACACACCCGTCTCAATAAACTTGCTCACTCTGGTAAACTTTTTT
TTTTTTTTTAATTTTCCCCACTTAAATCACCTCAAGTTAAGATATTTCTGGTGCTTTTTGCAATTTTA
ATCTGTACCCCGTACTGGGTATTTCAATTTGATTCATTTATTTAATTTTCTTATATTAATATAATAAT
AATAATAGTTTTTTTATTATGGTTATGAAATCTGGGTTCTGATTTTCTGGTCTTCTGAAGGATGTGT
GTGTGTATGTGTGGCTTTTTTCCCTTTTTTTCTGAATTACTTAAAATGCACAAGTGTTAGCTTGGAT
CTTTGTCTTTTGGTTGGTCATCCTAATAAATGGGGTTGCTTGGTGGTTTAGTATGGAGGGTTGTTC
TACCTGCTCTGTTTAACTGTTTTGAGGGTACGGTTTATAATATTAGGAGTATGGGGGTAGTTGTTG
TTCTTTCTTTGCTGAGGTTGATGGTGCACCTGTGTCACTTAATTAGGACATGTTGCTACAACACTA
TGCCATTGCCCATGCTTTTTGCTTTCATTTCCCATTCCTACTTCATTTCCTTGTATTTATCGTACAC
TCAGGTATCTACATAAAGTTCCCATTAAATATTGGTTAATGTTCGCATCATCACTTTGAATTTCTTG
CAATCTCGTTGAGGTAGTTCAGCATATTCACAGAATCAACTCAAACAAAAATAAATTTCTTTTGACT
TTTATTTTTTTTTTTTTTTTGCTTTTCTTCATGAAAATAATAATCACACTGAAGTGTTCTGAAGTTAA
CTACTATGTAAATATCGCCCCCACGTGGGATTACCTCTCTCTCCCTCTCTCTCTCGTTTTTTGTTT
CTCTATGTAAAATAATGGAGGTAAGTTATGCCTTTGTCATAAGCACACATAAGCAAGTCAGAGGC
TCCTTTTTTTTTTTTCCCTCAATTTTTTTTTTAAATTATCATGTAAAATAGATGATTCTGGCACAAAC
TATCCACAAGTTATTTTTGCTCCTAGTTCTTTAGTATGTTTTTAACACACTGGACAAGTCTACTACT
GCTGCATGCATGTCCCTGCCTGATGGGTTCTAGTATTCAGTGGTCTTGAGTGTTTTGATTTAAAG
CAAGGGATATAAGTTGGGTCTGATGAATTTTTCGAGGTTTCATAGCATTTTTGTTACCATAGTCTT
AGAATTACTACCTTGGATAAACCTAAGCATATTTTTGTTAATGGCTGGGGTTGCATTTCCTGCTCA
ACATTAATGTGCACCAAGGGTATGTTCTGTTAGGGGTTAAAAATGGGTTGGGAATTTACAATGGA
TGTGGCCCATGTGTAATTTCACTCATAGAACTTATTGTAAAAATTTCACCCCATTTCTACTTCCCAA
AAGAACATATCCTTATCATGATCATTGGAAACTACACATGCATCATGTTGAACTATAAGCTGTGGG
CCGCTCTTTCTTTGTATGCTTCTGATACGAATTTCTTGAATGATAGGCAGTTGCATGCAATTTTTAT
TATTGCTGACTTATTCCTCTGAATTGCATAACTATAAGGTAAAACAATGGCTGTAGCTGTGGAACC
TGCACATGAAGTCACAACAACGAAAAAACCTCCTACAAAACAAAGGCGAGTAGTTGTGACCGGG
TTGGGCGTTGTTACACCTCTTGGTCACGAACCAGATGTTTACTACAACAATTTACTTGATGGTGTT
AGCGGCATTAGTGAGATCGAGACATTTGATTGTGCTGAATATCCAACGGCAAGTTATATATAATT
GAAATTGTATTTTTTGTTGTCTGTTGTAGCTTTTGGTCTGAATATTCTATGTTTCCAGAGGATTGCT
GGTGAGATCAAGTCGTTCTCCACCGATGGCTGGGTTGCACCAAAACTTTCGAAGAGAATGGATA
AATTTATGCTCTATTTGCTAACAGCCGGTAAGAAGGCCTTAGTTGATGGTGGAATTACTGAAGAT
GTAATGGATGAGTTGAATAAGGAAAAATGTGGGGTTTTGATTGGCTCAGCAATGGGTGGCATGAA
GGTAACCTTGTCTTACTTTCATGTTGAGATACAAATCTTGTAAAACCAGCTGGTGTTGTGGACTGA
ATTTAATGCCACTGCAATCCAAATGGAGCAGAAGAGTGTGCTATATTTTCAACCTTGTGCCCTTGT
TTTATTGACTATGATTTTTGGTGCTTTTTCTATTGAGCAGGTTTTTAATGATGCCATTGAAGCTTTA
AGAGTATCATATAAGAAGATGAATCCTTTTTGTGTACCTTTTGCCACAACAAATATGGGATCTGCC
ATACTTGCGATGGATCTGGTCTGTTGTTGCATGGACACCATAAATTGGTGTTTTGTTTCGTACCAA
GCTTTTATTTTGGCTATGTTGTCTTCAGAGTCTAACAAACATTGTTATCTTTTATGGATTACAGGGA
TGGATGGGCCCTAATTATTCAATCTCTACAGCTTGTGCTACAAGTAACTTTTGTATATTGAATGCA
GCAAACCACATCATAAGAGGTGAAGCTGTAAGTATTCATGCTTTGATATTACTCTCATGATGGTGA
ATTTTTCGTATCCAATATTATACCTTCTATTTATTTATTTATTTAACTTTTTTTTCCTGAGACAGTTTG
GTGTAATGATATGGTCATTGCATCAAAATGTATTGATAAATTTAATATGAAAATATTCATGCAGGAT
GTGATGCTTTGTGGTGGCTCAGATGCTGCTATTATACCCATTGGTATGATCTCACCATTGATTCTT
TTTCACCTGCATACGGTTATCGTGCCATAAATCTAGCTGATATGAATCCACTAATCATTGGTACAG
GTTTGGGAGGTTTTGTGGCATGCAGAGCCCTATCACAGAGGAATACAGACCCTACCAAAGCTTC
ACGCCCTTGGGACATTGTAATACAACACCTGCTCATTCTTTCTGCTACTCACCATGTTTTGTATTG
CTGAGGCTTTCATCATTTTTTATTTTTGAAATAATACGACTATAATTATAATGAAATTTGATGACCAA
GTCTTGTATCCTTTCTTGTAATCTGAACAGCATAGTTTATTAAAAACTAACGTTACAATGGTCACAG
ATACAACTTTTCCTTTTGATCACAAAAATGCTGTCTTCTATGGATATGAGCTATGGCAAATTCAGTT
TTTCTAATTAGGCTGTTTATGATATTCCAGAACCGAGATGGATTTGTCATGGGGGAAGGAGCTGG
AGTTTTACTTTTGGAGGACTTGGAGCATGCAAAGGTACATGAAATCTGATCTAATAATTTTAGAAC
AGAACATCCCTTATGAAATGACAGTGCATCTTTTGATATTATTACTGCAGACTCGTGCATGTAATT
TATTTCGTTTAGTGCATCCTTCAGTTCCTTCAAACAGTATTAAAGGACTTCATACATCTGTTCTCTC
TTGAATTAGGATTGATTTTCAGGCTGCTGCTTGTCATAAAAATTCACCTGTTACTTCTCACATCAAA
TCTGCGCTTGACATTTGTCTTGCAACTGAATCAAATGCATCTCTTTTACATTGCAGAAAAGAGGTG
CAACCATATATGCTGAATTCCTTGGTGGAAGCTTCACCTGTGATGCATATCATGTGACAGAGCCA
CGTCCCGATGGTAAAATATCAAATCCTTTACTAGCAAAATTTAAAGCATTTATATGCATGCTATGAT
TACCATGCTTAAAATCCGAAGAGAACAAGGCAAAATAATGTGACAAGGGTATTCTTATATATTTAG
AATTATGTGTGCAATTTGCTGATGGTTAGGGTTAAGTATTTAGAAATATACTATATGTTGCATTTTT
CTTGTTTGGCACTGGAATTTCTTCTTCAGAGATCGTTATGGAATTTGAAACTGCATGTTGCGATCA
TGACTTGTCTCGACAATATGGACCTGGGATTTAATAATATGCATATATTAGTGGACCTAGTAATCC
TAGTCATAATTAGTATCTGCTTGTACTTTACCACATTTCCTTTATGATTCCATTCGGACTCTTCTTT
GAGTATTCTATGACTTGACAAGGAATGATTTGTATCTCCCTGCGGAACTTGCATTCATGTTTCTTT
ACTCCATCTTAAATAGGAGCTGGCGTTATACTTTGCATTGAAAAGGCATTAGCTCAGTCTGGAGT
ATCAAAAGAGGATGTGAACTACATAAATGCACATGCCACATCCACGCCTGCTGGTGATCTTAAGG
AGTTCCAGGCTCTGCTTCATTGTTTTGGTCAAAATCCTGAGGTACATCTATCTTTTTAAGTTACAC
CACTATTAACATTGTTATCTTGGTGATCAATTCTATGGCAAACATGATCTCTTGTTCTTCTGGAGTT
CATGTTGAGTAAAACACTGGTACTTCTGATATGGTCATCCATACTATTGTCTCTTGTTTTTTCAGCT
AAGAGTGAATTCTACAAAATCTATGATTGGTCATCTACTAGGAGCAGCTGGTGGCGTGGAAGCTG
TAGCAACAGTCCAGGTAAAACCCAATGGATCACTTAGTTCGGTACCCCGCGTTTTGTGACTAAAA
TGGTCCTGAGGTTTGCTCTCTCTTTATACAGGCAATTAGGACAGGGTGGGTGCATCCCAATATCA
ACCTGGAAAACCCAGATCAAGGAGTGGTAATGTTTCTTGTTGCATCATTCCATCCTTCATTTCAAA
ATTTTAATTTTAATCTTTTAATCTAACATGTCCAAATTGATGAGAAAAATTCAGGATGCCAAAGTGC
TTGTGGGATCAAAGAAAGAGAGACTAGATATCAAGGCTGCCCTATCCAATTCATTTGGTTTTGGG
GGCCACAATTCTTCAATCATATTTGCTCCGTATAAGTGAATGAATACATGTTTCAGAATAATACCTT
CCCTACCATTATACAGAAGGTATGTCTCTAAATATATCGTCCTAGTACGAGAAACATTTAATTTGG
ATGACTGTGGATGTTCCTTGAACATGTATTAAATTGTGTGTTGTTTTTATGTTAAGTTACTAAGGTA
GCCAGGCAATTGTTACACGCATGATCAAAGTCATGAATTGGATGAAAATATATGTGGAAATGGTT
GGAGCAGAAACTCTTGAGGACATTGTTAAGAGATGGTTTCGTGGTTTAGATTGTCTGGCCGAGAT
ACATGGTGGAGCCGGTTCTATCCACCGATGAAGAACATGAATGGCTGCTATTGCCTTTATAGAGT
AGGACTTGACCCACTTTGATGTGACTCTTAAACAAATCCACTTGGCAGTATATATAAGCTCTTCTG
TTTTATTTATTCCCTATGTACCCAAATAGTTTGTGCAATAACAAATTGCAATTTATTTTTTCGCTGC
CTTGTTCTATTGCCCTTAAACCAGACCATTT33333333TATCAAAGACAAATAACGTTTTCTTTAAA
ATAGATCTCAATTTATATTCCAATTGATGTTAGATATTATCTTCCATCATACAATATCTTTGGTTTTT
CCTTAACAGCATCAAATTGAGATTTATTTGCCGCCTTTCTATTAAAGGTCAGTTTCAAAACTTGAC
AATTGCTCATCAGAATCAGAAAAACCTTGAGATATTGCTCATGTTTAATGCGAGAA SEQ ID NO: 2
KASII Coding sequence (Arachis hypogaea)
ATGATACATTCCGCTGCTTCATCCATGGCCTCACCCCTCTGCACGTGGCTGGTGGCAGCTTGCA
TGTCAGTCACGTGCCACGCTGACCGCGCCGGGACTCCAAAGGCAGCGCTTCGCTCATCCAAGA
GGTCAAGGAGGACGAAGGCGCTGCCACCAAACACCTCCCAATTGAACATGAGATTGATTTCTTC
GCTCTACGGATCCAGCATTCAGGGACTCATAAGCTCCTTCGAGCCCTGCGATGACTACTGCAAC
ACTCACAAtgccttctcttctctctttcgaTCCACAACTCCCAATCGCAGACACACCCGTCTCAATAAACTTGCT
CACTCtgGTAAAACAATGGCTGTAGCTGTGGAACCTGCACATGAAGTCACAACAACGAAAAAACCT
CCTACAAAACAAAGGCGAGTAGTTGTGACCGGGTTGGGCGTTGTTACACCTCTTGGTCACGAAC
CAGATGTTTACTACAACAATTTACTTGATGGTGTTAGCGGCATTAGTGAGATCGAGACATTTGATT
GTGCTGAATATCCAACGAGGATTGCTGGTGAGATCAAGTCGTTCTCCACCGATGGCTGGGTTGC
ACCAAAACTTTCGAAGAGAATGGATAAATTTATGCTCTATTTGCTAACAGCCGGTAAGAAGGCCTT
AGTTGATGGTGGAATTACTGAAGATGTAATGGATGAGTTGAATAAGGAAAAATGTGGGGTTTTGA
TTGGCTCAGCAATGGGTGGCATGAAGGTTTTTAATGATGCCATTGAAGCTTTAAGAGTATCATATA
AGAAGATGAATCCTTTTTGTGTACCTTTTGCCACAACAAATATGGGATCTGCCATACTTGCGATGG
ATCTGGGATGGATGGGCCCTAATTATTCAATCTCTACAGCTTGTGCTACAAGTAACTTTTGTATAT
TGAATGCAGCAAACCACATCATAAGAGGTGAAGCTGATGTGATGCTTTGTGGTGGCTCAGATGCT
GCTATTATACCCATTGGTTTGGGAGGTTTTGTGGCATGCAGAGCCCTATCACAGAGGAATACAGA
CCCTACCAAAGCTTCACGCCCTTGGGACATTAACCGAGATGGATTTGTCATGGGGGAAGGAGCT
GGAGTTTTACTTTTGGAGGACTTGGAGCATGCAAAGAAAAGAGGTGCAACCATATATGCTGAATT
CCTTGGTGGAAGCTTCACCTGTGATGCATATCATGTGACAGAGCCACGTCCCGATGGAGCTGGC
GTTATACTTTGCATTGAAAAGGCATTAGCTCAGTCTGGAGTATCAAAAGAGGATGTGAACTACATA
AATGCACATGCCACATCCACGCCTGCTGGTGATCTTAAGGAGTTCCAGGCTCTGCTTCATTGTTT
TGGTCAAAATCCTGAGCTAAGAGTGAATTCTACAAAATCTATGATTGGTCATCTACTAGGAGCAG
CTGGTGGCGTGGAAGCTGTAGCAACAGTCCAGGCAATTAGGACAGGGTGGGTGCATCCCAATAT
CAACCTGGAAAACCCAGATCAAGGAGTGGATGCCAAAGTGCTTGTGGGATCAAAGAAAGAGAGA
CTAGATATCAAGGCTGCCCTATCCAATTCATTTGGTTTTGGGGGCCACAATTCTTCAATCATATTT
GCTCCGTATAAGTGA SEQ ID NO: 3
KASII polypeptide (Arachis hypogaea)
MIHSAASSMASPLCTWLVAACMSVTCHADRAGTPKAALRSSKRSRRTKALPPNTSQLNMRLISSLYG
SSIQGLISSFEPCDDYCNTHNAFSSLFRSTTPNRRHTRLNKLAHSGKTMAVAVEPAHEVTTTKKPPTK
QRRVVVTGLGVVTPLGHEPDVYYNNLLDGVSGISEIETFDCAEYPTRIAGEIKSFSTDGWVAPKLSKR
MDKFMLYLLTAGKKALVDGGITEDVMDELNKEKCGVLIGSAMGGMKVFNDAIEALRVSYKKMNPFCV
PFATTNMGSAILAMDLGWMGPNYSISTACATSNFCILNAANHIIRGEADVMLCGGSDAAIIPIGLGGFV
ACRALSQRNTDPTKASRPWDINRDGFVMGEGAGVLLLEDLEHAKKRGATIYAEFLGGSFTCDAYHVT
EPRPDGAGVILCIEKALAQSGVSKEDVNYINAHATSTPAGDLKEFQALLHCFGQNPELRVNSTKSMIG
HLLGAAGGVEAVATVQAIRTGWVHPNINLENPDQGVDAKVLVGSKKERLDIKAALSNSFGFGGHNSSI
IFAPYK* SEQ ID NO: 4
Regions
AACACCATAAAAAAAATACCTAATCTTCCAATAACAATATATTACACACATATTTAACCAATATAAA
AAAAATTAGCCTTCCATTCTCTTACCAAAAAAAAGTAATATTAATTTTCAATTTGCATCTCTGTTACA
TTATTTCAGGTCACGTAGCCAGCAAAAGGAGACACTGGTATGCACCAGCATTTTTCTTTTCCTTTT
CTTTTGTGGTCTTTATTTTTTAGAATATATATGATAATATATACGGATTAATTTCAATTTCTTTTGTAT
CATATTCAACTACTAGTTTACATATATGGGACTTTTTAATAATCATCAAGAGATAAAATTTAAATATT
TTTATTTATCATATAATTAGAGATAAGATAAATTTTGATTCATGAATTTGATCTGATAATTAATATAA
ATTTTAAACATACTTAATTTGATAATGAGTTTTATAACTATAATTCAATCAAGTCATTTTGTCACCCA
TTCTATTACTGTATTACATGGATACTTATGTGTGCAGATAACTATTAAGTATTAACAGCTCATCAGT
GGCTTGCAGATAGTAATGTCTCTCAAGTTGTTTACATGACATTTAACTAAATGAGCATCTCATAAG
TATTTTAAGCATTTTATTAATTAGATTGATTATTAAAAAAATTAATTTGATTTACGGTTAAAAATAGC
TTTTGTTTAAAATTTAAA SEQ ID NO: 5
TATTTAACCAATATAAAAAAAATTAGCCTTCCATTCTCTTACCAAAAAAAAGTAATATTAATTTTCAA
TTTGCATCTCTGTTACATTATTTCAGGTCACGTAGCCAGCAAAAGGAGACACTGGTATGCACCAG
CATTTTTCTTTTCCTTTTTTTTGTGGTCTTTATTTTTTAGAATATATATGATAATATATACGGATTAA
TTTCAATTTCTTTTGTATCATATTCAACTACTAGTTTACATATATGGGACTTTTTAATAATCATCAAG
AGATAAAATTTAAATATTTTTATTTATCATATAATTAGAGATAAGATAAATTTTGATTCATGAATTTG
ATCTGATAATTAATATAAATTTTAAACATACTTAATTTGATAATGAGTTTTATAACTATAATTCAATC
AAGTCATTTTGTCACCCATTCTATTACTGTATTACATGGATACTTATGTGTGCAGATAACTATTAAG
TATTAACAGCTCATCAGTGGCTTGCAGATAGTAATGTCTCTCAACTTGTTTACATGACATTTAACT
AAATGAGCATCTCATAAGTATTTTAAGCATTTTATTAATTAGATTGATTATTAA SEQ ID NO: 6
ACCAAAAAAAAGTAATATTAATTTTCAATTTGCATCTCTGTTACATTATTTCAGGTCACGTAGCCAG
CAAAAGGAGACACTGGTATGCACCAGCATTTTTCTTTTCCTTTTCTTTTGTGGTCTTTATTTTTTAG
AATATATATGATAATATATACGGATTAATTTCAATTTCTTTTGTATCATATTCAACTACTAGTTTACA
TATATGGGACTTTTTAATAATCATCAAGAGATAAAATTTAAATATTTTTATTTATCATATAATTAGAG
ATAAGATAAATTTTGATTCATGAATTTGATCTGATAATTAATATAAATTTTAAACATACTTAATTTGA
TAATGAGTTTTATAACTATAATTCAATGAAGTCATTTTGTCACCCATTCTATTACTGTATTACATGG
ATACTTATGTGTGCAGATAACTATTAAGTATTAACAGCTCATCAGTGGCTTGCAGATAGTAATGTC
TCTCAACTTGTTTACATGACATTTAACTAAATGAGCATCTCAT SEQ ID NO: 7
ATTTTCAATTTGCATCTCTGTTAGATTATTTCAGGTCACGTAGCCAGCAAAAGGAGACACTGGTAT
GCACCAGCATTTTTCTTTTCCTTTTCTTTTGTGGTCTTTATTTTTTAGAATATATATGATAATATATA
CGGATTAATTTCAATTTCTTTTGTATCATATTCAACTACTAGTTTACATATATGGGACTTTTTAATAA
TCATCAAGAGATAAAATTTAAATATTTTTATTTATCATATAATTAGAGATAAGATAAATTTTGATTCA
TGAATTTGATCTGATAATTAATATAAATTTTAAACATACTTAATTTGATAATGAGTTTTATAACTATA
ATTCAATCAAGTCATTTTGTCACCCATTCTATTACTGTATTACATGGATACTTATGTGTGCAGATAA
CTATTAAGTATTAACAGCTCATCAGTGGCTTGCAGATAGTAATGTCTCTCAACTTGTTTACATGAC
AT SEQ ID NO: 8
TTACATTATTTCAGGTCACGTAGCCAGCAAAAGGAGACACTGGTATGCACCAGCATTTTTCTTTTC
CTTTTCTTTTGTGGTCTTTATTTTTTAGAATATATATGATAATATATACGGATTAATTTCAATTTCTTT
TGTATCATATTCAACTACTAGTTTACATATATGGGACTTTTTAATAATCATCAAGAGATAAAATTTAA
ATATTTTTATTTATCATATAATTAGAGATAAGATAAATTTTGATTCATGAATTTGATCTGATAATTAA
TATAAATTTTAAACATACTTAATTTGATAATGAGTTTTATAACTATAATTCAATCAAGTCATTTTGTC
ACCCATTCTATTACTGTATTACATGGATACTTATGTGTGCAGATAACTATTAAGTATTAACAGCTCA
TCAGTGGCTTGCAGATAGTAATGTCTC SEQ ID NO: 9
TAGCCAGCAAAAGGAGACACTGGTATGCACCAGCATTTTTCTTTTCCTTTTCTTTTGTGGTCTTTA
TTTTTTAGAATATATATGATAATATATACGGATTAATTTCAATTTCTTTTGTATCATATTCAACTACTA
GTTTACATATATGGGACTTTTTAATAATCATCAAGAGATAAAATTTAAATATTTTTATTTATCATATA
ATTAGAGATAAGATAAATTTTGATTCATGAATTTGATCTGATAATTAATATAAATTTTAAACATACTT
AATTTGATAATGAGTTTTATAACTATAATTCAATCAAGTCATTTTGTCACCCATTCTATTACTGTATT
ACATGGATACTTATGTGTGCAGATAACTATTAAGTATTAACAGCTCATCAGTGG SEQ ID NO: 10
TATTTAACCAATATAAAAAAAATTAGCCTTCCATTCTCTTACCAAAAAAAAGTAATATTAATTTTCAA
TTTGCATCTCTGTTACATTATTTCAGGTCACGTAGCCAGCAAAAGGAGACACTGGTATGCACCAG
CATTTTTCTTTTCCTTTTCTTTTGTGGTCTTTATTTTTTAGAATATATATGATAATATATACGGATTAA
TTTCAATTTCTTTTGTATCATATTCAACTACTAGTTTACATATATGGGACTTTTTAATAATCATCAAG
AGATAAAATTTAAATATTTTTATTTATCATATAATTAGAGATAAGATAAATTTTGATTCATGAATTTG
ATCTGATAATT SEQ ID NO: 11
ACCAAAAAAAAGTAATATTAATTTTCAATTTGCATCTCTGTTACATTATTTCAGGTCACGTAGCCAG
CAAAAGGAGACACTGGTATGCACCAGCATTTTTCTTTTCCTTTTCTTTTGTGGTCTTTATTTTTTAG
AATATATATGATAATATATACGGATTAATTTCAATTTCTTTTGTATCATATTCAACTACTAGTTTACA
TATATGGGACTTTTTAATAATCATCAAGAGATAAAATTTAAATATTTTTATTTATCATATAATTAGA
SEQ ID NO: 12
ATTTTCAATTTGCATCTCTGTTACATTATTTCAGGTCACGTAGCCAGCAAAAGGAGACACTGGTAT
GCACCAGGATTTTTCTTTTCCTTTTCTTTTGTGGTCTTTATTTTTTAGAATATATATGATAATATATA
CGGATTAATTTCAATTTCTTTTGTATCATATTCAACTACTAGTTTACATATATGGGACTTTTTAATAA
TCATCAAGAGATAAAATTTAAATATTT SEQ ID NO: 13
TTAGATTATTTCAGGTCACGTAGCCAGCAAAAGGAGACACTGGTATGCACCAGCATTTTTCTTTTC
CTTTTCTTTTGTGGTCTTTATTTTTTAGAATATATATGATAATATATAGGGATTAATTTCAATTTCTTT
TGTATCATATTCAACTACTAGTTTACATATATGGGACTTTTTAATAATCATCAA SEQ ID NO: 14
TAGCCAGCAAAAGGAGACACTGGTATGCACCAGCATTTTTCTTTTCCTTTTCTTTTGTGGTCTTTA
TTTTTTAGAATATATATGATAATATATACGGATTAATTTCAATTTCTTTTGTATCATATTCAACTACTA
GTTTACATATATGG SEQ ID NO: 15
TATTTAACCAATATAAAAAAAATTAGCCTTCCATTCTCTTACCAAAAAAAAGTAATATTAATTTTCAA
TTTGCATCTCTGTTACATTATTTCAGGTCACGTAGCCAGCAAAAGGAGACACTGGTATGCACCAG
CATTTTTCTTTTCCTTTTCTTTTGTGGTCTTTATTTTTTAGAATATATATGATAATATATACGGATTAA
TTTCAATTTCTTTTGTATCAT SEQ ID NO: 16
ACCAAAAAAAAGTAATATTAATTTTCAATTTGCATCTCTGTTACATTATTTCAGGTCACGTAGCCAG
CAAAAGGAGACACTGGTATGCACCAGCATTTTTCTTTTCCTTTTCTTTTGTGGTCTTTATTTTTTAG
AATATATAT SEQ ID NO: 17
ATTTTCAATTTGCATCTCTGTTACATTATTTCAGGTCACGTAGCCAGCAAAAGGAGACACTGGTAT
GCACCAGCATTTTTCTTTTCCTTTTCTTTTGTGGTCT SEQ ID NO: 18
TTACATTATTICAGGTCACGTAGCCAGCAAAAGGAGACACTGGTATGCACCAGCATTTTTCTT
SEQ ID NO: 19
TTAACCAATATAAAAAAAATTAGCCTTCCATTCTCTTACCAAAAAAAAGTAATATTAATTTTCAATTT
GCATCTCTGTTACATTATTTCAGGTCACGTAGCCAGCAAAAGGAGACACTGGTATGCACCAGCAT
TTTTCTTTTCCTTTTCTTTTGTGGTCTTTATTTTTTAGAATATATATGATAATATATACGGATTAATTT
CAATTTCTTTTGTATCATATT SEQ ID NO: 20
ACCAAAAAAAAGTAATATTAATTTTCAATTTGCATCTCTGTTACATTATTTCAGGTCACGTAGCCAG
CAAAAGGAGACACTGGTATGCACCAGCATTTTTCTTTTCCTTTTCTTTTGTGGTCTTTATTTTTTAG
AATATATATGA SEQ ID NO: 21
ATTTTCAATTTGCATCTCTGTTACATTATTTCAGGTCACGTAGCCAGCAAAAGGAGACACTGGTAT
GCACCAGCATTTTTCTTTTCCTTTTCTTTTGTGGTCTTTA SEQ ID NO: 22
TTACATTATTTCAGGTCACGTAGCCAGCAAAAGGAGACACTGGTATGCACCAGCATTTTTCTTTTC
SEQ ID NO: 23
GCACCAGCATTTTTCTTTTCCTTTTCTTTTGTGGTCTTTATTTTTTAGAATATATATGATAATATATA
CGGATTAATTTCAATTTCTTTTGTATCATATTCAACTACTAGTTTACATATATGGGACTTTTTAATAA
TCATGAAGAGATAAAATTTAAATATTTTTATTTATCATATAATTAGAGATAAGATAAATTTTGATTCA
TGAATTTGATCTGATAATT SEQ ID NO: 24
TTTTTTAGAATATATATGATAATATATACGGATTAATTTCAATTTCTTTTGTATCATATTCAACTACTA
GTTTACATATATGGGACTTTTTAATAATCATCAAGAGATAAAATTTAAATATTTTTATTTATCATATA
ATTAGA SEQ ID NO: 25
ATATACGGATTAATTTCAATTTCTTTTGTATCATATTCAACTACTAGTTTACATATATGGGACTTTTT
AATAATCATGAAGAGATAAAATTTAAATATTT SEQ ID NO: 26
AATTTCTTTTGTATCATATTCAACTACTAGTTTACATATATGGGACTTTTTAATAATCATCAA SEQ
ID NO: 27
TAATTTGATAATGAGTTTTATAACTATAATTCAATCAAGTCATTTTGTCACCCATTCTATTACTGTAT
TACATGGATACTTATGTGTGCAGATAACTATTAAGTATTAACAGCTCATCAGTGGCTTGCAGATAG
TAATGTCTCTCAACTTGTTTACATGACATTTAACTAAATGAGCATCTCATAAGTATTTTAAGCATTT
TATTAATTAGATTGATTATTAA SEQ ID NO: 28
CATTTTGTCACCCATTCTATTACTGTATTACATGGATACTTATGTGTGCAGATAACTATTAAGTATT
AACAGCTCATCAGTGGCTTGCAGATAGTAATGTCTCTCAACTTGTTTACATGACATTTAACTAAAT
GAGCATCTCAT SEQ ID NO: 29
TACTGTATTACATGGATACTTATGTGTGCAGATAACTATTAAGTATTAACAGCTCATCAGTGGCTT
GCAGATAGTAATGTCTCTCAACTTGTTTACATGACAT SEQ ID NO: 30
TATGTGTGCAGATAACTATTAAGTATTAACAGCTCATCAGTGGCTTGCAGATAGTAATGTCTC
SEQ ID NO: 31
ATACATACATACATGTACATGTATCTTCACTGAAACGTCCTCGATTCTCTTGCGAGCTTCTGCTTT
AGACAACATGATACATTCCGCTGCTTCATCCATGGCCTCACCCCTCTGCACGTGGCTGGTGGCA
GCTTGCATGTCAGTCACGTGCCACGCTGACCGCGCCGGGACTCCAAAGGCAGCGCTTCGCTCA
TCCAAGAGGTCAAGGAGGACGAAGGCGCTGCCACCAAACACCTCCCAATTGAACATGAGATTGA
TTTCTTCGCTCTACGGATCCAGCATTCAGGGACTCATAAGCTCCTTCGAGCCCTGCGATGACTAC
TGCAACACTCACAATGCCTTCTCTTCTCTCTTTCGATCCACAACTCCCAATCGCAGACACACCCG
TCTCAATAAACTTGCTCACTCTGGTAAACTTTTTTTTTTTTTTTAATTTTCCCCACTTAAATCACCTC
AAGTTAAGATATTTCTG SEQ ID NO: 32
TGCGAGCTTCTGCTTTAGACAACATGATACATTCCGCTGCTTCATCCATGGCCTCACCCCTCTGC
ACGTGGCTGGTGGCAGCTTGCATGTCAGTCACGTGCCACGCTGACCGCGCCGGGACTCCAAAG
GCAGCGCTTCGCTCATCCAAGAGGTCAAGGAGGACGAAGGCGCTGCCACCAAACACCTCCCAA
TTGAACATGAGATTGATTTCTTCGCTCTACGGATCCAGCATTCAGGGACTCATAAGCTCCTTCGA
GCCCTGCGATGACTACTGCAACACTCACAATGCCTTCTCTTCTCTCTTTCGATCCACAACTCCCA
ATCGCAGACACACCCGTCTCAATAAACTTGCTCACTCTGGTAAACTTTTTT SEQ ID NO: 33
TTCATCCATGGCCTCACCCCTCTGCACGTGGCTGGTGGCAGCTTGCATGTCAGTCACGTGCCAC
GCTGACCGCGCCGGGACTCCAAAGGCAGCGCTTCGCTCATCCAAGAGGTCAAGGAGGACGAAG
GCGCTGCCACCAAACACCTCCCAATTGAACATGAGATTGATTTCTTCGCTCTACGGATCCAGCAT
TCAGGGACTCATAAGCTCCTTCGAGCCCTGCGATGACTACTGCAACACTCACAATGCCTTCTCTT
CTCTCTTTCGATCCACAACTCCCAATCGCAGACAC SEQ ID NO: 34
TCTGCACGTGGCTGGTGGCAGCTTGCATGTCAGTCACGTGCCACGCTGACCGCGCCGGGACTC
CAAAGGCAGCGCTTCGCTCATCCAAGAGGTCAAGGAGGACGAAGGCGCTGCCACCAAACACCT
CCCAATTGAACATGAGATTGATTTCTTCGCTCTACGGATCCAGCATTCAGGGACTCATAAGCTCC
TTCGAGCCCTGCGATGACTACTGCAACACTCACAATGCCTTCTCTTCTCTCTTTCGATCCA SEQ
ID NO: 35
GCTTGCATGTCAGTCACGTGCCACGCTGACCGCGCCGGGACTCCAAAGGCAGCGCTTCGCTCA
TCCAAGAGGTCAAGGAGGACGAAGGCGCTGCCACCAAACACCTCCCAATTGAACATGAGATTGA
TTTCTTCGCTCTACGGATCCAGCATTCAGGGACTCATAAGCTCCTTCGAGCCCTGCGATGACTAC
TGCAACACTCACAATGCCTT SEQ ID NO: 36
CCACGCTGACCGCGCCGGGACTCCAAAGGCAGCGCTTCGCTCATCCAAGAGGTCAAGGAGGAC
GAAGGCGCTGCCACCAAACACCTCCCAATTGAACATGAGATTGATTTCTTCGCTCTACGGATCCA
GCATTCAGGGACTCATAAGCTCCTTCGAGCCCTGCGATGACTAC SEQ ID NO: 37
CCACGCTGACCGCGCCGGGACTCCAAAGGCAGCGCTTCGCTCATCCAAGAGGTCAAGGAGGAC
GAAGGCGCTGCCACCAAACACCTCCCAATTGAACATGAGATTGATTTCTTCGCTCTACGG SEQ
ID NO: 38
AGATTGATTTCTTCGCTCTACGGATCCAGCATTCAGGGACTCATAAGCTCCTTCGAGCCCTGCGA
TGACTAC SEQ ID NO: 39
TGCGAGCTTCTGCTTTAGACAACATGATACATTCCGCTGCTTCATCCATGGCCTCACCCCTCTGC
ACGTGGCTGGTGGCAGCTTGCATGTCAGTCACGTGCCACGCTGACCGCGCCGGGACTCCAAAG
GCAGCGCTTCGCTCATCCAAGAGGTCAAGGAGGACGAAGGCGCTGCCACCAAACACCTCCCAA
TTGAACATGAGATTGATTTCTTCGCTCTACGG SEQ ID NO: 40
TTCATCCATGGCCTCACCCCTCTGCACGTGGCTGGTGGCAGCTTGCATGTCAGTCACGTGCCAC
GCTGACCGCGCCGGGACTCCAAAGGCAGCGCTTCGCTCATCCAAGAGGTCAAGGAGGACGAAG
GCGCTGCCACCAAAC SEQ ID NO: 41
TCTGCACGTGGCTGGTGGCAGCTTGCATGTCAGTCACGTGCCACGCTGACCGCGCCGGGACTC
CAAAGGCAGCGCTTCGCTCATCCAAGAGGTCAAGGAGGAC SEQ ID NO: 42
GCTTGCATGTCAGTCACGTGCCACGCTGACCGCGCCGGGACTCCAAAGGCAGCGCTTCGCTCA
SEQ ID NO: 43
CCACGCTGACCGCGCCGGGACTCCAAAGGCAGCGCTTCGCTCATCCAAGAGGTCAAGGAGGAC
GAAGGCGCTGCCACCAAACACCTCCCAATTGAACATGAGATTGATTTCTTCGCTCTACGGATCCA
GCATTCAGGGACTCATAAGCTCCTTCGAGCCCTGCGATGACTACTGCAACACTCACAATGCCTTC
TCTTCTCTCTTTCGATCCACAACTCCCAAT SEQ ID NO: 44
TCATCCAAGAGGTCAAGGAGGACGAAGGCGCTGCCACCAAACACCTCCCAATTGAACATGAGAT
TGATTTCTTCGCTCTACGGATCCAGCATTCAGGGACTCATAAGCTCCTTCGAGCCCTGCGATGAC
TACTGCAACACTCAC SEQ ID NO: 45
GACGAAGGCGCTGCCACCAAACACCTCCCAATTGAACATGAGATTGATTTCTTCGCTCTACGGAT
CCAGCATTCAGGGACTCATAAGCTCCTTCGAGCCCTGC SEQ ID NO: 46
ACACCTCCCAATTGAACATGAGATTGATTTCTTCGCTCTACGGATCCAGCATTCAGGGACTCA
SEQ ID NO: 47
AGGTCAAGGAGGACGAAGGCGCTGCCACCAAACACCTCCCAATTGAACATGAGATTGATTTCTT
CGCTCTACGGATCCAGCATTCAGGGACTCATAAGCTCCTTCGAGCCCTGCGATGACTACTGCAA
CACTCACAATGCCTTCTCTTCTCTCTTTCGATCCACAACTCCCAATCGCAGACACACCCGTCTCA
ATAAACTTGCTCACTCTGGTAAACTTTTTT SEQ ID NO: 48
AATTGAACATGAGATTGATTTCTTCGCTCTACGGATCCAGCATTCAGGGACTCATAAGCTCCTTC
GAGCCCTGCGATGACTACTGCAACACTCACAATGCCTTCTCTTCTCTCTTTCGATCCACAACTCC
CAATCGCAGACAC SEQ ID NO: 49
TCTTCGCTCTACGGATCCAGCATTCAGGGACTCATAAGCTCCTTCGAGCCCTGCGATGACTACTG
CAACACTCACAATGCCTTCTCTTCTCTCTTTCGATCCA SEQ ID NO: 50
CATTCAGGGACTCATAAGCTCCTTCGAGCCCTGCGATGACTACTGCAACACTCACAATGCCTT
SEQ ID NO: 51
ATACGAATTTCTTGAATGATAGGCAGTTGCATGCAATTTTTATTATTGCTGACTTATTCCTCTGAAT
TGCATAACTATAAGGTAAAACAATGGCTGTAGCTGTGGAACCTGCACATGAAGTCACAACAACGA
AAAAACCTCCTACAAAACAAAGGCGAGTAGTTGTGACCGGGTTGGGCGTTGTTACACCTCTTGGT
CACGAACCAGATGTTTACTACAACAATTTACTTGATGGTGTTAGCGGCATTAGTGAGATCGAGAC
ATTTGATTGTGCTGAATATCCAACGGCAAGTTATATATAATTGAAATTGTATTTTTTGTTGTCTGTT
GTAGCTTTTGGTCTGAATATTCTATGTTTCCA SEQ ID NO: 52
GACTTATTCCTCTGAATTGCATAACTATAAGGTAAAACAATGGCTGTAGCTGTGGAACCTGCACAT
GAAGTCACAACAACGAAAAAACCTCCTACAAAACAAAGGCGAGTAGTTGTGACCGGGTTGGGCG
TTGTTACACCTCTTGGTCACGAACCAGATGTTTACTACAACAATTTACTTGATGGTGTTAGCGGCA
TTAGTGAGATCGAGACATTTGATTGTGCTGAATATCCAACGGCAAGTTATATATAATTGAAATTG
SEQ ID NO: 53
TGGCTGTAGCTGTGGAACCTGCACATGAAGTCACAACAACGAAAAAACCTCCTACAAAACAAAGG
CGAGTAGTTGTGACCGGGTTGGGCGTTGTTACACCTCTTGGTCACGAACCAGATGTTTACTACAA
CAATTTACTTGATGGTGTTAGCGGCATTAGTGAGATCGAGACATTTGATTG SEQ ID NO: 54
GCACATGAAGTCACAACAACGAAAAAACCTCCTACAAAACAAAGGCGAGTAGTTGTGACCGGGT
TGGGCGTTGTTACACCTCTTGGTCACGAACCAGATGTTTACTACAACAATTTACTTGATGGTGTTA
GCGGCATTAGT SEQ ID NO: 55
GAAAAAACCTCCTACAAAACAAAGGCGAGTAGTTGTGACCGGGTTGGGCGTTGTTACACCTCTT
GGTCACGAACCAGATGTTTACTACAACAATTTACTTG SEQ ID NO: 56
AAAGGCGAGTAGTTGTGACCGGGTTGGGCGTTGTTACACCTCTTGGTCACGAACCAGATGT SEQ
ID NO: 57
GACTTATTCCTCTGAATTGCATAACTATAAGGTAAAACAATGGCTGTAGCTGTGGAACCTGCACAT
GAAGTCACAACAACGAAAAAACCTCCTACAAAACAAAGGCGAGTAGTTGTGACCGGGTTGGGCG
TTGTTACACCTCTTGGTCACGAACCAGATGTTTACTACAACAATTTACTTGATGGTGTTAGCGGCA
TTAGTGAGATCGAGACATTTGATTGTG SEQ ID NO: 58
TGGCTGTAGCTGTGGAACCTGCACATGAAGTCACAACAACGAAAAAACCTCCTACAAAACAAAGG
CGAGTAGTTGTGACCGGGTTGGGCGTTGTTACACCTCTTGGTCACGAACCAGATGTTTACTACAA
CAATTTACTTGAT SEQ ID NO: 59
GCACATGAAGTCACAACAACGAAAAAACCTCCTACAAAACAAAGGCGAGTAGTTGTGACCGGGT
TGGGCGTTGTTACACCTCTTGGTCACGAACCAGATGTTT SEQ ID NO: 60
GAAAAAACCTCCTACAAAACAAAGGCGAGTAGTTGTGACCGGGTTGGGCGTTGTTACACCTCT
SEQ ID NO: 61
GCATAACTATAAGGTAAAACAATGGCTGTAGCTGTGGAACCTGCACATGAAGTCACAACAACGAA
AAAACCTCCTACAAAACAAAGGCGAGTAGTTGTGACCGGGTTGGGCGTTGTTACACCTCTTGGT
CACGAACCAGATGTTTACTACAACAATTTACTTGATGGTGTTAGCGGCATTAGTGAGATCGAGAC
ATTTGATTGTGCTGAATATCCAACGGCAA SEQ ID NO: 62
CTGCACATGAAGTCACAACAACGAAAAAACCTCCTACAAAACAAAGGCGAGTAGTTGTGACCGG
GTTGGGCGTTGTTACACCTCTTGGTCACGAACCAGATGTTTACTACAACAATTTACTTGATGGTGT
TAGCGGCATTAGT SEQ ID NO: 63
AACGAAAAAACCTCCTACAAAACAAAGGCGAGTAGTTGTGACCGGGTTGGGCGTTGTTACACCT
CTTGGTCACGAACCAGATGTTTACTACAACAATTTACTTG SEQ ID NO: 64
ACAAAGGCGAGTAGTTGTGACCGGGTTGGGCGTTGTTACACCTCTTGGTCACGAACCAGATGT
SEQ ID NO: 65
AATGGCTGTAGCTGTGGAACCTGCACATGAAGTCACAACAACGAAAAAACCTCCTACAAAACAAA
GGCGAGTAGTTGTGACCGGGTTGGGCGTTGTTACACCTCTTGGTCACGAACCAGATGTTTACTA
CAACAATTTACTTGATGGTGTTAGCGGCATTAGTGAGATCGAGACATTTGATTGTGCTGAATATCC
AACGGCAAGTTATATATAATTGAAATTG SEQ ID NO: 66
ACGAAAAAACCTCCTACAAAACAAAGGCGAGTAGTTGTGACCGGGTTGGGCGTTGTTACACCTC
TTGGTCACGAACCAGATGTTTACTACAACAATTTACTTGATGGTGTTAGCGGCATTAGTGAGATC
GAGACATTTGATTG SEQ ID NO: 67
ACAAAGGCGAGTAGTTGTGACCGGGTTGGGCGTTGTTACACCTCTTGGTCACGAACCAGATGTT
TACTACAACAATTTACTTGATGGTGTTAGCGGCATTAGT SEQ ID NO: 68
CCGGGTTGGGCGTTGTTACACCTCTTGGTCACGAACCAGATGTTTACTACAACAATTTACTTG
SEQ ID NO: 69
GTGATGCTTTGTGGTGGCTCAGATGCTGCTATTATACCCATTGGTATGATCTCACCATTGATTCTT
TTTCACCTGCATACGGTTATCGTGCCATAAATCTAGCTGATATGAATCCACTAATCATTGGTACAG
GTTTGGGAGGTTTTGTGGCATGCAGAGCCCTATCACAGAGGAATACAGACCCTACCAAAGCTTC
ACGCCCTTGGGACATTGTAATACAACACCTGCTCATTCTTTCTGCTACTCACCATGTTTTGTATTG
CTGAGGCTTTCATCATTTTTTATTTTTGAAATAATACGACTATAATTATAATGAAATTTGATGACCAA
GTCTTGTATCCTTTCTTGTAATCT SEQ ID NO: 70
CTCACCATTGATTCTTTTTCACCTGCATACGGTTATCGTGCCATAAATCTAGCTGATATGAATCCA
CTAATCATTGGTACAGGTTTGGGAGGTTTTGTGGCATGCAGAGCCCTATCACAGAGGAATACAGA
CCCTACCAAAGCTTCACGCCCTTGGGACATTGTAATACAACACCTGCTCATTCTTTCTGCTACTCA
CCATGTTTTGTATTGCTGAGGCTTTCATCATTTTTTATTTTTGAAATAATACGACTA SEQ ID NO: 71
CCATAAATCTAGCTGATATGAATCCACTAATCATTGGTACAGGTTTGGGAGGTTTTGTGGCATGC
AGAGCCCTATCACAGAGGAATACAGACCCTACCAAAGCTTCACGCCCTTGGGACATTGTAATACA
ACACCTGCTCATTCTTTCTGCTACTCACCATGTTTTGTATTGCT SEQ ID NO: 72
AATCCACTAATCATTGGTACAGGTTTGGGAGGTTTTGTGGCATGCAGAGCCCTATCACAGAGGAA
TACAGACCCTACCAAAGCTTCACGCCCTTGGGACATTGTAATACAACACCTGCTCATTCTTTCTG
CTAC SEQ ID NO: 73
AGGTTTGGGAGGTTTTGTGGCATGCAGAGCCCTATCACAGAGGAATACAGACCCTACCAAAGCT
TCACGCCCTTGGGACATTGTAATACAACAC SEQ ID NO: 74
CATGCAGAGCCCTATCACAGAGGAATACAGACCCTACCAAAGCTTCACGCCCTT SEQ ID NO: 75
CTCACCATTGATTCTTTTTCACCTGCATACGGTTATCGTGCCATAAATCTAGCTGATATGAATCCA
CTAATCATTGGTACAGGTTTGGGAGGTTTTGTGGCATGCAGAGCCCTATCACAGAGGAATACAG
ACCCTACCAAAGCTTCACGCCCTTGGGACATTGTAATACAACACCTGCTCATTCTTTCTGCTACTC
ACCATGTTTTGTATTGCTGAGGCTTTC SEQ ID NO: 76
CATAAATCTAGCTGATATGAATCCACTAATCATTGGTACAGGTTTGGGAGGTTTTGTGGCATGCA
GAGCCCTATCACAGAGGAATACAGACCCTACCAAAGCTTCACGCCCTTGGGACATTGTAATACAA
CACCTGCTCATTCTTTCTGCTACTCACCATGTTTTGTATTGCT SEQ ID NO: 77
AATCCACTAATCATTGGTACAGGTTTGGGAGGTTTTGTGGCATGCAGAGCCCTATCACAGAGGAA
TACAGACCCTACCAAAGCTTCACGCCCTTGGGACATTGTAATACAACACCTGCTCATTCTTTCTG
CTAC SEQ ID NO: 78
AGGTTTGGGAGGTTTTGTGGCATGCAGAGCCCTATCACAGAGGAATACAGACCCTACCAAAGCT
TCACGCCCTTGGGACATTGTAATACAACAC SEQ ID NO: 79
GTTATCGTGCCATAAATCTAGCTGATATGAATCCACTAATCATTGGTACAGGTTTGGGAGGTTTTG
TGGCATGCAGAGCCCTATCACAGAGGAATACAGACCCTACCAAAGCTTCACGCCCTTGGGACAT
TGTAATACAACACCTGCTCATTCTTTCTGCTACTCACCATGTTTTGTATTGCTGAGGCTTTCATCAT
TTTTTATTTTTGAAATAATACGACTA SEQ ID NO: 80
CATTGGTACAGGTTTGGGAGGTTTTGTGGCATGCAGAGCCCTATCACAGAGGAATACAGACCCT
ACCAAAGCTTCACGCCCTTGGGACATTGTAATACAACACCTGCTCATTCTTTCTGCTACTCACCAT
GTTTTGTATTGCT SEQ ID NO: 81
GTTTTGTGGCATGCAGAGCCCTATCACAGAGGAATACAGACCCTACCAAAGCTTCACGCCCTTG
GGACATTGTAATACAACACCTGCTCATTCTTTCTGCTAC SEQ ID NO: 82
CTATCACAGAGGAATACAGACCCTACCAAAGCTTCACGCCCTTGGGACATTGTAATACAACAC
SEQ ID NO: 83
KASII genomic (Arachis hypogaea)
TTTGTATCATATTCAACTACTAGTTTACATATATGAGACTTTTTAATAACCATCAAGAGATAAAATTT
AAATATTTTTATTGATCATATAATACTTAATTGTATAGTTGTGTAAAATACTTTAAATAATACTATTAA
TATATTAAAATCAGCTATTATATATATTGTTTAATTTATTTTTAATATATATTTTATATTTTAATATGTA
TTTTATATTAATGTTTGATTTTGGTATATACTTAATAATACAGTTAATACATTGATAATATTTTAGACT
ATTTTTCTTAATTTTATTTTTTAAAGTTTTAATATCAACCATTCATTTATTAAAGATAAGATAAATTTT
GATTCATGAATTTGATCTGATAATTAATATAAATTTTAAACATGCTTAATTTGATAATGAGTTTTATA
ACTATAATTCAATCAAGTCATTTTGTCACCCATTCATGAATACTTATGTGTGCAGATAACTATTAAC
AGCTCATCAGTAGCTTGCTGATGGTAATGTCTCTCAACTTGTTTACATGACAGTTAACTAACCGG
GCATCTCATAAGTATTTTAATTTGTAAGCATTTTATTAATTAGATTGATTATAAAAAAAAATTAATTT
GATTTACGGTTAAAAATAGCTTTTGTTTAAAATTTAAAAGCCAAACTAACTATTAGATGAAAATTTA
TGAGCAAATTGAGTTTTAATGCTAGTTTGGATTGGTTTTTTTGAGTAAAAATTATTTTTTAAAAACA
AATTAAAGTACTTTTTACTCAATTTTAAATTTATTTAGATATATTTTTTTAATTTATTAAAAAATTAAA
CTATTTACTTTTTTAAAAGTTAACAATATTTTACTTTTAAAAAAATAGAAACAAAAAATATTTTTAATA
TTTGAAATTTTTTAAAAAGCTTATTTAAATGTAAAATAATTTAAAAAAATAAAAACTAAATATTTTTTT
AAAAATCAATTTAAACTGATCTTTATCTCTTAATTATTAACTAATTTTGGTATTTACTTTCAAATTTTC
GTAAAAAAAAGTCCTACAAGAGAATAAAGTATTATTTTGACTAAAGAAAATCTTATTTTGACTAAAG
AAAATCTGAAACCAAGAATAAAATAAATGCTATGTCACGTTTTACACTAATGATAAAAAAATTAAAG
TACAATATAAGTAGAAAATATGTTCTAAAACCAAACAAAAAAGTGCATTAAATTGCGAAAAGGATT
GATCCAAGGAGAAATGGCGTAATGAATTAGCATAGAAAGCAATTAGTTTTATATTGCATAAATAAT
GATGAATTATATTAGGTAAAGGTTCAGGTGGAGTGGAATTCACGTAAAGTTGATAACTGAGAGTC
GTTCGATAATTTGATTGATTTGACTAAATTTTTATTTAACGGCTCTCGACTATCAACTTTGCGATGC
ACCTGAATTTTACCATTATATTAGTAGCGAAGAAAAAGAAAAGAGGTTAGTAGAAAATTAAATTAA
ATTAATAAAGAAAGCAAATGAGAAGCGAAGGAGCGACTGAGACTCAGATTGCAGAAAAAGCAGA
GACTTTGAGACTCGACTCTTGAGACTTCACAACGCCTCCCTTTTCCTCCTTCTCTCAGATCGATTC
TCTTTCTCACTCTCTCAACTCTCTTCAAAACCCCCACAACTAACTTTCTTCTTCTTCTTCTTCTTGT
TCTTTGTATATACATACATACATACATACATGTACATGTATCTCCACCTTCCATATCCACCACTCCT
GACTTGCTCTTAATCTTCACTGAAACGTCCTCGATTCTCTTGCGAGCTTCTGCTTTAGACAACATG
ATACATTCCGCTGCTTCATCCATGGCCTCACCCCTCTGCACGTGGCTGGTGGCGGCTTGCATGT
CAGTCACGTGCCACACTGACCGCGCCGGGACTCCAAAGGCAGCGCTTCGGTCATCCAAGAGGT
CAAGGAGGACGAAGGCGCTGCCAGCAAACACCTCCCAATTGAACATGAGATTGATTTCTTCGCT
GTACGGATCCAGCATTCAGGGACTCATAAGCTCCTTCGAGCCCTGCGATGACTACTGCAACACT
CACAATGCCTTCTCTTCGCTCTTTCGATCCACAACTCCCAATCGCAGACACACCCGTCTCAATAA
ACTTTCTCATTCTGGTAAACTTTTTTTTTTTTTTTTTTAATTTTCCCCACTTAAATCACCTCAAGTTAA
GATATTTCTGGTGCTTTTTCCAATTTTAATCTGTACCCCGTACTGGGTATTTCAATTTGATTCATTT
ATTTATTTTTCTTATATTAATATAATAATAATAGTTTTTTTTTTTATTATGGTTATGAAATCTGGGTTC
TGATTTTCTGGTCTTCTGAAAGATATGTGTGTGTGTGTGGCTTTTTTCCCTTTTTTTTCTGAATTAC
TTAAAATGCACAAGTGTTAGCTTGGATCTTTGTCTTTTGGTTGGTCATCCTAATAAATGGGGTTGC
TTGGTGGTTTAGTATTGAGGGTTGTTCTACCTGCTCTGTTTAACTGTTTTGAGGGTACGGTTTATA
ATATTAGGAGTATGGGGGTAGTTGTTGTTCTTTCTTTGCTGAGATTGATGGTGCACCTGTGTCAC
TTAATTAGGACATGTTGCTACAACACTATGCCATTGCCCATGCTTTTTGCTTTCATTTCCCATTCCT
ACTTCATTTCCTTGTATTTATCGTACACCCAGGTATCTACATAAAGTTCCCATTAAATATTGGTTAA
TGTTCGCATCATCACTTTGAATTTCTTGCAATCTCGTTGAGGTAGTTCAGCATATTCACAGAATCA
ACTCAAACGAAAATAAATTTCTTTTGACTTTTATTTTGTTTTCTTTTCTTCATGAAAATAATAATCAC
ACTGAAGTGTTCTGAATTTAACTACTATGTAAATATCGCCCCCACGTGGGATTACCTCTCTCTCCC
TCTCTCTCTAGTTTTTTTGTTTCTCTATGTAAAATAGTGGAGGTAAGTTATGCCTTTGTCATAAGCA
CACATAAGCAAGCAGAGGCTCCTTTTTTTTTCCCTCATTTTTTTTTTAAATTATCATGTAAAATAGA
TGATTCTGGCACAAACTATCCACAAGTTATTTTTGCTCCTAGTTCTTTAGTATGTTTTTAACACACT
GGACAAGTCTACTACTGCTGCATGCATGTCCCTGACTGATGGGTTCTAGTATTCAGTGGTCTTGA
GTGTTTTGATTTAAAGCAAGGGATATAAGTTGGGTCTGATGAATTTTTCGAGGTTTCATAGCATTT
TTGTTACCATAGTCTTAGAATTACTACCTTGGATAAACCTAAGCATATTTTTGTTAATGGCTGGGG
TTGCATTTCCTGCTCAACATTAATGTGCAACAAGGGTATGTTCGGTTAGGGGTTAAAAATGGGGC
GGGAATTTTCAATGGATGTGGCCCATGTGTAATTTTACTCATAGAACTTATTGTAAAAATTTCACC
CCATTTCTACTTCCCAAAAGAACATATCCTTATCATGATCATTGGAAGCTACACATGCATCATGTT
GAACTATAAGCTGTGGGCCGTTCTTTCTTGTATGCTTCTGATATGAATTTCTTGAATGATAGGCGG
TTGCATGCAATTTTTATTATTGCTGACTTATTCCTCTGAATTGCAAAACTATAAGGTAAAACAATGG
CTGTAGCTGTGGAACCTGCACACGAAGTCACAGCAACGAAAAAACCTCCTACAAAACAAAGGCG
AGTAGTTGTGACTGGGTTGGGCGTTGTTACACCTCTTGGTCACGAACCAGATGTTTACTACAACA
ATTTACTTGATGGTGTTAGTGGCATAAGTGAGATCGAGACATTTGATTGTGCTGAATATCCAACG
GCAAGTTATATATAATTGAAATTGTATTTCTTGTTGTCTGTTGTAGCTTTTGTTCTGAACATTCTAT
GTTTCCAGAGGATTGCTGGTGAGATCAAGTCGTTCTCCACTGATGGCTGGGTTGCACCAAAACTT
TCGAAGAGAATGGATAAATTTATGCTCTATTTGCTAACAGCCGGTAAAAAGGCCTTAGTTGATGG
TGGAATTACTGAAGATGTAATGGATGAGTTGAATAAGGAAAAATGTGGAGTTTTGATTGGCTCAG
CGATGGGTGGCATGAAGGTAACCTTGTCTTTCTTTCATGTTGAGATACAAATCTTGTAAAACCAG
CTGGTGTTGTGGACTGAATTTAATGCCACTGCAATCCAAATGGAGCAGAAGAGTGTGCTATATTT
TCAACCTTGTGCCCTTGTTATATTGACTATGATTTTTGGTGCTTTTTCTACTGAGCAGGTTTTTAAT
GATGCCATTGAAGCTTTAAGAGTATCATATAAGAAGATGAATCCTTTTTGTGTACCTTTTGCCACA
ACAAATATGGGATCTGCCATACTTGCGATGGATCTGGTCTGTTGTTGCATGGACACCATAAATTG
GTGTTTTGTTTCGTACCAAGCTTTTATTTTGGCTATGTTATCTTCAGCTTCTAACAAACATTGTTAT
CTTTTATGGATTACAGGGATGGATGGGCCCTAATTATTCAATCTCTACAGCTTGTGCTACAAGTAA
CTTTTGTATATTGAATGCAGCAAACCACATCATAAGAGGTGAAGCTGTAAGTATTCATGCTTTGAT
ATTACTCTCAGGATGGTGAATTTTTCGTATCCAATATTATACCTTATATTTATTTATTTATTTAACTT
TCTTTTTCTGAGACAGTTTGGTGTAATGATATGGTCATTGCATCAAAATGTATTGATAAATTTAATA
TGAAAATATTCCTGCAGGATGTGATGCTTTGTGGTGGCTCAGATGCTGCTATTATACCCATTGGT
ATGATCTCACCATTGATTCTTTTTCACCTGCATACGGTTATCATGCCATAAATCTAGCTGATATGA
ATCCACTAATCATTGGTACAGGTTTGGGAGGTTTTGTGGCATGCAGAGCCCTATCGCAGAGGAAT
ACAGACCCTACCAAAGCTTCACGCCCTTGGGACATTGTAATACAACAGAAATCCTGCTCATTCTT
TCTGCTACTCACCATGTTTTTGTATTGCTGATCTTTCATCATTTTTTATTTTTGAAACAATACAACTA
TAATTATAATGAAATTTGATGACCAAGTCTTGTATCCTTTCTTGTAATCTGAACAGCATAGTTTATT
AAAAACTAACGTTTCAATGGTCACAGATACAACTTTTCCTTTTGATCACAAAAATGCTGTCTTCTAT
GGATATGAGCTATGGCAAATTCAGTTTTTCTAATTAGGCTGTTTATGATATTCCAGAATCGAGATG
GATTTGTCATGGGGGAAGGAGCTGGAGTTTTACTTTTGGAGGACTTGGAGCATGCAAAGGTACA
GGAAATCAGATCTAATAATTTTAAAACAGAACATCCCTTATGAAATGACAGTGCATGTTTTGATATT
ATTACTGCAGACTTGTGCATGTAATTTATTTCATTTAGTGCATCCTTCAGTTCCTTCAAACAGTATT
AAAGGACTTCATACATCTGTTCTCTCTTGAATTAGGATTGATTTTCAGGCTGCTGCTTGTCATAAA
AATTCACCTGTTACTTCTCATGTCAAATCTGCGCTTGACATTTGTCTTGCAACTAAATCAAATGCAT
CTCTTTTACATTGCAGAAAAGAGGTGCAACCATATATGCTGAATTCCTTGGTGGAAGCTTCACCT
GTGATGCATATCATGTGACAGAGCCACGTCCCGATGGTAAAATTTCAAATCTTTTACTACCAAAAT
TTAAAGCATTTATATGCATGCTATGATTACCATGCTTAAAATCCGAAGAGAACAAGGCAAAAGAAT
GTGACAAGGGTATTTTTATATATTTAGAATCATGTGTGCCATTTGCTGATGGTTAGGTTAAGTATTT
AGAAATATACTATATGTTGCATTTTTCTTGTTTGGCACTGGAATTTCTTCTTCCGAGATCGTGATG
GAATTTAAAACTGCATGTTGCGATCATGACTTGTCTCGACAATATGGACCTGGGATTTAATAATAG
TCCTATATTAGTGGACCTAGTAATCCTAGTCATAATTAGTATCTGCTTGTACTTTACCACATTTCCT
TTATGATTCCATTCGGACTCTTCTTTGAGCATTCTATGACTTGACAAGGAATGATTTGTATCTCCC
TGCGGAACTTGCATTCACGTTTCTTTACTCCATCTTAAATAGGAGCTGGCGTTATACTTTGCATTG
AAAAGGCATTAGCTCAGTCTGGAGTATCAAAAGAGGATGTGAACTACATAAATGCACATGCCACA
TCCACGCCTGCTGGGGATCTTAAGGAGTTCCAGGCTCTGCTTCATTGTTTTGGTCAAAATCCCGA
GGTACATCTATCTTTTTAAGTTACACCACCATTAACATTGTTATCTTGGTGATCAATTCTATGGTAA
ACATGATCTCTTGTTCTTCTGGAGTTCATGTTGAGTAAAACACTGGTACTTCTGATATGGTCATCC
ATACTAATGTCTCTTGTTTTTTCAGCTAAGAGTGAATTCTACAAAATCTATGATTGGTCATCTACTA
GGAGCAGCTGGTGGCGTGGAAGCTGTAGCAACAGTCCAGGTAAAACCCAATGGATCACTTAGTT
CGGTACCCCGCGATTGATGACTAAAATGGTCCTGAGGTTTGCTCTCTCTTCGTACAGGCAATTAG
GACAGGGTGGGTGCATCCCAATATCAACCTGGAAAACCCAGATCAAGGAGTGGTAATGTTTCTT
GTTGCATCATTCCATCCTTCATTTCAAAATTTTAATCTTTTAATCTAACATGTCCAAACTAATGACA
AAAATTCAGGATGCCAAAGTGCTTGTGGGCTCAAAGAAAGAGAGACTAGATATCAAGGCTGCCC
TATCCAATTCATTTGGTTTTGGAGGCCACAATTCTTCAATCATATTTGCTCCATATAAGTGAATGAA
TACATGTTTCAGAATAATACCTTCCCTACCATTATACAGAAGGTATGTCTCTAAATAAGAATAGAT
GACCATTTGTATTCATAAAAGATGAAAACACTGACATATGTATCCACACTAGATCGAAACTAAACT
TGTACCCACGCAAGATGTCCTCCGTGTGACAAAAGTACCCTACGTGGCACTCTAACCCTCCAAA
GACAGGATACTTTTGTCACACGGAGGGCATCTTTCGTGGGTACAAGTTTAGTTTCGATTTAATGT
GGGTACATATGTCAGCATTTTCATTTTTCATGGATACAAATGGTCATTTATTCCTCTAAATAATGAG
CAGGTCATATCGTCCTACTACGAGAAACACATTTAATTTGGATGACTGTGGATGTTCCTTGAACAT
GTATTAATTGTGTGTTGTTTTTATGTTAAGTTACTAAGGTAGCCAGGCAATTGTTACACGCATGAT
CAAAGTCATGAATTGGATGAAAATATATGTGGAAATGGTTGGAGCAGAAACTCTTGAGGACATTG
TTGAGAGATGGTTTGGTGGTTTAGATTGTCTGGCCGAGATACATGGTGGAGCCGGTTCTATCCA
CCGATGAAGAACATGAATGGCTGCTATTGCCTTTATAGAGTAGGACTTGACCCACTTTGATGTGA
CTCTTAAACAAATCCACTTGGCAGTATATATAAGCTCTTCTGTTTTATTTATTCCCTATGTACCCAA
ATAGTTTGTGCAATAACAAATTGCAATTTATTTTTTCGCT SEQ ID NO: 116
KASII genomic (Arachis hypogaea)
TTTGTATCATATTCAACTACTAGTTTACATATATGAGACTTTTTAATAACCATCAAGAGATAAAATTT
AAATATTTTTATTGATCATATAATACTTAATTGTATAGTTGTGTAAAATACTTTAAATAATACTATTAA
TATATTAAAATCAGCTATTATATATATTGTTTAATTTATTTTTAATATATATTTTATATTTTAATATGTA
TTTTATATTAATGTTTAATTTTGGTATATACTTAATAATACAGTTAATACATTGATAATATTTTAGACT
ATTTTTCTTAATTTTATTTTTTAAAGTTTTAATATCAACCATTCATTTATTAAAGATAAGATAAATTTT
GATTCATGAATTTGATCTGATAATTAATATAAATTTTAAACATGCTTAATTTGATAATGAGTTTTATA
ACTATAATTCAATCAAGTCATTTTGTCACCCATTCATGAATACTTATGTGTGCAGATAACTATTAAC
AGCTCATCAGTAGCTTGCTGATGGTAATGTCTCTCAACTTGTTTACATGACAGTTAACTAACCGG
GCATCTCATAAGTATTTTAATTTGTAAGCATTTTATTAATTAGATTGATTATAAAAAAAAATTAATTT
GATTTACGGTTAAAAATAGCTTTTGTTTAAAATTTAAAAGCCAAACTAACTATTAGATGAAAATTTA
TGAGCAAATTGAGTTTTAATGCTAGTTTGGATTGGTTTTTTTGAGTAAAAATTATTTTTTAAAAACA
AATTAAAGTACTTTTTACTCAATTTTAAATTTATTTAGATATATTTTTTTAATTTATTAAAAAATTAAA
CTATTTACTTTTTTAAAAGTTAACAATATTTTACTTTTAAAAAAATAGAAACAAAAAATATTTTTAATA
TTTGAAATTTTTTAAAAAGCTTATTTAAATGTAAAATAATTTAAAATAATAAAAACTAAATATTTTTTT
AAAAATCAATTTAAACTGATCTTTATCTCTTAATTATTAACTAATTTTGGTATTTACTTTCAAATTTTC
GTAAAAAAAAGTCCTACAAGAGAATAAAGTATTATTTTGACTAAAGAAAATCTTATTTTGACTAAAG
AAAATCTGAAACCAAGAATAAAATAAATGCTATGTCACGTTTTACACTAATGATAAAAAAATTAAAG
TACAATATAAGTAGAAAATATGTTCTAAAACCAAACAAAAAAGTGCATTAAATTGCGAAAAGGATT
GATCCAAGGAGAAATGGCGTAATGAATTAGCATAGAAAGCAATTAGTTTTATATTGCATAAATAAT
GATGAATTATATTAGGTAAAGGTTCAGGTGGAGTGGAATTCACGTAAAGTTGATAACTGAGAGTC
GTTAGATAATTTGATTGATTTGACTAAATTTTTATTTAACGGCTCTCGACTATCAACTTTGCGATGC
ACCTGAATTTTACCATTATATTAGTAGCGAAGAAAAAGAAAAGAGGTTAGTAGAAAATTAAATTAA
ATTAATAAAGAAAGCAAATGAGAAGCGAAGGAGCGACTGAGACTCAGATTGCAGAAAAAGCAGA
GACTTTGAGACTCGACTCTTGAGACTTCACAACGCCTCCCTTTTCCTCCTTCTCTCAGATCGATTC
TCTTTCTCACTCTCTCAACTCTCTTCAAAACCCCCACAACTAACTTTCTTCTTCTTCTTCTTCTTGT
TCTTTGTATATACATACATACATACATACATGTACATGTATCTCCACCTTCCATATCCACCACTCCT
GACTTGCTCTTAATCTTCACTGAAACGTCCTCGATTCTCTTGCGAGCTTCTGCTTTAGACAACATG
ATACATTCCGCTGCTTCATCCATGGCCTCACCCCTCTGCACGTGGCTGGTGGCGGCTTGCATGT
CAGTCACGTGCCACACTGACCGCGCCGGGACTCCAAAGGCAGCGCTTCGGTCATCCAAGAGGT
CAAGGAGGACGAAGGCGCTGCCAGCAAACACCTCCCAATTGAACATGAGATTGATTTCTTCGCT
GTACGGATCCAGCATTCAGGGACTCATAAGCTCCTTCGAGCCCTGCGATGACTACTGCAACACT
CACAATGCCTTCTCTTCGCTCTTTCGATCCACAACTCCCAATCGCAGACACACCCGTCTCAATAA
ACTTTCTCATTCTGGTAAACTTTTTTTTTTTTTTTTTTAATTTTCCCCACTTAAATCACCTCAAGTTAA
GATATTTCTGGTGCTTTTTCCAATTTTAATCTGTACCCCGTACTGGGTATTTCAATTTGATTCATTT
ATTTATTTTTCTTATATTAATATAATAATAATAGTTTTTTTTTTTATTATGGTTATGAAATCTGGGTTC
TGATTTTCTGGTCTTCTGAAAGATATGTGTGTGTGTGTGGCTTTTTTCCCTTTTTTTTCTGAATTAC
TTAAAATGCACAAGTGTTAGCTTGGATCTTTGTCTTTTGGTTGGTCATCCTAATAAATGGGGTTGC
TTGGTGGTTTAGTATTGAGGGTTGTTCTACCTGCTCTGTTTAACTGTTTTGAGGGTACGGTTTATA
ATATTAGGAGTATGGGGGTAGTTGTTGTTCTTTCTTTGCTGAGATTGATGGTGCACCTGTGTCAC
TTAATTAGGACATGTTGCTACAACACTATGCCATTGCCCATGCTTTTTGCTTTCATTTCCCATTCCT
ACTTCATTTCCTTGTATTTATCGTACACCCAGGTATCTACATAAAGTTCCCATTAAATATTGGTTAA
TGTTCGCATCATCACTTTGAATTTCTTGCAATCTCGTTGAGGTAGTTCAGCATATTCACAGAATCA
ACTCAAACGAAAATAAATTTCTTTTGACTTTTATTTTGTTTTCTTTTCTTCATGAAAATAATAATCAC
ACTGAAGTGTTCTGAATTTAACTACTATGTAAATATCGCCCCCACGTGGGATTACCTCTCTCTCCC
TCTCTCTCTAGTTTTTTTGTTTCTCTATGTAAAATAGTGGAGGTAAGTTATGCCTTTGTCATAAGCA
CACATAAGCAAGCAGAGGCTCCTTTTTTTTTCCCTCATTTTTTTTTTAAATTATCATGTAAAATAGA
TGATTCTGGCACAAACTATCCACAAGTTATTTTTGCTCCTAGTTCTTTAGTATGTTTTTAACACACT
GGACAAGTCTACTACTGCTGCATGCATGTCCCTGACTGATGGGTTCTAGTATTCAGTGGTCTTGA
GTGTTTTGATTTAAAGCAAGGGATATAAGTTGGGTCTGATGAATTTTTCGAGGTTTCATAGCATTT
TTGTTACCATAGTCTTAGAATTACTACCTTGGATAAACCTAAGCATATTTTTGTTAATGGCTGGGG
TTGCATTTCCTGCTCAACATTAATGTGCAACAAGGGTATGTTCGGTTAGGGGTTAAAAATGGGGC
GGGAATTTTCAATGGATGTGGCCCATGTGTAATTTTACTCATAGAACTTATTGTAAAAATTTCACC
CCATTTCTACTTCCCAAAAGAACATATCCTTATCATGATCATTGGAAGCTACACATGCATCATGTT
GAACTATAAGCTGTGGGCCGTTCTTTCTTGTATGCTTCTGATATGAATTTCTTGAATGATAGGCGG
TTGCATGCAATTTTTATTATTGCTGACTTATTCCTCTGAATTGCAAAACTATAAGGTAAAACAATGG
CTGTAGCTGTGGAACCTGCACACGAAGTCACAGCAACGAAAAAACCTCCTACAAAACAAAGGCG
AGTAGTTGTGACTGGGTTGGGCGTTGTTACACCTCTTGGTCACGAACCAGATGTTTACTACAACA
ATTTACTTGATGGTGTTAGTGGCATAAGTGAGATCGAGACATTTGATTGTGCTGAATATCCAACG
GCAAGTTATATATAATTGAAATTGTATTTCTTGTTGTCTGTTGTAGCTTTTGTTCTGAACATTCTAT
GTTTCCAGAGGATTGCTGGTGAGATCAAGTCGTTCTCCACTGATGGCTGGGTTGCACCAAAACTT
TCGAAGAGAATGGATAAATTTATGCTCTATTTGCTAACAGCCGGTAAAAAGGCCTTAGTTGATGG
TGGAATTACTGAAGATGTAATGGATGAGTTGAATAAGGAAAAATGTGGAGTTTTGATTGGCTCAG
CGATGGGTGGCATGAAGGTAACCTTGTCTTTCTTTCATGTTGAGATACAAATCTTGTAAAACCAG
CTGGTGTTGTGGACTGAATTTAATGCCACTGCAATCCAAATGGAGCAGAAGAGTGTGCTATATTT
TCAACCTTGTGCCCTTGTTATATTGACTATGATTTTTGGTGCTTTTTCTACTGAGCAGGTTTTTAAT
GATGCCATTGAAGCTTTAAGAGTATCATATAAGAAGATGAATCCTTTTTGTGTACCTTTTGCCACA
ACAAATATGGGATCTGCCATACTTGCGATGGATCTGGTCTGTTGTTGCATGGACACCATAAATTG
GTGTTTTGTTTCGTACCAAGCTTTTATTTTGGCTATGTTATCTTCAGCTTCTAACAAACATTGTTAT
CTTTTATGGATTACAGGGATGGATGGGCCCTAATTATTCAATCTCTACAGCTTGTGCTACAAGTAA
CTTTTGTATATTGAATGCAGCAAACCACATCATAAGAGGTGAAGCTGTAAGTATTCATGCTTTGAT
ATTACTCTCAGGATGGTGAATTTTTCGTATCCAATATTATACCTTATATTTATTTATTTATTTAACTT
TCTTTTTCTGAGACAGTTTGGTGTAATGATATGGTCATTGCATCAAAATGTATTGATAAATTTAATA
TGAAAATATTCCTGCAGGATGTGATGCTTTGTGGTGGCTCAGATGCTGCTATTATACCCATTGGT
ATGATCTCACCATTGATTCTTTTTCACCTGCATACGGTTATCATGCCATAAATCTAGCTGATATGA
ATCCACTAATCATTGGTACAGGTTTGGGAGGTTTTGTGGCATGCAGAGCCCTATCGCAGAGGAAT
ACAGACCCTACCAAAGCTTCACGCCCTTGGGACATTGTAATACAACAGAAATCCTGCTCATTCTT
TCTGCTACTCACCATGTTTTTGTATTGCTGATCTTTCATCATTTTTTATTTTTGAAACAATACAACTA
TAATTATAATGAAATTTGATGACCAAGTCTTGTATCCTTTCTTGTAATCTGAACAGCATAGTTTATT
AAAAACTAACGTTTCAATGGTCACAGATACAACTTTTCCTTTTGATCACAAAAATGCTGTCTTCTAT
GGATATGAGCTATGGCAAATTCAGTTTTTCTAATTAGGCTGTTTATGATATTCCAGAATCGAGATG
GATTTGTCATGGGGGAAGGAGCTGGAGTTTTACTTTTGGAGGACTTGGAGCATGCAAAGGTACA
GGAAATCAGATCTAATAATTTTAAAACAGAACATCCCTTATGAAATGACAGTGCATGTTTTGATATT
ATTACTGCAGACTTGTGCATGTAATTTATTTCATTTAGTGCATCCTTCAGTTCCTTCAAACAGTATT
AAAGGACTTCATACATCTGTTCTCTCTTGAATTAGGATTGATTTTCAGGCTGCTGCTTGTCATAAA
AATTCACCTGTTACTTCTCATGTCAAATCTGCGCTTGACATTTGTCTTGCAACTAAATCAAATGCAT
CTCTTTTACATTGCAGAAAAGAGGTGCAACCATATATGCTGAATTCCTTGGTGGAAGCTTCACCT
GTGATGCATATCATGTGACAGAGCCACGTCCCGATGGTAAAATTTCAAATCTTTTACTACCAAAAT
TTAAAGCATTTATATGCATGCTATGATTACCATGCTTAAAATCCGAAGAGAACAAGGCAAAAGAAT
GTGACAAGGGTATTTTTATATATTTAGAATCATGTGTGCCATTTGCTGATGGTTAGGTTAAGTATTT
AGAAATATACTATATGTTGCATTTTTCTTGTTTGGCACTGGAATTTCTTCTTCCGAGATCGTGATG
GAATTTAAAACTGCATGTTGCGATCATGACTTGTCTCGACAATATGGACCTGGGATTTAATAATAG
TCCTATATTAGTGGACCTAGTAATCCTAGTCATAATTAGTATCTGCTTGTACTTTACCACATTTCCT
TTATGATTCCATTCGGACTCTTCTTTGAGCATTCTATGACTTGACAAGGAATGATTTGTATCTCCC
TGCGGAACTTGCATTCACGTTTCTTTACTCCATCTTAAATAGGAGCTGGCGTTATACTTTGCATTG
AAAAGGCATTAGCTCAGTCTGGAGTATCAAAAGAGGATGTGAACTACATAAATGCACATGCCACA
TCCACGCCTGCTGGGGATCTTAAGGAGTTCCAGGCTCTGCTTCATTGTTTTGGTCAAAATCCCGA
GGTACATCTATCTTTTTAAGTTACACCACCATTAACATTGTTATCTTGGTGATCAATTCTATGGTAA
ACATGATCTCTTGTTCTTCTGGAGTTCATGTTGAGTAAAACACTGGTACTTCTGATATGGTCATCC
ATACTAATGTCTCTTGTTTTTTCAGCTAAGAGTGAATTCTACAAAATCTATGATTGGTCATCTACTA
GGAGCAGCTGGTGGCGTGGAAGCTGTAGCAACAGTCCAGGTAAAACCCAATGGATCACTTAGTT
CGGTACCCCGCGATTGATGACTAAAATGGTCCTGAGGTTTGCTCTCTCTTCGTACAGGCAATTAG
GACAGGGTGGGTGCATCCCAATATCAACCTGGAAAACCCAGATCAAGGAGTGGTAATGTTTCTT
GTTGCATCATTCCATCCTTCATTTCAAAATTTTAATCTTTTAATCTAACATGTCCAAACTAATGACA
AAAATTCAGGATGCCAAAGTGCTTGTGGGCTCAAAGAAAGAGAGACTAGATATCAAGGCTGCCC
TATCCAATTCATTTGGTTTTGGAGGCCACAATTCTTCAATCATATTTGCTCCATATAAGTGAATGAA
TACATGTTTCAGAATAATACCTTCCCTACCATTATACAGAAGGTATGTCTCTAAATAAGAATAGAT
GACCATTTGTATTCATAAAAGATGAAAACACTGACATATGTATCCACACTAGATCGAAACTAAACT
TGTACCCACGCAAGATGTCCTCCGTGTGACAAAAGTACCCTACGTGGCACTCTAACCCTCCAAA
GACAGGATACTTTTGTCACACGGAGGGCATCTTTCGTGGGTACAAGTTTAGTTTCGATTTAATGT
GGGTACATATGTCAGCATTTTCATTTTTCATGGATACAAATGGTCATTTATTCCTCTAAATAATGAG
CAGGTCATATCGTCCTACTACGAGAAACACATTTAATTTGGATGACTGTGGATGTTCCTTGAACAT
GTATTAATTGTGTGTTGTTTTTATGTTAAGTTACTAAGGTAGCCAGGCAATTGTTACACGCATGAT
CAAAGTCATGAATTGGATGAAAATATATGTGGAAATGGTTGGAGCAGAAACTCTTGAGGACATTG
TTGAGAGATGGTTTGGTGGTTTAGATTGTCTGGCCGAGATACATGGTGGAGCCGGTTCTATCCA
CCGATGAAGAACATGAATGGCTGCTATTGCCTTTATAGAGTAGGACTTGACCCACTTTGATGTGA
CTCTTAAACAAATCCACTTGGCAGTATATATAAGCTCTTCTGTTTTATTTATTCCCTATGTACCCAA
ATAGTTTGTGCAATAACAAATTGCAATTTATTTTTTCGCT SEQ ID NO: 117
KASII genomic (Arachis hypogaea)
TTTGTATCATATTCAACTACTAGTTTACATATATGAGACTTTTTAATAACCATCAAGAGATAAAATTT
AAATATTTTTATTGATCATATAATACTTAATTGTATAGTTGTGTAAAATACTTTAAATAATACTATTAA
TATATTAAAATCAGCTATTATATATATTGTTTAATTTATTTTTAATATATATTTTATATTTTAATATGTA
TTTTATATTAATGTTTGATTTTGGTATATACTTAATAATACAGTTAATACATTGATAATATTTTAGACT
ATTTTTCTTAATTTTATTTTTTAAAGTTTTAATATCAACCATTCATTTATTAAAGATAAGATAAATTTT
GATTCATGAATTTGATCTGATAATTAATATAAATTTTAAACATGCTTAATTTGATAATGAGTTTTATA
ACTATAATTCAATCAAGTCATTTTGTCACCCATTCATGAATACTTATGTGTGCAGATAACTATTAAC
AGCTCATCAGTAGCTTGCTGATGGTAATGTCTCTCAACTTGTTTACATGACAGTTAACTAACCGG
GCATCTCATAAGTATTTTAATTTGTAAGCATTTTATTAATTAGATTGATTATAAAAAAAAATTAATTT
GATTTACGGTTAAAAATAGCTTTTGTTTAAAATTTAAAAGCCAAACTAACTATTAGATGAAAATTTA
TGAGCAAATTGAGTTTTAATGCTAGTTTGGATTGGTTTTTTTGAGTAAAAATTATTTTTTAAAAACA
AATTAAAGTACTTTTTACTCAATTTTAAATTTATTTAGATATATTTTTTTAATTTATTAAAAAATTAAA
CTATTTACTTTTTTAAAAGTTAACAATATTTTACTTTTAAAAAAATAGAAACAAAAAATATTTTTAATA
TTTGAAATTTTTTAAAAAGCTTATTTAAATGTAAAATAATTTAAAAAAATAAAAACTAAATATTTTTTT
AAAAATCAATTTAAACTGATCTTTATCTCTTAATTATTAACTAATTTTGGTATTTACTTTCAAATTTTC
GTAAAAAAAAGTCCTACAAGAGAATAAAGTATTATTTTGACTAAAGAAAATCTTATTTTGACTAAAG
AAAATCTGAAACCAAGAATAAAATAAATGCTATGTCACGTTTTACACTAATGATAAAAAAATTAAAG
TACAATATAAGTAGAAAATATGTTCTAAAACCAAACAAAAAAGTGCATTAAATTGCGAAAAGGATT
GATCCAAGGAGAAATGGCGTAATGAATTAGCATAGAAAGCAATTAGTTTTATATTGCATAAATAAT
GATGAATTATATTAGGTAAAGGTTCAGGTGGAGTGGAATTCACGTAAAGTTGATAACTGAGAGTC
GTTAGATAATTTGATTGATTTGACTAAATTTTTATTTAACGGCTCTCGACTATCAACTTTGCGATGC
ACCTGAATTTTACCATTATATTAGTAGCGAAGAAAAAGAAAAGAGGTTAGTAGAAAATTAAATTAA
ATTAATAAAGAAAGCAAATGAGAAGCGAAGGAGCGACTGAGACTCAGATTGCAGAAAAAGCAGA
GACTTTGAGACTCGACTCTTGAGACTTCACAACGCCTCCCTTTTCCTCCTTCTCTCAGATCGATTC
TCTTTCTCACTCTCTCAACTCTCTTCAAAACCCCCACAACTAACTTTCTTCTTCTTCTTCTTCTTGT
TCTTTGTATATACATACATACATACATACATGTACATGTATCTCCACCTTCCATATCCACCACTCCT
GACTTGCTCTTAATCTTCACTGAAACGTCCTCGATTCTCTTGCGAGCTTCTGCTTTAGACAACATG
ATACATTCCGCTGCTTCATCCATGGCCTCACCCCTCTGCACGTGGCTGGTGGCGGCTTGCATGT
CAGTCACGTGCCACACTGACCGCGCCGGGACTCCAAAGGCAGCGCTTCGGTCATCCAAGAGGT
CAAGGAGGACGAAGGCGCTGCCAGCAAACACCTCCCAATTGAACATGAGATTGATTTCTTCGCT
GTACGGATCCAGCATTCAGGGACTCATAAGCTCCTTCGAGCCCTGCGATGACTACTGCAACACT
CACAATGCCTTCTCTTCGCTCTTTCGATCCACAACTCCCAATCGCAGACACACCCGTCTCAATAA
ACTTTCTCATTCTGGTAAACTTTTTTTTTTTTTTTTTTAATTTTCCCCACTTAAATCACCTCAAGTTAA
GATATTTCTGGTGCTTTTTCCAATTTTAATCTGTACCCCGTACTGGGTATTTCAATTTGATTCATTT
ATTTATTTTTCTTATATTAATATAATAATAATAGTTTTTTTTTTTATTATGGTTATGAAATCTGGGTTC
TGATTTTCTGGTCTTCTGAAAGATATGTGTGTGTGTGTGGCTTTTTTCCCTTTTTTTTCTGAATTAC
TTAAAATGCACAAGTGTTAGCTTGGATCTTTGTCTTTTGGTTGGTCATCCTAATAAATGGGGTTGC
TTGGTGGTTTAGTATTGAGGGTTGTTCTACCTGCTCTGTTTAACTGTTTTGAGGGTACGGTTTATA
ATATTAGGAGTATGGGGGTAGTTGTTGTTCTTTCTTTGCTGAGATTGATGGTGCACCTGTGTCAC
TTAATTAGGACATGTTGCTACAACACTATGCCATTGCCCATGCTTTTTGCTTTCATTTCCCATTCCT
ACTTCATTTCCTTGTATTTATCGTACACCCAGGTATCTACATAAAGTTCCCATTAAATATTGGTTAA
TGTTCGCATCATCACTTTGAATTTCTTGCAATCTCGTTGAGGTAGTTCAGCATATTCACAGAATCA
ACTCAAACGAAAATAAATTTCTTTTGACTTTTATTTTGTTTTCTTTTCTTCATGAAAATAATAATCAC
ACTGAAGTGTTCTGAATTTAACTACTATGTAAATATCGCCCCCACGTGGGATTACCTCTCTCTCCC
TCTCTCTCTAGTTTTTTTGTTTCTCTATGTAAAATAGTGGAGGTAAGTTATGCCTTTGTCATAAGCA
CACATAAGCAAGCAGAGGCTCCTTTTTTTTTCCCTCATTTTTTTTTTAAATTATCATGTAAAATAGA
TGATTCTGGCACAAACTATCCACAAGTTATTTTTGCTCCTAGTTCTTTAGTATGTTTTTAACACACT
GGACAAGTCTACTACTGCTGCATGCATGTCCCTGACTGATGGGTTCTAGTATTCAGTGGTCTTGA
GTGTTTTGATTTAAAGCAAGGGATATAAGTTGGGTCTGATGAATTTTTCGAGGTTTCATAGCATTT
TTGTTACCATAGTCTTAGAATTACTACCTTGGATAAACCTAAGCATATTTTTGTTAATGGCTGGGG
TTGCATTTCCTGCTCAACATTAATGTGCAACAAGGGTATGTTCGGTTAGGGGTTAAAAATGGGGC
GGGAATTTTCAATGGATGTGGCCCATGTGTAATTTTACTCATAGAACTTATTGTAAAAATTTCACC
CCATTTCTACTTCCCAAAAGAACATATCCTTATCATGATCATTGGAAGCTACACATGCATCATGTT
GAACTATAAGCTGTGGGCCGTTCTTTCTTGTATGCTTCTGATATGAATTTCTTGAATGATAGGCGG
TTGCATGCAATTTTTATTATTGCTGACTTATTCCTCTGAATTGCAAAACTATAAGGTAAAACAATGG
CTGTAGCTGTGGAACCTGCACACGAAGTCACAGCAACGAAAAAACCTCCTACAAAACAAAGGCG
AGTAGTTGTGACTGGGTTGGGCGTTGTTACACCTCTTGGTCACGAACCAGATGTTTACTACAACA
ATTTACTTGATGGTGTTAGTGGCATAAGTGAGATCGAGACATTTGATTGTGCTGAATATCCAACG
GCAAGTTATATATAATTGAAATTGTATTTCTTGTTGTCTGTTGTAGCTTTTGTTCTGAACATTCTAT
GTTTCCAGAGGATTGCTGGTGAGATCAAGTCGTTCTCCACTGATGGCTGGGTTGCACCAAAACTT
TCGAAGAGAATGGATAAATTTATGCTCTATTTGCTAACAGCCGGTAAAAAGGCCTTAGTTGATGG
TGGAATTACTGAAGATGTAATGGATGAGTTGAATAAGGAAAAATGTGGAGTTTTGATTGGCTCAG
CGATGGGTGGCATGAAGGTAACCTTGTCTTTCTTTCATGTTGAGATACAAATCTTGTAAAACCAG
CTGGTGTTGTGGACTGAATTTAATGCCACTGCAATCCAAATGGAGCAGAAGAGTGTGCTATATTT
TCAACCTTGTGCCCTTGTTATATTGACTATGATTTTTGGTGCTTTTTCTACTGAGCAGGTTTTTAAT
GATGCCATTGAAGCTTTAAGAGTATCATATAAGAAGATGAATCCTTTTTGTGTACCTTTTGCCACA
ACAAATATGGGATCTGCCATACTTGCGATGGATCTGGTCTGTTGTTGCATGGACACCATAAATTG
GTGTTTTGTTTCGTACCAAGCTTTTATTTTGGCTATGTTATCTTCAGCTTCTAACAAACATTGTTAT
CTTTTATGGATTACAGGGATGGATGGGCCCTAATTATTCAATCTCTACAGCTTGTGCTACAAGTAA
CTTTTGTATATTGAATGCAGCAAACCACATCATAAGAGGTGAAGCTGTAAGTATTCATGCTTTGAT
ATTACTCTCAGGATGGTGAATTTTTCGTATCCAATATTATACCTTATATTTATTTATTTATTTAACTT
TCTTTTTCTGAGACAGTTTGGTGTAATGATATGGTCATTGCATCAAAATGTATTGATAAATTTAATA
TGAAAATATTCCTGCAGGATGTGATGCTTTGTGGTGGCTCAGATGCTGCTATTATACCCATTGGT
ATGATCTCACCATTGATTCTTTTTCACCTGCATACGGTTATCATGCCATAAATCTAGCTGATATGA
ATCCACTAATCATTGGTACAGGTTTGGGAGGTTTTGTGGCATGCAGAGCCCTATCGCAGAGGAAT
ACAGACCCTACCAAAGCTTCACGCCCTTGGGACATTGTAATACAACAGAAATCCTGCTCATTCTT
TCTGCTACTCACCATGTTTTTGTATTGCTGATCTTTCATCATTTTTTATTTTTGAAACAATACAACTA
TAATTATAATGAAATTTGATGACCAAGTCTTGTATCCTTTCTTGTAATCTGAACAGCATAGTTTATT
AAAAACTAACGTTTCAATGGTCACAGATACAACTTTTCCTTTTGATCACAAAAATGCTGTCTTCTAT
GGATATGAGCTATGGCAAATTCAGTTTTTCTAATTAGGCTGTTTATGATATTCCAGAATCGAGATG
GATTTGTCATGGGGGAAGGAGCTGGAGTTTTACTTTTGGAGGACTTGGAGCATGCAAAGGTACA
GGAAATCAGATCTAATAATTTTAAAACAGAACATCCCTTATGAAATGACAGTGCATGTTTTGATATT
ATTACTGCAGACTTGTGCATGTAATTTATTTCATTTAGTGCATCCTTCAGTTCCTTCAAACAGTATT
AAAGGACTTCATACATCTGTTCTCTCTTGAATTAGGATTGATTTTCAGGCTGCTGCTTGTCATAAA
AATTCACCTGTTACTTCTCATGTCAAATCTGCGCTTGACATTTGTCTTGCAACTAAATCAAATGCAT
CTCTTTTACATTGCAGAAAAGAGGTGCAACCATATATGCTGAATTCCTTGGTGGAAGCTTCACCT
GTGATGCATATCATGTGACAGAGCCACGTCCCGATGGTAAAATTTCAAATCTTTTACTACCAAAAT
TTAAAGCATTTATATGCATGCTATGATTACCATGCTTAAAATCCGAAGAGAACAAGGCAAAAGAAT
GTGACAAGGGTATTTTTATATATTTAGAATCATGTGTGCCATTTGCTGATGGTTAGGTTAAGTATTT
AGAAATATACTATATGTTGCATTTTTCTTGTTTGGCACTGGAATTTCTTCTTCCGAGATCGTGATG
GAATTTAAAACTGCATGTTGCGATCATGACTTGTCTCGACAATATGGACCTGGGATTTAATAATAG
TCCTATATTAGTGGACCTAGTAATCCTAGTCATAATTAGTATCTGCTTGTACTTTACCACATTTCCT
TTATGATTCCATTCGGACTCTTCTTTGAGCATTCTATGACTTGACAAGGAATGATTTGTATCTCCC
TGCGGAACTTGCATTCACGTTTCTTTACTCCATCTTAAATAGGAGCTGGCGTTATACTTTGCATTG
AAAAGGCATTAGCTCAGTCTGGAGTATCAAAAGAGGATGTGAACTACATAAATGCACATGCCACA
TCCACGCCTGCTGGGGATCTTAAGGAGTTCCAGGCTCTGCTTCATTGTTTTGGTCAAAATCCCGA
GGTACATCTATCTTTTTAAGTTACACCACCATTAACATTGTTATCTTGGTGATCAATTCTATGGTAA
ACATGATCTCTTGTTCTTCTGGAGTTCATGTTGAGTAAAACACTGGTACTTCTGATATGGTCATCC
ATACTAATGTCTCTTGTTTTTTCAGCTAAGAGTGAATTCTACAAAATCTATGATTGGTCATCTACTA
GGAGCAGCTGGTGGCGTGGAAGCTGTAGCAACAGTCCAGGTAAAACCCAATGGATCACTTAGTT
CGGTACCCCGCGATTGATGACTAAAATGGTCCTGAGGTTTGCTCTCTCTTCGTACAGGCAATTAG
GACAGGGTGGGTGCATCCCAATATCAACCTGGAAAACCCAGATCAAGGAGTGGTAATGTTTCTT
GTTGCATCATTCCATCCTTCATTTCAAAATTTTAATCTTTTAATCTAACATGTCCAAACTAATGACA
AAAATTCAGGATGCCAAAGTGCTTGTGGGCTCAAAGAAAGAGAGACTAGATATCAAGGCTGCCC
TATCCAATTCATTTGGTTTTGGAGGCCACAATTCTTCAATCATATTTGCTCCATATAAGTGAATGAA
TACATGTTTCAGAATAATACCTTCCCTACCATTATACAGAAGGTATGTCTCTAAATAAGAATAGAT
GACCATTTGTATTCATAAAAGATGAAAACACTGACATATGTATCCACACTAGATCGAAACTAAACT
TGTACCCACGCAAGATGTCCTCCGTGTGACAAAAGTACCCTACGTGGCACTCTAACCCTCCAAA
GACAGGATACTTTTGTCACACGGAGGGCATCTTTCGTGGGTACAAGTTTAGTTTCGATTTAATGT
GGGTACATATGTCAGCATTTTCATTTTTCATGGATACAAATGGTCATTTATTCCTCTAAATAATGAG
CAGGTCATATCGTCCTACTACGAGAAACACATTTAATTTGGATGACTGTGGATGTTCCTTGAACAT
GTATTAATTGTGTGTTGTTTTTATGTTAAGTTACTAAGGTAGCCAGGCAATTGTTACACGCATGAT
CAAAGTCATGAATTGGATGAAAATATATGTGGAAATGGTTGGAGCAGAAACTCTTGAGGACATTG
TTGAGAGATGGTTTGGTGGTTTAGATTGTCTGGCCGAGATACATGGTGGAGCCGGTTCTATCCA
CCGATGAAGAACATGAATGGCTGCTATTGCCTTTATAGAGTAGGACTTGACCCACTTTGATGTGA
CTCTTAAACAAATCCACTTGGCAGTATATATAAGCTCTTCTGTTTTATTTATTCCCTATGTACCCAA
ATAGTTTGTGCAATAACAAATTGCAATTTATTTTTTCGCT SEQ ID NO: 118
KASII genomic (Arachis hypogaea)
TTTGTATCATATTCAACTACTAGTTTACATATATGAGACTTTTTAATAACCATCAAGAGATAAAATTT
AAATATTTTTATTGATCATATAATACTTAATTGTATAGTTGTGTAAAATACTTTAAATAATACTATTAA
TATATTAAAATCAGCTATTATATATATTGTTTAATTTATTTTTAATATATATTTTATATTTTAATATGTA
TTTTATATTAATGTTTGATTTTGGTATATACTTAATAATACAGTTAATACATTGATAATATTTTAGACT
ATTTTTCTTAATTTTATTTTTTAAAGTTTTAATATCAACCGTTCATTTATTAAAGATAAGATAAATTTT
GATTCATGAATTTGATCTGATAATTAATATAAATTTTAAACATGCTTAATTTGATAATGAGTTTTATA
ACTATAATTCAATCAAGTCATTTTGTCACCCATTCATGAATACTTATGTGTGCAGATAACTATTAAC
AGCTCATCAGTAGCTTGCTGATGGTAATGTCTCTCAACTTGTTTACATGACAGTTAACTAACCGG
GCATCTCATAAGTATTTTAATTTGTAAGCATTTTATTAATTAGATTGATTATAAAAAAAAATTAATTT
GATTTACGGTTAAAAATAGCTTTTGTTTAAAATTTAAAAGCCAAACTAACTATTAGATGAAAATTTA
TGAGCAAATTGAGTTTTAATGCTAGTTTGGATTGGTTTTTTTGAGTAAAAATTATTTTTTAAAAACA
AATTAAAGTACTTTTTACTCAATTTTAAATTTATTTAGATATATTTTTTTAATTTATTAAAAAATTAAA
CTATTTACTTTTTTAAAAGTTAACAATATTTTACTTTTAAAAAAATAGAAACAAAAAATATTTTTAATA
TTTGAAATTTTTTAAAAAGCTTATTTAAATGTAAAATAATTTAAAAAAATAAAAACTAAATATTTTTTT
AAAAATCAATTTAAACTGATCTTTATCTCTTAATTATTAACTAATTTTGGTATTTACTTTCAAATTTTC
GTAAAAAAAAGTCCTACAAGAGAATAAAGTATTATTTTGACTAAAGAAAATCTTATTTTGACTAAAG
AAAATCTGAAACCAAGAATAAAATAAATGCTATGTCACGTTTTACACTAATGATAAAAAAATTAAAG
TACAATATAAGTAGAAAATATGTTCTAAAACCAAACAAAAAAGTGCATTAAATTGCGAAAAGGATT
GATCCAAGGAGAAATGGCGTAATGAATTAGCATAGAAAGCAATTAGTTTTATATTGCATAAATAAT
GATGAATTATATTAGGTAAAGGTTCAGGTGGAGTGGAATTCACGTAAAGTTGATAACTGAGAGTC
GTTCGATAATTTGATTGATTTGACTAAATTTTTATTTAACGGCTCTCGACTATCAACTTTGCGATGC
ACCTGAATTTTACCATTATATTAGTAGCGAAGAAAAAGAAAAGAGGTTAGTAGAAAATTAAATTAA
ATTAATAAAGAAAGCAAATGAGAAGCGAAGGAGCGACTGAGACTCAGATTGCAGAAAAAGCAGA
GACTTTGAGACTCGACTCTTGAGACTTCACAACGCCTCCCTTTTCCTCCTTCTCTCAGATCGATTC
TCTTTCTCACTCTCTCAACTCTCTTCAAAACCCCCACAACTAACTTTCTTCTTCTTCTTCTTCTTGT
TCTTTGTATATACATACATACATACATACATGTACATGTATCTCCACCTTCCATATCCACCACTCCT
GACTTGCTCTTAATCTTCACTGAAACGTCCTCGATTCTCTTGCGAGCTTCTGCTTTAGACAACATG
ATACATTCCGCTGCTTCATCCATGGCCTCACCCCTCTGCACGTGGCTGGTGGCGGCTTGCATGT
CAGTCACGTGCCACACTGACCGCGCCGGGACTCCAAAGGCAGCGCTTCGGTCATCCAAGAGGT
CAAGGAGGACGAAGGCGCTGCCAGCAAACACCTCCCAATTGAACATGAGATTGATTTCTTCGCT
GTACGGATCCAGCATTCAGGGACTCATAAGCTCCTTCGAGCCCTGCGATGACTACTGCAACACT
CACAATGCCTTCTCTTCGCTCTTTCGATCCACAACTCCCAATCGCAGACACACCCGTCTCAATAA
ACTTTCTCATTCTGGTAAACTTTTTTTTTTTTTTTTTTAATTTTCCCCACTTAAATCACCTCAAGTTAA
GATATTTCTGGTGCTTTTTCCAATTTTAATCTGTACCCCGTACTGGGTATTTCAATTTGATTCATTT
ATTTATTTTTCTTATATTAATATAATAATAATAGTTTTTTTTTTTATTATGGTTATGAAATCTGGGTTC
TGATTTTCTGGTCTTCTGAAAGATATGTGTGTGTGTGTGGCTTTTTTCCCTTTTTTTTCTGAATTAC
TTAAAATGCACAAGTGTTAGCTTGGATCTTTGTCTTTTGGTTGGTCATCCTAATAAATGGGGTTGC
TTGGTGGTTTAGTATTGAGGGTTGTTCTACCTGCTCTGTTTAACTGTTTTGAGGGTACGGTTTATA
ATATTAGGAGTATGGGGGTAGTTGTTGTTCTTTCTTTGCTGAGATTGATGGTGCACCTGTGTCAC
TTAATTAGGACATGTTGCTACAACACTATGCCATTGCCCATGCTTTTTGCTTTCATTTCCCATTCCT
ACTTCATTTCCTTGTATTTATCGTACACCCAGGTATCTACATAAAGTTCCCATTAAATATTGGTTAA
TGTTCGCATCATCACTTTGAATTTCTTGCAATCTCGTTGAGGTAGTTCAGCATATTCACAGAATCA
ACTCAAACGAAAATAAATTTCTTTTGACTTTTATTTTGTTTTCTTTTCTTCATGAAAATAATAATCAC
ACTGAAGTGTTCTGAATTTAACTACTATGTAAATATCGCCCCCACGTGGGATTACCTCTCTCTCCC
TCTCTCTCTAGTTTTTTTGTTTCTCTATGTAAAATAGTGGAGGTAAGTTATGCCTTTGTCATAAGCA
CACATAAGCAAGCAGAGGCTCCTTTTTTTTTCCCTCATTTTTTTTTTAAATTATCATGTAAAATAGA
TGATTCTGGCACAAACTATCCACAAGTTATTTTTGCTCCTAGTTCTTTAGTATGTTTTTAACACACT
GGACAAGTCTACTACTGCTGCATGCATGTCCCTGACTGATGGGTTCTAGTATTCAGTGGTCTTGA
GTGTTTTGATTTAAAGCAAGGGATATAAGTTGGGTCTGATGAATTTTTCGAGGTTTCATAGCATTT
TTGTTACCATAGTCTTAGAATTACTACCTTGGATAAACCTAAGCATATTTTTGTTAATGGCTGGGG
TTGCATTTCCTGCTCAACATTAATGTGCAACAAGGGTATGTTCGGTTAGGGGTTAAAAATGGGGC
GGGAATTTTCAATGGATGTGGCCCATGTGTAATTTTACTCATAGAACTTATTGTAAAAATTTCACC
CCATTTCTACTTCCCAAAAGAACATATCCTTATCATGATCATTGGAAGCTACACATGCATCATGTT
GAACTATAAGCTGTGGGCCGTTCTTTCTTGTATGCTTCTGATATGAATTTCTTGAATGATAGGCGG
TTGCATGCAATTTTTATTATTGCTGACTTATTCCTCTGAATTGCAAAACTATAAGGTAAAACAATGG
CTGTAGCTGTGGAACCTGCACACGAAGTCACAGCAACGAAAAAACCTCCTACAAAACAAAGGCG
AGTAGTTGTGACTGGGTTGGGCGTTGTTACACCTCTTGGTCACGAACCAGATGTTTACTACAACA
ATTTACTTGATGGTGTTAGTGGCATAAGTGAGATCGAGACATTTGATTGTGCTGAATATCCAACG
GCAAGTTATATATAATTGAAATTGTATTTCTTGTTGTCTGTTGTAGCTTTTGTTCTGAACATTCTAT
GTTTCCAGAGGATTGCTGGTGAGATCAAGTCGTTCTCCACTGATGGCTGGGTTGCACCAAAACTT
TCGAAGAGAATGGATAAATTTATGCTCTATTTGCTAACAGCCGGTAAAAAGGCCTTAGTTGATGG
TGGAATTACTGAAGATGTAATGGATGAGTTGAATAAGGAAAAATGTGGAGTTTTGATTGGCTCAG
CGATGGGTGGCATGAAGGTAACCTTGTCTTTCTTTCATGTTGAGATACAAATCTTGTAAAACCAG
CTGGTGTTGTGGACTGAATTTAATGCCACTGCAATCCAAATGGAGCAGAAGAGTGTGCTATATTT
TCAACCTTGTGCCCTTGTTATATTGACTATGATTTTTGGTGCTTTTTCTACTGAGCAGGTTTTTAAT
GATGCCATTGAAGCTTTAAGAGTATCATATAAGAAGATGAATCCTTTTTGTGTACCTTTTGCCACA
ACAAATATGGGATCTGCCATACTTGCGATGGATCTGGTCTGTTGTTGCATGGACACCATAAATTG
GTGTTTTGTTTCGTACCAAGCTTTTATTTTGGCTATGTTATCTTCAGCTTCTAACAAACATTGTTAT
CTTTTATGGATTACAGGGATGGATGGGCCCTAATTATTCAATCTCTACAGCTTGTGCTACAAGTAA
CTTTTGTATATTGAATGCAGCAAACCACATCATAAGAGGTGAAGCTGTAAGTATTCATGCTTTGAT
ATTACTCTCAGGATGGTGAATTTTTCGTATCCAATATTATACCTTATATTTATTTATTTATTTAACTT
TCTTTTTCTGAGACAGTTTGGTGTAATGATATGGTCATTGCATCAAAATGTATTGATAAATTTAATA
TGAAAATATTCCTGCAGGATGTGATGCTTTGTGGTGGCTCAGATGCTGCTATTATACCCATTGGT
ATGATCTCACCATTGATTCTTTTTCACCTGCATACGGTTATCATGCCATAAATCTAGCTGATATGA
ATCCACTAATCATTGGTACAGGTTTGGGAGGTTTTGTGGCATGCAGAGCCCTATCGCAGAGGAAT
ACAGACCCTACCAAAGCTTCACGCCCTTGGGACATTGTAATACAACAGAAATCCTGCTCATTCTT
TCTGCTACTCACCATGTTTTTGTATTGCTGATCTTTCATCATTTTTTATTTTTGAAACAATACAACTA
TAATTATAATGAAATTTGATGACCAAGTCTTGTATCCTTTCTTGTAATCTGAACAGCATAGTTTATT
AAAAACTAACGTTTCAATGGTCACAGATACAACTTTTCCTTTTGATCACAAAAATGCTGTCTTCTAT
GGATATGAGCTATGGCAAATTCAGTTTTTCTAATTAGGCTGTTTATGATATTCCAGAATCGAGATG
GATTTGTCATGGGGGAAGGAGCTGGAGTTTTACTTTTGGAGGACTTGGAGCATGCAAAGGTACA
GGAAATCAGATCTAATAATTTTAAAACAGAACATCCCTTATGAAATGACAGTGCATGTTTTGATATT
ATTACTGCAGACTTGTGCATGTAATTTATTTCATTTAGTGCATCCTTCAGTTCCTTCAAACAGTATT
AAAGGACTTCATACATCTGTTCTCTCTTGAATTAGGATTGATTTTCAGGCTGCTGCTTGTCATAAA
AATTCACCTGTTACTTCTCATGTCAAATCTGCGCTTGACATTTGTCTTGCAACTAAATCAAATGCAT
CTCTTTTACATTGCAGAAAAGAGGTGCAACCATATATGCTGAATTCCTTGGTGGAAGCTTCACCT
GTGATGCATATCATGTGACAGAGCCACGTCCCGATGGTAAAATTTCAAATCTTTTACTACCAAAAT
TTAAAGCATTTATATGCATGCTATGATTACCATGCTTAAAATCCGAAGAGAACAAGGCAAAAGAAT
GTGACAAGGGTATTTTTATATATTTAGAATCATGTGTGCCATTTGCTGATGGTTAGGTTAAGTATTT
AGAAATATACTATATGTTGCATTTTTCTTGTTTGGCACTGGAATTTCTTCTTCCGAGATCGTGATG
GAATTTAAAACTGCATGTTGCGATCATGACTTGTCTCGACAATATGGACCTGGGATTTAATAATAG
TCCTATATTAGTGGACCTAGTAATCCTAGTCATAATTAGTATCTGCTTGTACTTTACCACATTTCCT
TTATGATTCCATTCGGACTCTTCTTTGAGCATTCTATGACTTGACAAGGAATGATTTGTATCTCCC
TGCGGAACTTGCATTCACGTTTCTTTACTCCATCTTAAATAGGAGCTGGCGTTATACTTTGCATTG
AAAAGGCATTAGCTCAGTCTGGAGTATCAAAAGAGGATGTGAACTACATAAATGCACATGCCACA
TCCACGCCTGCTGGGGATCTTAAGGAGTTCCAGGCTCTGCTTCATTGTTTTGGTCAAAATCCCGA
GGTACATCTATCTTTTTAAGTTACACCACCATTAACATTGTTATCTTGGTGATCAATTCTATGGTAA
ACATGATCTCTTGTTCTTCTGGAGTTCATGTTGAGTAAAACACTGGTACTTCTGATATGGTCATCC
ATACTAATGTCTCTTGTTTTTTCAGCTAAGAGTGAATTCTACAAAATCTATGATTGGTCATCTACTA
GGAGCAGCTGGTGGCGTGGAAGCTGTAGCAACAGTCCAGGTAAAACCCAATGGATCACTTAGTT
CGGTACCCCGCGATTGATGACTAAAATGGTCCTGAGGTTTGCTCTCTCTTCGTACAGGCAATTAG
GACAGGGGGGTGCATCCCAATATCAACCTGGAAAACCCAGATCAAGGAGTGGTAATGTTTCTT
GTTGCATCATTCCATCCTTCATTTCAAAATTTTAATCTTTTAATCTAACATGTCCAAACTAATGACA
AAAATTCAGGATGCCAAAGTGCTTGTGGGCTCAAAGAAAGAGAGACTAGATATCAAGGCTGCCC
TATCCAATTCATTTGGTTTTGGAGGCCACAATTCTTCAATCATATTTGCTCCATATAAGTGAATGAA
TACATGTTTCAGAATAATACCTTCCCTACCATTATACAGAAGGTATGTCTCTAAATAAGAATAGAT
GACCATTTGTATTCATAAAAGATGAAAACACTGACATATGTATCCACACTAGATCGAAACTAAACT
TGTACCCACGCAAGATGTCCTCCGTGTGACAAAAGTACCCTACGTGGCACTCTAACCCTCCAAA
GACAGGATACTTTTGTCACACGGAGGGCATCTTTCGTGGGTACAAGTTTAGTTTCGATTTAATGT
GGGTACATATGTCAGCATTTTCATTTTTCATGGATACAAATGGTCATTTATTCCTCTAAATAATGAG
CAGGTCATATCGTCCTACTACGAGAAACACATTTAATTTGGATGACTGTGGATGTTCCTTGAACAT
GTATTAATTGTGTGTTGTTTTTATGTTAAGTTACTAAGGTAGCCAGGCAATTGTTACACGCATGAT
CAAAGTCATGAATTGGATGAAAATATATGTGGAAATGGTTGGAGCAGAAACTCTTGAGGACATTG
TTGAGAGATGGTTTGGTGGTTTAGATTGTCTGGCCGAGATACATGGTGGAGCCGGTTCTATCCA
CCGATGAAGAACATGAATGGCTGCTATTGCCTTTATAGAGTAGGACTTGACCCACTTTGATGTGA
CTCTTAAACAAATCCACTTGGCAGTATATATAAGCTCTTCTGTTTTATTTATTCCCTATGTACCCAA
ATAGTTTGTGCAATAACAAATTGCAATTTATTTTTTCGCT SEQ ID NO: 119
KASII coding (CDS)(Arachis hypogaea)
ATGATACATTCCGCTGCTTCATCCATGGCCTCACCCCTCTGCACGTGGCTGGTGGCGGCTTGCA
TGTCAGTCACGTGCCACACTGACCGCGCCGGGACTCCAAAGGCAGCGCTTCGGTCATCCAAGA
GGTCAAGGAGGACGAAGGCGCTGCCAGCAAACACCTCCCAATTGAACATGAGATTGATTTCTTC
GCTGTACGGATCCAGCATTCAGGGACTCATAAGCTCCTTCGAGCCCTGCGATGACTACTGCAAC
ACTCACAATGCCTTCTCTTCGCTCTTTCGATCCACAACTCCCAATCGCAGACACACCCGTCTCAA
TAAACTTTCTCATTCTGGTAAAACAATGGCTGTAGCTGTGGAACCTGCACACGAAGTCACAGCAA
CGAAAAAACCTCCTACAAAACAAAGGCGAGTAGTTGTGACTGGGTTGGGCGTTGTTACACCTCTT
GGTCACGAACCAGATGTTTACTACAACAATTTACTTGATGGTGTTAGTGGCATAAGTGAGATCGA
GACATTTGATTGTGCTGAATATCCAACGAGGATTGCTGGTGAGATCAAGTCGTTCTCCACTGATG
GCTGGGTTGCACCAAAACTTTCGAAGAGAATGGATAAATTTATGCTCTATTTGCTAACAGCCGGT
AAAAAGGCCTTAGTTGATGGTGGAATTACTGAAGATGTAATGGATGAGTTGAATAAGGAAAAATG
TGGAGTTTTGATTGGCTCAGCGATGGGTGGCATGAAGGTTTTTAATGATGCCATTGAAGCTTTAA
GAGTATCATATAAGAAGATGAATCCTTTTTGTGTACCTTTTGCCACAACAAATATGGGATCTGCCA
TACTTGCGATGGATCTGGGATGGATGGGCCCTAATTATTCAATCTCTACAGCTTGTGCTACAAGT
AACTTTTGTATATTGAATGCAGCAAACCACATCATAAGAGGTGAAGCTGATGTGATGCTTTGTGGT
GGCTCAGATGCTGCTATTATACCCATTGGTTTGGGAGGTTTTGTGGCATGCAGAGCCCTATCGCA
GAGGAATACAGACCCTACCAAAGCTTCACGCCCTTGGGACATTAATCGAGATGGATTTGTCATGG
GGGAAGGAGCTGGAGTTTTACTTTTGGAGGACTTGGAGCATGCAAAGAAAAGAGGTGCAACCAT
ATATGCTGAATTCCTTGGTGGAAGCTTCACCTGTGATGCATATCATGTGACAGAGCCACGTCCCG
ATGGAGCTGGCGTTATACTTTGCATTGAAAAGGCATTAGCTCAGTCTGGAGTATCAAAAGAGGAT
GTGAACTACATAAATGCACATGCCACATCCACGCCTGCTGGGGATCTTAAGGAGTTCCAGGCTC
TGCTTCATTGTTTTGGTCAAAATCCCGAGCTAAGAGTGAATTCTACAAAATCTATGATTGGTCATC
TACTAGGAGCAGCTGGTGGCGTGGAAGCTGTAGCAACAGTCCAGGCAATTAGGACAGGGTGGG
TGCATCCCAATATCAACCTGGAAAACCCAGATCAAGGAGTGGATGCCAAAGTGCTTGTGGGCTC
AAAGAAAGAGAGACTAGATATCAAGGCTGCCCTATCCAATTCATTTGGTTTTGGAGGCCACAATT
CTTCAATCATATTTGCTCCATATAAGTGA SEQ ID NO: 120
KASII polypeptide (Arachis hypogaea)
MIHSAASSMASPLCTWLVAACMSVTCHTDRAGTPKAALRSSKRSRRTKALPANTSQLNMRLISSLYG
SSIQGLISSFEPCDDYCNTHNAFSSLFRSTTPNRRHTRLNKLSHSGKTMAVAVEPAHEVTATKKPPTK
QRRVVVTGLGVVTPLGHEPDVYYNNLLDGVSGISEIETFDCAEYPTRIAGEIKSFSTDGWVAPKLSKR
MDKFMLYLLTAGKKALVDGGITEDVMDELNKEKCGVLIGSAMGGMKVFNDAIEALRVSYKKMNPFCV
PFATTNMGSAILAMDLGWMGPNYSISTACATSNFCILNAANHIIRGEADVMLCGGSDAAIIPIGLGGFV
ACRALSQRNTDPTKASRPWDINRDGFVMGEGAGVLLLEDLEHAKKRGATIYAEFLGGSFTCDAYHVT
EPRPDGAGVILCIEKALAQSGVSKEDVNYINAHATSTPAGDLKEFQALLHCFGQNPELRVNSTKSMIG
HLLGAAGGVEAVATVQAIRTGWVHPNINLENPDQGVDAKVLVGSKKERLDIKAALSNSFGFGGHNSSI
IFAPYK* SEQ ID NO: 121
Regions
ATAAGATAAATTTTGATTCATGAATTTGATCTGATAATTAATATAAATTTTAAAGATGCTTAATTTGA
TAATGAGTTTTATAACTATAATTCAATCAAGTCATTTTGTCACCCATTCATGAATACTTATGTGTGC
AGATAACTATTAACAGCTCATCAGTAGCTTGCTGATGGTAATGTCTCTCAACTTGTTTACATGACA
GTTAACTAACCGGGCATCTCATAAGTATTTTAATTTGTAAGCATTTTATTAATTAGATTGATTATAA
AAAAAAATTAATTTGATTTACGGTTAAAAATAGCTTTTGTTTAAAATTTAAAAGCCAAACTAACTATT
AGATGAAAATTTATGAGCAAATTGAGTTTTAATGCTAGTTTGGATTG SEQ ID NO: 122
TAAACATGCTTAATTTGATAATGAGTTTTATAACTATAATTCAATCAAGTCATTTTGTCACCCATTCA
TGAATACTTATGTGTGCAGATAACTATTAACAGCTCATCAGTAGCTTGCTGATGGTAATGTCTCTC
AACTTGTTTACATGACAGTTAACTAACCGGGCATCTCATAAGTATTTTAATTTGTAAGCATTTTATT
AATTAGATTGATTATAAAAAAAAATTAATTTGATTTACGGTTAAAAATAGCTTTTGTTTAAAATTTAA
AAGCCAAACTAACTA SEQ ID NO: 123
TCAATCAAGTCATTTTGTCACCCATTCATGAATACTTATGTGTGCAGATAACTATTAACAGCTCATC
AGTAGCTTGCTGATGGTAATGTCTCTCAACTTGTTTACATGACAGTTAACTAACCGGGGATCTCAT
AAGTATTTTAATTTGTAAGCATTTTATTAATTAGATTGATTATAAAAAAAAATTAATTTGATTTACGG
TT SEQ ID NO: 124
CCCATTCATGAATACTTATGTGTGCAGATAACTATTAACAGCTCATCAGTAGCTTGCTGATGGTAA
TGTCTCTCAACTTGTTTACATGACAGTTAACTAACCGGGCATCTCATAAGTATTTTAATTTGTAAGC
ATTTTATTAATTAGATTGATTATAAAAAAA SEQ ID NO: 125
TGTGCAGATAACTATTAACAGCTCATCAGTAGCTTGCTGATGGTAATGTCTCTCAACTTGTTTACA
TGACAGTTAACTAACGGGGCATCTCATAAGTATTTTAATTTGTAAGCATTTTATTAA SEQ ID
NO: 126
GCTCATCAGTAGCTTGCTGATGGTAATGTCTCTCAACTTGTTTACATGACAGTTAACTAACCGGG
CATCTCATAAGTATTTTA SEQ ID NO: 127
TAAACATGCTTAATTTGATAATGAGTTTTATAACTATAATTCAATCAAGTCATTTTGTCACCCATTCA
TGAATACTTATGTGTGCAGATAACTATTAACAGCTCATCAGTAGCTTGCTGATGGTAATGTCTCTC
AACTTGTTTACATGACAGTTAACTAACCGGGCATCTCATAAGTATTTTAATTTGTAAGCATTTTATT
AATTAGATTGATTATAAAAAAA SEQ ID NO: 128
TCAATCAAGTCATTTTGTCACCCATTCATGAATACTTATGTGTGCAGATAACTATTAACAGCTCATC
AGTAGCTTGCTGATGGTAATGTCTCTCAACTTGTTTACATGACAGTTAACTAACCGGGGATCTCAT
AAGTATTTTA SEQ ID NO: 129
CCCATTCATGAATACTTATGTGTGCAGATAACTATTAACAGCTCATCAGTAGCTTGCTGATGGTAA
TGTCTCTCAACTTGTTTACATGACAGTTAACTAACCG SEQ ID NO: 130
TGTGCAGATAACTATTAACAGCTCATCAGTAGCTTGCTGATGGTAATGTCTCTCAACTTGTTT SEQ
ID NO: 131
CCCATTCATGAATACTTATGTGTGCAGATAACTATTAACAGCTCATCAGTAGCTTGCTGATGGTAA
TGTCTCTCAACTTGTTTACATGACAGTTAACTAACCGGGCATCTCATAAGTATTTTAATTTGTAAGC
ATTTTATTAATTAGATTGATTATAAAAAAAAATTAATTTGATTTACGGTTAAAAATAGGTTTTGTTTA
AAATTTAAAAGCCAAACTAACTA SEQ ID NO: 132
GCTCATCAGTAGCTTGCTGATGGTAATGTCTCTCAACTTGTTTACATGACAGTTAACTAACCGGG
CATCTCATAAGTATTTTAATTTGTAAGCATTTTATTAATTAGATTGATTATAAAAAAAAATTAATTTG
ATTTACGGTT SEQ ID NO: 133
ATGGTAATGTCTCTCAACTTGTTTACATGACAGTTAACTAACCGGGCATCTGATAAGTATTTTAATT
TGTAAGCATTTTATTAATTAGATTGATTATAAAAAAA SEQ ID NO: 134
TTTACATGACAGTTAACTAACCGGGCATCTCATAAGTATTTTAATTTGTAAGCATTTTATTAA SEQ
ID NO: 135
TTGACTAAAGAAAATCTTATTTTGACTAAAGAAAATCTGAAACCAAGAATAAAATAAATGCTATGTC
ACGTTTTACACTAATGATAAAAAAATTAAAGTACAATATAAGTAGAAAATATGTTCTAAAACCAAAC
AAAAAAGTGCATTAAATTGCGAAAAGGATTGATCCAAGGAGAAATGGCGTAATGAATTAGCATAG
AAAGCAATTAGTTTTATATTGCATAAATAATGATGAATTATATTAGGTAAAGGTTCAGGTGGAGTG
GAATTCACGTAAAGTTGATAACTGAGAGTCGTTCGATAATTTGATTGATTTGACTAAATTTTTATTT
AACGGCTCTCGACTATCAACTTTGCGATGCACCTGAATTTTACCATTATATTAGTAGCGAAGAAAA
AGAAAAGAGGTTAGTAGA SEQ ID NO: 136
AAAATAAATGCTATGTCACGTTTTACACTAATGATAAAAAAATTAAAGTACAATATAAGTAGAAAAT
ATGTTCTAAAACCAAACAAAAAAGTGCATTAAATTGCGAAAAGGATTGATCCAAGGAGAAATGGC
GTAATGAATTAGCATAGAAAGCAATTAGTTTTATATTGCATAAATAATGATGAATTATATTAGGTAA
AGGTTCAGGTGGAGTGGAATTCACGTAAAGTTGATAACTGAGAGTCGTTCGATAATTTGATTGAT
TTGACTAAATTTTTATTTAACGGCTCTCGACTATCAACTTTGCGATGCACCT SEQ ID NO: 137
AATTAAAGTACAATATAAGTAGAAAATATGTTCTAAAACCAAACAAAAAAGTGCATTAAATTGCGAA
AAGGATTGATCCAAGGAGAAATGGCGTAATGAATTAGGATAGAAAGCAATTAGTTTTATATTGCAT
AAATAATGATGAATTATATTAGGTAAAGGTTCAGGTGGAGTGGAATTCACGTAAAGTTGATAACTG
AGAGTCGTTCGATAATTTGATTGATTTGACTAAATTT SEQ ID NO: 138
TAGAAAATATGTTCTAAAACCAAACAAAAAAGTGCATTAAATTGCGAAAAGGATTGATCCAAGGAG
AAATGGCGTAATGAATTAGCATAGAAAGCAATTAGTTTTATATTGCATAAATAATGATGAATTATAT
TAGGTAAAGGTTCAGGTGGAGTGGAATTCACGTAAAGTTGATAACTGAGAGTCGTTCGATAATT
SEQ ID NO: 139
AAAAAAAAAGTGCATTAAATTGCGAAAAGGATTGATCCAAGGAGAAATGGCGTAATGAATTAGC
ATAGAAAGCAATTAGTTTTATATTGCATAAATAATGATGAATTATATTAGGTAAAGGTTCAGGTGGA
GTGGAATTCACGTAAAGTTGATAA SEQ ID NO: 140
TTGCGAAAAGGATTGATCCAAGGAGAAATGGCGTAATGAATTAGCATAGAAAGCAATTAGTTTTAT
ATTGCATAAATAATGATGAATTATATTAGGTAAAGGTTCAGGTGGAGTGG SEQ ID NO: 141
AAAATAAATGCTATGTCACGTTTTACACTAATGATAAAAAAATTAAAGTACAATATAAGTAGAAAAT
ATGTTCTAAAACCAAACAAAAAAGTGCATTAAATTGCGAAAAGGATTGATCCAAGGAGAAATGGC
GTAATGAATTAGCATAGAAAGCAATTAGTTTTATATTGCATAAATAATGATGAATTATATTAGGTAA
AGGTTCAGGTGGAGTGGAATTCAC SEQ ID NO: 142
AATTAAAGTACAATATAAGTAGAAAATATGTTCTAAAACCAAACAAAAAAGTGCATTAAATTGCGAA
AAGGATTGATCCAAGGAGAAATGGCGTAATGAATTAGCATAGAAAGCAATTAGTTTTATATTGCAT
AAATAATGAT SEQ ID NO: 143
TAGAAAATATGTTCTAAAACCAAACAAAAAAGTGCATTAAATTGCGAAAAGGATTGATCCAAGGAG
AAATGGCGTAATGAATTAGCATAGAAAGCAATTAGTTT SEQ ID NO: 144
AAACAAAAAAGTGCATTAAATTGCGAAAAGGATTGATCCAAGGAGAAATGGCGTAATGAATTA
CATTAAATTGCGAAAAGGATTGATCCAAGGAGAAATGGCGTAATGAATTAGCATAGAAAGCAATT
AGTTTTATATTGCATAAATAATGATGAATTATATTAGGTAAAGGTTCAGGTGGAGTGGAATTCACG
TAAAGTTGATAACTGAGAGTCGTTCGATAATTTGATTGATTTGACTAAATTTTTATTTAACGGCTCT
CGACTATCAACTTTGCGATGCACCT SEQ ID NO: 145
TAATGAATTAGCATAGAAAGCAATTAGTTTTATATTGCATAAATAATGATGAATTATATTAGGTAAA
GGTTCAGGTGGAGTGGAATTCACGTAAAGTTGATAACTGAGAGTCGTTCGATAATTTGATTGATT
TGACTAAATTT SEQ ID NO: 146
CAATTAGTTTTATATTGCATAAATAATGATGAATTATATTAGGTAAAGGTTCAGGTGGAGTGGAATT
CACGTAAAGTTGATAACTGAGAGTCGTTCGATAATT SEQ ID NO: 147
AAATAATGATGAATTATATTAGGTAAAGGTTCAGGTGGAGTGGAATTCACGTAAAGTTGATAA
SEQ ID NO: 148
TCTTAATCTTCACTGAAACGTCCTCGATTCTCTTGCGAGCTTCTGCTTTAGACAACATGATACATT
CCGCTGCTTCATCCATGGCCTCACCCCTCTGCACGTGGCTGGTGGCGGCTTGCATGTCAGTCAC
GTGCCACACTGACCGCGCCGGGACTCCAAAGGCAGCGCTTCGGTCATCCAAGAGGTCAAGGAG
GACGAAGGCGCTGCCAGCAAACACCTCCCAATTGAACATGAGATTGATTTCTTCGCTGTACGGAT
CCAGCATTCAGGGACTCATAAGCTCCTTCGAGCCCTGCGATGACTACTGCAACACTCACAATGC
CTTCTCTTCGCTCTTTCGATCCACAACTCCCAATCGCAGACACACCCGTCTCAATAAACTTTCTCA
TTCTGGTAAACTTTTTTTTTTTTTTTTTTAATTTTCCCCACTTAAATCACCTCAAGTTAAGATATTT
SEQ ID NO: 149
GACAACATGATACATTCCGCTGCTTCATCCATGGCCTCACCCCTCTGCACGTGGCTGGTGGCGG
CTTGCATGTCAGTCACGTGCCACACTGACCGCGCCGGGACTCCAAAGGCAGCGCTTCGGTCAT
CCAAGAGGTCAAGGAGGACGAAGGCGCTGCCAGCAAACACCTCCCAATTGAACATGAGATTGAT
TTCTTCGCTGTACGGATCCAGCATTCAGGGACTCATAAGCTCCTTCGAGCCCTGCGATGACTACT
GCAACACTCACAATGCCTTCTCTTCGCTCTTTCGATCCACAACTCCCAATCGCAGACACACCCGT
CTCAATAAACTTTCTCATTCTGGTAAACTTTTTT SEQ ID NO: 150
CCCTCTGCACGTGGCTGGTGGCGGCTTGCATGTCAGTCACGTGCCACACTGACCGCGCCGGGA
CTCCAAAGGCAGCGCTTCGGTCATCCAAGAGGTCAAGGAGGACGAAGGCGCTGCCAGCAAACA
CCTCCCAATTGAACATGAGATTGATTTCTTCGCTGTACGGATCCAGCATTCAGGGACTCATAAGC
TCCTTCGAGCCCTGCGATGACTACTGCAACACTCACAATGCCTTCTCTTCGCTCTTTCGATCCAC
AACTCCCAATCGCAGACAC SEQ ID NO: 151
GCGGCTTGCATGTCAGTCACGTGCCACACTGACCGCGCCGGGACTCCAAAGGCAGCGCTTCGG
TCATCCAAGAGGTCAAGGAGGACGAAGGCGCTGCCAGCAAACACCTCCCAATTGAACATGAGAT
TGATTTCTTCGCTGTACGGATCCAGCATTCAGGGACTCATAAGCTCCTTCGAGCCCTGCGATGAC
TACTGCAACACTCACAATGCCTTCTCTTCGCTCTTTCGATCCA SEQ ID NO: 152
GTGCCACACTGACCGCGCCGGGACTCCAAAGGCAGCGCTTCGGTCATCCAAGAGGTCAAGGAG
GACGAAGGCGCTGCCAGCAAACACCTCCCAATTGAACATGAGATTGATTTCTTCGCTGTACGGAT
CCAGCATTCAGGGACTCATAAGCTCCTTCGAGCCCTGCGATGACTACTGCAACACTCACAATGC
CTT SEQ ID NO: 153
GGACTCCAAAGGCAGCGCTTCGGTCATCCAAGAGGTCAAGGAGGACGAAGGCGCTGCCAGCAA
ACACCTCCCAATTGAACATGAGATTGATTTCTTCGCTGTACGGATCCAGCATTCAGGGACTCATA
AGCTCCTTCGAGCCCTGCGATGACTAC SEQ ID NO: 154
GGACTCCAAAGGCAGCGCTTCGGTCATCCAAGAGGTCAAGGAGGACGAAGGCGCTGCCAGCAA
ACACCTCCCAATTGAACATGAGATTGATTTCTTCGCTGTACGGATCCAGCATTCAGG SEQ ID
NO: 155
GCTGTACGGATCCAGCATTCAGGGACTCATAAGCTCCTTCGAGCCCTGCGATGACTAC SEQ ID
NO: 156
GACAACATGATACATTCCGCTGCTTCATCCATGGCCTCACCCCTCTGCACGTGGCTGGTGGCGG
CTTGCATGTCAGTCACGTGCCACACTGACCGCGCCGGGACTCCAAAGGCAGCGCTTCGGTCAT
CCAAGAGGTCAAGGAGGACGAAGGCGCTGCCAGCAAACACCTCCCAATTGAACATGAGATTGAT
TTCTTCGCTGTACGGATCCAGCATTCAGGGACT SEQ ID NO: 157
CCCTCTGCACGTGGCTGGTGGCGGCTTGCATGTCAGTCACGTGCCACACTGACCGCGCCGGGA
CTCCAAAGGCAGCGCTTCGGTCATCCAAGAGGTCAAGGAGGACGAAGGCGCTGCCAGCAAACA
CCTCCCAATTGAACATG SEQ ID NO: 158
GCGGCTTGCATGTCAGTCACGTGCCACACTGACCGCGCCGGGACTCCAAAGGCAGCGCTTCGG
TCATCCAAGAGGTCAAGGAGGACGAAGGCGCTGCCAGCAA SEQ ID NO: 159
GTGCCACACTGACCGCGCCGGGACTCCAAAGGCAGCGCTTCGGTCATCCAAGAGGTCAAGGAG
SEQ ID NO: 160
CCGGGACTCCAAAGGCAGCGCTTCGGTCATCCAAGAGGTCAAGGAGGACGAAGGCGCTGCCAG
CAAACACCTCCCAATTGAACATGAGATTGATTTCTTCGCTGTACGGATCCAGCATTCAGGGACTC
ATAAGCTCCTTCGAGCCCTGCGATGACTACTGCAACACTCACAATGCCTTCTCTTCGCTCTTTCG
ATCCACAACTCCCAATCGCAGACACACCCG SEQ ID NO: 161
AAGGAGGACGAAGGCGCTGCCAGCAAACACCTCCCAATTGAACATGAGATTGATTTCTTCGCTG
TACGGATCCAGCATTCAGGGACTCATAAGCTCCTTCGAGCCCTGCGATGACTACTGCAACACTC
ACAATGCCTTCTCTT SEQ ID NO: 162
CAGCAAACACCTCCCAATTGAACATGAGATTGATTTCTTCGCTGTACGGATCCAGCATTCAGGGA
CTCATAAGCTCCTTCGAGCCCTGCGATGACTACTGCAA SEQ ID NO: 163
AACATGAGATTGATTTCTTCGCTGTACGGATCCAGCATTCAGGGACTCATAAGCTCCTTCGAG
SEQ ID NO: 164
AGGTCAAGGAGGACGAAGGCGCTGCCAGCAAACACCTCCCAATTGAACATGAGATTGATTTCTT
CGCTGTACGGATCCAGCATTCAGGGACTCATAAGCTCCTTCGAGCCCTGCGATGACTACTGCAA
CACTCACAATGCCTTCTCTTCGCTCTTTCGATCCACAACTCCCAATCGCAGACACACCCGTCTCA
ATAAACTTTCTCATTCTGGTAAACTTTTTT SEQ ID NO: 165
AATTGAACATGAGATTGATTTCTTCGCTGTACGGATCCAGCATTCAGGGACTCATAAGCTCCTTC
GAGCCCTGCGATGACTACTGCAACACTCACAATGCCTTCTCTTCGCTCTTTCGATCCACAACTCC
CAATCGCAGACACA SEQ ID NO: 166
TCTTCGCTGTACGGATCCAGCATTCAGGGACTCATAAGCTCCTTCGAGCCCTGCGATGACTACT
GCAACACTCACAATGCCTTCTCTTCGCTCTTTCGATCCA SEQ ID NO: 167
CATTCAGGGACTCATAAGCTCCTTCGAGCCCTGCGATGACTACTGCAACACTCACAATGCCTT
SEQ ID NO: 168
GAATTTCTTGAATGATAGGCGGTTGCATGCAATTTTTATTATTGCTGACTTATTCCTCTGAATTGCA
AAACTATAAGGTAAAACAATGGCTGTAGCTGTGGAACCTGCACACGAAGTCACAGCAACGAAAAA
ACCTCCTACAAAACAAAGGCGAGTAGTTGTGACTGGGTTGGGCGTTGTTACACCTCTTGGTCAC
GAACCAGATGTTTACTACAACAATTTACTTGATGGTGTTAGTGGCATAAGTGAGATCGAGACATTT
GATTGTGCTGAATATCCAACGGCAAGTTATATATAATTGAAATTGTATTTCTTGTTGTCTGTTGTAG
CTTTTGTTCTGAACATTCTATGTTTCCAGAGGATTGCTGGTGAGATCAAGTC SEQ ID NO: 169
TATTCCTCTGAATTGCAAAACTATAAGGTAAAACAATGGCTGTAGCTGTGGAACCTGCACACGAA
GTCACAGCAACGAAAAAACCTCCTACAAAACAAAGGCGAGTAGTTGTGACTGGGTTGGGCGTTG
TTACACCTCTTGGTCACGAACCAGATGTTTACTACAACAATTTACTTGATGGTGTTAGTGGCATAA
GTGAGATCGAGACATTTGATTGTGCTGAATATCCAACGGCAAGTTATATATAATTGAAATTGTATT
TCTTGTTGTCTGTTGTAGCT SEQ ID NO: 170
TGTAGCTGTGGAACCTGCACACGAAGTCACAGCAACGAAAAAACCTCCTACAAAACAAAGGCGA
GTAGTTGTGACTGGGTTGGGCGTTGTTACACCTCTTGGTCACGAACCAGATGTTTACTACAACAA
TTTACTTGATGGTG SEQ ID NO: 171
ACGAAGTCACAGCAACGAAAAAACCTCCTACAAAACAAAGGCGAGTAGTTGTGACTGGGTTGGG
CGTTGTTACACCTCTTGGTCACGAACCAGATGTTTACTA SEQ ID NO: 172
AAACCTCCTACAAAACAAAGGCGAGTAGTTGTGACTGGGTTGGGCGTTGTTACACCTCTTGGT
SEQ ID NO: 173
GCGAGTAGTTGTGACTGGGTTGGGCGTTGTTACACCTCTTGGTCACGAACCAGATGTTTACTACA
ACAATTTACTTGATGG SEQ ID NO: 174
GCGAGTAGTTGTGACTGGGTTGGGCGTTGTTACACCTCTTGGTCACGAACCAGATGT SEQ ID
NO: 175
CCTCTTGGTCACGAACCAGATGTTTACTACAACAATTTACTTGATGG SEQ ID NO: 176
TATTCCTCTGAATTGCAAAACTATAAGGTAAAACAATGGCTGTAGCTGTGGAACCTGCACACGAA
GTCACAGCAACGAAAAAACCTCCTACAAAACAAAGGCGAGTAGTTGTGACTGGGTTGGGCGTTG
TTACACCTCTTGGTCACGAACCAGATGTTTACTACAACAATTTACTTGATGGTGTTAGTGGCATAA
GTGAGATCGAGACATTTGATTGTGCTGA SEQ ID NO: 177
TGTAGCTGTGGAACCTGCACACGAAGTCACAGCAACGAAAAAACCTCCTACAAAACAAAGGCGA
GTAGTTGTGACTGGGTTGGGCGTTGTTACACCTCTTGGTCACGAACCAGATGTTTACTACAACAA
TTTACTTGATGGTG SEQ ID NO: 178
ACGAAGTCACAGCAACGAAAAAACCTCCTACAAAACAAAGGCGAGTAGTTGTGACTGGGTTGGG
CGTTGTTACACCTCTTGGTCACGAACCAGATGTTTACTA SEQ ID NO: 179
AAACCTCCTACAAAACAAAGGCGAGTAGTTGTGACTGGGTTGGGCGTTGTTACACCTCTTGGT
SEQ ID NO: 180
AATGGCTGTAGCTGTGGAACCTGCACACGAAGTCACAGCAACGAAAAAACCTCCTACAAAACAAA
GGCGAGTAGTTGTGACTGGGTTGGGCGTTGTTACACCTCTTGGTCACGAACCAGATGTTTACTA
CAACAATTTACTTGATGGTGTTAGTGGCATAAGTGAGATCGAGACATTTGATTGTGCTGAATATCC
AACGGCAAGTTATATATAATTGAAATTG SEQ ID NO: 181
ACGAAAAAACCTCCTACAAAACAAAGGCGAGTAGTTGTGACTGGGTTGGGCGTTGTTACACCTCT
TGGTCACGAACCAGATGTTTACTACAACAATTTACTTGATGGTGTTAGTGGCATAAGTGAGATCG
AGACATTTGATTG SEQ ID NO: 182
ACAAAGGCGAGTAGTTGTGACTGGGTTGGGCGTTGTTACACCTCTTGGTCACGAACCAGATGTT
TACTACAACAATTTACTTGATGGTGTTAGTGGCATAAGT SEQ ID NO: 183
CTGGGTTGGGCGTTGTTACACCTCTTGGTCACGAACCAGATGTTTACTACAACAATTTACTTG
SEQ ID NO: 184
ACACGAAGTCACAGCAACGAAAAAACCTCCTACAAAACAAAGGCGAGTAGTTGTGACTGGGTTG
GGCGTTGTTACACCTCTTGGTCACGAACCAGATGTTTACTACAACAATTTACTTGATGGTGTTAGT
GGCATAAGTGAGATCGAGACATTTGATTGTGCTGAATATCCAACGGCAAGTTATATATAATTGAAA
TTGTATTTCTTGTTGTCTGTTGTAGCT SEQ ID NO: 185
AGGCGAGTAGTTGTGACTGGGTTGGGCGTTGTTACACCTCTTGGTCACGAACCAGATGTTTACTA
CAACAATTTACTTGATGGTGTTAGTGGCATAAGTGAGATCGAGACATTTGATTGTGCTGAATATCC
AACGGCAAGTTA SEQ ID NO: 186
GTTGGGCGTTGTTACACCTCTTGGTCACGAACCAGATGTTTACTACAACAATTTACTTGATGGTGT
TAGTGGCATAAGTGAGATCGAGACATTTGATTGTGCT SEQ ID NO: 187
TTGGTCACGAACCAGATGTTTACTACAACAATTTACTTGATGGTGTTAGTGGCATAAGTGAGA
SEQ ID NO: 188
AGTTATATATAATTGAAATTGTATTTCTTGTTGTCTGTTGTAGCTTTTGTTCTGAACATTCTATGTTT
CCAGAGGATTGCTGGTGAGATCAAGTCGTTCTCCACTGATGGCTGGGTTGCACCAAAACTTTCG
AAGAGAATGGATAAATTTATGCTCTATTTGCTAACAGCCGGTAAAAAGGCCTTAGTTGATGGTGG
AATTACTGAAGATGTAATGGATGAGTTGAATAAGGAAAAATGTGGAGTTTTGATTGGCTCAGCGA
TGGGTGGCATGAAGGTAACCTTGTCTTTCTTTCATGTTGAGATACAAATCTTGTAAAACCAGCTG
GTGTTGTGGACTGAATTTAATGCCACT SEQ ID NO: 189
TCTGAACATTCTATGTTTCCAGAGGATTGCTGGTGAGATCAAGTCGTTCTCCACTGATGGCTGGG
TTGCACCAAAACTTTCGAAGAGAATGGATAAATTTATGCTCTATTTGCTAACAGCCGGTAAAAAGG
CCTTAGTTGATGGTGGAATTACTGAAGATGTAATGGATGAGTTGAATAAGGAAAAATGTGGAGTT
TTGATTGGCTCAGCGATGGGTGGCATGAAGGTAACCTTGTCTTTCTTTCATGTTGAGA SEQ ID
NO: 190
AAGTCGTTCTCCACTGATGGCTGGGTTGCACCAAAACTTTCGAAGAGAATGGATAAATTTATGCT
CTATTTGCTAACAGCCGGTAAAAAGGCCTTAGTTGATGGTGGAATTACTGAAGATGTAATGGATG
AGTTGAATAAGGAAAAATGTGGAGTTTTGATTGGCTCAGCGATG SEQ ID NO: 191
CTGGGTTGCACCAAAACTTTCGAAGAGAATGGATAAATTTATGCTCTATTTGCTAACAGCCGGTA
AAAAGGCCTTAGTTGATGGTGGAATTACTGAAGATGTAATGGATGAGTTGAATAAGGAAAAATGT
GGAG SEQ ID NO: 192
CGAAGAGAATGGATAAATTTATGCTCTATTTGCTAACAGCCGGTAAAAAGGCCTTAGTTGATGGT
GGAATTACTGAAGATGTAATGGATGAGTT SEQ ID NO: 193
ATGCTCTATTTGCTAACAGCCGGTAAAAAGGCCTTAGTTGATGGTGGAATTACT SEQ ID NO: 194
ATGCTCTATTTGCTAACAGCCGGTAAAAAGGCCTTAGTTGATGG SEQ ID NO: 195
TAAAAAGGCCTTAGTTGATGGTGGAATTACT SEQ ID NO: 196
TCTGAACATTCTATGTTTCCAGAGGATTGCTGGTGAGATCAAGTCGTTCTCCACTGATGGCTGGG
TTGCACCAAAACTTTCGAAGAGAATGGATAAATTTATGCTCTATTTGCTAACAGCCGGTAAAAAGG
CCTTAGTTGATGGTGGAATTACTGAAGATGTAATGGATGAGTTGAATAAGGAAAAATGTGGAGTT
TTGATTGGCTCAGCGATGGGTGGCATG SEQ ID NO: 197
AAGTCGTTCTCCACTGATGGCTGGGTTGCACCAAAACTTTCGAAGAGAATGGATAAATTTATGCT
CTATTTGCTAACAGCCGGTAAAAAGGCCTTAGTTGATGGTGGAATTACTGAAGATGTAATGGATG
AGTTGAATAAGGA SEQ ID NO: 198
CTGGGTTGCACCAAAACTTTCGAAGAGAATGGATAAATTTATGCTCTATTTGCTAACAGCCGGTA
AAAAGGCCTTAGTTGATGGTGGAATTACTGAAGATGTA SEQ ID NO: 199
CGAAGAGAATGGATAAATTTATGCTCTATTTGCTAACAGCCGGTAAAAAGGCCTTAGTTGATG
SEQ ID NO: 200
GGATTGCTGGTGAGATCAAGTCGTTCTCCACTGATGGCTGGGTTGCACCAAAACTTTCGAAGAG
AATGGATAAATTTATGCTCTATTTGCTAACAGCCGGTAAAAAGGCCTTAGTTGATGGTGGAATTAC
TGAAGATGTAATGGATGAGTTGAATAAGGAAAAATGTGGAGTTTTGATTGGCTCAGCGATGGGTG
GCATGAAGGTAACCTTGTCTTTCTTT SEQ ID NO: 201
GGTTGCACCAAAACTTTCGAAGAGAATGGATAAATTTATGCTCTATTTGCTAACAGCCGGTAAAAA
GGCCTTAGTTGATGGTGGAATTACTGAAGATGTAATGGATGAGTTGAATAAGGAAAAATGTGGAG
TTTTGATTGG SEQ ID NO: 202
AGAGAATGGATAAATTTATGCTCTATTTGCTAACAGCCGGTAAAAAGGCCTTAGTTGATGGTGGA
ATTACTGAAGATGTAATGGATGAGTTGAATAAGGAA SEQ ID NO: 203
CTCTATTTGCTAACAGCCGGTAAAAAGGCCTTAGTTGATGGTGGAATTACTGAAGATGTAA SEQ
ID NO: 204
GGTGAGATCAAGTCGTTCTCCACTGATGGCTGGGTTGCACCAAAACTTTCGAAGAGAATGGATAA
ATTTATGCTCTATTTGCTAACAGCCGGTAAAAAGGCCTTAGTTGATGGTGGAATTACTGAAGATGT
AATGGATGAGTTGAATAAGGAAAAATGTGGAGTTTTGATTGGCTCAGCGATGGGTGGCATGAAG
GTAACCTTGTCTTTCTTTCATGTTGAGA SEQ ID NO: 205
CCAAAACTTTCGAAGAGAATGGATAAATTTATGCTCTATTTGCTAACAGCCGGTAAAAAGGCCTTA
GTTGATGGTGGAATTACTGAAGATGTAATGGATGAGTTGAATAAGGAAAAATGTGGAGTTTTGAT
TGGCTCAGCGATG SEQ ID NO: 206
GGATAAATTTATGCTCTATTTGCTAACAGCCGGTAAAAAGGCCTTAGTTGATGGTGGAATTACTGA
AGATGTAATGGATGAGTTGAATAAGGAAAAATGTGGAG SEQ ID NO: 207
GCTAACAGCCGGTAAAAAGGCCTTAGTTGATGGTGGAATTACTGAAGATGTAATGGATGAGTT
SEQ ID NO: 208
CTCACCATTGATTCTTTTTCACCTGCATACGGTTATCATGCCATAAATCTAGCTGATATGAATCCA
CTAATCATTGGTACAGGTTTGGGAGGTTTTGTGGCATGCAGAGCCCTATCGCAGAGGAATACAG
ACCCTACCAAAGCTTCACGCCCTTGGGACATTGTAATACAACAGAAATCCTGCTCATTCTTTCTG
CTACTCACCATGTTTTTGTATTGCTGATCTttcatcattttttatttttgaaacaatac SEQ ID NO: 209
GCCATAAATCTAGCTGATATGAATCCACTAATCATTGGTACAGGTTTGGGAGGTTTTGTGGCATG
CAGAGCCCTATCGCAGAGGAATACAGACCCTACCAAAGCTTCACGCCCTTGGGACATTGTAATA
CAACAGAAATCCTGCTCATTCTTTCTGCTACTCACCATGTTTTTGT SEQ ID NO: 210
AATCCACTAATCATTGGTACAGGTTTGGGAGGTTTTGTGGCATGCAGAGCCCTATCGCAGAGGA
ATACAGACCCTACCAAAGCTTCACGCCCTTGGGACATTGTAATACAACAGAAATCCTGCTCATTC
TTTCT SEQ ID NO: 211
AGGTTTGGGAGGTTTTGTGGCATGCAGAGCCCTATCGCAGAGGAATACAGACCCTACCAAAGCT
TCACGCCCTTGGGACATTGTAATACAACAG SEQ ID NO: 212
ATAATTAGTATCTGCTTGTACTTTACCACATTTCCTTTATGATTCCATTCGGACTCTTCTTTGAGTA
TTCTATGACTTGACAAGGAATGATTTGTATCTCCCTGCGGAACTTGCATTCATGTTTCTTTACTCC
ATCTTAAATAGGAGCTGGCGTTATACTTTGCATTGAAAAGGCATTAGCTCAGTCTGGAGTATCAAA
AGAGGATGTGAACTACATAAATGC SEQ ID NO: 275
GATTCCATTCGGACTCTTCTTTGAGTATTCTATGACTTGACAAGGAATGATTTGTATCTCCCTGCG
GAACTTGCATTCATGTTTCTTTACTCCATCTTAAATAGGAGCTGGCGTTATACTTTGCATTGAAAA
GGCATTAGCTC SEQ ID NO: 276
TTGAGTATTCTATGACTTGACAAGGAATGATTTGTATCTCCCTGCGGAACTTGCATTCATGTTTCT
TTACTCCATCTTAAATAGGAGCTGGCGTTATACTTTG SEQ ID NO: 277
CAAGGAATGATTTGTATCTCCCTGCGGAACTTGCATTCATGTTTCTTTACTCCATCTTAAATA SEQ
ID NO: 278
CTACAAAATCTATGATTGGTCATCTACTAGGAGCAGCTGGTGGCGTGGAAGCTGTAGCAACAGT
CCAGGTAAAACCCAATGGATCACTTAGTTCGGTACCCCGCGTTTTGTGACTAAAATGGTCCTGAG
GTTTGCTCTCTCTTTATACAGGCAATTAGGACAGGGTGGGTGCATCCCAATATCAACCTGGAAAA
CCCAGATCAAGGAGTGGTAATGTTTCTTG SEQ ID NO: 279
TGGCGTGGAAGCTGTAGCAACAGTCCAGGTAAAACCCAATGGATCACTTAGTTCGGTACCCCGC
GTTTTGTGACTAAAATGGTCCTGAGGTTTGCTCTCTCTTTATACAGGCAATTAGGACAGGGGGG
TGCATCCCAATATC SEQ ID NO: 280
CAGTCCAGGTAAAACCCAATGGATCACTTAGTTCGGTACCCCGCGTTTTGTGACTAAAATGGTCC
TGAGGTTTGCTCTCTCTTTATACAGGCAATTAGGACAG SEQ ID NO: 281
GGATCACTTAGTTCGGTACCCCGCGTTTTGTGACTAAAATGGTCCTGAGGTTTGCTCTCTCTT
SEQ ID NO: 282
TTAAATTGTGTGTTGTTTTTATGTTAAGTTACTAAGGTAGCCAGGCAATTGTTACACGCATGATCA
AAGTCATGAATTGGATGAAAATATATGTGGAAATGGTTGGAGCAGAAACTCTTGAGGACATTGTT
AAGAGATGGTTTCGTGGTTTAGATTGTCTGGCCGAGATACATGGTGGAGCCGGTTCTATCCACC
GATGAAGAACATGAATGGCTGCTATTGCCTTTATAGAGTAGGACTTGACCCACTTTGATGTGACT
CTTAAACAAATCCACTTGGCAGTATATATAAGCTCTTCTGTTTTATTTATTCCCTATGTACCCAAAT
AGTTTGTGCAATAACAAATTGCAATTTATTTTTTCGCTGCCTTGTTCTATTGCCCTTAAACCAGACC
ATTTAGGGAAAAGGGGATGATTTTAATCTCTCAGGAAA SEQ ID NO: 283
GGTTGGAGCAGAAACTCTTGAGGACATTGTTAAGAGATGGTTTCGTGGTTTAGATTGTCTGGCCG
AGATACATGGTGGAGCCGGTTCTATCCACCGATGAAGAACATGAATGGCTGCTATTGCCTTTATA
GAGTAGGACTTGACCCACTTTGATGTGACTCTTAAACAAATCCACTTGGCAGTATATATAAGCTCT
TCTGTTTTATTTATTCCCTATGTACCCAAATAGTTTGTGCAATAACAAATTGCAATTTATTTTTTCGC
TGCCTTGTTCTATTGCCCTTAAACCAGACCATTTAGGGAAAAGGGGATGATTTTAATCTCTCAGG
SEQ ID NO: 284
GGTTGGAGCAGAAACTCTTGAGGACATTGTTAAGAGATGGTTTCGTGGTTTAGATTGTCTGGCCG
AGATACATGGTGGAGCCGGTTCTATCCACCGATGAAGAACATGAATGGCTGCTATTGCCTTTATA
GAGTAGGACTTGACCCACTTTGATGTGACTCTTAAACAAATCCACTTGG SEQ ID NO: 285
GACTCTTAAACAAATCCACTTGGCAGTATATATAAGCTCTTCTGTTTTATTTATTCCCTATGTACCC
AAATAGTTTGTGCAATAACAAATTGCAATTTATTTTTTCGCTGCCTTGTTCTATTGCCCTTAAACCA
GACCATTTAGGGAAAAGGGGATGATTTTAATCTCTCAGG SEQ ID NO: 286
TTAAATTGTGTGTTGTTTTTATGTTAAGTTACTAAGGTAGCCAGGCAATTGTTACACGCATGATCA
AAGTCATGAATTGGATGAAAATATATGTGGAAATGGTTGGAGCAGAAACTCTTGAGGACATTGTT
AAGAGATGGTTTCGTGGTTTAGATTGTCTGGCCGAGATACATGGTGGAGCCGGTTCTATCCACC
GATGAAGAACATGAATGGCTGCTATTGC SEQ ID NO: 287
CCAGGCAATTGTTACACGCATGATCAAAGTCATGAATTGGATGAAAATATATGTGGAAATGGTTG
GAGCAGAAACTCTTGAGGACATTGTTAAGAGATGGTTTCGTGGTTTAGATTGTCTGGCCGAGATA
CATGGTGGAGCCG SEQ ID NO: 288
TGATCAAAGTCATGAATTGGATGAAAATATATGTGGAAATGGTTGGAGCAGAAACTCTTGAGGAC
ATTGTTAAGAGATGGTTTCGTGGTTTAGATTGTCTGGC SEQ ID NO: 289
ATGAAAATATATGTGGAAATGGTTGGAGCAGAAACTCTTGAGGACATTGTTAAGAGATGGTTT
SEQ ID NO: 290
GTCTGGCCGAGATACATGGTGGAGCCGGTTCTATCCACCGATGAAGAACATGAATGGCTGCTAT
TGCCTTTATAGAGTAGGACTTGACCCACTTTGATGTGACTCTTAAACAAATCCACTTGGCAGTATA
TATAAGCTCTTCTGTTTTATTTATTCCCTATGTACCCAAATAGTTTGTGCAATAACAAATTGCAATT
TATTTTTTCGCTGCCTTGTTCTATTG SEQ ID NO: 291
ATGAAGAACATGAATGGCTGCTATTGCCTTTATAGAGTAGGACTTGACCCACTTTGATGTGACTC
TTAAACAAATCCACTTGGCAGTATATATAAGCTCTTCTGTTTTATTTATTCCCTATGTACCCAAATA
GTTTGTGCAAT SEQ ID NO: 292
CTATTGCCTTTATAGAGTAGGACTTGACCCACTTTGATGTGACTCTTAAACAAATCCACTTGGCAG
TATATATAAGCTCTTCTGTTTTATTTATTCCCTATGT SEQ ID NO: 293
GACTTGACCCACTTTGATGTGACTCTTAAACAAATCCACTTGGCAGTATATATAAGCTCTTCT SEQ
ID NO: 294
ATAAGCTCTTCTGTTTTATTTATTCCCTATGTACCCAAATAGTTTGTGCAATAACAAATTGCAATTT
ATTTTTTCGCTGCCTTGTTCTATTGCCCTTAAACCAGACCATTTAGGGAAAAGGGGATGATTTTAA
TCTCTCAGGAAA SEQ ID NO: 295
AGTTTGTGCAATAACAAATTGCAATTTATTTTTTCGCTGCCTTGTTCTATTGCCCTTAAACCAGACC
ATTTAGGGAAAAGGGGATGATTTTAATCTCTCAGGAAA SEQ ID NO: 296
GCAATTTATTTTTTCGCTGCCTTGTTCTATTGCCCTTAAACCAGACCATTTAGGGAAAAGGGGATG
ATTTTAATCTCTCAGGAAA SEQ ID NO: 297
CTTGTTCTATTGCCCTTAAACCAGACCATTTAGGGAAAAGGGGATGATTTTAATCTCTCAGGAAA
SEQ ID NO: 298
AGCATTTATATGCATGCTATGATTACCATGCTTAAAATCCGAAGAGAACAAGGCAAAAGAATGTGA
CAAGGGTATTTTTATATATTTAGAATCATGTGTGCCATTTGCTGATGGTTAGGTTAAGTATTTAGAA
ATATACTATATGTTGCATTTTTCTTGTTTGGCACTGGAATTTCTTCTTCCGAGATCGTGATGGAATT
TAAAACTGCATGTTGCGATCATG SEQ ID NO: 299
GAAGAGAACAAGGCAAAAGAATGTGACAAGGGTATTTTTATATATTTAGAATCATGTGTGCCATTT
GCTGATGGTTAGGTTAAGTATTTAGAAATATACTATATGTTGCATTTTTCTTGTTTGGCACTGGAAT
TTCTTCTTCC SEQ ID NO: 300
ATGTGACAAGGGTATTTTTATATATTTAGAATCATGTGTGCCATTTGCTGATGGTTAGGTTAAGTA
TTTAGAAATATACTATATGTTGCATTTTTCTTGTTTG SEQ ID NO: 301
TATATTTAGAATCATGTGTGCCATTTGCTGATGGTTAGGTTAAGTATTTAGAAATATACTATA SEQ
ID NO: 302
AGGAGCAGCTGGTGGCGTGGAAGCTGTAGCAACAGTCCAGGTAAAACCCAATGGATCACTTAGT
TCGGTACCCCGCGATTGATGACTAAAATGGTCCTGAGGTTTGCTCTCTCTTCGTACAGGCAATTA
GGACAGGGTGGGTGCATCCCAATATCAACCTGGAAAACCCAGATCAAGGAGTGGTAATGTTTCT
TGTTGCATCATTCCATCCTTCATTTCAAAA SEQ ID NO: 303
GGTAAAACCCAATGGATCACTTAGTTCGGTACCCCGCGATTGATGACTAAAATGGTCCTGAGGTT
TGCTCTCTCTTCGTACAGGCAATTAGGACAGGGTGGGTGCATCCCAATATCAACCTGGAAAACC
CAGATCAAGGAGTGG SEQ ID NO: 304
TAGTTCGGTACCCCGCGATTGATGACTAAAATGGTCCTGAGGTTTGCTCTCTCTTCGTACAGGCA
ATTAGGACAGGGTGGGTGCATCCCAATATCAACCTGGA SEQ ID NO: 305
TGATGACTAAAATGGTCCTGAGGTTTGCTCTCTCTTCGTACAGGCAATTAGGACAGGGTGGGTG
SEQ ID NO: 306
ACACGGAGGGCATCTTTCGTGGGTACAAGTTTAGTTTCGATTTAATGTGGGTACATATGTCAGCA
TTTTCATTTTTCATGGATACAAATGGTCATTTATTCCTCTAAATAATGAGCAGGTCATATCGTCCTA
CTACGAGAAACACATTTAATTTGGATGACTGTGGATGTTCCTTGAACATGTATTAATTGTGTGTTG
TTTTTATGTTAAGTTACTAAGGTAGCCAGGCAATTGTTACACGCATGATCAAAGTCATGAATTGGA
TGAAAATATATGTGGAAATGGTTGGAGCAGAAACTCTTGAGGACATTGTTGAGAGATGGTTTGGT
GGTTTAGATTGTCTGGCCGAGATACATGGTGGAGCCGGTTCTATCCACCGATGAAGAACATGAA
TGGCTGCTATTGCCTTTATAGAGTAGGACTTGACCCACTTTGATGTGACTCTTAAACAAATCCACT
TGGCAGTATATATAAGCTCTTCTGTTTTATTTATTCCCTATGTACCCAAATAGTTTGTGCAATAACA
AATTGCAATTTATTTTTTCGCT SEQ ID NO: 307
CCTCTAAATAATGAGCAGGTCATATCGTCCTACTACGAGAAACACATTTAATTTGGATGACTGTGG
ATGTTCCTTGAACATGTATTAATTGTGTGTTGTTTTTATGTTAAGTTACTAAGGTAGCCAGGCAATT
GTTACACGCATGATCAAAGTCATGAATTGGATGAAAATATATGTGGAAATGGTTGGAGCAGAAAC
TCTTGAGGACATTGTTGAGAGATGGTTTGGTGGTTTAGATTGTCTGGCCGAGATACATGGTGGAG
CCGGTTCTATCCACCGATGAAGAACATGAATGGCTGCTATTGCCTTTATAGAGTAGGACTTGACC
CACTTTGATGTGACTCTTAAACAAATCCACTTGG SEQ ID NO: 308
CCTCTAAATAATGAGCAGGTCATATCGTCCTACTACGAGAAACACATTTAATTTGGATGACTGTGG
ATGTTCCTTGAACATGTATTAATTGTGTGTTGTTTTTATGTTAAGTTACTAAGGTAGCCAGGCAATT
GTTACACGCATGATCAAAGTCATGAATTGGATGAAAATATATGTGGAAATGGTTGGAGCAGAAAC
TCTTGAGG SEQ ID NO: 309
CCTCTAAATAATGAGCAGGTCATATCGTCCTACTACGAGAAACACATTTAATTTGGATGACTGTGG
ATGTTCCTTGAACATGTATTAATTGTGTGTTGTTTTTATGTTAAGTTACTAAGGTAGCCAGG SEQ
ID NO: 310
GTTAAGTTACTAAGGTAGCCAGGCAATTGTTACACGCATGATCAAAGTCATGAATTGGATGAAAA
TATATGTGGAAATGGTTGGAGCAGAAACTCTTGAGGACATTGTTGAGAGATGGTTTGGTGGTTTA
GATTGTCTGGCCGAGATACATGGTGGAGCCGGTTCTATCCACCGATGAAGAACATGAATGGCTG
CTATTGCCTTTATAGAGTAGGACTTGACCCACTTTGATGTGACTCTTAAACAAATCCACTTGG
SEQ ID NO: 311
GTTAAGTTACTAAGGTAGCCAGGCAATTGTTACACGCATGATCAAAGTCATGAATTGGATGAAAA
TATATGTGGAAATGGTTGGAGCAGAAACTCTTGAGG SEQ ID NO: 312
GGTTGGAGCAGAAACTCTTGAGGACATTGTTGAGAGATGGTTTGGTGGTTTAGATTGTCTGGCC
GAGATACATGGTGGAGCCGGTTCTATCCACCGATGAAGAACATGAATGGCTGCTATTGCCTTTAT
AGAGTAGGACTTGACCCACTTTGATGTGACTCTTAAACAAATCCACTTGG SEQ ID NO: 313
ACACGGAGGGCATCTTTCGTGGGTACAAGTTTAGTTTCGATTTAATGTGGGTACATATGTCAGCA
TTTTCATTTTTCATGGATACAAATGGTCATTTATTCCTCTAAATAATGAGCAGGTCATATCGTCCTA
CTACGAGAAACACATTTAATTTGGATGACTGTGGATGTTCCTTGAACATGTATTAATTGTGTGTTG
TTTTTATGTTAAGTTACTAAGGTAG SEQ ID NO: 314
TTTAATGTGGGTACATATGTCAGCATTTTCATTTTTCATGGATACAAATGGTCATTTATTCCTCTAA
ATAATGAGCAGGTCATATCGTCCTACTACGAGAAACACATTTAATTTGGATGACTGTGGATGTTCC
TTGAACATGT SEQ ID NO: 315
CAGCATTTTCATTTTTCATGGATACAAATGGTCATTTATTCCTCTAAATAATGAGCAGGTCATATCG
TCCTACTACGAGAAACACATTTAATTTGGATGACTG SEQ ID NO: 316
GATACAAATGGTCATTTATTCCTCTAAATAATGAGCAGGTCATATCGTCCTACTACGAGAAAC
SEQ ID NO: 317
AAATAATGAGCAGGTCATATCGTCCTACTACGAGAAACACATTTAATTTGGATGACTGTGGATGTT
CCTTGAACATGTATTAATTGTGTGTTGTTTTTATGTTAAGTTACTAAGGTAGCCAGGCAATTGTTAC
ACGCATGATCAAAGTCATGAATTGGATGAAAATATATGTGGAAATGGTTGGAGCAGAAACTCTTG
AGGACATTGTTGAGAGATGGTTTGG SEQ ID NO: 318
CATTTAATTTGGATGACTGTGGATGTTCCTTGAACATGTATTAATTGTGTGTTGTTTTTATGTTAAG
TTACTAAGGTAGCCAGGCAATTGTTACACGCATGATCAAAGTCATGAATTGGATGAAAATATATGT
GGAAATGGTTG SEQ ID NO: 319
GATGTTCCTTGAACATGTATTAATTGTGTGTTGTTTTTATGTTAAGTTACTAAGGTAGCCAGGCAA
TTGTTACACGCATGATCAAAGTCATGAATTGGATGAA SEQ ID NO: 320
TAATTGTGTGTTGTTTTTATGTTAAGTTACTAAGGTAGCCAGGCAATTGTTACACGCATGATC SEQ
ID NO: 321
ATTAATTGTGTGTTGTTTTTATGTTAAGTTACTAAGGTAGCCAGGCAATTGTTACACGCATGATCA
AAGTCATGAATTGGATGAAAATATATGTGGAAATGGTTGGAGCAGAAACTCTTGAGGACATTGTT
GAGAGATGGTTTGGTGGTTTAGATTGTCTGGCCGAGATACATGGTGGAGCCGGTTCTATCCACC
GATGAAGAACATGAATGGCTGCTATTGC SEQ ID NO: 322
CCAGGCAATTGTTACACGCATGATCAAAGTCATGAATTGGATGAAAATATATGTGGAAATGGTTG
GAGCAGAAACTCTTGAGGACATTGTTGAGAGATGGTTTGGTGGTTTAGATTGTCTGGCCGAGATA
CATGGTGGAGCCG SEQ ID NO: 323
TGATCAAAGTCATGAATTGGATGAAAATATATGTGGAAATGGTTGGAGCAGAAACTCTTGAGGAC
ATTGTTGAGAGATGGTTTGGTGGTTTAGATTGTCTGGC SEQ ID NO: 324
ATGAAAATATATGTGGAAATGGTTGGAGCAGAAACTCTTGAGGACATTGTTGAGAGATGGTTTG
SEQ ID NO: 325
GTCTGGCCGAGATACATGGTGGAGCCGGTTCTATCCACCGATGAAGAACATGAATGGCTGCTAT
TGCCTTTATAGAGTAGGACTTGACCCACTTTGATGTGACTCTTAAACAAATCCACTTGGCAGTATA
TATAAGCTCTTCTGTTTTATTTATTCCCTATGTACCCAAATAGTTTGTGCAATAACAAATTGCAATT
TATTTTTTCGCT SEQ ID NO: 326
ATGAAGAACATGAATGGCTGCTATTGCCTTTATAGAGTAGGACTTGACCCACTTTGATGTGACTC
TTAAACAAATCCACTTGGCAGTATATATAAGCTCTTCTGTTTTATTTATTCCCTATGTACCCAAATA
GTTTGTGCAAT SEQ ID NO: 292
CTATTGCCTTTATAGAGTAGGACTTGACCCACTTTGATGTGACTCTTAAACAAATCCACTTGGCAG
TATATATAAGCTCTTCTGTTTTATTTATTCCCTATGT SEQ ID NO: 293
GACTTGACCCACTTTGATGTGACTCTTAAACAAATCCACTTGGCAGTATATATAAGCTCTTCT SEQ
ID NO: 294

### Example 2

A single gene in the peanut genome is targeted to modify its oil profile to result in a modified peanut oil profile which does not separate in the peanut butter jar. This project has a greater chance of technical success and a fast path to commercialization. In addition, initial consultation revealed that this oil modified peanut line could be exempted from the USDA regulatory authority under the 2021 SECURE rule.

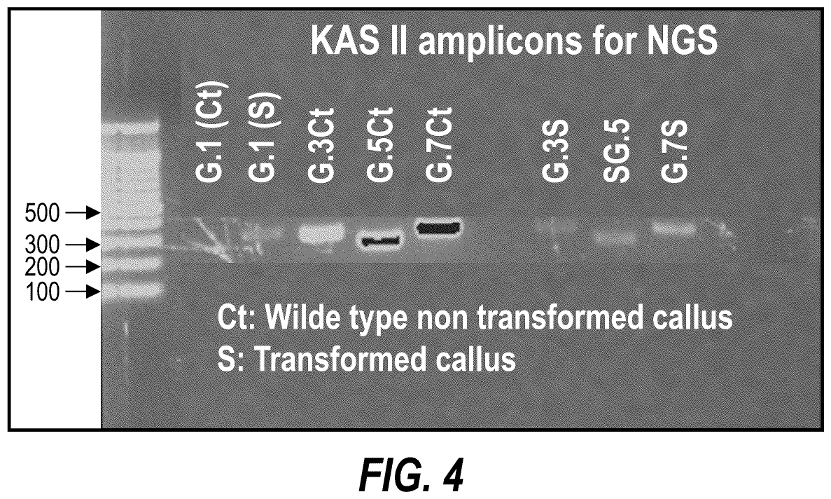

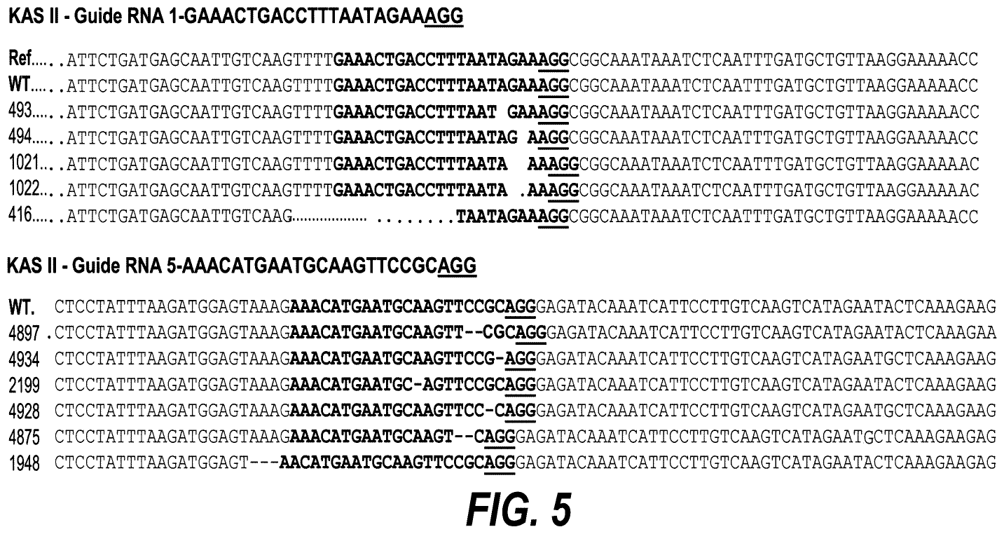

Using CRISPR/Cas9 nuclease and guide RNAs targeting the KAS II gene, deletions were obtained within the Kas II gene following transformation of peanut callus (FIGS. 4 and 5). As an alternative to the Cas9 nuclease, the CRISPR/LEG14 nuclease was also used to develop the oil modified peanut.

The foregoing is illustrative of the present invention and is not to be construed as limiting thereof. The invention is defined by the following claims, with equivalents of the claims to be included therein.

That which is claimed is:

1. A peanut plant or part thereof (e.g., seed) comprising at least one (one or more than one) mutation in one or more β-ketoacyl-ACP synthetase II (KASII) (also known as 3-oxoacyl-acyl carrier protein synthase II) genes encoding a KasII polypeptide.

2. The peanut plant or part thereof of claim 1, wherein the KASII gene (a) comprises a nucleotide sequence having at least 80% sequence identity to SEQ ID NO:1, SEQ ID NO:2, SEQ ID NO:116, SEQ ID NO:117, SEQ ID NO:118, and/or SEQ ID NO:119, (b) comprises a region of consecutive nucleotides having at least 80% sequence identity to any one of the nucleotide sequences of SEQ ID NOs:5-31, 32-51, 52-69, 70-83 and/or 275-298 and/or SEQ ID NOs:122-148, 149-168, 169-188, 189-208, 209-212 and/or 299-326, and/or (c) encodes an amino acid sequence having at least 80% sequence identity to SEQ ID NO:4 and/or SEQ ID NO:121.

3. The peanut plant or part thereof of claim 1 or claim 2, wherein the one or more KASII genes is in the A genome (GenBank Accession No. LOC112696350 (SEQ ID NO:1 or SEQ ID NO:2) and/or B genome (GenBank Accession No. LOC112740759 (SEQ ID NOs:116-119) of the peanut plant.

4. The peanut plant or part thereof any one of the preceding claims, wherein the at least one mutation is a base substitution, a base deletion, and/or a base insertion.

5. The peanut plant or part thereof of any one of the preceding claims, wherein the at least one mutation is located in the upstream region of the KASII gene (e.g., SEQ ID NOs:1, 2, or 116-119), optionally the mutation is located in a portion of the upstream region having at least 80% sequence identity to SEQ ID NOs:5-31 or 122-148.

6. The peanut plant or part thereof of claim 5, wherein the at least one mutation in the upstream region results in the KASII gene having reduced expression, thereby reducing production of KASII polypeptide.

7. The peanut plant or part thereof of any one of claim 1-4 or 6, wherein the at least one mutation is located in Exon 1, Exon 2, Exon 3, and/or Exon 7 of the KASII gene, optionally in Exon 1, Exon 2 and/or Exon 7, or region thereof, of any one of the nucleotide sequences of SEQ ID NO:1 or SEQ ID NO:2 (e.g., SEQ ID NOs:32-51, 52-69, or 70-83) or Exon 1, Exon 2, and/or Exon 3, or region thereof, of any one of the nucleotide sequences of SEQ ID NOs:116-119 (e.g., SEQ ID NOs:149-168, 169-188, 189-208).

8. The peanut plant or part thereof of any one of claim 1-4 or 6, wherein the at least one mutation is located in the 3′ region of the KASII gene, optionally in the 3′ region of any one of the nucleotide sequences of SEQ ID NO:1 or SEQ ID NO:2 (e.g., SEQ ID NOs:275-298) or of SEQ ID NOs:116-119 (e.g., SEQ ID NOs:292-294 and/or 299-326).

9. The peanut plant or part thereof of any one of claim 1-4 or 6-7, wherein the at least one mutation is located in Exon 7 of the KASII gene and results in KasII polypeptide comprising an amino acid substitution at L344 with reference to amino acid position numbering of SEQ ID NO:4 and/or SEQ ID NO:121, optionally wherein the mutation is L344F (Leu>Phe at residue 344).

10. The peanut plant or part thereof of claim 9, wherein the at least one mutation results in a modified KasII polypeptide that exhibits reduced activity or is devoid of activity, optionally wherein a reduction in activity may be about 5% to about 95%.

11. The peanut plant or part thereof of claim 7 or claim 8, wherein the at least one mutation results in a modified KasII polypeptide, a reduced amount of KasII polypeptide and/or no KasII polypeptide.

12. The peanut plant or part thereof of claim 11, wherein the modified KasII polypeptide comprises a truncated KasII polypeptide, optionally wherein the truncated KasII polypeptide exhibits reduced or no activity or the truncation of the KasII polypeptide results in undetectable levels of the KasII polypeptide.

13. The peanut plant or part thereof of any one of the claims 10-12, wherein the activity that is devoid or reduced is conversion of palmitic acid to stearic acid.

14. The peanut plant or part thereof of any one of the preceding claims, wherein the peanut plant comprising the at least one mutation produces seed (peanuts) (e.g., part thereof) having oil (seed oil) with an increased level of palmitic acid as compared to seed from a plant devoid of the at least one mutation.

15. The peanut plant or part thereof of claim 14, wherein the increase in palmitic acid in the seed oil is an increase from about 30% to about 65% [from a total amount of palmitic acid of about 10% to an amount of about 13% to 16.5%.

16. The peanut plant or part thereof of claim 14, wherein the peanut plant comprising the at least one mutation produces seed (peanuts) (e.g., part thereof) having oil (seed oil) with an increased ratio of palmitic acid to stearic acid, optionally wherein the increase is a doubling of the ratio of palmitic to stearic acid as compared to seed from a plant devoid of the at least one mutation, optionally wherein the ratio of palmitic acid to stearic acid is increased from a ratio of 4:1 to a ratio of 8:1.

17. The peanut plant or part thereof of any one of the preceding claims, wherein the at least one mutation results in a mutated KASII gene comprising a nucleotide sequence having a mutation as described herein.

18. The peanut plant or part thereof of any one of the preceding claims, wherein the part thereof is a peanut cell and/or a peanut seed.

19. A plant regenerated from the plant part of any one of claims 1-18.

20. A nucleic acid encoding a mutated KASII gene from peanut, optionally wherein the mutation is a base substitution, a base deletion, and/or a base insertion in an peanut KASII gene.

21. The nucleic acid of claim 20, wherein the peanut KASII gene:
(a) comprises a nucleotide sequence having at least 80% sequence identity to SEQ ID NO:1, SEQ ID NO:2, SEQ ID NO:116, SEQ ID NO:117, SEQ ID NO:118, and/or SEQ ID NO:119,
(b) comprises a region of consecutive nucleotides having at least 80% sequence identity to any one of the nucleotide sequences of SEQ ID NOs:5-31, 32-51, 52-69, 70-83 and/or 275-298, and/or SEQ ID NOs:122-148, 149-168, 169-188, 189-208, 209-212 and/or 299-326; and/or
(c) encodes an amino acid sequence having at least 80% sequence identity to SEQ ID NO:4 and/or SEQ ID NO:121.

22. The nucleic acid of claim 20 or claim 21, wherein the nucleic acid encodes an amino acid sequence as modified herein.

23. A peanut plant or part thereof comprising the nucleic acid of any one of claims 20-22.

24. A peanut seed oil produced from the seed of the peanut plant of any one of claim 1-18 or 22, the peanut seed oil comprising at least about 13% to about 16.5% total palmitic acid and/or a ratio of palmitic acid to stearic acid of about 4:1 to about 6:1.

25. A guide nucleic acid that binds within a target site in a KASII gene, the target site having at least 80% sequence identity to any one of SEQ ID NOs:5-31, 32-51, 52-69, 70-83 and/or 275-298, and/or SEQ ID NOs:122-148, 149-168, 169-188, 189-208, 209-212, and/or 299-326, or a portion of consecutive nucleotides thereof.

26. The guide nucleic acid of claim 25, wherein the guide nucleic acid comprises a spacer comprising the nucleotide sequence of SEQ ID NOs:84-95 and/or 241-246 and/or SEQ ID NOs:213-224 and/or 257-262 (e.g., SEQ ID NOs:84-86; 87-89; 90-91; 92-95; 213, 214 and 85; 215, 216 and 88; 217-219; 220-223; 224 and 91, 241-246 and/or 257-262).

27. An expression cassette comprising (a) a polynucleotide encoding CRISPR-Cas effector protein comprising a cleavage domain and (b) a guide nucleic acid that binds to a target site in a KASII gene, wherein the guide nucleic acid comprises a spacer sequence that is complementary to and binds to:
(i) a portion of consecutive nucleotides from a nucleic acid having at least 80% sequence identity to any one of SEQ ID NOs:1, 2, and/or 116-119;
(ii) a portion of consecutive nucleotides from a nucleic acid sequence having at least 80% sequence identity to any one of SEQ ID NOs:5-83 or 275-298 or SEQ ID NOs:122-212 or 299-326; and/or
(iii) a portion of a consecutive nucleotides from a nucleic acid encoding an amino acid sequence having at least 80% sequence identity to any one of SEQ ID NO:4 or SEQ ID NO:121.

28. A method of producing a peanut plant having seed oil with increased levels of palmitic acid, the method comprising:
(a) contacting a peanut plant cell comprising a KASII gene with a nuclease targeting the KASII gene, wherein the nuclease is linked to a nucleic acid binding domain (e.g., editing system) that binds to a target site in the KASII gene, wherein the KASII gene:
(i) comprises a nucleotide sequence having at least 80% sequence identity to any one of SEQ ID NO:1, SEQ ID NO:2, SEQ ID NO:116, SEQ ID NO:117, SEQ ID NO:118, and/or SEQ ID NO:119,
(ii) comprises a region of consecutive nucleotides having at least 80% sequence identity to any one of the nucleotide sequences of SEQ ID NOs:5-31, 32-51, 52-69, 70-83 and/or 275-298, and/or SEQ ID NOs:122-148, 149-168, 169-188, 189-208, 209-212, and/or 299-326, and/or
(iii) encodes an amino acid sequence having at least 80% sequence identity to SEQ ID NO:4 and/or SEQ ID NO:121; and
(b) growing the peanut plant cell into a peanut plant, wherein the peanut plant comprises a mutation in the KASII gene, thereby producing a peanut plant having seed oil with an increased level of palmitic acid.

29. A method of increasing the level of palmitic acid in the seed oil of a peanut plant, the method comprising:
(a) contacting a peanut plant cell comprising a KASII gene with a nuclease targeting the KASII gene, wherein the nuclease is linked to a nucleic acid binding domain (e.g., editing system) that binds to a target site in the KASII gene, wherein the KASII gene:
(i) comprises a nucleotide sequence having at least 80% sequence identity to SEQ ID NO:1, SEQ ID NO:2, SEQ ID NO:116, SEQ ID NO:117, SEQ ID NO:118, and/or SEQ ID NO:119,
(ii) comprises a region of consecutive nucleotides having at least 80% sequence identity to any one of the nucleotide sequences of SEQ ID NOs:5-31, 32-51, 52-69, 70-83 and/or 275-298, and/or SEQ ID NOs:122-148, 149-168, 169-188, 189-208, 209-212, and/or 299-326, and/or
(iii) encodes an amino acid sequence having at least 80% sequence identity to SEQ ID NO:4 and/or SEQ ID NO:121; and
(b) growing the peanut plant cell into a peanut plant comprising the mutation in the KASII gene, thereby producing a peanut plant have a mutated KASII gene and comprising seed oil with an increased level of palmitic acid.

30. A method of reducing oil separation in peanut butter, the method comprising preparing peanut butter from the seed of the plant of any one of claim 1-19 or 23.

31. A method of reducing oil separation in peanut butter, the method comprising
(a) contacting a peanut plant comprising an KASII gene with a nuclease targeting the KASII gene, wherein the nuclease is linked to a nucleic acid binding domain (e.g., editing system) that binds to a target site in the KASII gene, wherein the KASII gene:
(i) comprises a nucleotide sequence having at least 80% sequence identity to SEQ ID NO:1, SEQ ID NO:2, SEQ ID NO:116, SEQ ID NO:117, SEQ ID NO:118, and/or SEQ ID NO:119,
(ii) comprises a region of consecutive nucleotides having at least 80% sequence identity to any one of the nucleotide sequences of SEQ ID NOs:5-31, 32-51, 52-69, 70-83 and/or 275-298, and/or SEQ ID NOs:122-148, 149-168, 169-188, 189-208, 209-212, and/or 299-326, and/or
(iii) encodes an amino acid sequence having at least 80% sequence identity to SEQ ID NO:4 and/or SEQ ID NO:121; and
(b) growing the peanut plant cell into a peanut plant comprising the mutation in the KASII gene, thereby producing a plant have a mutated KASII gene and exhibiting an increase level of palmitic acid in the seed oil of the peanut plant; and
preparing peanut butter from the seed, wherein the peanut butter has reduced oil or no oil separation.

32. A method of providing peanut oil with increased levels of palmitic acid, the method comprising preparing peanut oil from the seed of the plant of any one of claim 1-19 or 23.

33. A method of providing peanut oil with increased levels of palmitic acid, the method comprising
(a) contacting a peanut plant comprising an KASII gene with a nuclease targeting the KASII gene, wherein the nuclease is linked to a nucleic acid binding domain (e.g., editing system) that binds to a target site in the KASII gene, wherein the KASII gene:
(i) comprises a nucleotide sequence having at least 80% sequence identity to SEQ ID NO:1, SEQ ID NO:2, SEQ ID NO:116, SEQ ID NO:117, SEQ ID NO:118, and/or SEQ ID NO:119,
(ii) comprises a region of consecutive nucleotides having at least 80% sequence identity to any one of the nucleotide sequences of SEQ ID NOs:5-31, 32-51, 52-69, 70-83 and/or 275-298, and/or SEQ ID NOs:122-148, 149-168, 169-188, 189-208, 209-212, and/or 299-326, and/or
(iii) encodes an amino acid sequence having at least 80% sequence identity to SEQ ID NO:4 and/or SEQ ID NO:121, and
(b) growing the peanut plant cell into a peanut plant comprising the mutation in the KASII gene, thereby producing a plant have a mutated KASII gene and exhibiting an increase level of palmitic acid in the seed oil of the peanut plant.

34. The method of any one of claims 28-33, wherein palmitic acid in the peanut seed oil is increased to about 13% to about 16.5% (as compared to a peanut plant devoid of the mutation).


SEQUENCE LISTING (364 sequences)

SEQ ID NO: 1 - DNA, 7502 bp, synthetic construct
  source 1..7502: mol_type = other DNA
ttatagaagctataaatcagaggcacagatttgttttatttttttccaaacaatcacaaatcgaacggttcaatctgtgatttcgaaaataaaaaaattttaatgttaaaatcgaactgtctgatttttattaaaaaataaaaaaataaaaaatataaaacagaccatgcgatttgtagtttttttaaacaaattggactctctgatttgtagttcattttttcttcaaacaaatcggaccgtccgatttaaataaaacaccataaaaaaaatacctaatcttccaataacaatatattacacacatatttaaccaatataaaaaaaattagccttccattctcttaccaaaaaaaagtaatattaattttcaatttgcatctctgttacattatttcaggtcacgtagccagcaaaaggagacactggtatgcaccagcatttttcttttccttttcttttgtggtctttattttttagaatatatatgataatatatacggattaatttcaatttcttttgtatcatattcaactactagtttacatatatgggactttttaataatcatcaagagataaaatttaaatatttttatttatcatataattagagataagataaattttgattcatgaatttgatctgataattaatataaattttaaacatacttaatttgataatgagttttataactataattcaatcaagtcattttgtcacccattctattactgtattacatggatacttatgtgtgcagataactattaagtattaacagctcatcagtggcttgcagatagtaatgtctctcaacttgtttacatgacatttaactaaatgagcatctcataagtattttaagcattttattaattagattgattattaaaaaaattaatttgatttacggttaaaaatagcttttgtttaaaatttaaaagcccaactaattattagatgaaaatttatgagcaaattaggttttagtgttgatttggattagttttttcgagtgaaaaatatttttttttaaaaaaaaaattaaaatattttttattcaattctaaatttatttagatatattttttaatttattaaaaaattaaacaatttacttttttaaaagttaacaatattttacttttaaaaaaatagaaacaaaaaacgtttttaatatttaaattttttaaaaatcttatttaaaaaaataaaaactaaatatttttttaaaaatcaatttaaactgatctttatctcttaattattaactaattttggtatttactttcaaattttcgtaaaaaaaagtcttactgagaataaagtattattttgactaaagaaaatctgaaagcaagaataaaataaatgctatgtcacgttttacactaatgatgaaaaaaaatatacaatataagtagaaaatattgcataaataatgatgaatagcgaagaaaaagaagagaggttagtagaaaattaaataaaataaaattaatagggaaagcaaatgaaatgagaagcgaaggagagagtgagagtcagattgcagaaaaagcagagactttgagactctgagacttcacaacgcctcccttttccttcttctctcagatcgattctctttctcactctctcaactctcaaccttcttcaaaacccccacaactcactttctttttcttcttcttcttctttgtatatacatacatacatacatacatgtacatgtatcttcactgaaacgtcctcgattctcttgcgagcttctgctttagacaacatgatacattccgctgcttcatccatggcctcacccctctgcacgtggctggtggcagcttgcatgtcagtcacgtgccacgctgaccgcgccgggactccaaaggcagcgcttcgctcatccaagaggtcaaggaggacgaaggcgctgccaccaaacacctcccaattgaacatgagattgatttcttcgctctacggatccagcattcagggactcataagctccttcgagccctgcgatgactactgcaacactcacaatgccttctcttctctctttcgatccacaactcccaatcgcagacacacccgtctcaataaacttgctcactctggtaaactttttttttttttttaattttccccacttaaatcacctcaagttaagatatttctggtgctttttgcaattttaatctgtaccccgtactgggtatttcaatttgattcatttatttaattttcttatattaatataataataataatagtttttttattatggttatgaaatctgggttctgattttctggtcttctgaaggatgtgtgtgtgtatgtgtggcttttttccctttttttctgaattacttaaaatgcacaagtgttagcttggatctttgtcttttggttggtcatcctaataaatggggttgcttggtggtttagtatggagggttgttctacctgctctgtttaactgttttgagggtacggtttataatattaggagtatgggggtagttgttgttctttctttgctgaggttgatggtgcacctgtgtcacttaattaggacatgttgctacaacactatgccattgcccatgctttttgctttcatttcccattcctacttcatttccttgtatttatcgtacactcaggtatctacataaagttcccattaaatattggttaatgttcgcatcatcactttgaatttcttgcaatctcgttgaggtagttcagcatattcacagaatcaactcaaacaaaaataaatttcttttgacttttatttttattttttttttgcttttcttcatgaaaataataatcacactgaagtgttctgaagttaactactatgtaaatatcgcccccacgtgggattacctctctctccctctctctctcgttttttgtttctctatgtaaaataatggaggtaagttatgcctttgtcataagcacacataagcaagtcagaggctccttttttttttttccctcaattttttttttaaattatcatgtaaaatagatgattctggcacaaactatccacaagttatttttgctcctagttctttagtatgtttttaacacactggacaagtctactactgctgcatgcatgtccctgcctgatgggttctagtattcagtggtcttgagtgttttgatttaaagcaagggatataagttgggtctgatgaatttttcgaggtttcatagcatttttgttaccatagtcttagaattactaccttggataaacctaagcatatttttgttaatggctggggttgcatttcctgctcaacattaatgtgcaccaagggtatgttctgttaggggttaaaaatgggttgggaatttacaatggatgtggcccatgtgtaatttcactcatagaacttattgtaaaaatttcaccccatttctacttcccaaaagaacatatccttatcatgatcattggaaactacacatgcatcatgttgaactataagctgtgggccgctctttctttgtatgcttctgatacgaatttcttgaatgataggcagttgcatgcaatttttattattgctgacttattcctctgaattgcataactataaggtaaaacaatggctgtagctgtggaacctgcacatgaagtcacaacaacgaaaaaacctcctacaaaacaaaggcgagtagttgtgaccgggttgggcgttgttacacctcttggtcacgaaccagatgtttactacaacaatttacttgatggtgttagcggcattagtgagatcgagacatttgattgtgctgaatatccaacggcaagttatatataattgaaattgtattttttgttgtctgttgtagcttttggtctgaatattctatgtttccagaggattgctggtgagatcaagtcgttctccaccgatggctgggttgcaccaaaactttcgaagagaatggataaatttatgctctatttgctaacagccggtaagaaggccttagttgatggtggaattactgaagatgtaatggatgagttgaataaggaaaaatgtggggttttgattggctcagcaatgggtggcatgaaggtaaccttgtcttactttcatgttgagatacaaatcttgtaaaaccagctggtgttgtggactgaatttaatgccactgcaatccaaatggagcagaagagtgtgctatattttcaaccttgtgcccttgttttattgactatgatttttggtgctttttctattgagcaggtttttaatgatgccattgaagctttaagagtatcatataagaagatgaatcctttttgtgtaccttttgccacaacaaatatgggatctgccatacttgcgatggatctggtctgttgttgcatggacaccataaattggtgttttgtttcgtaccaagcttttattttggctatgttgtcttcagagtctaacaaacattgttatcttttatggattacagggatggatgggccctaattattcaatctctacagcttgtgctacaagtaacttttgtatattgaatgcagcaaaccacatcataagaggtgaagctgtaagtattcatgctttgatattactctcatgatggtgaatttttcgtatccaatattataccttctatttatttatttatttaacttttttttcctgagacagtttggtgtaatgatatggtcattgcatcaaaatgtattgataaatttaatatgaaaatattcatgcaggatgtgatgctttgtggtggctcagatgctgctattatacccattggtatgatctcaccattgattctttttcacctgcatacggttatcgtgccataaatctagctgatatgaatccactaatcattggtacaggtttgggaggttttgtggcatgcagagccctatcacagaggaatacagaccctaccaaagcttcacgcccttgggacattgtaatacaacacctgctcattctttctgctactcaccatgttttgtattgctgaggctttcatcattttttatttttgaaataatacgactataattataatgaaatttgatgaccaagtcttgtatcctttcttgtaatctgaacagcatagtttattaaaaactaacgttacaatggtcacagatacaacttttccttttgatcacaaaaatgctgtcttctatggatatgagctatggcaaattcagtttttctaattaggctgtttatgatattccagaaccgagatggatttgtcatgggggaaggagctggagttttacttttggaggacttggagcatgcaaaggtacatgaaatctgatctaataattttagaacagaacatcccttatgaaatgacagtgcatcttttgatattattactgcagactcgtgcatgtaatttatttcgtttagtgcatccttcagttccttcaaacagtattaaaggacttcatacatctgttctctcttgaattaggattgattttcaggctgctgcttgtcataaaaattcacctgttacttctcacatcaaatctgcgcttgacatttgtcttgcaactgaatcaaatgcatctcttttacattgcagaaaagaggtgcaaccatatatgctgaattccttggtggaagcttcacctgtgatgcatatcatgtgacagagccacgtcccgatggtaaaatatcaaatcctttactagcaaaatttaaagcatttatatgcatgctatgattaccatgcttaaaatccgaagagaacaaggcaaaataatgtgacaagggtattcttatatatttagaattatgtgtgcaatttgctgatggttagggttaagtatttagaaatatactatatgttgcatttttcttgtttggcactggaatttcttcttcagagatcgttatggaatttgaaactgcatgttgcgatcatgacttgtctcgacaatatggacctgggatttaataatatgcatatattagtggacctagtaatcctagtcataattagtatctgcttgtactttaccacatttcctttatgattccattcggactcttctttgagtattctatgacttgacaaggaatgatttgtatctccctgcggaacttgcattcatgtttctttactccatcttaaataggagctggcgttatactttgcattgaaaaggcattagctcagtctggagtatcaaaagaggatgtgaactacataaatgcacatgccacatccacgcctgctggtgatcttaaggagttccaggctctgcttcattgttttggtcaaaatcctgaggtacatctatctttttaagttacaccactattaacattgttatcttggtgatcaattctatggcaaacatgatctcttgttcttctggagttcatgttgagtaaaacactggtacttctgatatggtcatccatactattgtctcttgttttttcagctaagagtgaattctacaaaatctatgattggtcatctactaggagcagctggtggcgtggaagctgtagcaacagtccaggtaaaacccaatggatcacttagttcggtaccccgcgttttgtgactaaaatggtcctgaggtttgctctctctttatacaggcaattaggacagggtgggtgcatcccaatatcaacctggaaaacccagatcaaggagtggtaatgtttcttgttgcatcattccatccttcatttcaaaattttaattttaatcttttaatctaacatgtccaaattgatgagaaaaattcaggatgccaaagtgcttgtgggatcaaagaaagagagactagatatcaaggctgccctatccaattcatttggttttgggggccacaattcttcaatcatatttgctccgtataagtgaatgaatacatgtttcagaataataccttccctaccattatacagaaggtatgtctctaaatatatcgtcctagtacgagaaacatttaatttggatgactgtggatgttccttgaacatgtattaaattgtgtgttgtttttatgttaagttactaaggtagccaggcaattgttacacgcatgatcaaagtcatgaattggatgaaaatatatgtggaaatggttggagcagaaactcttgaggacattgttaagagatggtttcgtggtttagattgtctggccgagatacatggtggagccggttctatccaccgatgaagaacatgaatggctgctattgcctttatagagtaggacttgacccactttgatgtgactcttaaacaaatccacttggcagtatatataagctcttctgttttatttattccctatgtacccaaatagtttgtgcaataacaaattgcaatttattttttcgctgccttgttctattgcccttaaaccagaccatttagggaaaaggggatgattttaatctctcaggaaa

SEQ ID NO: 2 - DNA, 7687 bp, synthetic construct
  source 1..7687: mol_type = other DNA
ttatagaagctataaatcagaggcacagatttgttttatttttttccaaacaatcacaaatcgaacggttcaatctgtgatttcgaaaataaaaaaattttaatgttaaaatcgaactgtctgatttttattaaaaaataaaaaaataaaaaatataaaacagaccatgcgatttgtagtttttttaaacaaattggactctctgatttgtagttcattttttcttcaaacaaatcggaccgtccgatttaaataaaacaccataaaaaaaatacctaatcttccaataacaatatattacacacatatttaaccaatataaaaaaaattagccttccattctcttaccaaaaaaaagtaatattaattttcaatttgcatctctgttacattatttcaggtcacgtagccagcaaaaggagacactggtatgcaccagcatttttcttttccttttcttttgtggtctttattttttagaatatatatgataatatatacggattaatttcaatttcttttgtatcatattcaactactagtttacatatatgggactttttaataatcatcaagagataaaatttaaatatttttatttatcatataattagagataagataaattttgattcatgaatttgatctgataattaatataaattttaaacatacttaatttgataatgagttttataactataattcaatcaagtcattttgtcacccattctattactgtattacatggatacttatgtgtgcagataactattaagtattaacagctcatcagtggcttgcagatagtaatgtctctcaacttgtttacatgacatttaactaaatgagcatctcataagtattttaagcattttattaattagattgattattaaaaaaattaatttgatttacggttaaaaatagcttttgtttaaaatttaaaagcccaactaattattagatgaaaatttatgagcaaattaggttttagtgttgatttggattagttttttcgagtgaaaaatatttttttttaaaaaaaaaattaaaatattttttattcaattctaaatttatttagatatattttttaatttattaaaaaattaaacaatttacttttttaaaagttaacaatattttacttttaaaaaaatagaaacaaaaaacgtttttaatatttaaattttttaaaaatcttatttaaaaaaataaaaactaaatatttttttaaaaatcaatttaaactgatctttatctcttaattattaactaattttggtatttactttcaaattttcgtaaaaaaaagtcttactgagaataaagtattattttgactaaagaaaatctgaaagcaagaataaaataaatgctatgtcacgttttacactaatgatgaaaaaaaatatacaatataagtagaaaatattgcataaataatgatgaatagcgaagaaaaagaagagaggttagtagaaaattaaataaaataaaattaatagggaaagcaaatgaaatgagaagcgaaggagagagtgagagtcagattgcagaaaaagcagagactttgagactctgagacttcacaacgcctcccttttccttcttctctcagatcgattctctttctcactctctcaactctcaaccttcttcaaaacccccacaactcactttctttttcttcttcttcttctttgtatatacatacatacatacatacatgtacatgtatcttcactgaaacgtcctcgattctcttgcgagcttctgctttagacaacatgatacattccgctgcttcatccatggcctcacccctctgcacgtggctggtggcagcttgcatgtcagtcacgtgccacgctgaccgcgccgggactccaaaggcagcgcttcgctcatccaagaggtcaaggaggacgaaggcgctgccaccaaacacctcccaattgaacatgagattgatttcttcgctctacggatccagcattcagggactcataagctccttcgagccctgcgatgactactgcaacactcacaatgccttctcttctctctttcgatccacaactcccaatcgcagacacacccgtctcaataaacttgctcactctggtaaactttttttttttttttaattttccccacttaaatcacctcaagttaagatatttctggtgctttttgcaattttaatctgtaccccgtactgggtatttcaatttgattcatttatttaattttcttatattaatataataataataatagtttttttattatggttatgaaatctgggttctgattttctggtcttctgaaggatgtgtgtgtgtatgtgtggcttttttccctttttttctgaattacttaaaatgcacaagtgttagcttggatctttgtcttttggttggtcatcctaataaatggggttgcttggtggtttagtatggagggttgttctacctgctctgtttaactgttttgagggtacggtttataatattaggagtatgggggtagttgttgttctttctttgctgaggttgatggtgcacctgtgtcacttaattaggacatgttgctacaacactatgccattgcccatgctttttgctttcatttcccattcctacttcatttccttgtatttatcgtacactcaggtatctacataaagttcccattaaatattggttaatgttcgcatcatcactttgaatttcttgcaatctcgttgaggtagttcagcatattcacagaatcaactcaaacaaaaataaatttcttttgacttttattttttttttttttttgcttttcttcatgaaaataataatcacactgaagtgttctgaagttaactactatgtaaatatcgcccccacgtgggattacctctctctccctctctctctcgttttttgtttctctatgtaaaataatggaggtaagttatgcctttgtcataagcacacataagcaagtcagaggctccttttttttttttccctcaattttttttttaaattatcatgtaaaatagatgattctggcacaaactatccacaagttatttttgctcctagttctttagtatgtttttaacacactggacaagtctactactgctgcatgcatgtccctgcctgatgggttctagtattcagtggtcttgagtgttttgatttaaagcaagggatataagttgggtctgatgaatttttcgaggtttcatagcatttttgttaccatagtcttagaattactaccttggataaacctaagcatatttttgttaatggctggggttgcatttcctgctcaacattaatgtgcaccaagggtatgttctgttaggggttaaaaatgggttgggaatttacaatggatgtggcccatgtgtaatttcactcatagaacttattgtaaaaatttcaccccatttctacttcccaaaagaacatatccttatcatgatcattggaaactacacatgcatcatgttgaactataagctgtgggccgctctttctttgtatgcttctgatacgaatttcttgaatgataggcagttgcatgcaatttttattattgctgacttattcctctgaattgcataactataaggtaaaacaatggctgtagctgtggaacctgcacatgaagtcacaacaacgaaaaaacctcctacaaaacaaaggcgagtagttgtgaccgggttgggcgttgttacacctcttggtcacgaaccagatgtttactacaacaatttacttgatggtgttagcggcattagtgagatcgagacatttgattgtgctgaatatccaacggcaagttatatataattgaaattgtattttttgttgtctgttgtagcttttggtctgaatattctatgtttccagaggattgctggtgagatcaagtcgttctccaccgatggctgggttgcaccaaaactttcgaagagaatggataaatttatgctctatttgctaacagccggtaagaaggccttagttgatggtggaattactgaagatgtaatggatgagttgaataaggaaaaatgtggggttttgattggctcagcaatgggtggcatgaaggtaaccttgtcttactttcatgttgagatacaaatcttgtaaaaccagctggtgttgtggactgaatttaatgccactgcaatccaaatggagcagaagagtgtgctatattttcaaccttgtgcccttgttttattgactatgatttttggtgctttttctattgagcaggtttttaatgatgccattgaagctttaagagtatcatataagaagatgaatcctttttgtgtaccttttgccacaacaaatatgggatctgccatacttgcgatggatctggtctgttgttgcatggacaccataaattggtgttttgtttcgtaccaagcttttattttggctatgttgtcttcagagtctaacaaacattgttatcttttatggattacagggatggatgggccctaattattcaatctctacagcttgtgctacaagtaacttttgtatattgaatgcagcaaaccacatcataagaggtgaagctgtaagtattcatgctttgatattactctcatgatggtgaatttttcgtatccaatattataccttctatttatttatttatttaacttttttttcctgagacagtttggtgtaatgatatggtcattgcatcaaaatgtattgataaatttaatatgaaaatattcatgcaggatgtgatgctttgtggtggctcagatgctgctattatacccattggtatgatctcaccattgattctttttcacctgcatacggttatcgtgccataaatctagctgatatgaatccactaatcattggtacaggtttgggaggttttgtggcatgcagagccctatcacagaggaatacagaccctaccaaagcttcacgcccttgggacattgtaatacaacacctgctcattctttctgctactcaccatgttttgtattgctgaggctttcatcattttttatttttgaaataatacgactataattataatgaaatttgatgaccaagtcttgtatcctttcttgtaatctgaacagcatagtttattaaaaactaacgttacaatggtcacagatacaacttttccttttgatcacaaaaatgctgtcttctatggatatgagctatggcaaattcagtttttctaattaggctgtttatgatattccagaaccgagatggatttgtcatgggggaaggagctggagttttacttttggaggacttggagcatgcaaaggtacatgaaatctgatctaataattttagaacagaacatcccttatgaaatgacagtgcatcttttgatattattactgcagactcgtgcatgtaatttatttcgtttagtgcatccttcagttccttcaaacagtattaaaggacttcatacatctgttctctcttgaattaggattgattttcaggctgctgcttgtcataaaaattcacctgttacttctcacatcaaatctgcgcttgacatttgtcttgcaactgaatcaaatgcatctcttttacattgcagaaaagaggtgcaaccatatatgctgaattccttggtggaagcttcacctgtgatgcatatcatgtgacagagccacgtcccgatggtaaaatatcaaatcctttactagcaaaatttaaagcatttatatgcatgctatgattaccatgcttaaaatccgaagagaacaaggcaaaataatgtgacaagggtattcttatatatttagaattatgtgtgcaatttgctgatggttagggttaagtatttagaaatatactatatgttgcatttttcttgtttggcactggaatttcttcttcagagatcgttatggaatttgaaactgcatgttgcgatcatgacttgtctcgacaatatggacctgggatttaataatatgcatatattagtggacctagtaatcctagtcataattagtatctgcttgtactttaccacatttcctttatgattccattcggactcttctttgagtattctatgacttgacaaggaatgatttgtatctccctgcggaacttgcattcatgtttctttactccatcttaaataggagctggcgttatactttgcattgaaaaggcattagctcagtctggagtatcaaaagaggatgtgaactacataaatgcacatgccacatccacgcctgctggtgatcttaaggagttccaggctctgcttcattgttttggtcaaaatcctgaggtacatctatctttttaagttacaccactattaacattgttatcttggtgatcaattctatggcaaacatgatctcttgttcttctggagttcatgttgagtaaaacactggtacttctgatatggtcatccatactattgtctcttgttttttcagctaagagtgaattctacaaaatctatgattggtcatctactaggagcagctggtggcgtggaagctgtagcaacagtccaggtaaaacccaatggatcacttagttcggtaccccgcgttttgtgactaaaatggtcctgaggtttgctctctctttatacaggcaattaggacagggtgggtgcatcccaatatcaacctggaaaacccagatcaaggagtggtaatgtttcttgttgcatcattccatccttcatttcaaaattttaattttaatcttttaatctaacatgtccaaattgatgagaaaaattcaggatgccaaagtgcttgtgggatcaaagaaagagagactagatatcaaggctgccctatccaattcatttggttttgggggccacaattcttcaatcatatttgctccgtataagtgaatgaatacatgtttcagaataataccttccctaccattatacagaaggtatgtctctaaatatatcgtcctagtacgagaaacatttaatttggatgactgtggatgttccttgaacatgtattaaattgtgtgttgtttttatgttaagttactaaggtagccaggcaattgttacacgcatgatcaaagtcatgaattggatgaaaatatatgtggaaatggttggagcagaaactcttgaggacattgttaagagatggtttcgtggtttagattgtctggccgagatacatggtggagccggttctatccaccgatgaagaacatgaatggctgctattgcctttatagagtaggacttgacccactttgatgtgactcttaaacaaatccacttggcagtatatataagctcttctgttttatttattccctatgtacccaaatagtttgtgcaataacaaattgcaatttattttttcgctgccttgttctattgcccttaaaccagaccattttatcaaagacaaataacgttttctttaaaatagatctcaatttatattccaattgatgttagatattatcttccatcatacaatatctttggtttttccttaacagcatcaaattgagatttatttgccgcctttctattaaaggtcagtttcaaaacttgacaattgctcatcagaatcagaaaaaccttgagatattgctcatgtttaatgcgagaa

SEQ ID NO: 3 - DNA, 1647 bp, synthetic construct
  source 1..1647: mol_type = other DNA
atgatacattccgctgcttcatccatggcctcacccctctgcacgtggctggtggcagcttgcatgtcagtcacgtgccacgctgaccgcgccgggactccaaaggcagcgcttcgctcatccaagaggtcaaggaggacgaaggcgctgccaccaaacacctcccaattgaacatgagattgatttcttcgctctacggatccagcattcagggactcataagctccttcgagccctgcgatgactactgcaacactcacaatgccttctcttctctctttcgatccacaactcccaatcgcagacacacccgtctcaataaacttgctcactctggtaaaacaatggctgtagctgtggaacctgcacatgaagtcacaacaacgaaaaaacctcctacaaaacaaaggcgagtagttgtgaccgggttgggcgttgttacacctcttggtcacgaaccagatgtttactacaacaatttacttgatggtgttagcggcattagtgagatcgagacatttgattgtgctgaatatccaacgaggattgctggtgagatcaagtcgttctccaccgatggctgggttgcaccaaaactttcgaagagaatggataaatttatgctctatttgctaacagccggtaagaaggccttagttgatggtggaattactgaagatgtaatggatgagttgaataaggaaaaatgtggggttttgattggctcagcaatgggtggcatgaaggtttttaatgatgccattgaagctttaagagtatcatataagaagatgaatcctttttgtgtaccttttgccacaacaaatatgggatctgccatacttgcgatggatctgggatggatgggccctaattattcaatctctacagcttgtgctacaagtaacttttgtatattgaatgcagcaaaccacatcataagaggtgaagctgatgtgatgctttgtggtggctcagatgctgctattatacccattggtttgggaggttttgtggcatgcagagccctatcacagaggaatacagaccctaccaaagcttcacgcccttgggacattaaccgagatggatttgtcatgggggaaggagctggagttttacttttggaggacttggagcatgcaaagaaaagaggtgcaaccatatatgctgaattccttggtggaagcttcacctgtgatgcatatcatgtgacagagccacgtcccgatggagctggcgttatactttgcattgaaaaggcattagctcagtctggagtatcaaaagaggatgtgaactacataaatgcacatgccacatccacgcctgctggtgatcttaaggagttccaggctctgcttcattgttttggtcaaaatcctgagctaagagtgaattctacaaaatctatgattggtcatctactaggagcagctggtggcgtggaagctgtagcaacagtccaggcaattaggacagggtgggtgcatcccaatatcaacctggaaaacccagatcaaggagtggatgccaaagtgcttgtgggatcaaagaaagagagactagatatcaaggctgccctatccaattcatttggttttgggggccacaattcttcaatcatatttgctccgtataagtga

SEQ ID NO: 4 - protein, 548 aa, synthetic construct
MIHSAASSMASPLCTWLVAACMSVTCHADRAGTPKAALRSSKRSRRTKALPPNTSQLNMRLISSLYGSSIQGLISSFEPCDDYCNTHNAFSSLFRSTTPNRRHTRLNKLAHSGKTMAVAVEPAHEVTTTKKPPTKQRRVVVTGLGVVTPLGHEPDVYYNNLLDGVSGISEIETFDCAEYPTRIAGEIKSFSTDGWVAPKLSKRMDKFMLYLLTAGKKALVDGGITEDVMDELNKEKCGVLIGSAMGGMKVFNDAIEALRVSYKKMNPFCVPFATTNMGSAILAMDLGWMGPNYSISTACATSNFCILNAANHIIRGEADVMLCGGSDAAIIPIGLGGFVACRALSQRNTDPTKASRPWDINRDGFVMGEGAGVLLLEDLEHAKKRGATIYAEFLGGSFTCDAYHVTEPRPDGAGVILCIEKALAQSGVSKEDVNYINAHATSTPAGDLKEFQALLHCFGQNPELRVNSTKSMIGHLLGAAGGVEAVATVQAIRTGWVHPNINLENPDQGVDAKVLVGSKKERLDIKAALSNSFGFGGHNSSIIFAPYK

SEQ ID NO: 5 - DNA, 693 bp, synthetic construct
  source 1..693: mol_type = other DNA
aacaccataaaaaaaatacctaatcttccaataacaatatattacacacatatttaaccaatataaaaaaaattagccttccattctcttaccaaaaaaaagtaatattaattttcaatttgcatctctgttacattatttcaggtcacgtagccagcaaaaggagacactggtatgcaccagcatttttcttttccttttcttttgtggtctttattttttagaatatatatgataatatatacggattaatttcaatttcttttgtatcatattcaactactagtttacatatatgggactttttaataatcatcaagagataaaatttaaatatttttatttatcatataattagagataagataaattttgattcatgaatttgatctgataattaatataaattttaaacatacttaatttgataatgagttttataactataattcaatcaagtcattttgtcacccattctattactgtattacatggatacttatgtgtgcagataactattaagtattaacagctcatcagtggcttgcagatagtaatgtctctcaacttgtttacatgacatttaactaaatgagcatctcataagtattttaagcattttattaattagattgattattaaaaaaattaatttgatttacggttaaaaatagcttttgtttaaaatttaaa

SEQ ID NO: 6 - DNA, 593 bp, synthetic construct
  source 1..593: mol_type = other DNA
tatttaaccaatataaaaaaaattagccttccattctcttaccaaaaaaaagtaatattaattttcaatttgcatctctgttacattatttcaggtcacgtagccagcaaaaggagacactggtatgcaccagcatttttcttttccttttcttttgtggtctttattttttagaatatatatgataatatatacggattaatttcaatttcttttgtatcatattcaactactagtttacatatatgggactttttaataatcatcaagagataaaatttaaatatttttatttatcatataattagagataagataaattttgattcatgaatttgatctgataattaatataaattttaaacatacttaatttgataatgagttttataactataattcaatcaagtcattttgtcacccattctattactgtattacatggatacttatgtgtgcagataactattaagtattaacagctcatcagtggcttgcagatagtaatgtctctcaacttgtttacatgacatttaactaaatgagcatctcataagtattttaagcattttattaattagattgattattaa

SEQ ID NO: 7 - DNA, 514 bp, synthetic construct
  source 1..514: mol_type = other DNA
accaaaaaaaagtaatattaattttcaatttgcatctctgttacattatttcaggtcacgtagccagcaaaaggagacactggtatgcaccagcatttttcttttccttttcttttgtggtctttattttttagaatatatatgataatatatacggattaatttcaatttcttttgtatcatattcaactactagtttacatatatgggactttttaataatcatcaagagataaaatttaaatatttttatttatcatataattagagataagataaattttgattcatgaatttgatctgataattaatataaattttaaacatacttaatttgataatgagttttataactataattcaatcaagtcattttgtcacccattctattactgtattacatggatacttatgtgtgcagataactattaagtattaacagctcatcagtggcttgcagatagtaatgtctctcaacttgtttacatgacatttaactaaatgagcatctcat

SEQ ID NO: 8 - DNA, 473 bp, synthetic construct
  source 1..473: mol_type = other DNA
attttcaatttgcatctctgttacattatttcaggtcacgtagccagcaaaaggagacactggtatgcaccagcatttttcttttccttttcttttgtggtctttattttttagaatatatatgataatatatacggattaatttcaatttcttttgtatcatattcaactactagtttacatatatgggactttttaataatcatcaagagataaaatttaaatatttttatttatcatataattagagataagataaattttgattcatgaatttgatctgataattaatataaattttaaacatacttaatttgataatgagttttataactataattcaatcaagtcattttgtcacccattctattactgtattacatggatacttatgtgtgcagataactattaagtattaacagctcatcagtggcttgcagatagtaatgtctctcaacttgtttacatgacat

SEQ ID NO: 9 - DNA, 433 bp, synthetic construct
  source 1..433: mol_type = other DNA
ttacattatttcaggtcacgtagccagcaaaaggagacactggtatgcaccagcatttttcttttccttttcttttgtggtctttattttttagaatatatatgataatatatacggattaatttcaatttcttttgtatcatattcaactactagtttacatatatgggactttttaataatcatcaagagataaaatttaaatatttttatttatcatataattagagataagataaattttgattcatgaatttgatctgataattaatataaattttaaacatacttaatttgataatgagttttataactataattcaatcaagtcattttgtcacccattctattactgtattacatggatacttatgtgtgcagataactattaagtattaacagctcatcagtggcttgcagatagtaatgtctc

SEQ ID NO: 10 - DNA, 393 bp, synthetic construct
  source 1..393: mol_type = other DNA
tagccagcaaaaggagacactggtatgcaccagcatttttcttttccttttcttttgtggtctttattttttagaatatatatgataatatatacggattaatttcaatttcttttgtatcatattcaactactagtttacatatatgggactttttaataatcatcaagagataaaatttaaatatttttatttatcatataattagagataagataaattttgattcatgaatttgatctgataattaatataaattttaaacatacttaatttgataatgagttttataactataattcaatcaagtcattttgtcacccattctattactgtattacatggatacttatgtgtgcagataactattaagtattaacagctcatcagtgg

SEQ ID NO: 11 - DNA, 349 bp, synthetic construct
  source 1..349: mol_type = other DNA
tatttaaccaatataaaaaaaattagccttccattctcttaccaaaaaaaagtaatattaattttcaatttgcatctctgttacattatttcaggtcacgtagccagcaaaaggagacactggtatgcaccagcatttttcttttccttttcttttgtggtctttattttttagaatatatatgataatatatacggattaatttcaatttcttttgtatcatattcaactactagtttacatatatgggactttttaataatcatcaagagataaaatttaaatatttttatttatcatataattagagataagataaattttgattcatgaatttgatctgataatt

SEQ ID NO: 12 - DNA, 269 bp, synthetic construct
  source 1..269: mol_type = other DNA
accaaaaaaaagtaatattaattttcaatttgcatctctgttacattatttcaggtcacgtagccagcaaaaggagacactggtatgcaccagcatttttcttttccttttcttttgtggtctttattttttagaatatatatgataatatatacggattaatttcaatttcttttgtatcatattcaactactagtttacatatatgggactttttaataatcatcaagagataaaatttaaatatttttatttatcatataattaga

SEQ ID NO: 13 - DNA, 229 bp, synthetic construct
  source 1..229: mol_type = other DNA
attttcaatttgcatctctgttacattatttcaggtcacgtagccagcaaaaggagacactggtatgcaccagcatttttcttttccttttcttttgtggtctttattttttagaatatatatgataatatatacggattaatttcaatttcttttgtatcatattcaactactagtttacatatatgggactttttaataatcatcaagagataaaatttaaatattt

SEQ ID NO: 14 - DNA, 189 bp, synthetic construct
  source 1..189: mol_type = other DNA
ttacattatttcaggtcacgtagccagcaaaaggagacactggtatgcaccagcatttttcttttccttttcttttgtggtctttattttttagaatatatatgataatatatacggattaatttcaatttcttttgtatcatattcaactactagtttacatatatgggactttttaataatcatcaa

SEQ ID NO: 15 - DNA, 149 bp, synthetic construct
  source 1..149: mol_type = other DNA
tagccagcaaaaggagacactggtatgcaccagcatttttcttttccttttcttttgtggtctttattttttagaatatatatgataatatatacggattaatttcaatttcttttgtatcatattcaactactagtttacatatatgg

SEQ ID NO: 16 - DNA, 223 bp, synthetic construct
  source 1..223: mol_type = other DNA
tatttaaccaatataaaaaaaattagccttccattctcttaccaaaaaaaagtaatattaattttcaatttgcatctctgttacattatttcaggtcacgtagccagcaaaaggagacactggtatgcaccagcatttttcttttccttttcttttgtggtctttattttttagaatatatatgataatatatacggattaatttcaatttcttttgtatcat

SEQ ID NO: 17 - DNA, 143 bp, synthetic construct
  source 1..143: mol_type = other DNA
accaaaaaaaagtaatattaattttcaatttgcatctctgttacattatttcaggtcacgtagccagcaaaaggagacactggtatgcaccagcatttttcttttccttttcttttgtggtctttattttttagaatatatat

SEQ ID NO: 18 - DNA, 103 bp, synthetic construct
  source 1..103: mol_type = other DNA
attttcaatttgcatctctgttacattatttcaggtcacgtagccagcaaaaggagacactggtatgcaccagcatttttcttttccttttcttttgtggtct

SEQ ID NO: 19 - DNA, 63 bp, synthetic construct
  source 1..63: mol_type = other DNA
ttacattatttcaggtcacgtagccagcaaaaggagacactggtatgcaccagcatttttctt

SEQ ID NO: 20 - DNA, 223 bp, synthetic construct
  source 1..223: mol_type = other DNA
ttaaccaatataaaaaaaattagccttccattctcttaccaaaaaaaagtaatattaattttcaatttgcatctctgttacattatttcaggtcacgtagccagcaaaaggagacactggtatgcaccagcatttttcttttccttttcttttgtggtctttattttttagaatatatatgataatatatacggattaatttcaatttcttttgtatcatatt

SEQ ID NO: 21 - DNA, 145 bp, synthetic construct
  source 1..145: mol_type = other DNA
accaaaaaaaagtaatattaattttcaatttgcatctctgttacattatttcaggtcacgtagccagcaaaaggagacactggtatgcaccagcatttttcttttccttttcttttgtggtctttattttttagaatatatatga

SEQ ID NO: 22 - DNA, 106 bp, synthetic construct
  source 1..106: mol_type = other DNA
attttcaatttgcatctctgttacattatttcaggtcacgtagccagcaaaaggagacactggtatgcaccagcatttttcttttccttttcttttgtggtcttta

SEQ ID NO: 23 - DNA, 66 bp, synthetic construct
  source 1..66: mol_type = other DNA
ttacattatttcaggtcacgtagccagcaaaaggagacactggtatgcaccagcatttttcttttc

SEQ ID NO: 24 - DNA, 223 bp, synthetic construct
  source 1..223: mol_type = other DNA
gcaccagcatttttcttttccttttcttttgtggtctttattttttagaatatatatgataatatatacggattaatttcaatttcttttgtatcatattcaactactagtttacatatatgggactttttaataatcatcaagagataaaatttaaatatttttatttatcatataattagagataagataaattttgattcatgaatttgatctgataatt

SEQ ID NO: 25 - DNA, 143 bp, synthetic construct
  source 1..143: mol_type = other DNA
ttttttagaatatatatgataatatatacggattaatttcaatttcttttgtatcatattcaactactagtttacatatatgggactttttaataatcatcaagagataaaatttaaatatttttatttatcatataattaga

SEQ ID NO: 26 - DNA, 100 bp, synthetic construct
  source 1..100: mol_type = other DNA
atatacggattaatttcaatttcttttgtatcatattcaactactagtttacatatatgggactttttaataatcatcaagagataaaatttaaatattt

SEQ ID NO: 27 - DNA, 63 bp, synthetic construct
  source 1..63: mol_type = other DNA
aatttcttttgtatcatattcaactactagtttacatatatgggactttttaataatcatcaa

SEQ ID NO: 28 - DNA, 223 bp, synthetic construct
  source 1..223: mol_type = other DNA
taatttgataatgagttttataactataattcaatcaagtcattttgtcacccattctattactgtattacatggatacttatgtgtgcagataactattaagtattaacagctcatcagtggcttgcagatagtaatgtctctcaacttgtttacatgacatttaactaaatgagcatctcataagtattttaagcattttattaattagattgattattaa

SEQ ID NO: 29 - DNA, 144 bp, synthetic construct
  source 1..144: mol_type = other DNA
cattttgtcacccattctattactgtattacatggatacttatgtgtgcagataactattaagtattaacagctcatcagtggcttgcagatagtaatgtctctcaacttgtttacatgacatttaactaaatgagcatctcat

SEQ ID NO: 30 - DNA, 103 bp, synthetic construct
  source 1..103: mol_type = other DNA
tactgtattacatggatacttatgtgtgcagataactattaagtattaacagctcatcagtggcttgcagatagtaatgtctctcaacttgtttacatgacat

SEQ ID NO: 31 - DNA, 63 bp, synthetic construct
  source 1..63: mol_type = other DNA
tatgtgtgcagataactattaagtattaacagctcatcagtggcttgcagatagtaatgtctc

SEQ ID NO: 32 - DNA, 472 bp, synthetic construct
  source 1..472: mol_type = other DNA
atacatacatacatgtacatgtatcttcactgaaacgtcctcgattctcttgcgagcttctgctttagacaacatgatacattccgctgcttcatccatggcctcacccctctgcacgtggctggtggcagcttgcatgtcagtcacgtgccacgctgaccgcgccgggactccaaaggcagcgcttcgctcatccaagaggtcaaggaggacgaaggcgctgccaccaaacacctcccaattgaacatgagattgatttcttcgctctacggatccagcattcagggactcataagctccttcgagccctgcgatgactactgcaacactcacaatgccttctcttctctctttcgatccacaactcccaatcgcagacacacccgtctcaataaacttgctcactctggtaaactttttttttttttttaattttccccacttaaatcacctcaagttaagatatttctg

SEQ ID NO: 33 - DNA, 372 bp, synthetic construct
  source 1..372: mol_type = other DNA
tgcgagcttctgctttagacaacatgatacattccgctgcttcatccatggcctcacccctctgcacgtggctggtggcagcttgcatgtcagtcacgtgccacgctgaccgcgccgggactccaaaggcagcgcttcgctcatccaagaggtcaaggaggacgaaggcgctgccaccaaacacctcccaattgaacatgagattgatttcttcgctctacggatccagcattcagggactcataagctccttcgagccctgcgatgactactgcaacactcacaatgccttctcttctctctttcgatccacaactcccaatcgcagacacacccgtctcaataaacttgctcactctggtaaactttttt

SEQ ID NO: 34 - DNA, 292 bp, synthetic construct
  source 1..292: mol_type = other DNA
ttcatccatggcctcacccctctgcacgtggctggtggcagcttgcatgtcagtcacgtgccacgctgaccgcgccgggactccaaaggcagcgcttcgctcatccaagaggtcaaggaggacgaaggcgctgccaccaaacacctcccaattgaacatgagattgatttcttcgctctacggatccagcattcagggactcataagctccttcgagccctgcgatgactactgcaacactcacaatgccttctcttctctctttcgatccacaactcccaatcgcagacac

SEQ ID NO: 35 - DNA, 252 bp, synthetic construct
  source 1..252: mol_type = other DNA
tctgcacgtggctggtggcagcttgcatgtcagtcacgtgccacgctgaccgcgccgggactccaaaggcagcgcttcgctcatccaagaggtcaaggaggacgaaggcgctgccaccaaacacctcccaattgaacatgagattgatttcttcgctctacggatccagcattcagggactcataagctccttcgagccctgcgatgactactgcaacactcacaatgccttctcttctctctttcgatcca

SEQ ID NO: 36 - DNA, 212 bp, synthetic construct
  source 1..212: mol_type = other DNA
gcttgcatgtcagtcacgtgccacgctgaccgcgccgggactccaaaggcagcgcttcgctcatccaagaggtcaaggaggacgaaggcgctgccaccaaacacctcccaattgaacatgagattgatttcttcgctctacggatccagcattcagggactcataagctccttcgagccctgcgatgactactgcaacactcacaatgcctt

SEQ ID NO: 37 - DNA, 172 bp, synthetic construct
  source 1..172: mol_type = other DNA
ccacgctgaccgcgccgggactccaaaggcagcgcttcgctcatccaagaggtcaaggaggacgaaggcgctgccaccaaacacctcccaattgaacatgagattgatttcttcgctctacggatccagcattcagggactcataagctccttcgagccctgcgatgactac

SEQ ID NO: 38 - DNA, 123 bp, synthetic construct
  source 1..123: mol_type = other DNA
ccacgctgaccgcgccgggactccaaaggcagcgcttcgctcatccaagaggtcaaggaggacgaaggcgctgccaccaaacacctcccaattgaacatgagattgatttcttcgctctacgg

SEQ ID NO: 39 - DNA, 72 bp, synthetic construct
  source 1..72: mol_type = other DNA
agattgatttcttcgctctacggatccagcattcagggactcataagctccttcgagccctgcgatgactac

SEQ ID NO: 40 - DNA, 223 bp, synthetic construct
  source 1..223: mol_type = other DNA
tgcgagcttctgctttagacaacatgatacattccgctgcttcatccatggcctcacccctctgcacgtggctggtggcagcttgcatgtcagtcacgtgccacgctgaccgcgccgggactccaaaggcagcgcttcgctcatccaagaggtcaaggaggacgaaggcgctgccaccaaacacctcccaattgaacatgagattgatttcttcgctctacgg

SEQ ID NO: 41 - DNA, 142 bp, synthetic construct
  source 1..142: mol_type = other DNA
ttcatccatggcctcacccctctgcacgtggctggtggcagcttgcatgtcagtcacgtgccacgctgaccgcgccgggactccaaaggcagcgcttcgctcatccaagaggtcaaggaggacgaaggcgctgccaccaaac

SEQ ID NO: 42 - DNA, 103 bp, synthetic construct
  source 1..103: mol_type = other DNA
tctgcacgtggctggtggcagcttgcatgtcagtcacgtgccacgctgaccgcgccgggactccaaaggcagcgcttcgctcatccaagaggtcaaggaggac

SEQ ID NO: 43 - DNA, 63 bp, synthetic construct
  source 1..63: mol_type = other DNA
gcttgcatgtcagtcacgtgccacgctgaccgcgccgggactccaaaggcagcgcttcgctca

SEQ ID NO: 44 - DNA, 223 bp, synthetic construct
  source 1..223: mol_type = other DNA
ccacgctgaccgcgccgggactccaaaggcagcgcttcgctcatccaagaggtcaaggaggacgaaggcgctgccaccaaacacctcccaattgaacatgagattgatttcttcgctctacggatccagcattcagggactcataagctccttcgagccctgcgatgactactgcaacactcacaatgccttctcttctctctttcgatccacaactcccaat

SEQ ID NO: 45 - DNA, 144 bp, synthetic construct
  source 1..144: mol_type = other DNA
tcatccaagaggtcaaggaggacgaaggcgctgccaccaaacacctcccaattgaacatgagattgatttcttcgctctacggatccagcattcagggactcataagctccttcgagccctgcgatgactactgcaacactcac

SEQ ID NO: 46 - DNA, 103 bp, synthetic construct
  source 1..103: mol_type = other DNA
gacgaaggcgctgccaccaaacacctcccaattgaacatgagattgatttcttcgctctacggatccagcattcagggactcataagctccttcgagccctgc

SEQ ID NO: 47 - DNA, 63 bp, synthetic construct
  source 1..63: mol_type = other DNA
acacctcccaattgaacatgagattgatttcttcgctctacggatccagcattcagggactca

SEQ ID NO: 48 - DNA, 223 bp, synthetic construct
  source 1..223: mol_type = other DNA
aggtcaaggaggacgaaggcgctgccaccaaacacctcccaattgaacatgagattgatttcttcgctctacggatccagcattcagggactcataagctccttcgagccctgcgatgactactgcaacactcacaatgccttctcttctctctttcgatccacaactcccaatcgcagacacacccgtctcaataaacttgctcactctggtaaactttttt

SEQ ID NO: 49 - DNA, 143 bp, synthetic construct
  source 1..143: mol_type = other DNA
aattgaacatgagattgatttcttcgctctacggatccagcattcagggactcataagctccttcgagccctgcgatgactactgcaacactcacaatgccttctcttctctctttcgatccacaactcccaatcgcagacac

SEQ ID NO: 50 - DNA, 103 bp, synthetic construct
  source 1..103: mol_type = other DNA
tcttcgctctacggatccagcattcagggactcataagctccttcgagccctgcgatgactactgcaacactcacaatgccttctcttctctctttcgatcca

SEQ ID NO: 51 - DNA, 63 bp, synthetic construct
  source 1..63: mol_type = other DNA
cattcagggactcataagctccttcgagccctgcgatgactactgcaacactcacaatgcctt

SEQ ID NO: 52 - DNA, 361 bp, synthetic construct
  source 1..361: mol_type = other DNA
atacgaatttcttgaatgataggcagttgcatgcaatttttattattgctgacttattcctctgaattgcataactataaggtaaaacaatggctgtagctgtggaacctgcacatgaagtcacaacaacgaaaaaacctcctacaaaacaaaggcgagtagttgtgaccgggttgggcgttgttacacctcttggtcacgaaccagatgtttactacaacaatttacttgatggtgttagcggcattagtgagatcgagacatttgattgtgctgaatatccaacggcaagttatatataattgaaattgtattttttgttgtctgttgtagcttttggtctgaatattctatgtttcca

SEQ ID NO: 53 - DNA, 261 bp, synthetic construct
  source 1..261: mol_type = other DNA
gacttattcctctgaattgcataactataaggtaaaacaatggctgtagctgtggaacctgcacatgaagtcacaacaacgaaaaaacctcctacaaaacaaaggcgagtagttgtgaccgggttgggcgttgttacacctcttggtcacgaaccagatgtttactacaacaatttacttgatggtgttagcggcattagtgagatcgagacatttgattgtgctgaatatccaacggcaagttatatataattgaaattg

SEQ ID NO: 54 - DNA, 181 bp, synthetic construct
  source 1..181: mol_type = other DNA
tggctgtagctgtggaacctgcacatgaagtcacaacaacgaaaaaacctcctacaaaacaaaggcgagtagttgtgaccgggttgggcgttgttacacctcttggtcacgaaccagatgtttactacaacaatttacttgatggtgttagcggcattagtgagatcgagacatttgattg

SEQ ID NO: 55 - DNA, 141 bp, synthetic construct
  source 1..141: mol_type = other DNA
gcacatgaagtcacaacaacgaaaaaacctcctacaaaacaaaggcgagtagttgtgaccgggttgggcgttgttacacctcttggtcacgaaccagatgtttactacaacaatttacttgatggtgttagcggcattagt

SEQ ID NO: 56 - DNA, 101 bp, synthetic construct
  source 1..101: mol_type = other DNA
gaaaaaacctcctacaaaacaaaggcgagtagttgtgaccgggttgggcgttgttacacctcttggtcacgaaccagatgtttactacaacaatttacttg

SEQ ID NO: 57 - DNA, 61 bp, synthetic construct
  source 1..61: mol_type = other DNA
aaaggcgagtagttgtgaccgggttgggcgttgttacacctcttggtcacgaaccagatgt

SEQ ID NO: 58 - DNA, 223 bp, synthetic construct
  source 1..223: mol_type = other DNA
gacttattcctctgaattgcataactataaggtaaaacaatggctgtagctgtggaacctgcacatgaagtcacaacaacgaaaaaacctcctacaaaacaaaggcgagtagttgtgaccgggttgggcgttgttacacctcttggtcacgaaccagatgtttactacaacaatttacttgatggtgttagcggcattagtgagatcgagacatttgattgtg

SEQ ID NO: 59 - DNA, 143 bp, synthetic construct
  source 1..143: mol_type = other DNA
tggctgtagctgtggaacctgcacatgaagtcacaacaacgaaaaaacctcctacaaaacaaaggcgagtagttgtgaccgggttgggcgttgttacacctcttggtcacgaaccagatgtttactacaacaatttacttgat

SEQ ID NO: 60 - DNA, 103 bp, synthetic construct
  source 1..103: mol_type = other DNA
gcacatgaagtcacaacaacgaaaaaacctcctacaaaacaaaggcgagtagttgtgaccgggttgggcgttgttacacctcttggtcacgaaccagatgttt

SEQ ID NO: 61 - DNA, 63 bp, synthetic construct
  source 1..63: mol_type = other DNA
gaaaaaacctcctacaaaacaaaggcgagtagttgtgaccgggttgggcgttgttacacctct

SEQ ID NO: 62 - DNA, 223 bp, synthetic construct
  source 1..223: mol_type = other DNA
gcataactataaggtaaaacaatggctgtagctgtggaacctgcacatgaagtcacaacaacgaaaaaacctcctacaaaacaaaggcgagtagttgtgaccgggttgggcgttgttacacctcttggtcacgaaccagatgtttactacaacaatttacttgatggtgttagcggcattagtgagatcgagacatttgattgtgctgaatatccaacggcaa

SEQ ID NO: 63 - DNA, 143 bp, synthetic construct
  source 1..143: mol_type = other DNA
ctgcacatgaagtcacaacaacgaaaaaacctcctacaaaacaaaggcgagtagttgtgaccgggttgggcgttgttacacctcttggtcacgaaccagatgtttactacaacaatttacttgatggtgttagcggcattagt

SEQ ID NO: 64 - DNA, 104 bp, synthetic construct
  source 1..104: mol_type = other DNA
aacgaaaaaacctcctacaaaacaaaggcgagtagttgtgaccgggttgggcgttgttacacctcttggtcacgaaccagatgtttactacaacaatttacttg

SEQ ID NO: 65 - DNA, 63 bp, synthetic construct
  source 1..63: mol_type = other DNA
acaaaggcgagtagttgtgaccgggttgggcgttgttacacctcttggtcacgaaccagatgt

SEQ ID NO: 66 - DNA, 223 bp, synthetic construct
  source 1..223: mol_type = other DNA
aatggctgtagctgtggaacctgcacatgaagtcacaacaacgaaaaaacctcctacaaaacaaaggcgagtagttgtgaccgggttgggcgttgttacacctcttggtcacgaaccagatgtttactacaacaatttacttgatggtgttagcggcattagtgagatcgagacatttgattgtgctgaatatccaacggcaagttatatataattgaaattg

SEQ ID NO: 67 - DNA, 143 bp, synthetic construct
  source 1..143: mol_type = other DNA
acgaaaaaacctcctacaaaacaaaggcgagtagttgtgaccgggttgggcgttgttacacctcttggtcacgaaccagatgtttactacaacaatttacttgatggtgttagcggcattagtgagatcgagacatttgattg

SEQ ID NO: 68 - DNA, 103 bp, synthetic construct
  source 1..103: mol_type = other DNA
acaaaggcgagtagttgtgaccgggttgggcgttgttacacctcttggtcacgaaccagatgtttactacaacaatttacttgatggtgttagcggcattagt

SEQ ID NO: 69 - DNA, 63 bp, synthetic construct
  source 1..63: mol_type = other DNA
ccgggttgggcgttgttacacctcttggtcacgaaccagatgtttactacaacaatttacttg

SEQ ID NO: 70 - DNA, 354 bp, synthetic construct
  source 1..354: mol_type = other DNA
gtgatgctttgtggtggctcagatgctgctattatacccattggtatgatctcaccattgattctttttcacctgcatacggttatcgtgccataaatctagctgatatgaatccactaatcattggtacaggtttgggaggttttgtggcatgcagagccctatcacagaggaatacagaccctaccaaagcttcacgcccttgggacattgtaatacaacacctgctcattctttctgctactcaccatgttttgtattgctgaggctttcatcattttttatttttgaaataatacgactataattataatgaaatttgatgaccaagtcttgtatcctttcttgtaatct

SEQ ID NO: 71 - DNA, 254 bp, synthetic construct
  source 1..254: mol_type = other DNA
ctcaccattgattctttttcacctgcatacggttatcgtgccataaatctagctgatatgaatccactaatcattggtacaggtttgggaggttttgtggcatgcagagccctatcacagaggaatacagaccctaccaaagcttcacgcccttgggacattgtaatacaacacctgctcattctttctgctactcaccatgttttgtattgctgaggctttcatcattttttatttttgaaataatacgacta

SEQ ID NO: 72 - DNA, 174 bp, synthetic construct
  source 1..174: mol_type = other DNA
ccataaatctagctgatatgaatccactaatcattggtacaggtttgggaggttttgtggcatgcagagccctatcacagaggaatacagaccctaccaaagcttcacgcccttgggacattgtaatacaacacctgctcattctttctgctactcaccatgttttgtattgct

SEQ ID NO: 73 - DNA, 134 bp, synthetic construct
  source 1..134: mol_type = other DNA
aatccactaatcattggtacaggtttgggaggttttgtggcatgcagagccctatcacagaggaatacagaccctaccaaagcttcacgcccttgggacattgtaatacaacacctgctcattctttctgctac

SEQ ID NO: 74 - DNA, 94 bp, synthetic construct
  source 1..94: mol_type = other DNA
aggtttgggaggttttgtggcatgcagagccctatcacagaggaatacagaccctaccaaagcttcacgcccttgggacattgtaatacaacac

SEQ ID NO: 75 - DNA, 54 bp, synthetic construct
  source 1..54: mol_type = other DNA
catgcagagccctatcacagaggaatacagaccctaccaaagcttcacgccctt

SEQ ID NO: 76 - DNA, 223 bp, synthetic construct
  source 1..223: mol_type = other DNA
ctcaccattgattctttttcacctgcatacggttatcgtgccataaatctagctgatatgaatccactaatcattggtacaggtttgggaggttttgtggcatgcagagccctatcacagaggaatacagaccctaccaaagcttcacgcccttgggacattgtaatacaacacctgctcattctttctgctactcaccatgttttgtattgctgaggctttc

SEQ ID NO: 77 - DNA, 173 bp, synthetic construct
  source 1..173: mol_type = other DNA
cataaatctagctgatatgaatccactaatcattggtacaggtttgggaggttttgtggcatgcagagccctatcacagaggaatacagaccctaccaaagcttcacgcccttgggacattgtaatacaacacctgctcattctttctgctactcaccatgttttgtattgct

SEQ ID NO: 78 - DNA, 134 bp, synthetic construct; identical to SEQ ID NO: 73
  source 1..134: mol_type = other DNA

SEQ ID NO: 79 - DNA, 94 bp, synthetic construct; identical to SEQ ID NO: 74
  source 1..94: mol_type = other DNA

SEQ ID NO: 80 - DNA, 223 bp, synthetic construct
  source 1..223: mol_type = other DNA
gttatcgtgccataaatctagctgatatgaatccactaatcattggtacaggtttgggaggttttgtggcatgcagagccctatcacagaggaatacagaccctaccaaagcttcacgcccttgggacattgtaatacaacacctgctcattctttctgctactcaccatgttttgtattgctgaggctttcatcattttttatttttgaaataatacgacta

SEQ ID NO: 81 - DNA, 143 bp, synthetic construct
  source 1..143: mol_type = other DNA
cattggtacaggtttgggaggttttgtggcatgcagagccctatcacagaggaatacagaccctaccaaagcttcacgcccttgggacattgtaatacaacacctgctcattctttctgctactcaccatgttttgtattgct

SEQ ID NO: 82 - DNA, 103 bp, synthetic construct
  source 1..103: mol_type = other DNA
gttttgtggcatgcagagccctatcacagaggaatacagaccctaccaaagcttcacgcccttgggacattgtaatacaacacctgctcattctttctgctac

SEQ ID NO: 83 - DNA, 63 bp, synthetic construct
  source 1..63: mol_type = other DNA
ctatcacagaggaatacagaccctaccaaagcttcacgcccttgggacattgtaatacaacac

SEQ ID NO: 84 - DNA, 23 bp, synthetic construct
  source 1..23: mol_type = other DNA
gagtcccggcgcggtcagcgtgg

SEQ ID NO: 85 - DNA, 23 bp, synthetic construct
  source 1..23: mol_type = other DNA
gtagtcatcgcagggctcgaagg

SEQ ID NO: 86 - DNA, 23 bp, synthetic construct
  source 1..23: mol_type = other DNA
agattgatttcttcgctctacgg

SEQ ID NO: 87 - DNA, 23 bp, synthetic construct
  source 1..23: mol_type = other DNA
aaaggcgagtagttgtgaccggg

SEQ ID NO: 88 - DNA, 23 bp, synthetic construct
  source 1..23: mol_type = other DNA
acatctggttcgtgaccaagagg

SEQ ID NO: 89 - DNA, 23 bp, synthetic construct
  source 1..23: mol_type = other DNA
aggtgtaacaacgcccaacccgg

SEQ ID NO: 90 - DNA, 23 bp, synthetic construct
  source 1..23: mol_type = other DNA
catgcagagccctatcacagagg

SEQ ID NO: 91 - DNA, 23 bp, synthetic construct
  source 1..23: mol_type = other DNA
aagggcgtgaagctttggtaggg

SEQ ID NO: 92 - DNA, 23 bp, synthetic construct
  source 1..23: mol_type = other DNA
ataccagtgtctccttttgctgg

SEQ ID NO: 93 - DNA, 23 bp, synthetic construct
  source 1..23: mol_type = other DNA
tagccagcaaaaggagacactgg

SEQ ID NO: 94 - DNA, 23 bp, synthetic construct
  source 1..23: mol_type = other DNA
caactactagtttacatatatgg

SEQ ID NO: 95 - DNA, 23 bp, synthetic construct
  source 1..23: mol_type = other DNA
aagtattaacagctcatcagtgg

SEQ ID NO: 96 - DNA, 22 bp, synthetic construct
  source 1..22: mol_type = other DNA
atgatacattccgctgcttcat

SEQ ID NO: 97 - DNA, 22 bp, synthetic construct
  source 1..22: mol_type = other DNA
gaaggagcttatgagtccctga

SEQ ID NO: 98 - DNA, 22 bp, synthetic construct
  source 1..22: mol_type = other DNA
acctcccaattgaacatgagat

SEQ ID NO: 99 - DNA, 22 bp, synthetic construct
  source 1..22: mol_type = other DNA
tttattgagacgggtgtgtctg

SEQ ID NO: 100 - DNA, 22 bp, synthetic construct
  source 1..22: mol_type = other DNA
agtgttgcagtagtcatcgcag

SEQ ID NO: 101 - DNA, 22 bp, synthetic construct
  source 1..22: mol_type = other DNA
aacctgcacatgaagtcacaac

SEQ ID NO: 102 - DNA, 22 bp, synthetic construct
  source 1..22: mol_type = other DNA
cagcacaatcaaatgtctcgat

SEQ ID NO: 103 - DNA, 22 bp, synthetic construct
  source 1..22: mol_type = other DNA
cacatgaagtcacaacaacgaa

SEQ ID NO: 104 - DNA, 22 bp, synthetic construct
  source 1..22: mol_type = other DNA
gatctcactaatgccgctaaca

SEQ ID NO: 105 - DNA, 22 bp, synthetic construct
  source 1..22: mol_type = other DNA
acggttatcgtgccataaatct

SEQ ID NO: 106 - DNA, 22 bp, synthetic construct
  source 1..22: mol_type = other DNA
tgaaagcctcagcaatacaaaa

SEQ ID NO: 107 - DNA, 22 bp, synthetic construct
  source 1..22: mol_type = other DNA
attctttttcacctgcatacgg

SEQ ID NO: 108 - DNA, 22 bp, synthetic construct
  source 1..22: mol_type = other DNA
gccttccattctcttaccaaaa

SEQ ID NO: 109 - DNA, 24 bp, synthetic construct
  source 1..24: mol_type = other DNA
caaaagaaattgaaattaatccgt

SEQ ID NO: 110 - DNA, 22 bp, synthetic construct
  source 1..22: mol_type = other DNA
ttttcttcaaacaaatcggacc

SEQ ID NO: 111 - DNA, 25 bp, synthetic construct
  source 1..25: mol_type = other DNA
aaataaagaccacaaaagaaaagga

SEQ ID NO: 112 - DNA, 24 bp, synthetic construct
  source 1..24: mol_type = other DNA
ttttcttttccttttcttttgtgg

SEQ ID NO: 113 - DNA, 22 bp, synthetic construct
  source 1..22: mol_type = other DNA
aatgggtgacaaaatgacttga

SEQ ID NO: 114 - DNA, 22 bp, synthetic construct
  source 1..22: mol_type = other DNA
tcaagtcattttgtcacccatt

SEQ ID NO: 115 - DNA, 25 bp, synthetic construct
  source 1..25: mol_type = other DNA
aaacaaaagctatttttaaccgtaa

SEQ ID NO: 116 - DNA, 7723 bp, synthetic construct
  source 1..7723: mol_type = other DNA
tttgtatcatattcaactactagtttacatatatgagactttttaataaccatcaagagataaaatttaaatatttttattgatcatataatacttaattgtatagttgtgtaaaatactttaaataatactattaatatattaaaatcagctattatatatattgtttaatttatttttaatatatattttatattttaatatgtattttatattaatgtttgattttggtatatacttaataatacagttaatacattgataatattttagactatttttcttaattttattttttaaagttttaatatcaaccattcatttattaaagataagataaattttgattcatgaatttgatctgataattaatataaattttaaacatgcttaatttgataatgagttttataactataattcaatcaagtcattttgtcacccattcatgaatacttatgtgtgcagataactattaacagctcatcagtagcttgctgatggtaatgtctctcaacttgtttacatgacagttaactaaccgggcatctcataagtattttaatttgtaagcattttattaattagattgattataaaaaaaaattaatttgatttacggttaaaaatagcttttgtttaaaatttaaaagccaaactaactattagatgaaaatttatgagcaaattgagttttaatgctagtttggattggtttttttgagtaaaaattattttttaaaaacaaattaaagtactttttactcaattttaaatttatttagatatatttttttaatttattaaaaaattaaactatttacttttttaaaagttaacaatattttacttttaaaaaaatagaaacaaaaaatatttttaatatttgaaattttttaaaaagcttatttaaatgtaaaataatttaaaaaaataaaaactaaatatttttttaaaaatcaatttaaactgatctttatctcttaattattaactaattttggtatttactttcaaattttcgtaaaaaaaagtcctacaagagaataaagtattattttgactaaagaaaatcttattttgactaaagaaaatctgaaaccaagaataaaataaatgctatgtcacgttttacactaatgataaaaaaattaaagtacaatataagtagaaaatatgttctaaaaccaaacaaaaaagtgcattaaattgcgaaaaggattgatccaaggagaaatggcgtaatgaattagcatagaaagcaattagttttatattgcataaataatgatgaattatattaggtaaaggttcaggtggagtggaattcacgtaaagttgataactgagagtcgttcgataatttgattgatttgactaaatttttatttaacggctctcgactatcaactttgcgatgcacctgaattttaccattatattagtagcgaagaaaaagaaaagaggttagtagaaaattaaattaaattaataaagaaagcaaatgagaagcgaaggagcgactgagactcagattgcagaaaaagcagagactttgagactcgactcttgagacttcacaacgcctcccttttcctccttctctcagatcgattctctttctcactctctcaactctcttcaaaacccccacaactaactttcttcttcttcttcttcttgttctttgtatatacatacatacatacatacatgtacatgtatctccaccttccatatccaccactcctgacttgctcttaatcttcactgaaacgtcctcgattctcttgcgagcttctgctttagacaacatgatacattccgctgcttcatccatggcctcacccctctgcacgtggctggtggcggcttgcatgtcagtcacgtgccacactgaccgcgccgggactccaaaggcagcgcttcggtcatccaagaggtcaaggaggacgaaggcgctgccagcaaacacctcccaattgaacatgagattgatttcttcgctgtacggatccagcattcagggactcataagctccttcgagccctgcgatgactactgcaacactcacaatgccttctcttcgctctttcgatccacaactcccaatcgcagacacacccgtctcaataaactttctcattctggtaaacttttttttttttttttttaattttccccacttaaatcacctcaagttaagatatttctggtgctttttccaattttaatctgtaccccgtactgggtatttcaatttgattcatttatttatttttcttatattaatataataataatagtttttttttttattatggttatgaaatctgggttctgattttctggtcttctgaaagatatgtgtgtgtgtgtggcttttttcccttttttttctgaattacttaaaatgcacaagtgttagcttggatctttgtcttttggttggtcatcctaataaatggggttgcttggtggtttagtattgagggttgttctacctgctctgtttaactgttttgagggtacggtttataatattaggagtatgggggtagttgttgttctttctttgctgagattgatggtgcacctgtgtcacttaattaggacatgttgctacaacactatgccattgcccatgctttttgctttcatttcccattcctacttcatttccttgtatttatcgtacacccaggtatctacataaagttcccattaaatattggttaatgttcgcatcatcactttgaatttcttgcaatctcgttgaggtagttcagcatattcacagaatcaactcaaacgaaaataaatttcttttgacttttattttgttttcttttcttcatgaaaataataatcacactgaagtgttctgaatttaactactatgtaaatatcgcccccacgtgggattacctctctctccctctctctctagtttttttgtttctctatgtaaaatagtggaggtaagttatgcctttgtcataagcacacataagcaagcagaggctcctttttttttccctcattttttttttaaattatcatgtaaaatagatgattctggcacaaactatccacaagttatttttgctcctagttctttagtatgtttttaacacactggacaagtctactactgctgcatgcatgtccctgactgatgggttctagtattcagtggtcttgagtgttttgatttaaagcaagggatataagttgggtctgatgaatttttcgaggtttcatagcatttttgttaccatagtcttagaattactaccttggataaacctaagcatatttttgttaatggctggggttgcatttcctgctcaacattaatgtgcaacaagggtatgttcggttaggggttaaaaatggggcgggaattttcaatggatgtggcccatgtgtaattttactcatagaacttattgtaaaaatttcaccccatttctacttcccaaaagaacatatccttatcatgatcattggaagctacacatgcatcatgttgaactataagctgtgggccgttctttcttgtatgcttctgatatgaatttcttgaatgataggcggttgcatgcaatttttattattgctgacttattcctctgaattgcaaaactataaggtaaaacaatggctgtagctgtggaacctgcacacgaagtcacagcaacgaaaaaacctcctacaaaacaaaggcgagtagttgtgactgggttgggcgttgttacacctcttggtcacgaaccagatgtttactacaacaatttacttgatggtgttagtggcataagtgagatcgagacatttgattgtgctgaatatccaacggcaagttatatataattgaaattgtatttcttgttgtctgttgtagcttttgttctgaacattctatgtttccagaggattgctggtgagatcaagtcgttctccactgatggctgggttgcaccaaaactttcgaagagaatggataaatttatgctctatttgctaacagccggtaaaaaggccttagttgatggtggaattactgaagatgtaatggatgagttgaataaggaaaaatgtggagttttgattggctcagcgatgggtggcatgaaggtaaccttgtctttctttcatgttgagatacaaatcttgtaaaaccagctggtgttgtggactgaatttaatgccactgcaatccaaatggagcagaagagtgtgctatattttcaaccttgtgcccttgttatattgactatgatttttggtgctttttctactgagcaggtttttaatgatgccattgaagctttaagagtatcatataagaagatgaatcctttttgtgtaccttttgccacaacaaatatgggatctgccatacttgcgatggatctggtctgttgttgcatggacaccataaattggtgttttgtttcgtaccaagcttttattttggctatgttatcttcagcttctaacaaacattgttatcttttatggattacagggatggatgggccctaattattcaatctctacagcttgtgctacaagtaacttttgtatattgaatgcagcaaaccacatcataagaggtgaagctgtaagtattcatgctttgatattactctcaggatggtgaatttttcgtatccaatattataccttatatttatttatttatttaactttctttttctgagacagtttggtgtaatgatatggtcattgcatcaaaatgtattgataaatttaatatgaaaatattcctgcaggatgtgatgctttgtggtggctcagatgctgctattatacccattggtatgatctcaccattgattctttttcacctgcatacggttatcatgccataaatctagctgatatgaatccactaatcattggtacaggtttgggaggttttgtggcatgcagagccctatcgcagaggaatacagaccctaccaaagcttcacgcccttgggacattgtaatacaacagaaatcctgctcattctttctgctactcaccatgtttttgtattgctgatctttcatcattttttatttttgaaacaatacaactataattataatgaaatttgatgaccaagtcttgtatcctttcttgtaatctgaacagcatagtttattaaaaactaacgtttcaatggtcacagatacaacttttccttttgatcacaaaaatgctgtcttctatggatatgagctatggcaaattcagtttttctaattaggctgtttatgatattccagaatcgagatggatttgtcatgggggaaggagctggagttttacttttggaggacttggagcatgcaaaggtacaggaaatcagatctaataattttaaaacagaacatcccttatgaaatgacagtgcatgttttgatattattactgcagacttgtgcatgtaatttatttcatttagtgcatccttcagttccttcaaacagtattaaaggacttcatacatctgttctctcttgaattaggattgattttcaggctgctgcttgtcataaaaattcacctgttacttctcatgtcaaatctgcgcttgacatttgtcttgcaactaaatcaaatgcatctcttttacattgcagaaaagaggtgcaaccatatatgctgaattccttggtggaagcttcacctgtgatgcatatcatgtgacagagccacgtcccgatggtaaaatttcaaatcttttactaccaaaatttaaagcatttatatgcatgctatgattaccatgcttaaaatccgaagagaacaaggcaaaagaatgtgacaagggtatttttatatatttagaatcatgtgtgccatttgctgatggttaggttaagtatttagaaatatactatatgttgcatttttcttgtttggcactggaatttcttcttccgagatcgtgatggaatttaaaactgcatgttgcgatcatgacttgtctcgacaatatggacctgggatttaataatagtcctatattagtggacctagtaatcctagtcataattagtatctgcttgtactttaccacatttcctttatgattccattcggactcttctttgagcattctatgacttgacaaggaatgatttgtatctccctgcggaacttgcattcacgtttctttactccatcttaaataggagctggcgttatactttgcattgaaaaggcattagctcagtctggagtatcaaaagaggatgtgaactacataaatgcacatgccacatccacgcctgctggggatcttaaggagttccaggctctgcttcattgttttggtcaaaatcccgaggtacatctatctttttaagttacaccaccattaacattgttatcttggtgatcaattctatggtaaacatgatctcttgttcttctggagttcatgttgagtaaaacactggtacttctgatatggtcatccatactaatgtctcttgttttttcagctaagagtgaattctacaaaatctatgattggtcatctactaggagcagctggtggcgtggaagctgtagcaacagtccaggtaaaacccaatggatcacttagttcggtaccccgcgattgatgactaaaatggtcctgaggtttgctctctcttcgtacaggcaattaggacagggtgggtgcatcccaatatcaacctggaaaacccagatcaaggagtggtaatgtttcttgttgcatcattccatccttcatttcaaaattttaatcttttaatctaacatgtccaaactaatgacaaaaattcaggatgccaaagtgcttgtgggctcaaagaaagagagactagatatcaaggctgccctatccaattcatttggttttggaggccacaattcttcaatcatatttgctccatataagtgaatgaatacatgtttcagaataataccttccctaccattatacagaaggtatgtctctaaataagaatagatgaccatttgtattcataaaagatgaaaacactgacatatgtatccacactagatcgaaactaaacttgtacccacgcaagatgtcctccgtgtgacaaaagtaccctacgtggcactctaaccctccaaagacaggatacttttgtcacacggagggcatctttcgtgggtacaagtttagtttcgatttaatgtgggtacatatgtcagcattttcatttttcatggatacaaatggtcatttattcctctaaataatgagcaggtcatatcgtcctactacgagaaacacatttaatttggatgactgtggatgttccttgaacatgtattaattgtgtgttgtttttatgttaagttactaaggtagccaggcaattgttacacgcatgatcaaagtcatgaattggatgaaaatatatgtggaaatggttggagcagaaactcttgaggacattgttgagagatggtttggtggtttagattgtctggccgagatacatggtggagccggttctatccaccgatgaagaacatgaatggctgctattgcctttatagagtaggacttgacccactttgatgtgactcttaaacaaatccacttggcagtatatataagctcttctgttttatttattccctatgtacccaaatagtttgtgcaataacaaattgcaatttattttttcgct

SEQ ID NO: 117 - DNA, 7723 bp, synthetic construct
  source 1..7723: mol_type = other DNA
tttgtatcatattcaactactagtttacatatatgagactttttaataaccatcaagagataaaatttaaatatttttattgatcatataatacttaattgtatagttgtgtaaaatactttaaataatactattaatatattaaaatcagctattatatatattgtttaatttatttttaatatatattttatattttaatatgtattttatattaatgtttaattttggtatatacttaataatacagttaatacattgataatattttagactatttttcttaattttattttttaaagttttaatatcaaccattcatttattaaagataagataaattttgattcatgaatttgatctgataattaatataaattttaaacatgcttaatttgataatgagttttataactataattcaatcaagtcattttgtcacccattcatgaatacttatgtgtgcagataactattaacagctcatcagtagcttgctgatggtaatgtctctcaacttgtttacatgacagttaactaaccgggcatctcataagtattttaatttgtaagcattttattaattagattgattataaaaaaaaattaatttgatttacggttaaaaatagcttttgtttaaaatttaaaagccaaactaactattagatgaaaatttatgagcaaattgagttttaatgctagtttggattggtttttttgagtaaaaattattttttaaaaacaaattaaagtactttttactcaattttaaatttatttagatatatttttttaatttattaaaaaattaaactatttacttttttaaaagttaacaatattttacttttaaaaaaatagaaacaaaaaatatttttaatatttgaaattttttaaaaagcttatttaaatgtaaaataatttaaaataataaaaactaaatatttttttaaaaatcaatttaaactgatctttatctcttaattattaactaattttggtatttactttcaaattttcgtaaaaaaaagtcctacaagagaataaagtattattttgactaaagaaaatcttattttgactaaagaaaatctgaaaccaagaataaaataaatgctatgtcacgttttacactaatgataaaaaaattaaagtacaatataagtagaaaatatgttctaaaaccaaacaaaaaagtgcattaaattgcgaaaaggattgatccaaggagaaatggcgtaatgaattagcatagaaagcaattagttttatattgcataaataatgatgaattatattaggtaaaggttcaggtggagtggaattcacgtaaagttgataactgagagtcgttagataatttgattgatttgactaaatttttatttaacggctctcgactatcaactttgcgatgcacctgaattttaccattatattagtagcgaagaaaaagaaaagaggttagtagaaaattaaattaaattaataaagaaagcaaatgagaagcgaaggagcgactgagactcagattgcagaaaaagcagagactttgagactcgactcttgagacttcacaacgcctcccttttcctccttctctcagatcgattctctttctcactctctcaactctcttcaaaacccccacaactaactttcttcttcttcttcttcttgttctttgtatatacatacatacatacatacatgtacatgtatctccaccttccatatccaccactcctgacttgctcttaatcttcactgaaacgtcctcgattctcttgcgagcttctgctttagacaacatgatacattccgctgcttcatccatggcctcacccctctgcacgtggctggtggcggcttgcatgtcagtcacgtgccacactgaccgcgccgggactccaaaggcagcgcttcggtcatccaagaggtcaaggaggacgaaggcgctgccagcaaacacctcccaattgaacatgagattgatttcttcgctgtacggatccagcattcagggactcataagctccttcgagccctgcgatgactactgcaacactcacaatgccttctcttcgctctttcgatccacaactcccaatcgcagacacacccgtctcaataaactttctcattctggtaaacttttttttttttttttttaattttccccacttaaatcacctcaagttaagatatttctggtgctttttccaattttaatctgtaccccgtactgggtatttcaatttgattcatttatttatttttcttatattaatataataataatagtttttttttttattatggttatgaaatctgggttctgattttctggtcttctgaaagatatgtgtgtgtgtgtggcttttttcccttttttttctgaattacttaaaatgcacaagtgttagcttggatctttgtcttttggttggtcatcctaataaatggggttgcttggtggtttagtattgagggttgttctacctgctctgtttaactgttttgagggtacggtttataatattaggagtatgggggtagttgttgttctttctttgctgagattgatggtgcacctgtgtcacttaattaggacatgttgctacaacactatgccattgcccatgctttttgctttcatttcccattcctacttcatttccttgtatttatcgtacacccaggtatctacataaagttcccattaaatattggttaatgttcgcatcatcactttgaatttcttgcaatctcgttgaggtagttcagcatattcacagaatcaactcaaacgaaaataaatttcttttgacttttattttgttttcttttcttcatgaaaataataatcacactgaagtgttctgaatttaactactatgtaaatatcgcccccacgtgggattacctctctctccctctctctctagtttttttgtttctctatgtaaaatagtggaggtaagttatgcctttgtcataagcacacataagcaagcagaggctcctttttttttccctcattttttttttaaattatcatgtaaaatagatgattctggcacaaactatccacaagttatttttgctcctagttctttagtatgtttttaacacactggacaagtctactactgctgcatgcatgtccctgactgatgggttctagtattcagtggtcttgagtgttttgatttaaagcaagggatataagttgggtctgatgaatttttcgaggtttcatagcatttttgttaccatagtcttagaattactaccttggataaacctaagcatatttttgttaatggctggggttgcatttcctgctcaacattaatgtgcaacaagggtatgttcggttaggggttaaaaatggggcgggaattttcaatggatgtggcccatgtgtaattttactcatagaacttattgtaaaaatttcaccccatttctacttcccaaaagaacatatccttatcatgatcattggaagctacacatgcatcatgttgaactataagctgtgggccgttctttcttgtatgcttctgatatgaatttcttgaatgataggcggttgcatgcaatttttattattgctgacttattcctctgaattgcaaaactataaggtaaaacaatggctgtagctgtggaacctgcacacgaagtcacagcaacgaaaaaacctcctacaaaacaaaggcgagtagttgtgactgggttgggcgttgttacacctcttggtcacgaaccagatgtttactacaacaatttacttgatggtgttagtggcataagtgagatcgagacatttgattgtgctgaatatccaacggcaagttatatataattgaaattgtatttcttgttgtctgttgtagcttttgttctgaacattctatgtttccagaggattgctggtgagatcaagtcgttctccactgatggctgggttgcaccaaaactttcgaagagaatggataaatttatgctctatttgctaacagccggtaaaaaggccttagttgatggtggaattactgaagatgtaatggatgagttgaataaggaaaaatgtggagttttgattggctcagcgatgggtggcatgaaggtaaccttgtctttctttcatgttgagatacaaatcttgtaaaaccagctggtgttgtggactgaatttaatgccactgcaatccaaatggagcagaagagtgtgctatattttcaaccttgtgcccttgttatattgactatgatttttggtgctttttctactgagcaggtttttaatgatgccattgaagctttaagagtatcatataagaagatgaatcctttttgtgtaccttttgccacaacaaatatgggatctgccatacttgcgatggatctggtctgttgttgcatggacaccataaattggtgttttgtttcgtaccaagcttttattttggctatgttatcttcagcttctaacaaacattgttatcttttatggattacagggatggatgggccctaattattcaatctctacagcttgtgctacaagtaacttttgtatattgaatgcagcaaaccacatcataagaggtgaagctgtaagtattcatgctttgatattactctcaggatggtgaatttttcgtatccaatattataccttatatttatttatttatttaactttctttttctgagacagtttggtgtaatgatatggtcattgcatcaaaatgtattgataaatttaatatgaaaatattcctgcaggatgtgatgctttgtggtggctcagatgctgctattatacccattggtatgatctcaccattgattctttttcacctgcatacggttatcatgccataaatctagctgatatgaatccactaatcattggtacaggtttgggaggttttgtggcatgcagagccctatcgcagaggaatacagaccctaccaaagcttcacgcccttgggacattgtaatacaacagaaatcctgctcattctttctgctactcaccatgtttttgtattgctgatctttcatcattttttatttttgaaacaatacaactataattataatgaaatttgatgaccaagtcttgtatcctttcttgtaatctgaacagcatagtttattaaaaactaacgtttcaatggtcacagatacaacttttccttttgatcacaaaaatgctgtcttctatggatatgagctatggcaaattcagtttttctaattaggctgtttatgatattccagaatcgagatggatttgtcatgggggaaggagctggagttttacttttggaggacttggagcatgcaaaggtacaggaaatcagatctaataattttaaaacagaacatcccttatgaaatgacagtgcatgttttgatattattactgcagacttgtgcatgtaatttatttcatttagtgcatccttcagttccttcaaacagtattaaaggacttcatacatctgttctctcttgaattaggattgattttcaggctgctgcttgtcataaaaattcacctgttacttctcatgtcaaatctgcgcttgacatttgtcttgcaactaaatcaaatgcatctcttttacattgcagaaaagaggtgcaaccatatatgctgaattccttggtggaagcttcacctgtgatgcatatcatgtgacagagccacgtcccgatggtaaaatttcaaatcttttactaccaaaatttaaagcatttatatgcatgctatgattaccatgcttaaaatccgaagagaacaaggcaaaagaatgtgacaagggtatttttatatatttagaatcatgtgtgccatttgctgatggttaggttaagtatttagaaatatactatatgttgcatttttcttgtttggcactggaatttcttcttccgagatcgtgatggaatttaaaactgcatgttgcgatcatgacttgtctcgacaatatggacctgggatttaataatagtcctatattagtggacctagtaatcctagtcataattagtatctgcttgtactttaccacatttcctttatgattccattcggactcttctttgagcattctatgacttgacaaggaatgatttgtatctccctgcggaacttgcattcacgtttctttactccatcttaaataggagctggcgttatactttgcattgaaaaggcattagctcagtctggagtatcaaaagaggatgtgaactacataaatgcacatgccacatccacgcctgctggggatcttaaggagttccaggctctgcttcattgttttggtcaaaatcccgaggtacatctatctttttaagttacaccaccattaacattgttatcttggtgatcaattctatggtaaacatgatctcttgttcttctggagttcatgttgagtaaaacactggtacttctgatatggtcatccatactaatgtctcttgttttttcagctaagagtgaattctacaaaatctatgattggtcatctactaggagcagctggtggcgtggaagctgtagcaacagtccaggtaaaacccaatggatcacttagttcggtaccccgcgattgatgactaaaatggtcctgaggtttgctctctcttcgtacaggcaattaggacagggtgggtgcatcccaatatcaacctggaaaacccagatcaaggagtggtaatgtttcttgttgcatcattccatccttcatttcaaaattttaatcttttaatctaacatgtccaaactaatgacaaaaattcaggatgccaaagtgcttgtgggctcaaagaaagagagactagatatcaaggctgccctatccaattcatttggttttggaggccacaattcttcaatcatatttgctccatataagtgaatgaatacatgtttcagaataataccttccctaccattatacagaaggtatgtctctaaataagaatagatgaccatttgtattcataaaagatgaaaacactgacatatgtatccacactagatcgaaactaaacttgtacccacgcaagatgtcctccgtgtgacaaaagtaccctacgtggcactctaaccctccaaagacaggatacttttgtcacacggagggcatctttcgtgggtacaagtttagtttcgatttaatgtgggtacatatgtcagcattttcatttttcatggatacaaatggtcatttattcctctaaataatgagcaggtcatatcgtcctactacgagaaacacatttaatttggatgactgtggatgttccttgaacatgtattaattgtgtgttgtttttatgttaagttactaaggtagccaggcaattgttacacgcatgatcaaagtcatgaattggatgaaaatatatgtggaaatggttggagcagaaactcttgaggacattgttgagagatggtttggtggtttagattgtctggccgagatacatggtggagccggttctatccaccgatgaagaacatgaatggctgctattgcctttatagagtaggacttgacccactttgatgtgactcttaaacaaatccacttggcagtatatataagctcttctgttttatttattccctatgtacccaaatagtttgtgcaataacaaattgcaatttattttttcgct

SEQ ID NO: 118 - DNA, 7723 bp, synthetic construct
  source 1..7723: mol_type = other DNA
tttgtatcatattcaactactagtttacatatatgagactttttaataaccatcaagagataaaatttaaatatttttattgatcatataatacttaattgtatagttgtgtaaaatactttaaataatactattaatatattaaaatcagctattatatatattgtttaatttatttttaatatatattttatattttaatatgtattttatattaatgtttgattttggtatatacttaataatacagttaatacattgataatattttagactatttttcttaattttattttttaaagttttaatatcaaccattcatttattaaagataagataaattttgattcatgaatttgatctgataattaatataaattttaaacatgcttaatttgataatgagttttataactataattcaatcaagtcattttgtcacccattcatgaatacttatgtgtgcagataactattaacagctcatcagtagcttgctgatggtaatgtctctcaacttgtttacatgacagttaactaaccgggcatctcataagtattttaatttgtaagcattttattaattagattgattataaaaaaaaattaatttgatttacggttaaaaatagcttttgtttaaaatttaaaagccaaactaactattagatgaaaatttatgagcaaattgagttttaatgctagtttggattggtttttttgagtaaaaattattttttaaaaacaaattaaagtactttttactcaattttaaatttatttagatatatttttttaatttattaaaaaattaaactatttacttttttaaaagttaacaatattttacttttaaaaaaatagaaacaaaaaatatttttaatatttgaaattttttaaaaagcttatttaaatgtaaaataatttaaaaaaataaaaactaaatatttttttaaaaatcaatttaaactgatctttatctcttaattattaactaattttggtatttactttcaaattttcgtaaaaaaaagtcctacaagagaataaagtattattttgactaaagaaaatcttattttgactaaagaaaatctgaaaccaagaataaaataaatgctatgtcacgttttacactaatgataaaaaaattaaagtacaatataagtagaaaatatgttctaaaaccaaacaaaaaagtgcattaaattgcgaaaaggattgatccaaggagaaatggcgtaatgaattagcatagaaagcaattagttttatattgcataaataatgatgaattatattaggtaaaggttcaggtggagtggaattcacgtaaagttgataactgagagtcgttagataatttgattgatttgactaaatttttatttaacggctctcgactatcaactttgcgatgcacctgaattttaccattatattagtagcgaagaaaaagaaaagaggttagtagaaaattaaattaaattaataaagaaagcaaatgagaagcgaaggagcgactgagactcagattgcagaaaaagcagagactttgagactcgactcttgagacttcacaacgcctcccttttcctccttctctcagatcgattctctttctcactctctcaactctcttcaaaacccccacaactaactttcttcttcttcttcttcttgttctttgtatatacatacatacatacatacatgtacatgtatctccaccttccatatccaccactcctgacttgctcttaatcttcactgaaacgtcctcgattctcttgcgagcttctgctttagacaacatgatacattccgctgcttcatccatggcctcacccctctgcacgtggctggtggcggcttgcatgtcagtcacgtgccacactgaccgcgccgggactccaaaggcagcgcttcggtcatccaagaggtcaaggaggacgaaggcgctgccagcaaacacctcccaattgaacatgagattgatttcttcgctgtacggatccagcattcagggactcataagctccttcgagccctgcgatgactactgcaacactcacaatgccttctcttcgctctttcgatccacaactcccaatcgcagacacacccgtctcaataaactttctcattctggtaaacttttttttttttttttttaattttccccacttaaatcacctcaagttaagatatttctggtgctttttccaattttaatctgtaccccgtactgggtatttcaatttgattcatttatttatttttcttatattaatataataataatagtttttttttttattatggttatgaaatctgggttctgattttctggtcttctgaaagatatgtgtgtgtgtgtggcttttttcccttttttttctgaattacttaaaatgcacaagtgttagcttggatctttgtcttttggttggtcatcctaataaatggggttgcttggtggtttagtattgagggttgttctacctgctctgtttaactgttttgagggtacggtttataatattaggagtatgggggtagttgttgttctttctttgctgagattgatggtgcacctgtgtcacttaattaggacatgttgctacaacactatgccattgcccatgctttttgctttcatttcccattcctacttcatttccttgtatttatcgtacacccaggtatctacataaagttcccattaaatattggttaatgttcgcatcatcactttgaatttcttgcaatctcgttgaggtagttcagcatattcacagaatcaactcaaacgaaaataaatttcttttgacttttattttgttttcttttcttcatgaaaataataatcacactgaagtgttctgaatttaactactatgtaaatatcgcccccacgtgggattacctctctctccctctctctctagtttttttgtttctctatgtaaaatagtggaggtaagttatgcctttgtcataagcacacataagcaagcagaggctcctttttttttccctcattttttttttaaattatcatgtaaaatagatgattctggcacaaactatccacaagttatttttgctcctagttctttagtatgtttttaacacactggacaagtctactactgctgcatgcatgtccctgactgatgggttctagtattcagtggtcttgagtgttttgatttaaagcaagggatataagttgggtctgatgaatttttcgaggtttcatagcatttttgttaccatagtcttagaattactaccttggataaacctaagcatatttttgttaatggctggggttgcatttcctgctcaacattaatgtgcaacaagggtatgttcggttaggggttaaaaatggggcgggaattttcaatggatgtggcccatgtgtaattttactcatagaacttattgtaaaaatttcaccccatttctacttcccaaaagaacatatccttatcatgatcattggaagctacacatgcatcatgttgaactataagctgtgggccgttctttcttgtatgcttctgatatgaatttcttgaatgataggcggttgcatgcaatttttattattgctgacttattcctctgaattgcaaaactataaggtaaaacaatggctgtagctgtggaacctgcacacgaagtcacagcaacgaaaaaacctcctacaaaacaaaggcgagtagttgtgactgggttgggcgttgttacacctcttggtcacgaaccagatgtttactacaacaatttacttgatggtgttagtggcataagtgagatcgagacatttgattgtgctgaatatccaacggcaagttatatataattgaaattgtatttcttgttgtctgttgtagcttttgttctgaacattctatgtttccagaggattgctggtgagatcaagtcgttctccactgatggctgggttgcaccaaaactttcgaagagaatggataaatttatgctctatttgctaacagccggtaaaaaggccttagttgatggtggaattactgaagatgtaatggatgagttgaataaggaaaaatgtggagttttgattggctcagcgatgggtggcatgaaggtaaccttgtctttctttcatgttgagatacaaatcttgtaaaaccagctggtgttgtggactgaatttaatgccactgcaatccaaatggagcagaagagtgtgctatattttcaaccttgtgcccttgttatattgactatgatttttggtgctttttctactgagcaggtttttaatgatgccattgaagctttaagagtatcatataagaagatgaatcctttttgtgtaccttttgccacaacaaatatgggatctgccatacttgcgatggatctggtctgttgttgcatggacaccataaattggtgttttgtttcgtaccaagcttttattttggctatgttatcttcagcttctaacaaacattgttatcttttatggattacagggatggatgggccctaattattcaatctctacagcttgtgctacaagtaacttttgtatattgaatgcagcaaaccacatcataagaggtgaagctgtaagtattcatgctttgatattactctcaggatggtgaatttttcgtatccaatattataccttatatttatttatttatttaactttctttttctgagacagtttggtgtaatgatatggtcattgcatcaaaatgtattgataaatttaatatgaaaatattcctgcaggatgtgatgctttgtggtggctcagatgctgctattatacccattggtatgatctcaccattgattctttttcacctgcatacggttatcatgccataaatctagctgatatgaatccactaatcattggtacaggtttgggaggttttgtggcatgcagagccctatcgcagaggaatacagaccctaccaaagcttcacgcccttgggacattgtaatacaacagaaatcctgctcattctttctgctactcaccatgtttttgtattgctgatctttcatcattttttatttttgaaacaatacaactataattataatgaaatttgatgaccaagtcttgtatcctttcttgtaatctgaacagcatagtttattaaaaactaacgtttcaatggtcacagatacaacttttccttttgatcacaaaaatgctgtcttctatggatatgagctatggcaaattcagtttttctaattaggctgtttatgatattccagaatcgagatggatttgtcatgggggaaggagctggagttttacttttggaggacttggagcatgcaaaggtacaggaaatcagatctaataattttaaaacagaacatcccttatgaaatgacagtgcatgttttgatattattactgcagacttgtgcatgtaatttatttcatttagtgcatccttcagttccttcaaacagtattaaaggacttcatacatctgttctctcttgaattaggattgattttcaggctgctgcttgtcataaaaattcacctgttacttctcatgtcaaatctgcgcttgacatttgtcttgcaactaaatcaaatgcatctcttttacattgcagaaaagaggtgcaaccatatatgctgaattccttggtggaagcttcacctgtgatgcatatcatgtgacagagccacgtcccgatggtaaaatttcaaatcttttactaccaaaatttaaagcatttatatgcatgctatgattaccatgcttaaaatccgaagagaacaaggcaaaagaatgtgacaagggtatttttatatatttagaatcatgtgtgccatttgctgatggttaggttaagtatttagaaatatactatatgttgcatttttcttgtttggcactggaatttcttcttccgagatcgtgatggaatttaaaactgcatgttgcgatcatgacttgtctcgacaatatggacctgggatttaataatagtcctatattagtggacctagtaatcctagtcataattagtatctgcttgtactttaccacatttcctttatgattccattcggactcttctttgagcattctatgacttgacaaggaatgatttgtatctccctgcggaacttgcattcacgtttctttactccatcttaaataggagctggcgttatactttgcattgaaaaggcattagctcagtctggagtatcaaaagaggatgtgaactacataaatgcacatgccacatccacgcctgctggggatcttaaggagttccaggctctgcttcattgttttggtcaaaatcccgaggtacatctatctttttaagttacaccaccattaacattgttatcttggtgatcaattctatggtaaacatgatctcttgttcttctggagttcatgttgagtaaaacactggtacttctgatatggtcatccatactaatgtctcttgttttttcagctaagagtgaattctacaaaatctatgattggtcatctactaggagcagctggtggcgtggaagctgtagcaacagtccaggtaaaacccaatggatcacttagttcggtaccccgcgattgatgactaaaatggtcctgaggtttgctctctcttcgtacaggcaattaggacagggtgggtgcatcccaatatcaacctggaaaacccagatcaaggagtggtaatgtttcttgttgcatcattccatccttcatttcaaaattttaatcttttaatctaacatgtccaaactaatgacaaaaattcaggatgccaaagtgcttgtgggctcaaagaaagagagactagatatcaaggctgccctatccaattcatttggttttggaggccacaattcttcaatcatatttgctccatataagtgaatgaatacatgtttcagaataataccttccctaccattatacagaaggtatgtctctaaataagaatagatgaccatttgtattcataaaagatgaaaacactgacatatgtatccacactagatcgaaactaaacttgtacccacgcaagatgtcctccgtgtgacaaaagtaccctacgtggcactctaaccctccaaagacaggatacttttgtcacacggagggcatctttcgtgggtacaagtttagtttcgatttaatgtgggtacatatgtcagcattttcatttttcatggatacaaatggtcatttattcctctaaataatgagcaggtcatatcgtcctactacgagaaacacatttaatttggatgactgtggatgttccttgaacatgtattaattgtgtgttgtttttatgttaagttactaaggtagccaggcaattgttacacgcatgatcaaagtcatgaattggatgaaaatatatgtggaaatggttggagcagaaactcttgaggacattgttgagagatggtttggtggtttagattgtctggccgagatacatggtggagccggttctatccaccgatgaagaacatgaatggctgctattgcctttatagagtaggacttgacccactttgatgtgactcttaaacaaatccacttggcagtatatataagctcttctgttttatttattccctatgtacccaaatagtttgtgcaataacaaattgcaatttattttttcgct

SEQ ID NO: 119 - DNA, 7723 bp, synthetic construct
  source 1..7723: mol_type = other DNA
tttgtatcatattcaactactagtttacatatatgagactttttaataaccatcaagagataaaatttaaatatttttattgatcatataatacttaattgtatagttgtgtaaaatactttaaataatactattaatatattaaaatcagctattatatatattgtttaatttatttttaatatatattttatattttaatatgtattttatattaatgtttgattttggtatatacttaataatacagttaatacattgataatattttagactatttttcttaattttattttttaaagttttaatatcaaccgttcatttattaaagataagataaattttgattcatgaatttgatctgataattaatataaattttaaacatgcttaatttgataatgagttttataactataattcaatcaagtcattttgtcacccattcatgaatacttatgtgtgcagataactattaacagctcatcagtagcttgctgatggtaatgtctctcaacttgtttacatgacagttaactaaccgggcatctcataagtattttaatttgtaagcattttattaattagattgattataaaaaaaaattaatttgatttacggttaaaaatagcttttgtttaaaatttaaaagccaaactaactattagatgaaaatttatgagcaaattgagttttaatgctagtttggattggtttttttgagtaaaaattattttttaaaaacaaattaaagtactttttactcaattttaaatttatttagatatatttttttaatttattaaaaaattaaactatttacttttttaaaagttaacaatattttacttttaaaaaaatagaaacaaaaaatatttttaatatttgaaattttttaaaaagcttatttaaatgtaaaataatttaaaaaaataaaaactaaatatttttttaaaaatcaatttaaactgatctttatctcttaattattaactaattttggtatttactttcaaattttcgtaaaaaaaagtcctacaagagaataaagtattattttgactaaagaaaatcttattttgactaaagaaaatctgaaaccaagaataaaataaatgctatgtcacgttttacactaatgataaaaaaattaaagtacaatataagtagaaaatatgttctaaaaccaaacaaaaaagtgcattaaattgcgaaaaggattgatccaaggagaaatggcgtaatgaattagcatagaaagcaattagttttatattgcataaataatgatgaattatattaggtaaaggttcaggtggagtggaattcacgtaaagttgataactgagagtcgttcgataatttgattgatttgactaaatttttatttaacggctctcgactatcaactttgcgatgcacctgaattttaccattatattagtagcgaagaaaaagaaaagaggttagtagaaaattaaattaaattaataaagaaagcaaatgagaagcgaaggagcgactgagactcagattgcagaaaaagcagagactttgagactcgactcttgagacttcacaacgcctcccttttcctccttctctcagatcgattctctttctcactctctcaactctcttcaaaacccccacaactaactttcttcttcttcttcttcttgttctttgtatatacatacatacatacatacatgtacatgtatctccaccttccatatccaccactcctgacttgctcttaatcttcactgaaacgtcctcgattctcttgcgagcttctgctttagacaacatgatacattccgctgcttcatccatggcctcacccctctgcacgtggctggtggcggcttgcatgtcagtcacgtgccacactgaccgcgccgggactccaaaggcagcgcttcggtcatccaagaggtcaaggaggacgaaggcgctgccagcaaacacctcccaattgaacatgagattgatttcttcgctgtacggatccagcattcagggactcataagctccttcgagccctgcgatgactactgcaacactcacaatgccttctcttcgctctttcgatccacaactcccaatcgcagacacacccgtctcaataaactttctcattctggtaaacttttttttttttttttttaattttccccacttaaatcacctcaagttaagatatttctggtgctttttccaattttaatctgtaccccgtactgggtatttcaatttgattcatttatttatttttcttatattaatataataataatagtttttttttttattatggttatgaaatctgggttctgattttctggtcttctgaaagatatgtgtgtgtgtgtggcttttttcccttttttttctgaattacttaaaatgcacaagtgttagcttggatctttgtcttttggttggtcatcctaataaatggggttgcttggtggtttagtattgagggttgttctacctgctctgtttaactgttttgagggtacggtttataatattaggagtatgggggtagttgttgttctttctttgctgagattgatggtgcacctgtgtcacttaattaggacatgttgctacaacactatgccattgcccatgctttttgctttcatttcccattcctacttcatttccttgtatttatcgtacacccaggtatctacataaagttcccattaaatattggttaatgttcgcatcatcactttgaatttcttgcaatctcgttgaggtagttcagcatattcacagaatcaactcaaacgaaaataaatttcttttgacttttattttgttttcttttcttcatgaaaataataatcacactgaagtgttctgaatttaactactatgtaaatatcgcccccacgtgggattacctctctctccctctctctctagtttttttgtttctctatgtaaaatagtggaggtaagttatgcctttgtcataagcacacataagcaagcagaggctcctttttttttccctcattttttttttaaattatcatgtaaaatagatgattctggcacaaactatccacaagttatttttgctcctagttctttagtatgtttttaacacactggacaagtctactactgctgcatgcatgtccctgactgatgggttctagtattcagtggtcttgagtgttttgatttaaagcaagggatataagttgggtctgatgaatttttcgaggtttcatagcatttttgttaccatagtcttagaattactaccttggataaacctaagcatatttttgttaatggctggggttgcatttcctgctcaacattaatgtgcaacaagggtatgttcggttaggggttaaaaatggggcgggaattttcaatggatgtggcccatgtgtaattttactcatagaacttattgtaaaaatttcaccccatttctacttcccaaaagaacatatccttatcatgatcattggaagctacacatgcatcatgttgaactataagctgtgggccgttctttcttgtatgcttctgatatgaatttcttgaatgataggcggttgcatgcaatttttattattgctgacttattcctctgaattgcaaaactataaggtaaaacaatggctgtagctgtggaacctgcacacgaagtcacagcaacgaaaaaacctcctacaaaacaaaggcgagtagttgtgactgggttgggcgttgttacacctcttggtcacgaaccagatgtttactacaacaatttacttgatggtgttagtggcataagtgagatcgagacatttgattgtgctgaatatccaacggcaagttatatataattgaaattgtatttcttgttgtctgttgtagcttttgttctgaacattctatgtttccagaggattgctggtgagatcaagtcgttctccactgatggctgggttgcaccaaaactttcgaagagaatggataaatttatgctctatttgctaacagccggtaaaaaggccttagttgatggtggaattactgaagatgtaatggatgagttgaataaggaaaaatgtggagttttgattggctcagcgatgggtggcatgaaggtaaccttgtctttctttcatgttgagatacaaatcttgtaaaaccagctggtgttgtggactgaatttaatgccactgcaatccaaatggagcagaagagtgtgctatattttcaaccttgtgcccttgttatattgactatgatttttggtgctttttctactgagcaggtttttaatgatgccattgaagctttaagagtatcatataagaagatgaatcctttttgtgtaccttttgccacaacaaatatgggatctgccatacttgcgatggatctggtctgttgttgcatggacaccataaattggtgttttgtttcgtaccaagcttttattttggctatgttatcttcagcttctaacaaacattgttatcttttatggattacagggatggatgggccctaattattcaatctctacagcttgtgctacaagtaacttttgtatattgaatgcagcaaaccacatcataagaggtgaagctgtaagtattcatgctttgatattactctcaggatggtgaatttttcgtatccaatattataccttatatttatttatttatttaactttctttttctgagacagtttggtgtaatgatatggtcattgcatcaaaatgtattgataaatttaatatgaaaatattcctgcaggatgtgatgctttgtggtggctcagatgctgctattatacccattggtatgatctcaccattgattctttttcacctgcatacggttatcatgccataaatctagctgatatgaatccactaatcattggtacaggtttgggaggttttgtggcatgcagagccctatcgcagaggaatacagaccctaccaaagcttcacgcccttgggacattgtaatacaacagaaatcctgctcattctttctgctactcaccatgtttttgtattgctgatctttcatcattttttatttttgaaacaatacaactataattataatgaaatttgatgaccaagtcttgtatcctttcttgtaatctgaacagcatagtttattaaaaactaacgtttcaatggtcacagatacaacttttccttttgatcacaaaaatgctgtcttctatggatatgagctatggcaaattcagtttttctaattaggctgtttatgatattccagaatcgagatggatttgtcatgggggaaggagctggagttttacttttggaggacttggagcatgcaaaggtacaggaaatcagatctaataattttaaaacagaacatcccttatgaaatgacagtgcatgttttgatattattactgcagacttgtgcatgtaatttatttcatttagtgcatccttcagttccttcaaacagtattaaaggacttcatacatctgttctctcttgaattaggattgattttcaggctgctgcttgtcataaaaattcacctgttacttctcatgtcaaatctgcgcttgacatttgtcttgcaactaaatcaaatgcatctcttttacattgcagaaaagaggtgcaaccatatatgctgaattccttggtggaagcttcacctgtgatgcatatcatgtgacagagccacgtcccgatggtaaaatttcaaatcttttactaccaaaatttaaagcatttatatgcatgctatgattaccatgcttaaaatccgaagagaacaaggcaaaagaatgtgacaagggtatttttatatatttagaatcatgtgtgccatttgctgatggttaggttaagtatttagaaatatactatatgttgcatttttcttgtttggcactggaatttcttcttccgagatcgtgatggaatttaaaactgcatgttgcgatcatgacttgtctcgacaatatggacctgggatttaataatagtcctatattagtggacctagtaatcctagtcataattagtatctgcttgtactttaccacatttcctttatgattccattcggactcttctttgagcattctatgacttgacaaggaatgatttgtatctccctgcggaacttgcattcacgtttctttactccatcttaaataggagctggcgttatactttgcattgaaaaggcattagctcagtctggagtatcaaaagaggatgtgaactacataaatgcacatgccacatccacgcctgctggggatcttaaggagttccaggctctgcttcattgttttggtcaaaatcccgaggtacatctatctttttaagttacaccaccattaacattgttatcttggtgatcaattctatggtaaacatgatctcttgttcttctggagttcatgttgagtaaaacactggtacttctgatatggtcatccatactaatgtctcttgttttttcagctaagagtgaattctacaaaatctatgattggtcatctactaggagcagctggtggcgtggaagctgtagcaacagtccaggtaaaacccaatggatcacttagttcggtaccccgcgattgatgactaaaatggtcctgaggtttgctctctcttcgtacaggcaattaggacagggtgggtgcatcccaatatcaacctggaaaacccagatcaaggagtggtaatgtttcttgttgcatcattccatccttcatttcaaaattttaatcttttaatctaacatgtccaaactaatgacaaaaattcaggatgccaaagtgcttgtgggctcaaagaaagagagactagatatcaaggctgccctatccaattcatttggttttggaggccacaattcttcaatcatatttgctccatataagtgaatgaatacatgtttcagaataataccttccctaccattatacagaaggtatgtctctaaataagaatagatgaccatttgtattcataaaagatgaaaacactgacatatgtatccacactagatcgaaactaaacttgtacccacgcaagatgtcctccgtgtgacaaaagtaccctacgtggcactctaaccctccaaagacaggatacttttgtcacacggagggcatctttcgtgggtacaagtttagtttcgatttaatgtgggtacatatgtcagcattttcatttttcatggatacaaatggtcatttattcctctaaataatgagcaggtcatatcgtcctactacgagaaacacatttaatttggatgactgtggatgttccttgaacatgtattaattgtgtgttgtttttatgttaagttactaaggtagccaggcaattgttacacgcatgatcaaagtcatgaattggatgaaaatatatgtggaaatggttggagcagaaactcttgaggacattgttgagagatggtttggtggtttagattgtctggccgagatacatggtggagccggttctatccaccgatgaagaacatgaatggctgctattgcctttatagagtaggacttgacccactttgatgtgactcttaaacaaatccacttggcagtatatataagctcttctgttttatttattccctatgtacccaaatagtttgtgcaataacaaattgcaatttattttttcgct

SEQ ID NO: 120 - DNA, 1647 bp, synthetic construct
  source 1..1647: mol_type = other DNA
atgatacattccgctgcttcatccatggcctcacccctctgcacgtggctggtggcggcttgcatgtcagtcacgtgccacactgaccgcgccgggactccaaaggcagcgcttcggtcatccaagaggtcaaggaggacgaaggcgctgccagcaaacacctcccaattgaacatgagattgatttcttcgctgtacggatccagcattcagggactcataagctccttcgagccctgcgatgactactgcaacactcacaatgccttctcttcgctctttcgatccacaactcccaatcgcagacacacccgtctcaataaactttctcattctggtaaaacaatggctgtagctgtggaacctgcacacgaagtcacagcaacgaaaaaacctcctacaaaacaaaggcgagtagttgtgactgggttgggcgttgttacacctcttggtcacgaaccagatgtttactacaacaatttacttgatggtgttagtggcataagtgagatcgagacatttgattgtgctgaatatccaacgaggattgctggtgagatcaagtcgttctccactgatggctgggttgcaccaaaactttcgaagagaatggataaatttatgctctatttgctaacagccggtaaaaaggccttagttgatggtggaattactgaagatgtaatggatgagttgaataaggaaaaatgtggagttttgattggctcagcgatgggtggcatgaaggtttttaatgatgccattgaagctttaagagtatcatataagaagatgaatcctttttgtgtaccttttgccacaacaaatatgggatctgccatacttgcgatggatctgggatggatgggccctaattattcaatctctacagcttgtgctacaagtaacttttgtatattgaatgcagcaaaccacatcataagaggtgaagctgatgtgatgctttgtggtggctcagatgctgctattatacccattggtttgggaggttttgtggcatgcagagccctatcgcagaggaatacagaccctaccaaagcttcacgcccttgggacattaatcgagatggatttgtcatgggggaaggagctggagttttacttttggaggacttggagcatgcaaagaaaagaggtgcaaccatatatgctgaattccttggtggaagcttcacctgtgatgcatatcatgtgacagagccacgtcccgatggagctggcgttatactttgcattgaaaaggcattagctcagtctggagtatcaaaagaggatgtgaactacataaatgcacatgccacatccacgcctgctggggatcttaaggagttccaggctctgcttcattgttttggtcaaaatcccgagctaagagtgaattctacaaaatctatgattggtcatctactaggagcagctggtggcgtggaagctgtagcaacagtccaggcaattaggacagggtgggtgcatcccaatatcaacctggaaaacccagatcaaggagtggatgccaaagtgcttgtgggctcaaagaaagagagactagatatcaaggctgccctatccaattcatttggttttggaggccacaattcttcaatcatatttgctccatataagtga

SEQ ID NO: 121 - protein, 548 aa, synthetic construct
MIHSAASSMASPLCTWLVAACMSVTCHTDRAGTPKAALRSSKRSRRTKALPANTSQLNMRLISSLYGSSIQGLISSFEPCDDYCNTHNAFSSLFRSTTPNRRHTRLNKLSHSGKTMAVAVEPAHEVTATKKPPTKQRRVVVTGLGVVTPLGHEPDVYYNNLLDGVSGISEIETFDCAEYPTRIAGEIKSFSTDGWVAPKLSKRMDKFMLYLLTAGKKALVDGGITEDVMDELNKEKCGVLIGSAMGGMKVFNDAIEALRVSYKKMNPFCVPFATTNMGSAILAMDLGWMGPNYSISTACATSNFCILNAANHIIRGEADVMLCGGSDAAIIPIGLGGFVACRALSQRNTDPTKASRPWDINRDGFVMGEGAGVLLLEDLEHAKKRGATIYAEFLGGSFTCDAYHVTEPRPDGAGVILCIEKALAQSGVSKEDVNYINAHATSTPAGDLKEFQALLHCFGQNPELRVNSTKSMIGHLLGAAGGVEAVATVQAIRTGWVHPNINLENPDQGVDAKVLVGSKKERLDIKAALSNSFGFGGHNSSIIFAPYK

SEQ ID NO: 122 - DNA, 383 bp, synthetic construct
  source 1..383: mol_type = other DNA
ataagataaattttgattcatgaatttgatctgataattaatataaattttaaacatgcttaatttgataatgagttttataactataattcaatcaagtcattttgtcacccattcatgaatacttatgtgtgcagataactattaacagctcatcagtagcttgctgatggtaatgtctctcaacttgtttacatgacagttaactaaccgggcatctcataagtattttaatttgtaagcattttattaattagattgattataaaaaaaaattaatttgatttacggttaaaaatagcttttgtttaaaatttaaaagccaaactaactattagatgaaaatttatgagcaaattgagttttaatgctagtttggattg

SEQ ID NO: 123 - DNA, 284 bp, synthetic construct
  source 1..284: mol_type = other DNA
taaacatgcttaatttgataatgagttttataactataattcaatcaagtcattttgtcacccattcatgaatacttatgtgtgcagataactattaacagctcatcagtagcttgctgatggtaatgtctctcaacttgtttacatgacagttaactaaccgggcatctcataagtattttaatttgtaagcattttattaattagattgattataaaaaaaaattaatttgatttacggttaaaaatagcttttgtttaaaatttaaaagccaaactaacta

SEQ ID NO: 124 - DNA, 203 bp, synthetic construct
  source 1..203: mol_type = other DNA
tcaatcaagtcattttgtcacccattcatgaatacttatgtgtgcagataactattaacagctcatcagtagcttgctgatggtaatgtctctcaacttgtttacatgacagttaactaaccgggcatctcataagtattttaatttgtaagcattttattaattagattgattataaaaaaaaattaatttgatttacggtt

SEQ ID NO: 125 - DNA, 163 bp, synthetic construct
  source 1..163: mol_type = other DNA
cccattcatgaatacttatgtgtgcagataactattaacagctcatcagtagcttgctgatggtaatgtctctcaacttgtttacatgacagttaactaaccgggcatctcataagtattttaatttgtaagcattttattaattagattgattataaaaaaa

SEQ ID NO: 126 - DNA, 123 bp, synthetic construct
  source 1..123: mol_type = other DNA
tgtgcagataactattaacagctcatcagtagcttgctgatggtaatgtctctcaacttgtttacatgacagttaactaaccgggcatctcataagtattttaatttgtaagcattttattaa

SEQ ID NO: 127 - DNA, 83 bp, synthetic construct
  source 1..83: mol_type = other DNA
gctcatcagtagcttgctgatggtaatgtctctcaacttgtttacatgacagttaactaaccgggcatctcataagtatttta

SEQ ID NO: 128 - DNA, 223 bp, synthetic construct
  source 1..223: mol_type = other DNA
taaacatgcttaatttgataatgagttttataactataattcaatcaagtcattttgtcacccattcatgaatacttatgtgtgcagataactattaacagctcatcagtagcttgctgatggtaatgtctctcaacttgtttacatgacagttaactaaccgggcatctcataagtattttaatttgtaagcattttattaattagattgattataaaaaaa

SEQ ID NO: 129 - DNA, 143 bp, synthetic construct
  source 1..143: mol_type = other DNA
tcaatcaagtcattttgtcacccattcatgaatacttatgtgtgcagataactattaacagctcatcagtagcttgctgatggtaatgtctctcaacttgtttacatgacagttaactaaccgggcatctcataagtatttta

SEQ ID NO: 130 - DNA, 103 bp, synthetic construct
  source 1..103: mol_type = other DNA
cccattcatgaatacttatgtgtgcagataactattaacagctcatcagtagcttgctgatggtaatgtctctcaacttgtttacatgacagttaactaaccg

SEQ ID NO: 131 - DNA, 63 bp, synthetic construct
  source 1..63: mol_type = other DNA
tgtgcagataactattaacagctcatcagtagcttgctgatggtaatgtctctcaacttgttt

SEQ ID NO: 132 - DNA, 224 bp, synthetic construct
  source 1..224: mol_type = other DNA
cccattcatgaatacttatgtgtgcagataactattaacagctcatcagtagcttgctgatggtaatgtctctcaacttgtttacatgacagttaactaaccgggcatctcataagtattttaatttgtaagcattttattaattagattgattataaaaaaaaattaatttgatttacggttaaaaatagcttttgtttaaaatttaaaagccaaactaacta

SEQ ID NO: 133 - DNA, 143 bp, synthetic construct
  source 1..143: mol_type = other DNA
gctcatcagtagcttgctgatggtaatgtctctcaacttgtttacatgacagttaactaaccgggcatctcataagtattttaatttgtaagcattttattaattagattgattataaaaaaaaattaatttgatttacggtt

SEQ ID NO: 134 - DNA, 104 bp, synthetic construct
  source 1..104: mol_type = other DNA
atggtaatgtctctcaacttgtttacatgacagttaactaaccgggcatctcataagtattttaatttgtaagcattttattaattagattgattataaaaaaa

SEQ ID NO: 135 - DNA, 63 bp, synthetic construct
  source 1..63: mol_type = other DNA
tttacatgacagttaactaaccgggcatctcataagtattttaatttgtaagcattttattaa

SEQ ID NO: 136 - DNA, 416 bp, synthetic construct
  source 1..416: mol_type = other DNA
ttgactaaagaaaatcttattttgactaaagaaaatctgaaaccaagaataaaataaatgctatgtcacgttttacactaatgataaaaaaattaaagtacaatataagtagaaaatatgttctaaaaccaaacaaaaaagtgcattaaattgcgaaaaggattgatccaaggagaaatggcgtaatgaattagcatagaaagcaattagttttatattgcataaataatgatgaattatattaggtaaaggttcaggtggagtggaattcacgtaaagttgataactgagagtcgttcgataatttgattgatttgactaaatttttatttaacggctctcgactatcaactttgcgatgcacctgaattttaccattatattagtagcgaagaaaaagaaaagaggttagtaga

SEQ ID NO: 137 - DNA, 316 bp, synthetic construct
  source 1..316: mol_type = other DNA
aaaataaatgctatgtcacgttttacactaatgataaaaaaattaaagtacaatataagtagaaaatatgttctaaaaccaaacaaaaaagtgcattaaattgcgaaaaggattgatccaaggagaaatggcgtaatgaattagcatagaaagcaattagttttatattgcataaataatgatgaattatattaggtaaaggttcaggtggagtggaattcacgtaaagttgataactgagagtcgttcgataatttgattgatttgactaaatttttatttaacggctctcgactatcaactttgcgatgcacct

SEQ ID NO: 138 - DNA, 236 bp, synthetic construct
  source 1..236: mol_type = other DNA
aattaaagtacaatataagtagaaaatatgttctaaaaccaaacaaaaaagtgcattaaattgcgaaaaggattgatccaaggagaaatggcgtaatgaattagcatagaaagcaattagttttatattgcataaataatgatgaattatattaggtaaaggttcaggtggagtggaattcacgtaaagttgataactgagagtcgttcgataatttgattgatttgactaaattt

SEQ ID NO: 139 - DNA, 197 bp, synthetic construct
  source 1..197: mol_type = other DNA
tagaaaatatgttctaaaaccaaacaaaaaagtgcattaaattgcgaaaaggattgatccaaggagaaatggcgtaatgaattagcatagaaagcaattagttttatattgcataaataatgatgaattatattaggtaaaggttcaggtggagtggaattcacgtaaagttgataactgagagtcgttcgataatt

SEQ ID NO: 140 - DNA, 156 bp, synthetic construct
  source 1..156: mol_type = other DNA
aaacaaaaaagtgcattaaattgcgaaaaggattgatccaaggagaaatggcgtaatgaattagcatagaaagcaattagttttatattgcataaataatgatgaattatattaggtaaaggttcaggtggagtggaattcacgtaaagttgataa

SEQ ID NO: 141 - DNA, 116 bp, synthetic construct
  source 1..116: mol_type = other DNA
ttgcgaaaaggattgatccaaggagaaatggcgtaatgaattagcatagaaagcaattagttttatattgcataaataatgatgaattatattaggtaaaggttcaggtggagtgg

SEQ ID NO: 142 - DNA, 223 bp, synthetic construct
  source 1..223: mol_type = other DNA
aaaataaatgctatgtcacgttttacactaatgataaaaaaattaaagtacaatataagtagaaaatatgttctaaaaccaaacaaaaaagtgcattaaattgcgaaaaggattgatccaaggagaaatggcgtaatgaattagcatagaaagcaattagttttatattgcataaataatgatgaattatattaggtaaaggttcaggtggagtggaattcac

SEQ ID NO: 143 - DNA, 143 bp, synthetic construct
  source 1..143: mol_type = other DNA
aattaaagtacaatataagtagaaaatatgttctaaaaccaaacaaaaaagtgcattaaattgcgaaaaggattgatccaaggagaaatggcgtaatgaattagcatagaaagcaattagttttatattgcataaataatgat

SEQ ID NO: 144 - DNA, 104 bp, synthetic construct
  source 1..104: mol_type = other DNA
tagaaaatatgttctaaaaccaaacaaaaaagtgcattaaattgcgaaaaggattgatccaaggagaaatggcgtaatgaattagcatagaaagcaattagttt

SEQ ID NO: 145 - DNA, 286 bp, synthetic construct
  source 1..286: mol_type = other DNA
aaacaaaaaagtgcattaaattgcgaaaaggattgatccaaggagaaatggcgtaatgaattacattaaattgcgaaaaggattgatccaaggagaaatggcgtaatgaattagcatagaaagcaattagttttatattgcataaataatgatgaattatattaggtaaaggttcaggtggagtggaattcacgtaaagttgataactgagagtcgttcgataatttgattgatttgactaaatttttatttaacggctctcgactatcaactttgcgatgcacct

SEQ ID NO: 146 - DNA, 143 bp, synthetic construct
  source 1..143: mol_type = other DNA
taatgaattagcatagaaagcaattagttttatattgcataaataatgatgaattatattaggtaaaggttcaggtggagtggaattcacgtaaagttgataactgagagtcgttcgataatttgattgatttgactaaattt

SEQ ID NO: 147 - DNA, 103 bp, synthetic construct
  source 1..103: mol_type = other DNA
caattagttttatattgcataaataatgatgaattatattaggtaaaggttcaggtggagtggaattcacgtaaagttgataactgagagtcgttcgataatt

SEQ ID NO: 148 - DNA, 63 bp, synthetic construct
  source 1..63: mol_type = other DNA
aaataatgatgaattatattaggtaaaggttcaggtggagtggaattcacgtaaagttgataa

SEQ ID NO: 149 - DNA, 455 bp, synthetic construct
  source 1..455: mol_type = other DNA
tcttaatcttcactgaaacgtcctcgattctcttgcgagcttctgctttagacaacatgatacattccgctgcttcatccatggcctcacccctctgcacgtggctggtggcggcttgcatgtcagtcacgtgccacactgaccgcgccgggactccaaaggcagcgcttcggtcatccaagaggtcaaggaggacgaaggcgctgccagcaaacacctcccaattgaacatgagattgatttcttcgctgtacggatccagcattcagggactcataagctccttcgagccctgcgatgactactgcaacactcacaatgccttctcttcgctctttcgatccacaactcccaatcgcagacacacccgtctcaataaactttctcattctggtaaacttttttttttttttttttaattttccccacttaaatcacctcaagttaagatattt

SEQ ID NO: 150 - DNA, 355 bp, synthetic construct
  source 1..355: mol_type = other DNA
gacaacatgatacattccgctgcttcatccatggcctcacccctctgcacgtggctggtggcggcttgcatgtcagtcacgtgccacactgaccgcgccgggactccaaaggcagcgcttcggtcatccaagaggtcaaggaggacgaaggcgctgccagcaaacacctcccaattgaacatgagattgatttcttcgctgtacggatccagcattcagggactcataagctccttcgagccctgcgatgactactgcaacactcacaatgccttctcttcgctctttcgatccacaactcccaatcgcagacacacccgtctcaataaactttctcattctggtaaactttttt

SEQ ID NO: 151 - DNA, 275 bp, synthetic construct
  source 1..275: mol_type = other DNA
ccctctgcacgtggctggtggcggcttgcatgtcagtcacgtgccacactgaccgcgccgggactccaaaggcagcgcttcggtcatccaagaggtcaaggaggacgaaggcgctgccagcaaacacctcccaattgaacatgagattgatttcttcgctgtacggatccagcattcagggactcataagctccttcgagccctgcgatgactactgcaacactcacaatgccttctcttcgctctttcgatccacaactcccaatcgcagacac

SEQ ID NO: 152 - DNA, 235 bp, synthetic construct
  source 1..235: mol_type = other DNA
gcggcttgcatgtcagtcacgtgccacactgaccgcgccgggactccaaaggcagcgcttcggtcatccaagaggtcaaggaggacgaaggcgctgccagcaaacacctcccaattgaacatgagattgatttcttcgctgtacggatccagcattcagggactcataagctccttcgagccctgcgatgactactgcaacactcacaatgccttctcttcgctctttcgatcca

SEQ ID NO: 153 - DNA, 195 bp, synthetic construct
  source 1..195: mol_type = other DNA
gtgccacactgaccgcgccgggactccaaaggcagcgcttcggtcatccaagaggtcaaggaggacgaaggcgctgccagcaaacacctcccaattgaacatgagattgatttcttcgctgtacggatccagcattcagggactcataagctccttcgagccctgcgatgactactgcaacactcacaatgcctt

SEQ ID NO: 154 - DNA, 155 bp, synthetic construct
  source 1..155: mol_type = other DNA
ggactccaaaggcagcgcttcggtcatccaagaggtcaaggaggacgaaggcgctgccagcaaacacctcccaattgaacatgagattgatttcttcgctgtacggatccagcattcagggactcataagctccttcgagccctgcgatgactac

SEQ ID NO: 155 - DNA, 120 bp, synthetic construct
  source 1..120: mol_type = other DNA
ggactccaaaggcagcgcttcggtcatccaagaggtcaaggaggacgaaggcgctgccagcaaacacctcccaattgaacatgagattgatttcttcgctgtacggatccagcattcagg

SEQ ID NO: 156 - DNA, 58 bp, synthetic construct
  source 1..58: mol_type = other DNA
gctgtacggatccagcattcagggactcataagctccttcgagccctgcgatgactac

SEQ ID NO: 157 - DNA, 224 bp, synthetic construct
  source 1..224: mol_type = other DNA
gacaacatgatacattccgctgcttcatccatggcctcacccctctgcacgtggctggtggcggcttgcatgtcagtcacgtgccacactgaccgcgccgggactccaaaggcagcgcttcggtcatccaagaggtcaaggaggacgaaggcgctgccagcaaacacctcccaattgaacatgagattgatttcttcgctgtacggatccagcattcagggact

SEQ ID NO: 158 - DNA, 143 bp, synthetic construct
  source 1..143: mol_type = other DNA
ccctctgcacgtggctggtggcggcttgcatgtcagtcacgtgccacactgaccgcgccgggactccaaaggcagcgcttcggtcatccaagaggtcaaggaggacgaaggcgctgccagcaaacacctcccaattgaacatg

SEQ ID NO: 159 - DNA, 103 bp, synthetic construct
  source 1..103: mol_type = other DNA
gcggcttgcatgtcagtcacgtgccacactgaccgcgccgggactccaaaggcagcgcttcggtcatccaagaggtcaaggaggacgaaggcgctgccagcaa

SEQ ID NO: 160 - DNA, 63 bp, synthetic construct
  source 1..63: mol_type = other DNA
gtgccacactgaccgcgccgggactccaaaggcagcgcttcggtcatccaagaggtcaaggag

SEQ ID NO: 161 - DNA, 223 bp, synthetic construct
  source 1..223: mol_type = other DNA
ccgggactccaaaggcagcgcttcggtcatccaagaggtcaaggaggacgaaggcgctgccagcaaacacctcccaattgaacatgagattgatttcttcgctgtacggatccagcattcagggactcataagctccttcgagccctgcgatgactactgcaacactcacaatgccttctcttcgctctttcgatccacaactcccaatcgcagacacacccg

SEQ ID NO: 162 - DNA, 143 bp, synthetic construct
  source 1..143: mol_type = other DNA
aaggaggacgaaggcgctgccagcaaacacctcccaattgaacatgagattgatttcttcgctgtacggatccagcattcagggactcataagctccttcgagccctgcgatgactactgcaacactcacaatgccttctctt

SEQ ID NO: 163 - DNA, 103 bp, synthetic construct
  source 1..103: mol_type = other DNA
cagcaaacacctcccaattgaacatgagattgatttcttcgctgtacggatccagcattcagggactcataagctccttcgagccctgcgatgactactgcaa

SEQ ID NO: 164 - DNA, 63 bp, synthetic construct
  source 1..63: mol_type = other DNA
aacatgagattgatttcttcgctgtacggatccagcattcagggactcataagctccttcgag

SEQ ID NO: 165 - DNA, 223 bp, synthetic construct
  source 1..223: mol_type = other DNA
aggtcaaggaggacgaaggcgctgccagcaaacacctcccaattgaacatgagattgatttcttcgctgtacggatccagcattcagggactcataagctccttcgagccctgcgatgactactgcaacactcacaatgccttctcttcgctctttcgatccacaactcccaatcgcagacacacccgtctcaataaactttctcattctggtaaactttttt

SEQ ID NO: 166 - DNA, 144 bp, synthetic construct
  source 1..144: mol_type = other DNA
aattgaacatgagattgatttcttcgctgtacggatccagcattcagggactcataagctccttcgagccctgcgatgactactgcaacactcacaatgccttctcttcgctctttcgatccacaactcccaatcgcagacaca

SEQ ID NO: 167 - DNA, 103 bp, synthetic construct
  source 1..103: mol_type = other DNA
tcttcgctgtacggatccagcattcagggactcataagctccttcgagccctgcgatgactactgcaacactcacaatgccttctcttcgctctttcgatcca

SEQ ID NO: 168 - DNA, 63 bp, synthetic construct; identical to SEQ ID NO: 51
  source 1..63: mol_type = other DNA

SEQ ID NO: 169 - DNA, 381 bp, synthetic construct
  source 1..381: mol_type = other DNA
gaatttcttgaatgataggcggttgcatgcaatttttattattgctgacttattcctctgaattgcaaaactataaggtaaaacaatggctgtagctgtggaacctgcacacgaagtcacagcaacgaaaaaacctcctacaaaacaaaggcgagtagttgtgactgggttgggcgttgttacacctcttggtcacgaaccagatgtttactacaacaatttacttgatggtgttagtggcataagtgagatcgagacatttgattgtgctgaatatccaacggcaagttatatataattgaaattgtatttcttgttgtctgttgtagcttttgttctgaacattctatgtttccagaggattgctggtgagatcaagtc

SEQ ID NO: 170 - DNA, 281 bp, synthetic construct
  source 1..281: mol_type = other DNA
tattcctctgaattgcaaaactataaggtaaaacaatggctgtagctgtggaacctgcacacgaagtcacagcaacgaaaaaacctcctacaaaacaaaggcgagtagttgtgactgggttgggcgttgttacacctcttggtcacgaaccagatgtttactacaacaatttacttgatggtgttagtggcataagtgagatcgagacatttgattgtgctgaatatccaacggcaagttatatataattgaaattgtatttcttgttgtctgttgtagct

SEQ ID NO: 171 - DNA, 143 bp, synthetic construct
  source 1..143: mol_type = other DNA
tgtagctgtggaacctgcacacgaagtcacagcaacgaaaaaacctcctacaaaacaaaggcgagtagttgtgactgggttgggcgttgttacacctcttggtcacgaaccagatgtttactacaacaatttacttgatggtg

SEQ ID NO: 172 - DNA, 103 bp, synthetic construct
  source 1..103: mol_type = other DNA
acgaagtcacagcaacgaaaaaacctcctacaaaacaaaggcgagtagttgtgactgggttgggcgttgttacacctcttggtcacgaaccagatgtttacta

SEQ ID NO: 173 - DNA, 63 bp, synthetic construct
  source 1..63: mol_type = other DNA
aaacctcctacaaaacaaaggcgagtagttgtgactgggttgggcgttgttacacctcttggt

SEQ ID NO: 174 - DNA, 81 bp, synthetic construct
  source 1..81: mol_type = other DNA
gcgagtagttgtgactgggttgggcgttgttacacctcttggtcacgaaccagatgtttactacaacaatttacttgatgg

SEQ ID NO: 175 - DNA, 57 bp, synthetic construct
  source 1..57: mol_type = other DNA
gcgagtagttgtgactgggttgggcgttgttacacctcttggtcacgaaccagatgt

SEQ ID NO: 176 - DNA, 47 bp, synthetic construct
  source 1..47: mol_type = other DNA
cctcttggtcacgaaccagatgtttactacaacaatttacttgatgg

SEQ ID NO: 177 - DNA, 223 bp, synthetic construct
  source 1..223: mol_type = other DNA
tattcctctgaattgcaaaactataaggtaaaacaatggctgtagctgtggaacctgcacacgaagtcacagcaacgaaaaaacctcctacaaaacaaaggcgagtagttgtgactgggttgggcgttgttacacctcttggtcacgaaccagatgtttactacaacaatttacttgatggtgttagtggcataagtgagatcgagacatttgattgtgctga

SEQ ID NO: 178 - DNA, 143 bp, synthetic construct; identical to SEQ ID NO: 171
  source 1..143: mol_type = other DNA

SEQ ID NO: 179 - DNA, 103 bp, synthetic construct; identical to SEQ ID NO: 172
  source 1..103: mol_type = other DNA

SEQ ID NO: 180 - DNA, 63 bp, synthetic construct; identical to SEQ ID NO: 173
  source 1..63: mol_type = other DNA

SEQ ID NO: 181 - DNA, 223 bp, synthetic construct
  source 1..223: mol_type = other DNA
aatggctgtagctgtggaacctgcacacgaagtcacagcaacgaaaaaacctcctacaaaacaaaggcgagtagttgtgactgggttgggcgttgttacacctcttggtcacgaaccagatgtttactacaacaatttacttgatggtgttagtggcataagtgagatcgagacatttgattgtgctgaatatccaacggcaagttatatataattgaaattg

SEQ ID NO: 182 - DNA, 143 bp, synthetic construct
  source 1..143: mol_type = other DNA
acgaaaaaacctcctacaaaacaaaggcgagtagttgtgactgggttgggcgttgttacacctcttggtcacgaaccagatgtttactacaacaatttacttgatggtgttagtggcataagtgagatcgagacatttgattg

SEQ ID NO: 183 - DNA, 103 bp, synthetic construct
  source 1..103: mol_type = other DNA
acaaaggcgagtagttgtgactgggttgggcgttgttacacctcttggtcacgaaccagatgtttactacaacaatttacttgatggtgttagtggcataagt

SEQ ID NO: 184 - DNA, 63 bp, synthetic construct
  source 1..63: mol_type = other DNA
ctgggttgggcgttgttacacctcttggtcacgaaccagatgtttactacaacaatttacttg

SEQ ID NO: 185 - DNA, 223 bp, synthetic construct
  source 1..223: mol_type = other DNA
acacgaagtcacagcaacgaaaaaacctcctacaaaacaaaggcgagtagttgtgactgggttgggcgttgttacacctcttggtcacgaaccagatgtttactacaacaatttacttgatggtgttagtggcataagtgagatcgagacatttgattgtgctgaatatccaacggcaagttatatataattgaaattgtatttcttgttgtctgttgtagct

SEQ ID NO: 186 - DNA, 143 bp, synthetic construct
  source 1..143: mol_type = other DNA
aggcgagtagttgtgactgggttgggcgttgttacacctcttggtcacgaaccagatgtttactacaacaatttacttgatggtgttagtggcataagtgagatcgagacatttgattgtgctgaatatccaacggcaagtta

SEQ ID NO: 187 - DNA, 103 bp, synthetic construct
  source 1..103: mol_type = other DNA
gttgggcgttgttacacctcttggtcacgaaccagatgtttactacaacaatttacttgatggtgttagtggcataagtgagatcgagacatttgattgtgct

SEQ ID NO: 188 - DNA, 63 bp, synthetic construct
  source 1..63: mol_type = other DNA
ttggtcacgaaccagatgtttactacaacaatttacttgatggtgttagtggcataagtgaga

SEQ ID NO: 189 - DNA, 354 bp, synthetic construct
  source 1..354: mol_type = other DNA
agttatatataattgaaattgtatttcttgttgtctgttgtagcttttgttctgaacattctatgtttccagaggattgctggtgagatcaagtcgttctccactgatggctgggttgcaccaaaactttcgaagagaatggataaatttatgctctatttgctaacagccggtaaaaaggccttagttgatggtggaattactgaagatgtaatggatgagttgaataaggaaaaatgtggagttttgattggctcagcgatgggtggcatgaaggtaaccttgtctttctttcatgttgagatacaaatcttgtaaaaccagctggtgttgtggactgaatttaatgccact

SEQ ID NO: 190 - DNA, 254 bp, synthetic construct
  source 1..254: mol_type = other DNA
tctgaacattctatgtttccagaggattgctggtgagatcaagtcgttctccactgatggctgggttgcaccaaaactttcgaagagaatggataaatttatgctctatttgctaacagccggtaaaaaggccttagttgatggtggaattactgaagatgtaatggatgagttgaataaggaaaaatgtggagttttgattggctcagcgatgggtggcatgaaggtaaccttgtctttctttcatgttgaga

SEQ ID NO: 191 - DNA, 174 bp, synthetic construct
  source 1..174: mol_type = other DNA
aagtcgttctccactgatggctgggttgcaccaaaactttcgaagagaatggataaatttatgctctatttgctaacagccggtaaaaaggccttagttgatggtggaattactgaagatgtaatggatgagttgaataaggaaaaatgtggagttttgattggctcagcgatg

SEQ ID NO: 192 - DNA, 134 bp, synthetic construct
  source 1..134: mol_type = other DNA
ctgggttgcaccaaaactttcgaagagaatggataaatttatgctctatttgctaacagccggtaaaaaggccttagttgatggtggaattactgaagatgtaatggatgagttgaataaggaaaaatgtggag

SEQ ID NO: 193 - DNA, 94 bp, synthetic construct
  source 1..94: mol_type = other DNA
cgaagagaatggataaatttatgctctatttgctaacagccggtaaaaaggccttagttgatggtggaattactgaagatgtaatggatgagtt

SEQ ID NO: 194 - DNA, 54 bp, synthetic construct
  source 1..54: mol_type = other DNA
atgctctatttgctaacagccggtaaaaaggccttagttgatggtggaattact

SEQ ID NO: 195 - DNA, 44 bp, synthetic construct
  source 1..44: mol_type = other DNA
atgctctatttgctaacagccggtaaaaaggccttagttgatgg

SEQ ID NO: 196 - DNA, 31 bp, synthetic construct
  source 1..31: mol_type = other DNA
taaaaaggccttagttgatggtggaattact

SEQ ID NO: 197 - DNA, 223 bp, synthetic construct
  source 1..223: mol_type = other DNA
tctgaacattctatgtttccagaggattgctggtgagatcaagtcgttctccactgatggctgggttgcaccaaaactttcgaagagaatggataaatttatgctctatttgctaacagccggtaaaaaggccttagttgatggtggaattactgaagatgtaatggatgagttgaataaggaaaaatgtggagttttgattggctcagcgatgggtggcatg

SEQ ID NO: 198 - DNA, 143 bp, synthetic construct
  source 1..143: mol_type = other DNA
aagtcgttctccactgatggctgggttgcaccaaaactttcgaagagaatggataaatttatgctctatttgctaacagccggtaaaaaggccttagttgatggtggaattactgaagatgtaatggatgagttgaataagga

SEQ ID NO: 199 - DNA, 103 bp, synthetic construct
  source 1..103: mol_type = other DNA
ctgggttgcaccaaaactttcgaagagaatggataaatttatgctctatttgctaacagccggtaaaaaggccttagttgatggtggaattactgaagatgta

SEQ ID NO: 200 - DNA, 63 bp, synthetic construct
  source 1..63: mol_type = other DNA
cgaagagaatggataaatttatgctctatttgctaacagccggtaaaaaggccttagttgatg

SEQ ID NO: 201 - DNA, 221 bp, synthetic construct
  source 1..221: mol_type = other DNA
ggattgctggtgagatcaagtcgttctccactgatggctgggttgcaccaaaactttcgaagagaatggataaatttatgctctatttgctaacagccggtaaaaaggccttagttgatggtggaattactgaagatgtaatggatgagttgaataaggaaaaatgtggagttttgattggctcagcgatgggtggcatgaaggtaaccttgtctttcttt

SEQ ID NO: 202 - DNA, 141 bp, synthetic construct
  source 1..141: mol_type = other DNA
ggttgcaccaaaactttcgaagagaatggataaatttatgctctatttgctaacagccggtaaaaaggccttagttgatggtggaattactgaagatgtaatggatgagttgaataaggaaaaatgtggagttttgattgg

SEQ ID NO: 203 - DNA, 101 bp, synthetic construct
  source 1..101: mol_type = other DNA
agagaatggataaatttatgctctatttgctaacagccggtaaaaaggccttagttgatggtggaattactgaagatgtaatggatgagttgaataaggaa

SEQ ID NO: 204 - DNA, 61 bp, synthetic construct
  source 1..61: mol_type = other DNA
ctctatttgctaacagccggtaaaaaggccttagttgatggtggaattactgaagatgtaa

SEQ ID NO: 205 - DNA, 223 bp, synthetic construct
  source 1..223: mol_type = other DNA
ggtgagatcaagtcgttctccactgatggctgggttgcaccaaaactttcgaagagaatggataaatttatgctctatttgctaacagccggtaaaaaggccttagttgatggtggaattactgaagatgtaatggatgagttgaataaggaaaaatgtggagttttgattggctcagcgatgggtggcatgaaggtaaccttgtctttctttcatgttgaga

SEQ ID NO: 206 - DNA, 144 bp, synthetic construct
  source 1..144: mol_type = other DNA
ccaaaactttcgaagagaatggataaatttatgctctatttgctaacagccggtaaaaaggccttagttgatggtggaattactgaagatgtaatggatgagttgaataaggaaaaatgtggagttttgattggctcagcgatg

SEQ ID NO: 207 - DNA, 104 bp, synthetic construct
  source 1..104: mol_type = other DNA
ggataaatttatgctctatttgctaacagccggtaaaaaggccttagttgatggtggaattactgaagatgtaatggatgagttgaataaggaaaaatgtggag

SEQ ID NO: 208 - DNA, 63 bp, synthetic construct
  source 1..63: mol_type = other DNA
gctaacagccggtaaaaaggccttagttgatggtggaattactgaagatgtaatggatgagtt

SEQ ID NO: 209 - DNA, 254 bp, synthetic construct
  source 1..254: mol_type = other DNA
ctcaccattgattctttttcacctgcatacggttatcatgccataaatctagctgatatgaatccactaatcattggtacaggtttgggaggttttgtggcatgcagagccctatcgcagaggaatacagaccctaccaaagcttcacgcccttgggacattgtaatacaacagaaatcctgctcattctttctgctactcaccatgtttttgtattgctgatctttcatcattttttatttttgaaacaatac

SEQ ID NO: 210 - DNA, 175 bp, synthetic construct
  source 1..175: mol_type = other DNA
gccataaatctagctgatatgaatccactaatcattggtacaggtttgggaggttttgtggcatgcagagccctatcgcagaggaatacagaccctaccaaagcttcacgcccttgggacattgtaatacaacagaaatcctgctcattctttctgctactcaccatgtttttgt

SEQ ID NO: 211 - DNA, 134 bp, synthetic construct
  source 1..134: mol_type = other DNA
aatccactaatcattggtacaggtttgggaggttttgtggcatgcagagccctatcgcagaggaatacagaccctaccaaagcttcacgcccttgggacattgtaatacaacagaaatcctgctcattctttct

SEQ ID NO: 212 - DNA, 94 bp, synthetic construct
  source 1..94: mol_type = other DNA
aggtttgggaggttttgtggcatgcagagccctatcgcagaggaatacagaccctaccaaagcttcacgcccttgggacattgtaatacaacag

SEQ ID NO: 213 - DNA, 23 bp, synthetic construct
  source 1..23: mol_type = other DNA
ggactccaaaggcagcgcttcgg

SEQ ID NO: 214 - DNA, 23 bp, synthetic construct
  source 1..23: mol_type = other DNA
gctgtacggatccagcattcagg

SEQ ID NO: 215 - DNA, 23 bp, synthetic construct
  source 1..23: mol_type = other DNA
gcgagtagttgtgactgggttgg

SEQ ID NO: 216 - DNA, 23 bp, synthetic construct
  source 1..23: mol_type = other DNA
tactacaacaatttacttgatgg

SEQ ID NO: 217 - DNA, 23 bp, synthetic construct
  source 1..23: mol_type = other DNA
ggtaaaaaggccttagttgatgg

SEQ ID NO: 218 - DNA, 23 bp, synthetic construct
  source 1..23: mol_type = other DNA
atgctctatttgctaacagccgg

SEQ ID NO: 219 - DNA, 23 bp, synthetic construct
  source 1..23: mol_type = other DNA
agtaattccaccatcaactaagg

SEQ ID NO: 220 - DNA, 23 bp, synthetic construct
  source 1..23: mol_type = other DNA
gctcatcagtagcttgctgatgg

SEQ ID NO: 221 - DNA, 23 bp, synthetic construct
  source 1..23: mol_type = other DNA
taaaatacttatgagatgcccgg

SEQ ID NO: 222 - DNA, 23 bp, synthetic construct
  source 1..23: mol_type = other DNA
ttgcgaaaaggattgatccaagg

SEQ ID NO: 223 - DNA, 23 bp, synthetic construct
  source 1..23: mol_type = other DNA
aggtaaaggttcaggtggagtgg

SEQ ID NO: 224 - DNA, 22 bp, synthetic construct
  source 1..22: mol_type = other DNA
aaaacaatggctgtagctgtgg

SEQ ID NO: 225 - DNA, 21 bp, synthetic construct
  source 1..21: mol_type = other DNA
aaacaatggctgtagctgtgg

SEQ ID NO: 226 - DNA, 22 bp, synthetic construct
  source 1..22: mol_type = other DNA
cgttggatattcagcacaatca

SEQ ID NO: 227 - DNA, 21 bp, synthetic construct
  source 1..21: mol_type = other DNA
caatggctgtagctgtggaac

SEQ ID NO: 228 - DNA, 22 bp, synthetic construct
  source 1..22: mol_type = other DNA
aggattgctggtgagatcaagt

SEQ ID NO: 229 - DNA, 22 bp, synthetic construct
  source 1..22: mol_type = other DNA
gccaatcaaaactccacatttt

SEQ ID NO: 230 - DNA, 22 bp, synthetic construct
  source 1..22: mol_type = other DNA
atcaagtcgttctccactgatg

SEQ ID NO: 231 - DNA, 22 bp, synthetic construct
  source 1..22: mol_type = other DNA
agccaatcaaaactccacattt

SEQ ID NO: 232 - DNA, 22 bp, synthetic construct
  source 1..22: mol_type = other DNA
caagtcattttgtcacccattc

SEQ ID NO: 233 - DNA, 22 bp, synthetic construct
  source 1..22: mol_type = other DNA
gcattaaaactcaatttgctca

SEQ ID NO: 234 - DNA, 22 bp, synthetic construct
  source 1..22: mol_type = other DNA
caatcaagtcattttgtcaccc

SEQ ID NO: 235 - DNA, 25 bp, synthetic construct
  source 1..25: mol_type = other DNA
tttgactaaagaaaatctgaaacca

SEQ ID NO: 236 - DNA, 22 bp, synthetic construct
  source 1..22: mol_type = other DNA
tccactccacctgaacctttac

SEQ ID NO: 237 - DNA, 22 bp, synthetic construct
  source 1..22: mol_type = other DNA
agtgcattaaattgcgaaaagg

SEQ ID NO: 238 - DNA, 22 bp, synthetic construct
  source 1..22: mol_type = other DNA
catcgcaaagttgatagtcgag

SEQ ID NO: 239 - DNA, 22 bp, synthetic construct
  source 1..22: mol_type = other DNA
tttcacctgcatacggttatca

SEQ ID NO: 240 - DNA, 22 bp, synthetic construct
  source 1..22: mol_type = other DNA
gcagaaagaatgagcaggattt

SEQ ID NO: 241 - DNA, 22 bp, synthetic construct
  source 1..22: mol_type = other DNA
agcagaaagaatgagcaggatt

SEQ ID NO: 242 - DNA, 23 bp, synthetic construct
  source 1..23: mol_type = other DNA
gaaactgacctttaatagaaagg

SEQ ID NO: 243 - DNA, 23 bp, synthetic construct
  source 1..23: mol_type = other DNA
ccccttttccctaaatggtctgg

SEQ ID NO: 244 - DNA, 23 bp, synthetic construct
  source 1..23: mol_type = other DNA
ggggatgattttaatctctcagg

SEQ ID NO: 245 - DNA, 23 bp, synthetic construct
  source 1..23: mol_type = other DNA
accattttagtcacaaaacgcgg

SEQ ID NO: 246 - DNA, 23 bp, synthetic construct
  source 1..23: mol_type = other DNA
aacatgaatgcaagttccgcagg

SEQ ID NO: 247 - DNA, 23 bp, synthetic construct
  source 1..23: mol_type = other DNA
tcgcattaaacatgagcaatatc

SEQ ID NO: 248 - DNA, 22 bp, synthetic construct
  source 1..22: mol_type = other DNA
gctgttaaggaaaaaccaaaga

SEQ ID NO: 249 - DNA, 22 bp, synthetic construct
  source 1..22: mol_type = other DNA
agaaaggcggcaaataaatctc

SEQ ID NO: 250 - DNA, 22 bp, synthetic construct
  source 1..22: mol_type = other DNA
ttaccactccttgatctgggtt

SEQ ID NO: 251 - DNA, 22 bp, synthetic construct
  source 1..22: mol_type = other DNA
tcaatgcaaagtataacgccag

SEQ ID NO: 252 - DNA, 23 bp, synthetic construct
  source 1..23: mol_type = other DNA
caggaaatgccctctttagtttt

SEQ ID NO: 253 - DNA, 23 bp, synthetic construct
  source 1..23: mol_type = other DNA
tgtacccaaatagtttgtgcaat

SEQ ID NO: 254 - DNA, 22 bp, synthetic construct
  source 1..22: mol_type = other DNA
tttttcgctgccttgttctatt

SEQ ID NO: 255 - DNA, 22 bp, synthetic construct
  source 1..22: mol_type = other DNA
aagctgtagcaacagtccaggt

SEQ ID NO: 256 - DNA, 22 bp, synthetic construct
  source 1..22: mol_type = other DNA
ttgtctcgacaatatggacctg

SEQ ID NO: 257 - DNA, 23 bp, synthetic construct
  source 1..23: mol_type = other DNA
ttaacctaaccatcagcaaatgg

SEQ ID NO: 258 - DNA, 23 bp, synthetic construct
  source 1..23: mol_type = other DNA
gactcttaaacaaatccacttgg

SEQ ID NO: 259 - DNA, 23 bp, synthetic construct
  source 1..23: mol_type = other DNA
gttaagttactaaggtagccagg

SEQ ID NO: 260 - DNA, 23 bp, synthetic construct
  source 1..23: mol_type = other DNA
ggttggagcagaaactcttgagg

SEQ ID NO: 261 - DNA, 23 bp, synthetic construct
  source 1..23: mol_type = other DNA
atgacctgctcattatttagagg

SEQ ID NO: 262 - DNA, 23 bp, synthetic construct
  source 1..23: mol_type = other DNA
ggtttgctctctcttcgtacagg

SEQ ID NO: 263 - DNA, 22 bp, synthetic construct
  source 1..22: mol_type = other DNA
aagaagaaattccagtgccaaa

SEQ ID NO: 264 - DNA, 22 bp, synthetic construct
  source 1..22: mol_type = other DNA
aatagaacaaggcagcgaaaaa

SEQ ID NO: 265 - DNA, 22 bp, synthetic construct
  source 1..22: mol_type = other DNA
tcacatcaaagtgggtcaagtc

SEQ ID NO: 266 - DNA, 22 bp, synthetic construct
  source 1..22: mol_type = other DNA
tcacatcaaagtgggtcaagtc

SEQ ID NO: 267 - DNA, 22 bp, synthetic construct
  source 1..22: mol_type = other DNA
tgttcaaggaacatccacagtc

SEQ ID NO: 268 - DNA, 22 bp, synthetic construct
  source 1..22: mol_type = other DNA
gatggaatgatgcaacaagaaa

SEQ ID NO: 269 - DNA, 22 bp, synthetic construct
  source 1..22: mol_type = other DNA
atgcatatcatgtgacagagcc

SEQ ID NO: 270 - DNA, 22 bp, synthetic construct
  source 1..22: mol_type = other DNA
gaaatggttggagcagaaactc

SEQ ID NO: 271 - DNA, 22 bp, synthetic construct
  source 1..22: mol_type = other DNA
gatgactgtggatgttccttga

SEQ ID NO: 272 - DNA, 22 bp, synthetic construct
  source 1..22: mol_type = other DNA
gatgactgtggatgttccttga

SEQ ID NO: 273 - DNA, 22 bp, synthetic construct
  source 1..22: mol_type = other DNA
caggatacttttgtcacacgga

SEQ ID NO: 274 - DNA, 22 bp, synthetic construct
  source 1..22: mol_type = other DNA
ccaatggatcacttagttcggt

SEQ ID NO: 275 - DNA, 223 bp, synthetic construct
  source 1..223: mol_type = other DNA
ataattagtatctgcttgtactttaccacatttcctttatgattccattcggactcttctttgagtattctatgacttgacaaggaatgatttgtatctccctgcggaacttgcattcatgtttctttactccatcttaaataggagctggcgttatactttgcattgaaaaggcattagctcagtctggagtatcaaaagaggatgtgaactacataaatgc

SEQ ID NO: 276 - DNA, 143 bp, synthetic construct
  source 1..143: mol_type = other DNA
gattccattcggactcttctttgagtattctatgacttgacaaggaatgatttgtatctccctgcggaacttgcattcatgtttctttactccatcttaaataggagctggcgttatactttgcattgaaaaggcattagctc

SEQ ID NO: 277 - DNA, 103 bp, synthetic construct
  source 1..103: mol_type = other DNA
ttgagtattctatgacttgacaaggaatgatttgtatctccctgcggaacttgcattcatgtttctttactccatcttaaataggagctggcgttatactttg

SEQ ID NO: 278 - DNA, 63 bp, synthetic construct
  source 1..63: mol_type = other DNA
caaggaatgatttgtatctccctgcggaacttgcattcatgtttctttactccatcttaaata

SEQ ID NO: 279 - DNA, 223 bp, synthetic construct
  source 1..223: mol_type = other DNA
ctacaaaatctatgattggtcatctactaggagcagctggtggcgtggaagctgtagcaacagtccaggtaaaacccaatggatcacttagttcggtaccccgcgttttgtgactaaaatggtcctgaggtttgctctctctttatacaggcaattaggacagggtgggtgcatcccaatatcaacctggaaaacccagatcaaggagtggtaatgtttcttg

SEQ ID NO: 280 - DNA, 143 bp, synthetic construct
  source 1..143: mol_type = other DNA
tggcgtggaagctgtagcaacagtccaggtaaaacccaatggatcacttagttcggtaccccgcgttttgtgactaaaatggtcctgaggtttgctctctctttatacaggcaattaggacagggtgggtgcatcccaatatc

SEQ ID NO: 281 - DNA, 103 bp, synthetic construct
  source 1..103: mol_type = other DNA
cagtccaggtaaaacccaatggatcacttagttcggtaccccgcgttttgtgactaaaatggtcctgaggtttgctctctctttatacaggcaattaggacag

SEQ ID NO: 282 - DNA, 63 bp, synthetic construct
  source 1..63: mol_type = other DNA
ggatcacttagttcggtaccccgcgttttgtgactaaaatggtcctgaggtttgctctctctt

SEQ ID NO: 283 - DNA, 432 bp, synthetic construct
  source 1..432: mol_type = other DNA
ttaaattgtgtgttgtttttatgttaagttactaaggtagccaggcaattgttacacgcatgatcaaagtcatgaattggatgaaaatatatgtggaaatggttggagcagaaactcttgaggacattgttaagagatggtttcgtggtttagattgtctggccgagatacatggtggagccggttctatccaccgatgaagaacatgaatggctgctattgcctttatagagtaggacttgacccactttgatgtgactcttaaacaaatccacttggcagtatatataagctcttctgttttatttattccctatgtacccaaatagtttgtgcaataacaaattgcaatttattttttcgctgccttgttctattgcccttaaaccagaccatttagggaaaaggggatgattttaatctctcaggaaa

SEQ ID NO: 284 - DNA, 329 bp, synthetic construct
  source 1..329: mol_type = other DNA
ggttggagcagaaactcttgaggacattgttaagagatggtttcgtggtttagattgtctggccgagatacatggtggagccggttctatccaccgatgaagaacatgaatggctgctattgcctttatagagtaggacttgacccactttgatgtgactcttaaacaaatccacttggcagtatatataagctcttctgttttatttattccctatgtacccaaatagtttgtgcaataacaaattgcaatttattttttcgctgccttgttctattgcccttaaaccagaccatttagggaaaaggggatgattttaatctctcagg

SEQ ID NO: 285 - DNA, 179 bp, synthetic construct
  source 1..179: mol_type = other DNA
ggttggagcagaaactcttgaggacattgttaagagatggtttcgtggtttagattgtctggccgagatacatggtggagccggttctatccaccgatgaagaacatgaatggctgctattgcctttatagagtaggacttgacccactttgatgtgactcttaaacaaatccacttgg

SEQ ID NO: 286 - DNA, 173 bp, synthetic construct
  source 1..173: mol_type = other DNA
gactcttaaacaaatccacttggcagtatatataagctcttctgttttatttattccctatgtacccaaatagtttgtgcaataacaaattgcaatttattttttcgctgccttgttctattgcccttaaaccagaccatttagggaaaaggggatgattttaatctctcagg

SEQ ID NO: 287 - DNA, 223 bp, synthetic construct
  source 1..223: mol_type = other DNA
ttaaattgtgtgttgtttttatgttaagttactaaggtagccaggcaattgttacacgcatgatcaaagtcatgaattggatgaaaatatatgtggaaatggttggagcagaaactcttgaggacattgttaagagatggtttcgtggtttagattgtctggccgagatacatggtggagccggttctatccaccgatgaagaacatgaatggctgctattgc

SEQ ID NO: 288 - DNA, 143 bp, synthetic construct
  source 1..143: mol_type = other DNA
ccaggcaattgttacacgcatgatcaaagtcatgaattggatgaaaatatatgtggaaatggttggagcagaaactcttgaggacattgttaagagatggtttcgtggtttagattgtctggccgagatacatggtggagccg

SEQ ID NO: 289 - DNA, 103 bp, synthetic construct
  source 1..103: mol_type = other DNA
tgatcaaagtcatgaattggatgaaaatatatgtggaaatggttggagcagaaactcttgaggacattgttaagagatggtttcgtggtttagattgtctggc

SEQ ID NO: 290 - DNA, 63 bp, synthetic construct
  source 1..63: mol_type = other DNA
atgaaaatatatgtggaaatggttggagcagaaactcttgaggacattgttaagagatggttt

SEQ ID NO: 291 - DNA, 223 bp, synthetic construct
  source 1..223: mol_type = other DNA
gtctggccgagatacatggtggagccggttctatccaccgatgaagaacatgaatggctgctattgcctttatagagtaggacttgacccactttgatgtgactcttaaacaaatccacttggcagtatatataagctcttctgttttatttattccctatgtacccaaatagtttgtgcaataacaaattgcaatttattttttcgctgccttgttctattg

SEQ ID NO: 292 - DNA, 143 bp, synthetic construct
  source 1..143: mol_type = other DNA
atgaagaacatgaatggctgctattgcctttatagagtaggacttgacccactttgatgtgactcttaaacaaatccacttggcagtatatataagctcttctgttttatttattccctatgtacccaaatagtttgtgcaat

SEQ ID NO: 293 - DNA, 103 bp, synthetic construct
  source 1..103: mol_type = other DNA
ctattgcctttatagagtaggacttgacccactttgatgtgactcttaaacaaatccacttggcagtatatataagctcttctgttttatttattccctatgt

SEQ ID NO: 294 - DNA, 63 bp, synthetic construct
  source 1..63: mol_type = other DNA
gacttgacccactttgatgtgactcttaaacaaatccacttggcagtatatataagctcttct

SEQ ID NO: 295 - DNA, 145 bp, synthetic construct
  source 1..145: mol_type = other DNA
ataagctcttctgttttatttattccctatgtacccaaatagtttgtgcaataacaaattgcaatttattttttcgctgccttgttctattgcccttaaaccagaccatttagggaaaaggggatgattttaatctctcaggaaa

SEQ ID NO: 296 - DNA, 105 bp, synthetic construct
  source 1..105: mol_type = other DNA
agtttgtgcaataacaaattgcaatttattttttcgctgccttgttctattgcccttaaaccagaccatttagggaaaaggggatgattttaatctctcaggaaa

SEQ ID NO: 297 - DNA, 85 bp, synthetic construct
  source 1..85: mol_type = other DNA
gcaatttattttttcgctgccttgttctattgcccttaaaccagaccatttagggaaaaggggatgattttaatctctcaggaaa

SEQ ID NO: 298 - DNA, 65 bp, synthetic construct
  source 1..65: mol_type = other DNA
cttgttctattgcccttaaaccagaccatttagggaaaaggggatgattttaatctctcaggaaa

SEQ ID NO: 299 - DNA, 223 bp, synthetic construct
  source 1..223: mol_type = other DNA
agcatttatatgcatgctatgattaccatgcttaaaatccgaagagaacaaggcaaaagaatgtgacaagggtatttttatatatttagaatcatgtgtgccatttgctgatggttaggttaagtatttagaaatatactatatgttgcatttttcttgtttggcactggaatttcttcttccgagatcgtgatggaatttaaaactgcatgttgcgatcatg

SEQ ID NO: 300 - DNA, 143 bp, synthetic construct
  source 1..143: mol_type = other DNA
gaagagaacaaggcaaaagaatgtgacaagggtatttttatatatttagaatcatgtgtgccatttgctgatggttaggttaagtatttagaaatatactatatgttgcatttttcttgtttggcactggaatttcttcttcc

SEQ ID NO: 301 - DNA, 103 bp, synthetic construct
  source 1..103: mol_type = other DNA
atgtgacaagggtatttttatatatttagaatcatgtgtgccatttgctgatggttaggttaagtatttagaaatatactatatgttgcatttttcttgtttg

SEQ ID NO: 302 - DNA, 63 bp, synthetic construct
  source 1..63: mol_type = other DNA
tatatttagaatcatgtgtgccatttgctgatggttaggttaagtatttagaaatatactata

SEQ ID NO: 303 - DNA, 223 bp, synthetic construct
  source 1..223: mol_type = other DNA
aggagcagctggtggcgtggaagctgtagcaacagtccaggtaaaacccaatggatcacttagttcggtaccccgcgattgatgactaaaatggtcctgaggtttgctctctcttcgtacaggcaattaggacagggtgggtgcatcccaatatcaacctggaaaacccagatcaaggagtggtaatgtttcttgttgcatcattccatccttcatttcaaaa

SEQ ID NO: 304 - DNA, 144 bp, synthetic construct
  source 1..144: mol_type = other DNA
ggtaaaacccaatggatcacttagttcggtaccccgcgattgatgactaaaatggtcctgaggtttgctctctcttcgtacaggcaattaggacagggtgggtgcatcccaatatcaacctggaaaacccagatcaaggagtgg

SEQ ID NO: 305 - DNA, 103 bp, synthetic construct
  source 1..103: mol_type = other DNA
tagttcggtaccccgcgattgatgactaaaatggtcctgaggtttgctctctcttcgtacaggcaattaggacagggtgggtgcatcccaatatcaacctgga

SEQ ID NO: 306 - DNA, 64 bp, synthetic construct
  source 1..64: mol_type = other DNA
tgatgactaaaatggtcctgaggtttgctctctcttcgtacaggcaattaggacagggtgggtg

SEQ ID NO: 307 - DNA, 548 bp, synthetic construct
  source 1..548: mol_type = other DNA
acacggagggcatctttcgtgggtacaagtttagtttcgatttaatgtgggtacatatgtcagcattttcatttttcatggatacaaatggtcatttattcctctaaataatgagcaggtcatatcgtcctactacgagaaacacatttaatttggatgactgtggatgttccttgaacatgtattaattgtgtgttgtttttatgttaagttactaaggtagccaggcaattgttacacgcatgatcaaagtcatgaattggatgaaaatatatgtggaaatggttggagcagaaactcttgaggacattgttgagagatggtttggtggtttagattgtctggccgagatacatggtggagccggttctatccaccgatgaagaacatgaatggctgctattgcctttatagagtaggacttgacccactttgatgtgactcttaaacaaatccacttggcagtatatataagctcttctgttttatttattccctatgtacccaaatagtttgtgcaataacaaattgcaatttattttttcgct

SEQ ID NO: 308 - DNA, 362 bp, synthetic construct
  source 1..362: mol_type = other DNA
cctctaaataatgagcaggtcatatcgtcctactacgagaaacacatttaatttggatgactgtggatgttccttgaacatgtattaattgtgtgttgtttttatgttaagttactaaggtagccaggcaattgttacacgcatgatcaaagtcatgaattggatgaaaatatatgtggaaatggttggagcagaaactcttgaggacattgttgagagatggtttggtggtttagattgtctggccgagatacatggtggagccggttctatccaccgatgaagaacatgaatggctgctattgcctttatagagtaggacttgacccactttgatgtgactcttaaacaaatccacttgg

SEQ ID NO: 309 - DNA, 206 bp, synthetic construct
  source 1..206: mol_type = other DNA
cctctaaataatgagcaggtcatatcgtcctactacgagaaacacatttaatttggatgactgtggatgttccttgaacatgtattaattgtgtgttgtttttatgttaagttactaaggtagccaggcaattgttacacgcatgatcaaagtcatgaattggatgaaaatatatgtggaaatggttggagcagaaactcttgagg

SEQ ID NO: 310 - DNA, 128 bp, synthetic construct
  source 1..128: mol_type = other DNA
cctctaaataatgagcaggtcatatcgtcctactacgagaaacacatttaatttggatgactgtggatgttccttgaacatgtattaattgtgtgttgtttttatgttaagttactaaggtagccagg

SEQ ID NO: 311 - DNA, 257 bp, synthetic construct
  source 1..257: mol_type = other DNA
gttaagttactaaggtagccaggcaattgttacacgcatgatcaaagtcatgaattggatgaaaatatatgtggaaatggttggagcagaaactcttgaggacattgttgagagatggtttggtggtttagattgtctggccgagatacatggtggagccggttctatccaccgatgaagaacatgaatggctgctattgcctttatagagtaggacttgacccactttgatgtgactcttaaacaaatccacttgg

SEQ ID NO: 312 - DNA, 101 bp, synthetic construct
  source 1..101: mol_type = other DNA
gttaagttactaaggtagccaggcaattgttacacgcatgatcaaagtcatgaattggatgaaaatatatgtggaaatggttggagcagaaactcttgagg

SEQ ID NO: 313 - DNA, 179 bp, synthetic construct
  source 1..179: mol_type = other DNA
ggttggagcagaaactcttgaggacattgttgagagatggtttggtggtttagattgtctggccgagatacatggtggagccggttctatccaccgatgaagaacatgaatggctgctattgcctttatagagtaggacttgacccactttgatgtgactcttaaacaaatccacttgg

SEQ ID NO: 314 - DNA, 223 bp, synthetic construct
  source 1..223: mol_type = other DNA
acacggagggcatctttcgtgggtacaagtttagtttcgatttaatgtgggtacatatgtcagcattttcatttttcatggatacaaatggtcatttattcctctaaataatgagcaggtcatatcgtcctactacgagaaacacatttaatttggatgactgtggatgttccttgaacatgtattaattgtgtgttgtttttatgttaagttactaaggtag

SEQ ID NO: 315 - DNA, 143 bp, synthetic construct
  source 1..143: mol_type = other DNA
tttaatgtgggtacatatgtcagcattttcatttttcatggatacaaatggtcatttattcctctaaataatgagcaggtcatatcgtcctactacgagaaacacatttaatttggatgactgtggatgttccttgaacatgt

SEQ ID NO: 316 - DNA, 103 bp, synthetic construct
  source 1..103: mol_type = other DNA
cagcattttcatttttcatggatacaaatggtcatttattcctctaaataatgagcaggtcatatcgtcctactacgagaaacacatttaatttggatgactg

SEQ ID NO: 317 - DNA, 63 bp, synthetic construct
  source 1..63: mol_type = other DNA
gatacaaatggtcatttattcctctaaataatgagcaggtcatatcgtcctactacgagaaac

SEQ ID NO: 318 - DNA, 223 bp, synthetic construct
  source 1..223: mol_type = other DNA
aaataatgagcaggtcatatcgtcctactacgagaaacacatttaatttggatgactgtggatgttccttgaacatgtattaattgtgtgttgtttttatgttaagttactaaggtagccaggcaattgttacacgcatgatcaaagtcatgaattggatgaaaatatatgtggaaatggttggagcagaaactcttgaggacattgttgagagatggtttgg

SEQ ID NO: 319 - DNA, 144 bp, synthetic construct
  source 1..144: mol_type = other DNA
catttaatttggatgactgtggatgttccttgaacatgtattaattgtgtgttgtttttatgttaagttactaaggtagccaggcaattgttacacgcatgatcaaagtcatgaattggatgaaaatatatgtggaaatggttg

SEQ ID NO: 320 - DNA, 103 bp, synthetic construct
  source 1..103: mol_type = other DNA
gatgttccttgaacatgtattaattgtgtgttgtttttatgttaagttactaaggtagccaggcaattgttacacgcatgatcaaagtcatgaattggatgaa

SEQ ID NO: 321 - DNA, 63 bp, synthetic construct
  source 1..63: mol_type = other DNA
taattgtgtgttgtttttatgttaagttactaaggtagccaggcaattgttacacgcatgatc

SEQ ID NO: 322 - DNA, 223 bp, synthetic construct
  source 1..223: mol_type = other DNA
attaattgtgtgttgtttttatgttaagttactaaggtagccaggcaattgttacacgcatgatcaaagtcatgaattggatgaaaatatatgtggaaatggttggagcagaaactcttgaggacattgttgagagatggtttggtggtttagattgtctggccgagatacatggtggagccggttctatccaccgatgaagaacatgaatggctgctattgc

SEQ ID NO: 323 - DNA, 143 bp, synthetic construct
  source 1..143: mol_type = other DNA
ccaggcaattgttacacgcatgatcaaagtcatgaattggatgaaaatatatgtggaaatggttggagcagaaactcttgaggacattgttgagagatggtttggtggtttagattgtctggccgagatacatggtggagccg

SEQ ID NO: 324 - DNA, 103 bp, synthetic construct
  source 1..103: mol_type = other DNA
tgatcaaagtcatgaattggatgaaaatatatgtggaaatggttggagcagaaactcttgaggacattgttgagagatggtttggtggtttagattgtctggc

SEQ ID NO: 325 - DNA, 64 bp, synthetic construct
  source 1..64: mol_type = other DNA
atgaaaatatatgtggaaatggttggagcagaaactcttgaggacattgttgagagatggtttg

SEQ ID NO: 326 - DNA, 209 bp, synthetic construct
  source 1..209: mol_type = other DNA
gtctggccgagatacatggtggagccggttctatccaccgatgaagaacatgaatggctgctattgcctttatagagtaggacttgacccactttgatgtgactcttaaacaaatccacttggcagtatatataagctcttctgttttatttattccctatgtacccaaatagtttgtgcaataacaaattgcaatttattttttcgct

SEQ ID NO: 327 - DNA, 138 bp, synthetic construct
  source 1..138: mol_type = other DNA
tcaaggtttttctgattctgatgagcaattgtcaagttttgaaactgacctttaatagaaaggcggcaaataaatctcaatttgatgctgttaaggaaaaaccaaagatattgtatgatggaagataatatctaacat

SEQ ID NO: 328 - DNA, 137 bp, synthetic construct
  source 1..137: mol_type = other DNA
tcaaggtttttctgattctgatgagcaattgtcaagttttgaaactgacctttaatgaaaggcggcaaataaatctcaatttgatgctgttaaggaaaaaccaaagatattgtatgatggaagataatatctaacat

SEQ ID NO: 329 - DNA, 137 bp, synthetic construct; identical to SEQ ID NO: 328
  source 1..137: mol_type = other DNA

SEQ ID NO: 330 - DNA, 137 bp, synthetic construct; identical to SEQ ID NO: 328
  source 1..137: mol_type = other DNA

SEQ ID NO: 331 - DNA, 137 bp, synthetic construct
  source 1..137: mol_type = other DNA
tcaaggtttttctgattctgatgagcaattgtcaagttttgaaactgacctttaatagaaggcggcaaataaatctcaatttgatgctgttaaggaaaaaccaaagatattgtatgatggaagataatatctaacat

SEQ ID NO: 332 - DNA, 137 bp, synthetic construct
  source 1..137: mol_type = other DNA
tcaaggtttttctgattctgatgagcaattgtcaagttttgaaactgacctttaataaaaggcggcaaataaatctcaatttgatgctgttaaggaaaaaccaaagatattgtatgatggaagataatatctaacat

SEQ ID NO: 333 - DNA, 137 bp, synthetic construct; identical to SEQ ID NO: 332
  source 1..137: mol_type = other DNA

SEQ ID NO: 334 - DNA, 137 bp, synthetic construct
  source 1..137: mol_type = other DNA
tcaaggtttttctgatctgatgagcaattgtcaagttttgaaactgacctttaatagaaaggcggcaaataaatctcaatttgatgctgttaaggaaaaaccaaagatattgtatgatggaagataatatctaacat

SEQ ID NO: 335 - DNA, 122 bp, synthetic construct
  source 1..122: mol_type = other DNA
tcaaggtttttctgattctgatgagcaattgtcaagtaatagaaaggcggcaaataaatctcaatttgatgctgttaaggaaaaaccaaagatattgtatgatggaagataatatctaacat

SEQ ID NO: 336 - DNA, 150 bp, synthetic construct
  source 1..150: mol_type = other DNA
tttatgttaagttactaaggtagccaggcaattgttacacgcatgatcaaagtcatgaattggatgaaaatatatgtggaaatggttggagcagaaactcttgaggacattgttaagagatggtttcgtggtttagattgtctggccgag

SEQ ID NO: 337 - DNA, 123 bp, synthetic construct
  source 1..123: mol_type = other DNA
tttatgttaagttactaaggtagccaggcaattgttacacgcatgatcaaagtcatgaattggatgaaaatatatgaggacattgttaagagatggtttcgtggtttagattgtctggccgag

SEQ ID NO: 338 - DNA, 141 bp, synthetic construct
  source 1..141: mol_type = other DNA
tttatgttaagttactaaggtagccaggcaattgttacacgcatgatcaaagtcatgaattggatgaaaatatatgtggaaatggttggagcagaaactcttgaggacattgttaagagatggtttcgtggtttggcccag

SEQ ID NO: 339 - DNA, 101 bp, synthetic construct
  source 1..101: mol_type = other DNA
tttatgttaagttactaaggtagccaggcaattgttacacgcatgatcaaagtcatgaatttgttaagagatggtttcgtggtttagattgtctggccgag

SEQ ID NO: 340 - DNA, 123 bp, synthetic construct
  source 1..123: mol_type = other DNA
tttatgttaagttactaaggtagccaggcaattgttacacgcatgatcaaagtcatgaattggatgaaaatatatgtggaaatggttgagagatggtttcgtggtttagattgtctggccgag

SEQ ID NO: 341 - DNA, 137 bp, synthetic construct
  source 1..137: mol_type = other DNA
tttatgttaagttactaaggtagccaggcaattgttacacgcatgatcaaagtcatgaattggatgaaaatatatgtggaaaaactcttgaggacattgttaagagatggtttcgtggtttagattgtctggccgag

SEQ ID NO: 342 - DNA, 123 bp, synthetic construct
  source 1..123: mol_type = other DNA
tttatgttaagttactaaggtagccaggcaattgttacacgcatgatcaaagtcatgaattggatgaaaatatatgtggaaatggttgggagatggtttcgtggtttagattgtctggccgag

SEQ ID NO: 343 - DNA, 23 bp, synthetic construct
  source 1..23: mol_type = other DNA
gaaactgacctttaatagaaagg

SEQ ID NO: 344 - DNA, 89 bp, synthetic construct
  source 1..89: mol_type = other DNA
attctgatgagcaattgtcaagttttgaaactgacctttaatagaaaggcggcaaataaatctcaatttgatgctgttaaggaaaaacc

SEQ ID NO: 345 - DNA, 89 bp, synthetic construct; identical to SEQ ID NO: 344
  source 1..89: mol_type = other DNA

SEQ ID NO: 346 - DNA, 88 bp, synthetic construct
  source 1..88: mol_type = other DNA
attctgatgagcaattgtcaagttttgaaactgacctttaatgaaaggcggcaaataaatctcaatttgatgctgttaaggaaaaacc

SEQ ID NO: 347 - DNA, 88 bp, synthetic construct
  source 1..88: mol_type = other DNA
attctgatgagcaattgtcaagttttgaaactgacctttaatagaaggcggcaaataaatctcaatttgatgctgttaaggaaaaacc

SEQ ID NO: 348 - DNA, 87 bp, synthetic construct
  source 1..87: mol_type = other DNA
attctgatgagcaattgtcaagttttgaaactgacctttaataaaaggcggcaaataaatctcaatttgatgctgttaaggaaaaac

SEQ ID NO: 349 - DNA, 87 bp, synthetic construct; identical to SEQ ID NO: 348
  source 1..87: mol_type = other DNA

SEQ ID NO: 350 - DNA, 73 bp, synthetic construct
  source 1..73: mol_type = other DNA
attctgatgagcaattgtcaagtaatagaaaggcggcaaataaatctcaatttgatgctgttaaggaaaaacc

SEQ ID NO: 351 - DNA, 24 bp, synthetic construct
  source 1..24: mol_type = other DNA
aaacatgaatgcaagttccgcagg

SEQ ID NO: 352 - DNA, 92 bp, synthetic construct
  source 1..92: mol_type = other DNA
ctcctatttaagatggagtaaagaaacatgaatgcaagttccgcagggagatacaaatcattccttgtcaagtcatagaatactcaaagaag

SEQ ID NO: 353 - DNA, 90 bp, synthetic construct
  source 1..90: mol_type = other DNA
ctcctatttaagatggagtaaagaaacatgaatgcaagttcgcagggagatacaaatcattccttgtcaagtcatagaatactcaaagaa

SEQ ID NO: 354 - DNA, 91 bp, synthetic construct
  source 1..91: mol_type = other DNA
ctcctatttaagatggagtaaagaaacatgaatgcaagttccgagggagatacaaatcattccttgtcaagtcatagaatgctcaaagaag

SEQ ID NO: 355 - DNA, 91 bp, synthetic construct
  source 1..91: mol_type = other DNA
ctcctatttaagatggagtaaagaaacatgaatgcagttccgcagggagatacaaatcattccttgtcaagtcatagaatactcaaagaag

SEQ ID NO: 356 - DNA, 91 bp, synthetic construct
  source 1..91: mol_type = other DNA
ctcctatttaagatggagtaaagaaacatgaatgcaagttcccagggagatacaaatcattccttgtcaagtcatagaatgctcaaagaag

SEQ ID NO: 357 - DNA, 90 bp, synthetic construct
  source 1..90: mol_type = other DNA
ctcctatttaagatggagtaaagaaacatgaatgcaagtcagggagatacaaatcattccttgtcaagtcatagaatgctcaaagaagag

SEQ ID NO: 358 - DNA, 89 bp, synthetic construct
  source 1..89: mol_type = other DNA
ctcctatttaagatggagtaacatgaatgcaagttccgcagggagatacaaatcattccttgtcaagtcatagaatactcaaagaagag

SEQ ID NO: 359 - DNA, 31 bp, synthetic construct
  source 1..31: mol_type = other DNA
tgggcgttgttacacctcttggtcacgaacc

SEQ ID NO: 360 - DNA, 86 bp, synthetic construct
  source 1..86: mol_type = other DNA
ggcgagtagttgtgaccgggttgggcgttgttacacctcttggtcacgaaccagatgtttactacaacaatttacttgatggtgtt

SEQ ID NO: 361 - DNA, 59 bp, synthetic construct
  source 1..59: mol_type = other DNA
ggcgagtagttgtgaccgggttgggcgttgttacacctcttggtcacgaaccaggtgtt

SEQ ID NO: 362 - DNA, 73 bp, synthetic construct
  source 1..73: mol_type = other DNA
ggcgagtagttgtgaccgggttgggcgttgttacacctcttggttactacaacaagtcacttgatggtgttac

SEQ ID NO: 363 - DNA, 77 bp, synthetic construct
  source 1..77: mol_type = other DNA
ggcgatgagttgggagtgggttgggcgtgggtacacgtcccagatgagtactacaacaatttacttgatggtgttag

SEQ ID NO: 364 - DNA, 73 bp, synthetic construct
  source 1..73: mol_type = other DNA
ggcgagtagttgtgactgggttgggcgttgttacacctcttggtcacgacacccatttacttgctggtgttcg